(19) United States
(12) Patent Application Publication
(10) Pub. No.: US 2026/0193378 A1
(43) Pub. Date: Jul. 9, 2026

(54) ANTIBODIES AND ANTIGEN BINDING FRAGMENTS THEREOF BINDING TO CHIMERIC RECEPTORS AND USES THEREOF

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US)

(72) Inventor: Michel Sadelain, Ny, NY (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US)

(21) Appl. No.: 19/429,466

(22) Filed: Dec. 22, 2025

Related U.S. Application Data
(63) Continuation of application No. PCT/US2023/028562, filed on Jul. 25, 2023.
(60) Provisional application No. 63/510,285, filed on Jun. 26, 2023.

(51) Int. Cl. C07K 16/42

(52) U.S. Cl. CPC C07K 16/4208; C07K 2317/24; C07K 2317/31; C07K 2317/565; C07K 2317/567; C07K 2317/622

(57) ABSTRACT

The presently disclosed subject matter provides antibodies or antigen-binding fragments thereof that bind to chimeric receptors (e.g., CAR, HIT, etc.). In certain embodiments, the presently disclosed antibodies or antigen-binding fragments thereof are anti-idiotype antibodies or antigen-binding fragment thereof. In certain embodiments, the presently disclosed antibodies or antigen-binding fragments thereof bind to an antigen-binding domain (e.g., an extracellular antigen-binding domain of a CAR).

### CROSS-REFERENCE TO RELATED APPLICATIONS

This application is a continuation application of International Patent Application No. PCT/US2023/028562, filed Jul. 25, 2023, which claims priority to U.S. Provisional Application No. 63/510,285, filed Jun. 26, 2023, the contents of each of which are incorporated by reference in their entirety, and to each of which priority is claimed.

### SEQUENCE LISTING

A Sequence Listing conforming to the rules of WIPO Standard ST.26 is hereby incorporated by reference. Said Sequence Listing has been filed as an electronic document via PatentCenter encoded as XML. The electronic document, created on Dec. 22, 2025, is entitled “0933640103.xml”, and is 73,617 bytes in size.

### FIELD OF THE INVENTION

The presently disclosed subject matter provides antibodies and antigen-binding fragments thereof that bind to a chimeric receptor (e.g., CAR, HIT). In certain embodiments, the antibodies and antigen-binding fragments thereof are anti-idiotype antibodies and antigen-binding fragments thereof.

### BACKGROUND OF THE INVENTION

Autologous chimeric antigen receptor (CAR) T cell therapy can provide substantial clinical benefit to patients with refractory hematological malignancies. This approach is however challenged by costly and sometimes delayed or unsuccessful cell manufacturing. Readily available CAR T cells that can be produced on a large scale are direly needed. Genetically engineered, T cell-derived induced pluripotent stem cells (TiPS) are a promising source for ‘off-the-shelf’ immunotherapeutic CAR T cells. However, in vitro TiPS differentiation often yields αβTCR-T cells with innate features. Therefore, there remains a need in the art for additional methods and reagents that could be useful in manufacturing processes.

### SUMMARY OF THE INVENTION

The presently disclosed subject matter provides antibodies and antigen-binding fragments thereof that bind to a chimeric receptor (e.g., CAR, HIT). In certain embodiments, the antibodies and antigen-binding fragments thereof are anti-idiotype antibodies and antigen-binding fragments thereof. In certain embodiments, the presently disclosed antibodies bind to an antigen-binding domain (e.g., an extracellular antigen-binding domain of a CAR).

In certain non-limiting embodiments, the presently disclosed subject matter provides an antibody or an antigen-binding fragment thereof, comprising a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7 or SEQ ID NO: 17.

In certain non-limiting embodiments, the presently disclosed subject matter provides an antibody or an antigen-binding fragment thereof, comprising a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8 or SEQ ID NO: 18.

In certain non-limiting embodiments, the presently disclosed subject matter provides an antibody or an antigen-binding fragment thereof, comprising:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7 or SEQ ID NO: 17; and
b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 8 or SEQ ID NO: 18.

In certain non-limiting embodiments, the presently disclosed subject matter provides an antibody or an antigen-binding fragment thereof, comprising a heavy chain variable region and a light chain variable region, wherein the heavy chain variable region and the light chain variable region are selected from the group consisting of:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO:7, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8; and
b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 17, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 18.

In certain non-limiting embodiments, the presently disclosed subject matter provides an antibody or an antigen-binding fragment thereof, comprising a heavy chain variable region comprising the amino acid sequence set forth in SEQ ID NO: 7 or SEQ ID NO: 17.

In certain non-limiting embodiments, the presently disclosed subject matter provides an antibody or an antigen-binding fragment thereof, comprising a light chain variable region comprising the amino acid sequence set forth in SEQ ID NO: 8 or SEQ ID NO: 18.

In certain non-limiting embodiments, the presently disclosed subject matter provides an antibody or an antigen-binding fragment thereof, comprising
a) a heavy chain variable region comprising the amino acid sequence set forth in SEQ ID NO: 7 or SEQ ID NO: 17; and
b) a light chain variable region comprising the amino acid sequence set forth in SEQ ID NO: 8 or SEQ ID NO: 18.

In certain embodiments,
a) the heavy chain variable region comprises the amino acid sequence set forth in SEQ ID NO: 7, and the light chain variable region comprises the amino acid sequence set forth in SEQ ID NO: 8; or
b) the heavy chain variable region comprises the amino acid sequence set forth in SEQ ID NO: 17, and the light chain variable region comprises the amino acid sequence set forth in SEQ ID NO: 18.

In certain non-limiting embodiments, the presently disclosed subject matter provides an antibody or an antigen-binding fragment thereof, comprising a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
a) a heavy chain variable region CDR3 comprising the amino acid sequence set forth in SEQ ID NO: 3 and a conservative modification thereof, and a light chain variable region CDR3 comprising the amino acid sequence set forth in SEQ ID NO: 6 and a conservative modification thereof; and
b) a heavy chain variable region CDR3 comprising the amino acid sequence set forth in SEQ ID NO: 13 and a conservative modification thereof, and a light chain variable region CDR3 comprising the amino acid sequence set forth in SEQ ID NO: 16 and a conservative modification thereof.

In certain embodiments, the heavy chain variable region and light chain variable region CDR2 domains are selected from the group consisting of:
a) a heavy chain variable region CDR2 comprising the amino acid sequence set forth in SEQ ID NO: 2 and a conservative modification thereof, and a light chain variable region CDR2 comprising the amino acid sequence set forth in SEQ ID NO: 5 and a conservative modification thereof; and
b) a heavy chain variable region CDR2 comprising the amino acid sequence set forth in SEQ ID NO: 12 and a conservative modification thereof, and a light chain variable region CDR2 comprising the amino acid sequence set forth in SEQ ID NO: 15 and a conservative modification thereof.

In certain embodiments, the heavy chain variable region and light chain variable region CDR1 domains are selected from the group consisting of:
a) a heavy chain variable region CDR1 comprising the amino acid sequence set forth in SEQ ID NO: 1 and a conservative modification thereof, and a light chain variable region CDR1 comprising the amino acid sequence set forth in SEQ ID NO: 4 and a conservative modification thereof; and
b) a heavy chain variable region CDR1 comprising the amino acid sequence set forth in SEQ ID NO: 11 and a conservative modification thereof, and a light chain variable region CDR1 comprising the amino acid sequence set forth in SEQ ID NO: 14 and a conservative modification thereof.

In certain embodiments, one or more of the CDR sequences have up to about 5 amino acid substitutions. In certain embodiments, one or more of the CDR sequences have up to about 3 amino acid substitutions.

In certain non-limiting embodiments, the presently disclosed subject matter provides an antibody or an antigen-binding fragment thereof comprising:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; or
b) a heavy chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13.

In certain non-limiting embodiments, the presently disclosed subject matter provides an antibody or an antigen-binding fragment thereof comprising:
a) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
b) a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain non-limiting embodiments, the presently disclosed subject matter provides an antibody or an antigen-binding fragment thereof comprising:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain non-limiting embodiments, the presently disclosed subject matter provides an antibody or an antigen-binding fragment thereof comprising a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain non-limiting embodiments, the presently disclosed subject matter provides an antibody or an antigen-binding fragment thereof comprising a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the antibody or antigen-binding fragment thereof is an anti-idiotype antibody. In certain embodiments, the antibody comprises a human variable region framework region. In certain embodiments, the antibody or antigen-fragment thereof is a fully human or an antigen-binding fragment thereof. In certain embodiments, the antibody or antigen-fragment thereof is a chimeric antibody or an antigen-binding fragment thereof. In certain embodiments, the antibody or antigen-fragment thereof is a humanized antibody or an antigen-binding fragment thereof. In certain embodiments, the antigen-binding fragment is a Fab, Fab′, F(ab′)2, variable fragment (Fv), or single chain variable region (scFv). In certain embodiments, the antigen-binding fragment is an scFv.

In certain non-limiting embodiments, the presently disclosed subject matter provides an antibody or an antigen-binding fragment thereof, which cross-competes for binding to the antibody or an antigen-binding fragment thereof disclosed herein. Additionally, in certain non-limiting embodiments, the presently disclosed subject matter provides an antibody or an antigen-binding fragment thereof, which binds to the same epitope region of an antibody or an antigen-binding fragment thereof disclosed herein.

In certain non-limiting embodiments, the presently disclosed subject matter further provides a composition comprising the antibody or antigen-binding fragment thereof disclosed herein. In certain embodiments, the composition is a pharmaceutical composition that further comprises a pharmaceutically acceptable carrier.

In certain non-limiting embodiments, the presently disclosed subject matter also provides a multi-specific molecule comprising the antibody or antigen-binding fragment thereof disclosed herein. In certain embodiments, the multi-specific molecule is linked to one or more functional moieties. In certain embodiments, the one or more functional moieties have a different binding specificity than the antibody or antigen binding fragment thereof.

Moreover, in certain non-limiting embodiments, the presently disclosed subject matter provides a composition comprising the multi-specific molecule disclosed herein.

In certain non-limiting embodiments, the presently disclosed subject matter provides a kit comprising the antibody or antigen-binding fragment thereof, the multi-specific molecule, or the composition disclosed herein. In certain embodiments, the kit further comprises written instructions for using the antibody or antigen-binding fragment thereof, multi-specific molecule, or composition.

### BRIEF DESCRIPTION OF THE DRAWINGS

The following Detailed Description, given by way of example, but not intended to limit the invention to specific embodiments described, may be understood in conjunction with the accompanying drawings.

FIGS. 1A-1G illustrate that DLL4 supports in vitro αβTCR-T cell development of WT-TiPS but not CAR-TiPS. FIG. 1A shows a schematic representation of in vitro T cell differentiation protocol. Microscope images are at 4× magnification. FIG. 1B shows flow cytometric analysis of T lineage commitment of H1, FiPS, and WT-TiPS on OP9-mDLL1, gated on live CD45⁺CD7⁺ cells at day 40 (D40) in the differentiation. FIG. 1C shows flow cytometric analysis of T lineage commitment of WT-TiPS and TRAC^{-/-}-TiPS on OP9-mDLL1, gated on live CD45⁺ cells at D40 in the differentiation. FIGS. 1D and 1F show representative flow cytometric analysis of T lineage commitment of WT-TiPS and CAR-TiPS on D35 in differentiation on OP9 expressing the indicated human Notch ligand, gated on live CD45⁺CD7⁺ cells. FIGS. 1E and 1G show phenotype distribution of WT-TiPS (e, n=6 biological replicates) or CAR-TiPS (g, n=6 biological replicates) on D35 of differentiation on OP9-DLL4, gated on live CD45⁺CD7⁺ cells. All data are means±s.d.

FIGS. 2A-2E illustrate that TRAC-controlled 1928z-1XX CAR expression facilitates DP T cell development. FIG. 2A shows induction of αβTCR (upper panel) and CAR (lower panel) expression in WT-TiPS, CAR-TiPS, and TRAC-CAR-TiPS throughout T lymphoid development on OP9-DLL4 at the indicated timepoints. Gated on live CD45⁺CD7⁺ cells. FIGS. 2B and 2D show representative flow cytometric analysis of T lineage commitment markers of TRAC-1928z-TiPS (FIG. 2B) and TRAC-1XX-TiPS (FIG. 2D) gated on live CD45⁺ cells at D35 in differentiation on OP9-DLL4. FIGS. 2C and 2E show D35 phenotype distribution of TRAC-1928z-TiPS (Fig, n=3 biological replicates) and TRAC-1XX-TiPS (FIG. 2E, n=11 biological replicates) at D35 gated on live CD45⁺CD7⁺ cells. All data are means±s.d.

FIGS. 3A-3D illustrate that CAR regulation influences Notch and TCR target gene induction. FIG. 3A shows a schematic representation of Notch and (pre)TCR signaling interactions as reported in the literature. FIG. 3B shows ddPCR analysis of Notch and (pre)TCR target genes at D24, 27, 31, and 35 of T cell differentiation (normalized to RPL13A) (n=3 technical replicates). FIG. 3C shows tSNE analysis of cell surface expression of CD4, CD8a, CD8b, and pTα on D35 TRAC-1XX-TiPS iT cells. The color scale represents the level of marker expression. FIG. 3D shows distribution of pTα expression on D35 TRAC-1XX-TiPS (n=3 biological replicates). All data are means±s.d.

FIGS. 4A-4P illustrate that 4-1BBL costimulation enhances CD8αβ TRAC-1XX-iT proliferation and function. FIG. 4A shows representative phenotype of TRAC-1XX-iT cells matured on 3T3-CD19 for 7 days (D35-D42), gated on live CD45⁺CD7⁺ (left and right) and CD45⁺CD7⁺CD8α⁺ (middle). FIG. 4B shows distribution of CD8αα and CD8αβ phenotype in the CD8α⁺ compartment (n=3 biological replicates). FIG. 4C shows expansion of TRAC-1XX-iT cells from D35-D42 after maturation on 3T3-CD19 (n=6 biological replicates). FIG. 4D shows 4-1BB cell-surface expression on TRAC-1XX-iT cells on D35 8 h after exposure to parental 3T3 (black), 3T3-CD19 (red) or left unstimulated (grey). FIG. 4E shows representative phenotype of TRAC-1XX-iT cells matured on 3T3-CD19-41BBL, gated on live CD45⁺CD7⁺ (left and right) and CD45⁺CD7⁺CD8α⁺ (middle). FIG. 4F shows distribution of CD8αα and CD8αβ phenotype in the CD8α⁺ compartment (n=6 biological replicates). FIG. 4G shows expansion of TRAC-1XX-iT cells from D35-D42 after maturation on 3T3-CD19-41BBL (n=6 biological replicates). FIG. 4H shows total cell expansion from DO-D42 in iT differentiation with maturation on 3T3-CD19±41BBL (n=6 biological replicates for each group). FIG. 4I shows cytotoxic activity measured in an 18 h bioluminescence assay, using firefly luciferase (FFLuc)-expressing NALM6 at the indicated effector-to-target (E:T) ratios (n=3; technical replicates).

FIG. 4J shows 4 h intracellular cytokine detection of 3T3-CD19±41BBL matured cells in response to NALM6 (n=3 technical replicates). FIG. 4K shows representative expansion of D42 cells matured on 3T3-CD19±41BBL upon repeated weekly antigen exposure on 3T3-CD19.

FIG. 4L shows schematic representation of NALM6 in vivo tumor model. FIG. 4M shows tumor burden (total flux in photons per second) of NALM6-bearing mice treated with 2×10⁶ D42 TRAC-1XX-iT cells (n=5, line=one mouse). FIG. 4N shows Kaplan-Meier analysis of mouse survival. FIG. 4O shows flow cytometric quantification of CAR T cells (left panel) and tumor cells (right panel) in bone marrow 6 days after T cell infusion (n=3). FIG. 4P shows cytotoxic activity measured in a 6 h flow cytometry assay, using primary CD19T CLL cells at the indicated E:Ts with 3T3-CD19-41BBL-matured TRAC-1XX-iT cells (n=3 technical replicates) * P<0.05, ** P<0.001, *** P<0.001, Welch's 2-sample t test (h, o), log-rank Mantel-Cox test (n). All data are means±s.d.

FIGS. 5A-5C illustrate that CD8αβ TRAC-1XX-iT cells resemble peripheral-blood derived CD8αβ T cells. FIG. 5A shows phenotype analysis of 3T3-CD19-41BBL matured D42 CD8αβ TRAC-1XX-iT cells for TCR-T cell markers (left and middle panel) and NK-cell markers (right panel). Data is representative of four independent experiments, gated on live CD45⁺CD7⁺CD8ab+ cells. FIG. 5B shows dendrogram of hierarchical clustering analysis based on Euclidian distance matrix comparing the transcriptome of TRAC-1XX CD8αβ αβTCR-T cells (CD8, blue, n=4 biological replicates), TRAC-1XX CD4 αβTCR-T cells (CD4, orange, n =3 biological replicates), γRV-1XX γδTCR-T cells (γδ, green, n=4 biological replicates), γRV-1XX NK cells (NK, purple, n=4 biological replicates) and CD8αβ⁺ TRAC-1XX-iT cells (iT CD8αβ, red, n=4 biological replicates). FIG. 5C shows correlation matrix using Pearson's statistics comparing same groups as in FIG. 5B.

FIGS. 6A-6I illustrate that TRAC-1XX-iT cells cure systemic NALM6 tumor model. Functional comparison of healthy-donor peripheral blood TRAC-1XX CD8αβ αβTCR-T cells (CD8 TRAC-1XX), CAR-iT, and TRAC-1XX-iT cells (matured on 3T3-CD19-41BBL). CD8 TRAC-1XX doses reflect number of CART T cells utilized in the assay. FIG. 6A shows cytotoxic activity using a 18 h Incucyte assay, using NLR-expressing NALM6 as target cells (n=3 technical replicates). FIG. 6B shows four h intracellular cytokine detection using NALM6 as target cells at a 1:1 E:T ratio (n=3 technical replicates). FIG. 6C shows NALM6 rechallenge assay. NLRT NALM6 and T cells were co-cultured at a 1:1 E:T. Every 72 h T cells were rechallenged with 1×NLRTNALM6 and cytokines. NALM6 clearance was measured in NLRT surface area reduction compared to the timepoint of rechallenge (n=3 technical replicates). FIG. 6D shows twenty-four h cytokine secretion using NALM6 as target cells at a 1:1 E:T ratio (CD8 TRAC-1XX n=15, TRAC-1XX-iT n=18, CAR-iT n=11 biological replicates). FIG. 6E shows schematic representation of systemic NALM6 tumor model. FIG. 6F shows tumor burden (total flux in photons per second) of NALM6-bearing untreated mice, or mice treated with 4×10⁶ CD8 TRAC-1XX or TRAC-1XX-iT cells (n=7, line=one mouse). FIG. 6G shows Kaplan-Meier analysis of tumor-free survival. FIG. 6H shows flow cytometric quantification of tumor cells (left) and T cells (right) in bone marrow 12 days after T cell infusion (n=2-3 mice). FIG. 6I shows phenotype of persisting iT cells prior to infusion (day 0, n=1) and of cells derived from the bone marrow on day 6 and 12 days after iT cell infusion (n=3 mice). * P<0.05, ** P<0.001, *** P<0.001, Chi-Square test (FIG. 6B), Welch's 2-sample t test (FIGS. 6D and 6H), log-rank Mantel-Cox test (FIG. 6G). All data are means±s.d FIGS. 7A-7D illustrate T lymphoid commitment of hES, FiPS and TiPS on OP9-mDLL1.

FIG. 7A shows flow cytometric analysis of pluripotency marker expression on H1, FiPS and WT-TiPS. FIG. 7B shows flow cytometric analysis of T lymphoid markers of H1 during differentiation on OP9-mDLL1 at indicated timepoints. FIG. 7C shows flow cytometric analysis of T lymphoid markers of FiPS during differentiation on OP9-mDLL1 at indicated timepoints.

FIG. 7D shows flow cytometric analysis of T lymphoid markers of WT-TiPS during differentiation on OP9-mDLL1 at indicated timepoints. Plots depicting CD7/CD5 are gated on live CD45⁺ cells, plots depicting CD3/TCRαβ, CD4/CD8α and CD8αβ/CD8β are gated on live CD45⁺CD7⁺ cells. CD3/TCRαβ and CD4/CD8α at D40 are as presented in FIG. 1B.

FIGS. 8A-8D illustrate generation, validation, and differentiation of TRAC^{-/-}-TiPS. FIG. 8A shows CRISPR/Cas9-targeted integration of EF1a-GFP-P2A-Puromycing-bGHpA (G2AP) expression unit into the TRAC locus. Top, TRAC locus; middle, plasmid containing the G2AP expression unit flanked by homology arms; bottom, edited TRAC locus. ‘FWD’ and ‘REV’ indicate the location of the forward and reverse primers used in FIG. 8B. FIG. 8B shows PCR validation of G2AP integration into the TRAC locus of TiPS clones. FIG. 8C shows flow cytometric analysis of pluripotency marker expression on TRAC^{-/-}-TiPS. Gated on live cells. FIG. 8D shows T lymphoid makers of WT-TiPS and TRAC^{-/-}-TiPS during differentiation on OP9-mDLL1 at the indicated timepoints. Gated on live CD45⁺ cells. D40 is as presented in FIG. 1C.

FIGS. 9A-9H illustrate early T lymphoid commitment of WT-TiPS and CAR-TiPS on human Notch ligands. FIG. 9A shows SFG γRV plasmid design to transduce human Notch ligands (DLL1, DLL4, JAG1 or JAG2) into parental OP9 cells. FIG. 9B shows Notch ligand expression on engineered OP9 lines. Filled grey histogram are stained parental OP9 cells, open black histogram are transduced OP9 cells. FIG. 9C shows DTX1 induction in WT-TiPS by OP9 expressing indicated Notch ligand. D20 differentiating WT-TiPS cells were co-cultured with indicated OP9. DTX1 induction was measured by ddPCR, relative to endogenous RPL13A. The fold change was calculated relative to 0 h. Data shown is average of n=2 technical replicates. FIGS. 9D and 9G show flow cytometric analysis of T lymphoid commitment marker expression (CD7, CD5, TCRαβ and CD56) of WT-TiPS (FIG. 9D) and CAR-TiPS (FIG. 9G) differentiated on OP9 expressing indicated human Notch ligands. Gated on live CD45⁺ cells. FIG. 9E shows flow cytometric analysis of pluripotency marker expression on CAR-TiPS. Gated on live cells. FIG. 9F shows phosphorylated-ERK1/2 levels in WT-TiPS (blue) and CAR-TiPS (red) on D35 (n=3 technical replicates). FIG. 9H shows phenotype (left panels) and apoptosis levels (right panels) of WT-TiPS (top) and CAR-TiPS (bottom) from D27-D35 of differentiation on OP9-DLL4. Percentage of apoptotic cells in each T lineage developmental stage was based on percentage live Annexin-V+ cells. * P<0.05, ** P<0.001, *** P<0.001, Welch's 2-sample t test, data are means±s.d (FIG. 9F) FIG. 10A-10G illustrate CD8αβ single positive CAR+ iT cell development. WT-TiPS were differentiated on OP9-DLL4 and transduced to express the 1928z CAR at D35 utilizing γRV SFG-1928z-P2A-LNGFR. Cells were expanded for 7 days in expansion media supplemented with IL-2. FIG. 10A shows CD4/CD8αβ expression prior to transduction (D35) and on D42 in LNGFRT cells, LNGFR− cells and untransduced control cells which remained in differentiation on OP9-DLL4. Gated on live CD45T cells. FIG. 10B shows cytotoxic activity of CART iT cells in a 18 h bioluminescence assay, using FFLuc− NALM6 as target cells (n=3 technical replicates, data are mean±s.d). FIG. 10C shows CRISPR/Cas9-targeted integration of CAR transgene into the TRAC locus. Top, TRAC locus; middle, plasmid containing the CAR transgene cassette flanked by homology arms; bottom, edited TRAC locus. FIGS. 10D and 10F show PCR validation of CAR integration into the TRAC locus of TRAC-1928z-TiPS (FIG. 10D) and TRAC-1XX-TiPS (FIG. 10F) clones. FIGS. 10E and 10G show pluripotency marker expression on TRAC-1928z-TiPS (FIG. 10E) and TRAC-1XX-TiPS (FIG. 10G), gated on live cells.

FIGS. 11A-11C illustrate T lineage commitment of TRAC-CAR-TiPS. FIG. 11A shows T lineage commitment marker expression (CD7/CD5, CD4/CD8a, CD8a/CD8b) of WT-TiPS (left), TRAC-1928z-TiPS (middle) and TRAC-1XX-TiPS on OP9-DLL4 at the indicated timepoints. CD7/CD5 is gated on live CD45⁺ cells, others are gated on live CD45⁺CD7⁺ cells. FIG. 11B shows flow cytometric analysis of T cell phenotype markers of D35 DP TRAC-1XX-iT cells. Gated on live CD45⁺CD7⁺CD4⁺CD8αβ⁺ cells. FIG. 11C shows intracellular and cell-surface expression of CD3 and TCRαβ on D35 TRAC-1XX-iT cells.

FIGS. 12A-12C illustrate tonic ITAM phosphorylation in CAR⁺ T cells. FIG. 12A shows representative flow cytometry plot of CAR expression and pITAM1 (top panel) or pITAM3 (bottom panel) in PBMC-derived T cells expressing γRV-1928z, TRAC-1928z or TRAC-1XX (gated on live CAR+), or in control TRAC^{-/-} cells (gated on live CAR−). FIG. 12B shows percentage of pITAM1⁺ in the populations shown in FIG. 12A (n=4-5 biological replicates, data are means±s.d.). FIG. 12C shows percentage of pITAM3⁺ in the populations shown in FIG. 12A (n=4-5 biological replicates, data are means±s.d.).

FIGS. 13A-13F illustrate DP TRAC-1XX-iT cell mature to CD8αβ SP iT cells on 3T3-CD19-41BBL. FIGS. 13A and 13C show flow cytometric analysis of D42 cells matured on 3T3-CD19 (FIG. 13A) or 3T3-CD19-41BBL (FIG. 13C). Gated on live CD45⁺CD7⁺ cells. FIG. 13B shows flow cytometric analysis of D35 and D42 phenotypes of stimulated DP TRAC-1XX-iT cells. D35 TRAC-1XX-iT cells were sorted for a CD4⁺CD8αβ⁺DP phenotype, stimulated on 3T3-CD19-41BBL and expanded for seven days. Gated on live CD45⁺CD7⁺ cells. FIG. 13D shows fold Expansion and T cell phenotype marker expression of TRAC-1XX-iT cells matured on 3T3-CD19-41BBL (3T3) or recombinant CD19-Fc. FIG. 13E shows 4 h cytotoxicity assay of 3T3-CD19-41BBL stimulated TRAC-1XX-iT cells in response to NALM6-CD19⁺ an NALM6-CD19^{-/-} target cells (n=3 technical replicates, data are means±s.d.). FIG. 13F shows CD19 expression on primary CLL cells.

FIGS. 14A-14C illustrate comparison of CD8αβ TRAC-1XX-iT cells and peripheral blood lymphocytes. FIG. 14A shows representative examples of lymphoid phenotype marker expression in CD8αβ TRAC-1XX-iT (red), CD8ab αβTCR-T (blue), CD4 αβTCR-T (orange), γδTCR-T (green) and NK cells (purple). CD8αβ TRAC-1XX-iT cells are the same as represented in FIG. 5A. FIG. 14B shows variability of lymphoid phenotype marker expression in CD8αβ TRAC-1XX-iT cells (n=34 biological replicates, data are means±s.d). Biological replicates shown are samples utilized in RNA analysis. FIG. 14C shows principal Component Analysis comparing TRAC-1XX CD8αβ αβTCR-T cells (CD8, n=4), TRAC-1XX CD4 αβTCR-T cells (CD4, n=3), γRV-1XX γδTCR-T cells (γδ, n=4), γRV-1XX NK cells (NK, n=4) and CD8αβ⁺ TRAC-1XX-iT cells (iT CD8αβ, n=4).

FIGS. 15A-15K illustrate TRAC-1XX-iT have improved persistence and function over CAR-iT cells. Functional comparison of healthy-donor peripheral blood TRAC-1XX CD8αβ αβTCR-T (CD8 TRAC-1XX), CAR-iT and TRAC-1XX-iT cells. CD8 TRAC-1XX cell doses represent number of CAR⁺ cells utilized in the assay. FIG. 15A shows CAR and CD3 expression in CD8 TRAC-1XX, CAR-iT and TRAC-1XX-iT cells (black line) compared to unstained control (grey filled histogram). FIG. 15B shows 18 h Incucyte cytotoxicity assay with NLR⁺ CD19^{-/-} NALM6 target cells (n=3 technical replicates). FIG. 15C shows 4 h intracellular cytokine detection in unstimulated control (left panel), NALM6 CD19^{-/-} target cells (at a 1:1 E:T, middle panel) or PMA/Ionomycin activated (right panel) (n=3 technical replicates). FIG. 15D shows 24 h cytokine secretion using NALM6-CD19^{-/-} as target cells at a 1:1 E:T (n=11-18 biological replicates, upper panel) or unstimulated control (n=11-18 biological replicates, lower panel). FIG. 15E shows schematic representation of the NALM6 in vivo tumor model. FIG. 15F shows tumor burden (total flux in photons per second) of NALM6-bearing, untreated mice or mice treated with 4×10⁶ TRAC-1XX-iT (middle) or CAR-iT (right) cells (n=7, line=one mouse). FIG. 15G shows Kaplan-Meier analysis of overall survival. FIGS. 15H and 15I show enumeration of T cells (FIG. 15H) and tumor cells (FIG. 15I) in the bone marrow, spleen and blood 12 days post T cell infusion (n=4 mice). FIG. 15J shows schematic representation of the NALM6 in vivo tumor model. FIG. 15K shows Kaplan-Meier analysis of overall survival. * P<0.05, ** P<0.001, *** P<0.001, Welch's 2-sample t test (FIGS. 15H and 15I) log-rank Mantel-Cox test (FIGS. 15G and 15K). All data are means±s.d.

FIGS. 16A-16D illustrate TRAC-1XX-iT function compared to healthy donor peripheral blood-derived CD8 TRAC-1XX T cells. In vivo functional comparison of healthy-donor peripheral blood TRAC-1XX CD8αβ αβTCR-T (CD8 TRAC-1XX), TRAC-1XX-iT cells. CD8 TRAC-1XX cell doses represent number of CART cells utilized in the assay. FIG. 16A shows CAR and CD3 expression in CD8 TRAC-1XX and TRAC-1XX-iT cells (black line) compared to unstained control (grey filled histogram). FIG. 16B shows enumeration of tumor cells in the bone marrow and T cells in bone marrow, spleen and blood 6- or 12-days post T cell infusion (n=2-3 mice). FIG. 16C shows phenotype of CD8 cells prior to infusion (day 0, n=1) and of cells derived from the bone marrow on day 6 (n=3 mice) and 12 (n=3 mice). FIG. 16D shows Kaplan-Meier analysis of overall survival. * P<0.05, ** P<0.001, *** P<0.001 Welch's 2-sample t test (FIG. 16B) log-rank Mantel-Cox test (FIG. 16D). All data are means±s.d.

FIGS. 17A and 17B illustrate flow cytometry gating strategy for iT cells. FIG. 17A shows gating strategy applied to identify live, CD45⁺CD7⁺ iT cells. FIG. 17B shows gating strategy applied to identify CD4, CD8α and CD8β expression levels. Gating is set based on stained PBMC controls (left panels) and stained in CD45⁺ lymphoid precursor cells (D20, WT-TiPS and CAR-TiPS respectively) to adjust for autofluorescence.

FIGS. 18A-18C illustrate the development and specificity of the presently disclosed antibodies. FIG. 18A shows the flow cytometric assessment of antibody specificity against CAR⁺ PG13 fibroblast. Expressions of the CD19-targeting second-generation 1928z CAR, first-generation 19z1 CAR, and first-generation PSMA-targeting Pz1 CAR were confirmed with the polyclonal goat-anti-mouse F(ab)′ fragment. FIG. 18B shows staining with 19E3 antibody specific to SJ25C1 recognition in the 1928z and 19z1 CARs. FIG. 18C shows staining with 12D11 antibody, having scFv-independent CAR recognition, for detecting both CD19 and PSMA-targeting CARs.

FIGS. 19A-19C illustrate the development and differentiation of iPSC-derived CAR⁺ T cells contacted with the presently disclosed antibodies. FIG. 19A shows flow cytometric analysis of end-stage differentiated T cells contacted with 19E3 or 12D11 anti-idiotype antibodies and resulting in the maturation and development of effector phenotype (e.g, CD2+, CD56^{lo}, CD45RA⁺, CD62L⁺, CCR7^{lo}, CXCR4⁺, and CD25⁺) compared to stronger stimulation with CD19-protein expressing feeder cells K562-CD19. FIG. 19B shows 19E3-maturated T cells having superior cytolytic capacity over T cells developed in the presence of K562-CD19 cells. FIG. 19C shows 19E3-maturated T cells produced cytokines (e.g., IFNγ, Granzyme B, CD107a, and TNFα) in a stimulation-dependent manner.

FIGS. 20A-20C illustrate the effects of the presently disclosed antibodies on the development of iPSC-derived CAR⁺ T cells. FIG. 20A shows a schematic outline of the experimental approach for in vitro evaluation of iPSC-derived CAR⁺ T cell maturation. Premature T cells were subjected to various dosages of 19E3 antibody (from 0 μg/ml to 9 μg/ml) over 42 days. FIG. 20B shows a flow cytometry analysis of T cells subjected to various dosages of 19E3 antibodies. Analyzed cell surface expression markers included CD4, CD8a, and CD8ab. FIG. 20C shows that 19E3 increased cell expansion and cell viability of T cells.

FIGS. 21A-21C illustrate that T cell proliferation is enhanced by co-stimulation with a 4-1BB agonist. FIG. 21A shows a schematic outlining the in vitro experimental approach to evaluate the effects of the presently disclosed antibodies and urelumab. FIG. 21B shows that 19E3 and urelumab increased iT cell expansion and viability compared to 19E3 or urelumab alone and that these increases were comparable to iT cells obtained using 3T3-CD19-41BBL feeder-cells. FIG. 21C shows a heatmap of cell expansion (expressed as fold change) in response to various dosages of 19E3 and urelumab.

FIGS. 22A-22D illustrate that 19E3 and urelumab induced CAR T maturation in a dose-dependent manner. FIG. 22A shows a schematic of the experimental approach to assess the effects of 19E3 and Urelumab. FIG. 22B shows heatmaps of cell expansion and viability presence of various dosages of 19E3 and urelumab. FIG. 22C shows a flow cytometric analysis of the T cells obtained in presence of 19E3 and urelumab. FIG. 22D shows the cell growth and effector phenotype in response to 19E3 and different doses of urelumab.

FIGS. 23A and 23B illustrate the polyfunctionality of T cells obtained using the presently disclosed antibodies. FIG. 23A shows cytotoxic activity of iT cells obtained using the presently disclosed antibodies (e.g., 19E3 or 12D111) and different doses of urelumab against firefly luciferase (FFLuc)-expressing NALM6 CD19⁺ at the indicated effector-to-target (E:T) ratios. FIG. 23B shows cytokine secretion profiles of iT cells obtained using the presently disclosed antibodies (e.g., 19E3 or 12D11) and different doses of urelumab.

FIGS. 24A-24E illustrate in vivo effects of iT cells obtained using the presently disclosed antibodies (e.g., 19E3 or 12D11) and different doses of urelumab. FIG. 24A shows a schematic representation of the in vivo tumor model and experimental conditions. FIGS. 24B-24D show the results of tumor burden analysis using bioluminescence imaging (total flux) in response to iT cells obtained using the presently disclosed antibodies (e.g., 19E3 or 12D11) and different doses of urelumab. FIG. 24B shows the tumor burden effect using 8×10⁶ cells. FIG. 24C shows the tumor burden effect using 4×10⁶ cells. FIG. 24D shows the tumor burden effect using 2×10⁶ cells. FIG. 24E shows Kaplan-Meier analysis of overall survival.

### DETAILED DESCRIPTION OF EMBODIMENTS OF THE INVENTION

The presently disclosed subject matter provides improved antibodies and antigen-binding fragments thereof useful to generate cells with enhanced activity and efficacy for immunotherapy (e.g., T cell immunotherapy). The presently disclosed subject matter is based, in part, on the unexpected discovery that the presently disclosed antibodies can engage a chimeric antigen receptor (CAR) in order to improve the differentiation of pluripotent stem cells into T cells (e.g., induced T cells). Surprisingly, despite lacking expression of their endogenous TCR, T cells contacted by the presently disclosed antibodies or antigen-binding fragment thereof acquire conventional CD4 or CD8 T cell phenotype and do not show exhaustion markers. The presently disclosed subject matter also provides compositions used in the methods disclosed herein as well as cells generated using the methods disclosed herein.

Non-limiting embodiments of the presently disclosed subject matter are described by the present specification and Examples.

For purposes of clarity of disclosure and not by way of limitation, the detailed description is divided into the following subsections:
1. Definitions;
2. Antibodies and Antigen-binding Fragments Thereof;
3. Nucleic Acids encoding the Antibodies or Antigen-binding Fragments;
4. Pharmaceutical Compositions;
5. Diagnostic and Prognostic Methods;
6. Kits; and
7. Exemplary Embodiments.

### 1. Definitions

In the description that follows, certain conventions will be followed as regards the usage of terminology. Generally, terms used herein are intended to be interpreted consistently with the meaning of those terms as they are known to those of skill in the art.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “comprises”, “comprising”, and are intended to have the broad meaning ascribed to them in U.S. Patent Law and can mean “includes”, “including” and the like.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding fragment” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

The term “antigen-binding fragment” or “antigen-binding region” of an antibody, as used herein, refers to that region or fragment of the antibody that binds to the antigen and which confers antigen specificity to the antibody; fragments of antigen-binding proteins, for example, antibodies include one or more fragments of an antibody that retain the ability to specifically bind to an antigen-binding domain (e.g., an antigen-binding domain of a CAR). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., Nature 1989; 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science (1988); 242:423-426; and Huston et al., Proc NatlAcad Sci (1998); 85:5879-5883. These antibody fragments are obtained using conventional techniques known to those of skill in the art, and the fragments are screened for utility in the same manner as are intact antibodies.

An “antigen-binding protein” is a polypeptide that has been identified and separated and/or recovered from a component of its natural environment. “Synthetic antibodies” or “recombinant antibodies” are generally generated using recombinant technology or using peptide synthetic techniques known to those of skill in the art.

“CDRs” are defined as the complementarity determining region amino acid sequences of an antibody which are the hypervariable regions of immunoglobulin heavy and light chains. See, e. g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope region. In certain embodiments, the CDRs are identified according to the IMGT system. In certain embodiments, the CDRs are identified using the IMGT numbering system accessible at http://www.imgt.org/IMGT_vquest/input.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain.

As used herein, the term “Linker” shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). Non-limiting examples of linkers are disclosed in Shen et al., Anal Chem (2008); 80(6):1910-1917 and WO 2014/087010, the contents of which are hereby incorporated by reference in their entireties. In certain embodiments, the linker is a G4S linker. In certain embodiments, the linker comprises or consists of the amino acid sequence set forth in SEQ ID NO: 21, which is provided below:

[SEQ ID NO: 21]

GGGGSGGGGSGGGSGGGGS

In certain embodiments, the linker comprises or consists of the amino acid sequence set forth in SEQ ID NO: 22, which is provided below:

[SEQ ID NO: 22]

GGGGSGGGGSGGGGS

In certain embodiments, the linker comprises or consists of the amino acid sequence set forth in SEQ ID NO: 23, which is provided below:

[SEQ ID NO: 23]

GGGGSGGGGSGGGGSGGGSGGGGS

In certain embodiments, the linker comprises or consists of the amino acid sequence set forth in SEQ ID NO: 24, which is provided below:

[SEQ ID NO: 24]

GGGGSGGGGSGGGGSGGGGSGGGSGGGGS

In certain embodiments, the linker comprises or consists of the amino acid sequence set forth in SEQ ID NO: 25, which is provided below:

[SEQ ID NO: 25]

GGGGS

In certain embodiments, the linker comprises or consists of the amino acid sequence set forth in SEQ ID NO: 26, which is provided below:

[SEQ ID NO: 26]

GSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 1988; 85:5879-5883). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008; 27(6):455-51; Peter et al., J Cachexia Sarcopenia Muscle 2012 August 12; Shieh et al., J Imunol 2009; 183(4):2277-85; Giomarelli et al., Thromb Haemost 2007; 97(6):955-63; Fife eta., J Clin Invst 2006; 116(8):2252-61; Brocks et al., Immunotechnology 1997; 3(3):173-84; Moosmayer et al., Ther Immunol 1995; 2(10:31-40). Agonistic scFvs having stimulatory activity have been described (see, e.g., Peter et al., J Bioi Chem 2003; 25278(38):36740-7; Xie et al., Nat Biotech 1997; 15(8):768-71; Ledbetter et al., Crit Rev Immunol 1997; 17(5-6):427-55; Ho et al., BioChim Biophys Acta 2003; 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “Fc fusion protein” refers to homodimers in which an Fc domain of an antibody is covalently linked to another protein. Fc is the crystallizable fragment derived from Ig which has five classes including IgG, IgA, IgD, IgM, and IgE in human (Schroeder and Cavacini L, J Allergy Clin Immunol (2010) 125(2 Suppl 2):541-52). Fc plays multiple roles in activation and recruiting of immune leukocytes, triggering of antibody-dependent cell-mediated cytotoxicity (ADCC) and antibody-dependent cell-mediated phagocytosis (Ravetch and Bolland, Annu Rev Immunol (2001) 19:275-90; Woof and Burton, Nat Rev Immunol (2004) 4(2):89-99). In addition, Fc can bind to the serum complement molecule (C1q) to initiate the assembly of membrane attack complex formed by complement cascade proteins to destroy target cells, which is termed complement-dependent cytotoxicity (CDC) (Walport, N Engl J Med (2001) 344(14):1058-66; Walport, N Engl J Med (2001) 344(15):1140-4). Overall, Fc plays important roles in biological and pharmacological properties including, among other functions, increased stability and aggregation resistance, acquired multivalent binding to the target, enhanced Fc-mediated effector functions, extended serum half-life, and modulated immunogenicity. Additional information regarding Fc proteins can be found in Beck and Reichert, MAbs. Vol. 3. No. 5. Taylor & Francis (2011)., the content of which is incorporated by reference in its entirety.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis), and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to a chimeric receptor” or “specifically binds to a CAR” is intended to refer to an antibody that binds to an antigen-binding domain of a CAR (e.g., the extracellular antigen-binding domain of a CAR) with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, about 1×10⁻¹¹ M or less, about 5×10¹² M or less, or about 1×10¹² M or less.

An “antibody that competes for binding” or “antibody that cross-competes for binding” with a reference antibody for binding to an antigen, e.g., an antigen-binding domain (e.g., the extracellular antigen-binding domain of a CAR), refers to an antibody that blocks binding of the reference antibody to the antigen (e.g., an antigen-binding domain, e.g., the extracellular antigen-binding domain of a CAR) in a competition assay by 50% or more, and conversely, the reference antibody blocks binding of the antibody to the antigen (e.g., an antigen-binding domain, e.g., the extracellular antigen-binding domain of a CAR) in a competition assay by 50% or more. An exemplary competition assay is described in “Antibodies”, Harlow and Lane (Cold Spring Harbor Press, Cold Spring Harbor, NY).

As used herein, “isotype” refers to the antibody class (e.g., IgM or IgG1) that is encoded by the heavy chain constant region genes.

As used herein, the term “affinity” is meant as a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after the formation of reversible complexes. Methods for calculating the affinity of an antibody for an antigen are known in the art, including, but not limited to, various antigen-binding experiments, e.g., functional assays (e.g., flow cytometry assay).

An “immunoconjugate” is an antibody conjugated to one or more heterologous molecule(s), including, but not limited to, a cytotoxic agent.

As used herein, the term “derivative” refers to a compound that is derived from some other compound and maintains its general structure. For example, but without any limitation, trichloromethane (chloroform) is a derivative of methane.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

An “isolated antibody” is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr (2007); B 848:79-87.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an antibody” refers to one or more nucleic acid molecules encoding antibody heavy and light chains (or fragments thereof), including such nucleic acid molecule(s) in a single vector separate vectors, and such nucleic acid molecule(s) present at one or more locations in a host cell.

By “isolated cell” is meant a cell that is separated from the molecular and/or cellular components that naturally accompany the cell.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences, by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv703) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage. In certain embodiments, the immunoresponsive cell is a monocyte.

The terms “induced pluripotent stem cells” or “iPS,” as used herein, refer to stem cells that are produced from fully differentiated cells which have been reprogrammed into cells capable of differentiation into tissues of the three germs layers (e.g., ectoderm, mesoderm, endoderm). The resulting iPS are not naturally occurring as they include genomic engineering and reprogramming. Non-limiting examples of methods to obtain induced pluripotent stem cells can be found in Valamehr et al., Stem Cell Reports 2, 366-381 (2014); Themeli et al., Nat Biotechnol 31, 928-933 (2013), Mansilla-Soto et al., Nat Med 28, 345-352 (2022), and Eyquem et al., Nature 543, 113-117 (2017), the content of each of which is incorporated by reference in their entirety. In certain embodiments, the pluripotent stem cells are induced pluripotent stem cells (iPS). In certain embodiments, the pluripotent stem cells are T cell-derived induced pluripotent stem cells (TiPS).

As used herein, the terms “induced T cell” or “iT” refers to differentiated T cells produced from induced pluripotent stem cells (e.g., T cell-derived pluripotent stem cells). These induced T cells (iTs) are not pluripotent stem cells as they have acquired specialized features and functions. For example, but without any limitation, induced T cells are capable of eliciting an immune response upon binding with a non-self antigen.

The term “hematopoietic precursor,” as used herein, refers to CD34+ cells capable of giving rise to both mature myeloid and lymphoid cell types (e.g., T cells, NK cells, and B cells).

The term “antigen-recognizing receptor” as used herein refers to a receptor that is capable of activating an immune or immunoresponsive cell (e.g., a T-cell) in response to its binding to an antigen.

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

The term “antigen-binding domain,” as used herein, refers to a domain capable of specifically binding a particular antigenic determinant or set of antigenic determinants. For example, but without any limitation, an antigen-binding domain of a CAR refers to a domain capable of specifically binding a particular antigenic determinant or set of antigenic determinants present on a cell.

As used herein, a “co-stimulatory molecule” refers to a cell surface molecule other than an antigen receptor or its ligand that can provide an efficient response of lymphocytes to an antigen. In certain embodiments, a co-stimulatory molecule can provide optimal lymphocyte activation.

As used herein, a “co-stimulatory ligand” refers to a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen-recognizing receptor or capable of suppressing an immune response via receptor-ligand binding.

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “differentiation” refers to a process whereby an unspecialized cell (e.g., a pluripotent stem cell) acquires the features of a specialized cell such as a lymphocyte, a neuron cell, a hepatocyte, or a muscle cell. Differentiation is controlled by the interaction of a cell's genes with the physical and chemical conditions outside the cell, usually through signaling pathways involving proteins embedded in the cell surface.

As used herein, the term “feeder cells” refers to a first type of cells that can be co-cultured with cells of a second type of cells in order to provide an environment that improves differentiation and growth of the second type of cells. In certain embodiments, the feeder cells can generate a conditioned medium that is enriched in growth factors, nutrients, and signals responsible for the growth and differentiation of the second type of cells. In certain embodiments, the feeder cells can be from the same species or from a different species as the cells they are supporting. For example, but without any limitation, certain types of human cells, including pluripotent stem cells, can be supported by primary cultures of mouse embryonic fibroblasts, or immortalized mouse embryonic fibroblasts. In certain embodiments, the feeder cells can be inactivated (e.g., by irradiation). Non-limiting examples of feeder cells include endothelial cells, stromal cells (e.g., fibroblasts), and leukemic cells.

As used herein, the term “feeder-free” refers to a cell culture system or environment (e.g., cell culture medium) that lacks feeder cells. In certain embodiments, a feeder free system has not been pre-conditioned by the cultivation of feeder cells. As used herein, the term “pre-conditioned medium” refers to a medium or cell culture system which has been harvested after feeder cells have been cultivated for a period of time (e.g., 2 days). This pre-conditioned medium contains growth factors and cytokines secreted by the feeder cells. In certain embodiments, however, even such pre-conditioning is unnecessary in view fo the strategies described herein.

As used herein, the term “contacting” a cell or cells with a compound (e.g., at least one inhibitor, activator, and/or inducer) refers to providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “disease” is meant any condition, disease, or disorder that damages or interferes with the normal function of a cell, tissue, or organ, e.g., neoplasm, and pathogen infection of cell.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters; guinea pigs; rabbits; dogs; cats; sheep; pigs; goats; cattle; horses; and non-human primates such as apes and monkeys.

An “effective amount” (or, “therapeutically effective amount”) is an amount sufficient to effect a beneficial or desired result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition, and the form and effective concentration of the cells administered.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

Other aspects of the presently disclosed subject matter are described in the following disclosure and are within the ambit of the presently disclosed subject matter.

### 2. Antibodies and Antigen-Binding Thereof

The antibodies of the presently disclosed subject matter are characterized by particular functional features or properties of the antibodies. In certain embodiments, the presently disclosed antibodies bind to an extracellular domain of a chimeric receptor (e.g., a CAR, a HIT, etc.). In certain embodiments, the presently disclosed antibodies bind to an scFv of the extracellular domain of a chimeric receptor (e.g., a CAR, a HIT, etc.). In certain embodiments, for example and without any limitation, the antibodies are anti-idiotype antibodies.

An “anti-idiotype antibody” is an antibody or antigen-binding fragment thereof that binds to an idiotope of an antibody or an antigen-binding fragment thereof. Additionally, an anti-idiotype antibody is an antibody or antigen-binding fragment thereof that binds to an idiotope of an antigen-binding domain (e.g., an antigen-binding domain of a CAR). In certain non-limiting embodiments, the idiotopes of an antibody or an antigen-binding fragment thereof, or the idiotopes of an antigen-binding domain (e.g., an antigen-binding domain of a CAR), can include residues of the complementarity determining regions or fragments thereof, variable regions or fragments thereof, or a combination thereof. Additional information on anti-idiotype antibodies can be found in Ying et al., The FASEB journal 9.1 (1995): 43-49, the content of which is incorporated by reference in its entirety.

In certain embodiments, a presently disclosed antibody or antigen-binding fragment binds to an extracellular domain of a chimeric receptor (e.g., a CAR, a HIT, etc.) with a binding affinity, for example with a dissociation constant (K_{D}) of about 1×10⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less, about 5×10⁻¹² M or less, or about 1×10⁻¹² M or less. In certain embodiments, a presently disclosed antibody or antigen-binding fragment binds to an extracellular domain of a chimeric receptor (e.g., a CAR, a HIT, etc.) with a binding affinity, for example with a dissociation constant (K_{D}) of about 5×10⁻⁹ M or less. In certain embodiments, a presently disclosed antibody or antigen-binding fragment binds to an extracellular domain of a chimeric receptor (e.g., a CAR, a HIT, etc.) with a binding affinity, for example with a dissociation constant (K_{D}) of about 1×10⁻⁹ M or less. In certain embodiments, a presently disclosed antibody or antigen-binding fragment binds to an extracellular domain of a chimeric receptor (e.g., a CAR, a HIT, etc.) with a binding affinity, for example with a dissociation constant (K_{D}) of about 1×10⁻¹² M.

In certain embodiments, a presently disclosed antibody or antigen-binding fragment binds to an antigen-binding domain (e.g., an antigen-binding domain of a CAR) with a binding affinity, for example with a dissociation constant (K_{D}) of about 1×10⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less, about 5×10⁻¹² M or less, or about 1×10⁻¹² M or less. In certain embodiments, a presently disclosed antibody or antigen-binding fragment binds to an antigen-binding domain (e.g., an antigen-binding domain of a CAR) with a binding affinity, for example with a dissociation constant (K_{D}) of about 5×10⁻⁹ M or less. In certain embodiments, a presently disclosed antibody or antigen-binding fragment binds to an antigen-binding domain (e.g., an antigen-binding domain of a CAR) with a binding affinity, for example with a dissociation constant (K_{D}) of about 1×10⁻⁹ M or less. In certain embodiments, a presently disclosed antibody or antigen-binding fragment binds to an antigen-binding domain (e.g., an antigen-binding domain of a CAR) with a binding affinity, for example with a dissociation constant (K_{D}) of about 1×10⁻¹² M.

The heavy and light chains of a presently disclosed antibody or antigen-binding fragment can be full-length (e.g., an antibody can include at least one (e.g., one or two) complete heavy chains, and at least one (e.g., one or two) complete light chains) or can include an antigen-binding fragment (a Fab, F(ab′)₂, Fv or a single chain Fv fragment (“scFv”)). In certain embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4. In certain embodiments, the immunoglobulin isotype is IgG1 (e.g., human IgG1). The choice of antibody isotype can depend on the immune effector function that the antibody is designed to elicit. In certain embodiments, the antibody light chain constant region is chosen from, e.g., kappa or lambda, particularly kappa.

In constructing a recombinant immunoglobulin, appropriate amino acid sequences for constant regions of various immunoglobulin isotypes and methods for the production of a wide array of antibodies are known to those of skill in the art.

### 2.1. Chimeric Receptors

In certain non-limiting embodiments, the presently disclosed subject matter provides antibodies or antigen-binding fragments thereof that specifically bind to an antigen-binding domain of a chimeric receptor.

### 2.1.1. Chimeric Antigen Receptors

In certain non-limiting embodiments, the presently disclosed subject matter provides antibodies or antigen-binding fragments thereof that specifically bind to an antigen-binding domain of a chimeric antigen receptor (CAR). In certain non-limiting embodiments, the presently disclosed antibodies or antigen-binding fragments thereof bind to an extracellular domain of a chimeric antigen receptor (CAR). In certain non-limiting embodiments, the presently disclosed antibodies or antigen-binding fragments thereof are anti-idiotype antibodies.

CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv) that binds to a target antigen, and an intracellular signaling domain. In certain embodiments, the CAR further comprises a transmembrane domain. “First generation” CARs can provide de novo antigen recognition and cause activation of both CD4+ and CD8⁺ T cells through their CD3ζ chain signaling domain in a single fusion molecule, independent of HLA-mediated antigen presentation. “Second generation” CARs include a signaling domain of a co-stimulatory molecule (e.g., CD28, 4-1BB, ICOS, OX40, CD27, CD40, NKG2D, DAP-10, CD2, CD150, CD226) to the intracellular signaling domain of the CAR to provide co-stimulation signals to the cell (e.g., T cell or NK cell). “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ).

In certain embodiments, the CAR comprises an extracellular antigen-binding domain that binds to an antigen, and an intracellular signaling domain. In certain embodiments, the CAR further comprises a transmembrane domain. In certain embodiments, the CAR further comprises a hinger/spacer region. The extracellular antigen-binding domain can comprise or be an scFv, a Fab (which is optionally crosslinked), or a F(ab)₂. In certain embodiments, any of the foregoing molecules may be comprised in a fusion protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

In certain embodiments, the CAR comprises a transmembrane domain. Different transmembrane domains result in different receptor stability. After antigen recognition, receptors cluster and a signal is transmitted to the cell. In accordance with the presently disclosed subject matter, the transmembrane domain of the CAR can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof.

In certain embodiments, the CAR further comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof. The hinge/spacer region can be the hinge region from IgG1, the CH₂CH₃ region of an immunoglobulin and portions of CD3, a portion of a CD28 polypeptide, a portion of a CD8 polypeptide, or a synthetic spacer sequence.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof.

In certain embodiments, the CAR comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3∂ comprises three functional immunoreceptor tyrosine-based activation motifs (ITAMs), three functional basic-rich stretch (BRS) regions (BRS1, BRS2, and BRS3). CD3ζ transmits an activation signal to the cell (e.g., a cell of the lymphoid lineage, e.g., a T-cell) after the antigen is bound. The intracellular signaling domain of the CD3ζ-chain is the primary transmitter of signals from endogenous TCRs.

In certain embodiments, the intracellular signaling domain of the CAR comprises a native or a modified CD3ζ. Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of costimulatory molecules include CD28, 4-1BB, OX40, CD27, CD40, CD154, CD97, CD11a/CD18, ICOS, DAP-10, CD2, CD150, CD226, and NKG2D.

In addition, the extracellular antigen-binding domain of the CAR can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain of the CAR. Exemplary leader sequences include, but is not limited to, a human IL-2 signal sequence (e.g., a human IL-2 signal sequence comprising or consisting of the amino acid sequence set forth in SEQ ID NO: 27), a mouse IL-2 signal sequence (e.g., a mouse IL-2 signal sequence comprising or consisting of the amino acid sequence set forth in SEQ ID NO: 28); a human kappa leader sequence (e.g., a human kappa leader sequence comprising or consisting of the amino acid sequence set forth in SEQ ID NO: 29), a mouse kappa leader sequence (e.g., a mouse kappa leader sequence comprising or consisting of the amino acid sequence set forth in SEQ ID NO: 30); a human CD8 leader sequence (e.g., a human CD8 leader sequence comprising or consisting of the amino acid sequence set forth in SEQ ID NO: 31); a truncated human CD8 signal peptide (e.g., a truncated human CD8 signal peptide comprising or consisting of the amino acid sequence set forth in SEQ ID NO: 32); a human albumin signal sequence (e.g., a human albumin signal sequence comprising or consisting of the amino acid sequence set forth in SEQ ID NO: 33); and a human prolactin signal sequence (e.g., a human prolactin signal sequence comprising or consisting of the amino acid sequence set forth in SEQ ID NO: 34). SEQ ID Nos: 28-34 are provided below.

[SEQ ID NO: 27]

MYRMOLLSCIALSLALVINS

[SEQ ID NO: 28]

MYSMQLASCVTLTLVLLVNS

[SEQ ID NO: 29]

METPAQLLFLLLLWLPDTTG

[SEQ ID NO: 30]

METDTLLLWVLLLWVPGSTG

[SEQ ID NO: 31]

MALPVTALLLPLALLLHAARP

[SEQ ID NO: 32]

MALPVTALLLPLALLLHA

[SEQ ID NO: 33]

MKWVTFISLLESSAYS

[SEQ ID NO: 34]

MDSKGSSQKGSRLLLLLVVSNLLLCQGVVS

### 2.1.2. CCR

In certain non-limiting embodiments, the presently disclosed subject matter provides antibodies or antigen-binding fragments thereof that specifically bind to an antigen-binding domain of a CCR. In certain non-limiting embodiments, the presently disclosed antibodies or antigen-binding fragments thereof bind to an extracellular domain of a CCR. In certain non-limiting embodiments, the presently disclosed antibodies or antigen-binding fragments thereof are anti-idiotype antibodies.

The term “chimeric co-stimulating receptor” or “CCR” refers to a chimeric receptor that binds to an antigen and provides a co-stimulatory signal, but does not provide a T-cell activation signal to a cell comprising the CCR. Various CCRs are described in U.S. Patent Publication No. 2002/0018783, the contents of which are incorporated by reference in their entirety. CCRs mimic co-stimulatory signals, but unlike, CARs, do not provide a T-cell activation signal. In certain embodiments, the CCR lacks a CD3ζ polypeptide.

CCRs provide co-stimulation signal (e.g., a CD28-like signal or 4-1BB-like signal), in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013); 31(1):71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to an antigen and an intracellular domain that is capable of delivering a costimulatory signal to the cell but does not alone deliver an activation signal to the cell. In certain embodiments, the CCR further comprises a transmembrane domain. In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD150, CD226, and NKG2D.

### 2.1.3. TCR Fusion Molecules

In certain non-limiting embodiments, the presently disclosed subject matter provides antibodies or antigen-binding fragments thereof that specifically bind to an antigen-binding domain of a TCR fusion molecules (HIT). In certain non-limiting embodiments, the presently disclosed antibodies or antigen-binding fragments thereof bind to an extracellular domain of a (HIT). In certain non-limiting embodiments, the presently disclosed antibodies or antigen-binding fragments thereof are anti-idiotype antibodies.

Non-limiting examples of TCR fusion molecules include HLA-Independent TCR-based Chimeric Antigen Receptor (also known as “HIT”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR-like fusion molecule is a recombinant T cell receptor (TCR). In certain embodiments, the recombinant TCR comprises at least one antigen-binding chain. In certain embodiments, the antigen-binding domain of the recombinant TCR comprises a ligand for a cell-surface receptor, a receptor for a cell surface ligand, an antigen binding portion of an antibody or a fragment thereof, or an antigen binding portion of a TCR. In certain embodiments, the recombinant TCR comprises two antigen binding chains, i.e., a first antigen binding chain and a second antigen binding chain. In certain embodiments, the first and second antigen-binding chains each comprises a constant domain. In certain embodiments, the recombinant TCR binds to an antigen (e.g., a first antigen or a second antigen) in an HLA-independent manner. Thus, in certain embodiments, the recombinant TCR is an HLA-independent (or non-HLA restricted) TCR (referred to as “HIT”).

In certain embodiments, the first antigen-binding chain comprises an antigen-binding fragment of a heavy chain variable region (V_{H}) of an antibody. In certain embodiments, the second antigen-binding chain comprises an antigen-binding fragment of a light chain variable region (V_{L}) of an antibody. In certain embodiments, the first antigen-binding chain comprises an antigen-binding fragment of a V_{H} of an antibody, and the second antigen-binding chain comprises an antigen-binding fragment of a V_{L} of the antibody.

In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the first antigen-binding chain comprises a TRAC polypeptide, and the second antigen-binding chain comprises a TRBC polypeptide. In certain embodiments, the first antigen-binding chain comprises a TRBC polypeptide, and the second antigen-binding chain comprises a TRAC polypeptide.

In certain embodiments, the first antigen-binding chain comprises a V_{H} of an antibody and a TRAC polypeptide, and the second antigen-binding chain comprises a V_{L} of an antibody and a TRBC polypeptide.

In certain embodiments, the first antigen-binding chain comprises a V_{H} of an antibody and a TRBC polypeptide, and the second antigen-binding chain comprises a V_{L} of an antibody and a TRAC polypeptide.

In certain embodiments, at least one of the TRAC polypeptide and the TRBC polypeptide is endogenous. In certain embodiments, the TRAC polypeptide is endogenous. In certain embodiments, the TRBC polypeptide is endogenous. In certain embodiments, both the TRAC polypeptide and the TRBC polypeptide are endogenous.

In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR-like fusion molecule is capable of integrating with a CD3 complex and providing HLA-independent antigen recognition. In certain embodiments, the TCR-like fusion molecule replaces an endogenous TCR in a CD3/TCR complex.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the first antigen binding chain or the second antigen binding chain comprises a constant domain that comprises a native or modified TRAC polypeptide. In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 35 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 35. SEQ ID NO: 35 is provided below.

[SEQ ID NO: 35]
IQNPDPAVYQLRDSKSSDKSVCLFTDFDSQTNVSQSKDSDVYITDKTVLD
MRSMDFKSNSAVAWSNKSDEACANAFNNSIIPEDTFFPSPESSCDVKLVE
KSFETDTNLNFQNLSVIGFRILLLKVAGENLLMTLRLWSS

An exemplary nucleotide sequence encoding the amino acid sequence of SEQ ID NO: 35 is set forth in SEQ ID NO: 36, which is provided below.

[SEQ ID NO: 36]
ATCCAGAACCCTGACCCTGCCGTGTACCAGCTGAGAGACTCTAAATCCAG
TGACAAGTCTGTCTGCCTATTCACCGATTTTGATTCTCAAACAAATGTGT
CACAAAGTAAGGATTCTGATGTGTATATCACAGACAAAACTGTGCTAGAC
ATGAGGTCTATGGACTTCAAGAGCAACAGTGCTGTGGCCTGGAGCAACAA
ATCTGACTTTGCATGTGCAAACGCCTTCAACAACAGCATTATTCCAGAAG
ACACCTTCTTCCCCAGCCCAGAAAGTTCCTGTGATGTCAAGCTGGTCGAG
AAAAGCTTTGAAACAGATACGAACCTAAACTTTCAAAACCTGTCAGTGAT
TGGGTTCCGAATCCTCCTCCTGAAAGTGGCCGGGTTTAATCTGCTCATGA
CGCTGCGGCTGTGGTCCAGC

In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 37 or a fragment thereof, and/or may optionally comprise up to one or up to two or up to three conservative amino acid substitutions.

In certain embodiments, the TRAC polypeptide comprises or consists of the amino acid sequence set forth in SEQ ID NO: 37. SEQ ID NO: 37 is provided below.

[SEQ ID NO: 37]
IPNIQNPDPAVYQLRDSKSSDKSVCLFTDFDSQTNVSQSKDSDVYITDKT
VLDMRSMDFKSNSAVAWSNKSDFACANAFNNSIIPEDTFFPSPESSCDVK
LVEKSFETDTNLNFQNLSVIGFRILLLKVAGENLLMTLRLWSS

An exemplary nucleotide sequence encoding the amino acid sequence of SEQ ID NO: 37 is set forth in SEQ ID NO: 38, which is provided below.

[SEQ ID NO: 38]
ATTCCCAATATCCAGAACCCTGACCCTGCCGTGTACCAGCTGAGAGACTC
TAAATCCAGTGACAAGTCTGTCTGCCTATTCACCGATTTTGATTCTCAAA
CAAATGTGTCACAAAGTAAGGATTCTGATGTGTATATCACAGACAAAACT
GTGCTAGACATGAGGTCTATGGACTTCAAGAGCAACAGTGCTGTGGCCTG
GAGCAACAAATCTGACTTTGCATGTGCAAACGCCTTCAACAACAGCATTA
TTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCT
GTCAGTGATTGGGTTCCGAATCCTCCTCCTGAAAGTGGCCGGGTTTAATC
TGCTCATGACGCTGCGGCTGTGGTCCAGC

In certain embodiments, the first antigen binding chain or the second antigen binding chain comprises a constant domain comprising a native or modified TRBC polypeptide. In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 39 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 39. SEQ ID NO: 39 is provided below.

[SEQ ID NO: 39]
DLKNVFPPEVAVFEPSEAEISHTQKATLVCLATGFYPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSESYQQGVLSATILYEI
LLGKATLYAVLVSALVLMAMVKRKDSRG

An exemplary nucleotide sequence encoding the amino acid sequence of SEQ ID NO: 39 is set forth in SEQ ID NO: 40, which is provided below.

[SEQ ID NO: 40]
GATCTGAAAAACGTGTTCCCTCCTGAAGTGGCTGTCTTTGAACCATCCGA
GGCCGAGATTTCCCATACCCAGAAAGCAACTCTGGTCTGTCTGGCCACTG
GATTCTACCCCGATCACGTGGAACTGTCTTGGTGGGTGAACGGCAAGGAA
GTCCATTCCGGAGTCTCTACCGACCCTCAGCCCCTCAAGGAGCAGCCTGC
TCTCAACGATTCTCGGTACTGCCTGTCATCTCGACTGAGAGTGTCTGCCA
CCTTCTGGCAGAACCCTAGAAACCACTTTCGGTGTCAGGTCCAGTTTTAC
GGCCTGAGCGAGAACGATGAGTGGACACAGGATAGAGCCAAACCTGTGAC
ACAGATTGTGAGCGCCGAGGCTTGGGGACGAGCCGATTGTGGCTTCACAT
CCGAGTCTTACCAGCAGGGAGTGCTGTCTGCTACAATCCTCTACGAAATT
CTCCTGGGGAAGGCCACCCTGTACGCTGTCCTCGTGTCTGCTCTGGTGCT
CATGGCTATGGTCAAACGAAAGGACTCTAGAGGC

In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 41 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 41. SEQ ID NO: 41 is provided below.

[SEQ ID NO: 41]
LEDLKNVFPPEVAVFEPSEAEISHTQKATLVCLATGFYPDHVELSWWVNG
KEVHSGVSTDPQPLKEQPALNDSRYCLSSRLRVSATFWQNPRNHFRCQVQ
FYGLSENDEWTQDRAKPVTQIVSAEAWGRADCGFTSESYQQGVLSATILY
EILLGKATLYAVLVSALVLMAMVKRKDSRG

An exemplary nucleotide sequence encoding the amino acid sequence of SEQ ID NO: 41 is set forth in SEQ ID NO: 42, which is provided below.

[SEQ ID NO: 42]
CTGGAGGATCTGAAAAACGTGTTCCCTCCTGAAGTGGCTGTCTTTGAACC
ATCCGAGGCCGAGATTTCCCATACCCAGAAAGCAACTCTGGTCTGTCTGG
CCACTGGATTCTACCCCGATCACGTGGAACTGTCTTGGTGGGTGAACGGC
AAGGAAGTCCATTCCGGAGTCTCTACCGACCCTCAGCCCCTCAAGGAGCA
GCCTGCTCTCAACGATTCTCGGTACTGCCTGTCATCTCGACTGAGAGTGT
CTGCCACCTTCTGGCAGAACCCTAGAAACCACTTTCGGTGTCAGGTCCAG
TTTTACGGCCTGAGCGAGAACGATGAGTGGACACAGGATAGAGCCAAACC
TGTGACACAGATTGTGAGCGCCGAGGCTTGGGGACGAGCCGATTGTGGCT
TCACATCCGAGTCTTACCAGCAGGGAGTGCTGTCTGCTACAATCCTCTAC
GAAATTCTCCTGGGGAAGGCCACCCTGTACGCTGTCCTCGTGTCTGCTCT
GGTGCTCATGGCTATGGTCAAACGAAAGGACTCTAGAGGC

In certain embodiments, the TRBC polypeptide is a TRBC1 polypeptide. In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 43 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 43. SEQ ID NO: 43 is provided below.

[SEQ ID NO: 43]
LNKVFPPEVAVFEPSEAEISHTQKATLVCLATGFFPDHVELSWWVNGKEV
HSGVSTDPQPLKEQPALNDSRYCLSSRLRVSATFWQNPRNHFRCQVQFYG
LSENDEWTQDRAKPVTQIVSAEAWGRADCGFTSVSYQQGVLSATILYEIL
LGKATLYAVLVSALVLMAMVKRKDF

In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 44 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 44. SEQ ID NO: 44 is provided below.

[SEQ ID NO: 44]
DLNKVFPPEVAVFEPSEAEISHTQKATLVCLATGFFPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSVSYQQGVLSATILYEI
LLGKATLYAVLVSALVLMAMVKRKDF

An exemplary nucleotide sequence encoding the amino acid sequence of SEQ ID NO: 44 is set forth in SEQ ID NO: 45, which is provided below.

[SEQ ID NO: 45]
GACCTGAACAAGGTGTTCCCACCCGAGGTCGCTGTGTTTGAGCCATCAGA
AGCAGAGATCTCCCACACCCAAAAGGCCACACTGGTGTGCCTGGCCACAG
GCTTCTTCCCCGACCACGTGGAGCTGAGCTGGTGGGTGAATGGGAAGGAG
GTGCACAGTGGGGTCAGCACAGACCCGCAGCCCCTCAAGGAGCAGCCCGC
CCTCAATGACTCCAGATACTGCCTGAGCAGCCGCCTGAGGGTCTCGGCCA
CCTTCTGGCAGAACCCCCGCAACCACTTCCGCTGTCAAGTCCAGTTCTAC
GGGCTCTCGGAGAATGACGAGTGGACCCAGGATAGGGCCAAACCCGTCAC
CCAGATCGTCAGCGCCGAGGCCTGGGGTAGAGCAGACTGTGGCTTTACCT
CGGTGTCCTACCAGCAAGGGGTCCTGTCTGCCACCATCCTCTATGAGATC
TCGCTAGGGAAGGCCACCCTGTATGCTGTGCTGGTCAGCGCCCTTGTGTT
GATGGCCATGGTCAAGAGAAAGGATTTC

In certain embodiments, the first antigen binding chain or the second antigen binding chain comprises a constant domain comprising a native or modified TRGC polypeptide. In certain embodiments, the TRGC polypeptide is a native or modified TRGC1 polypeptide. In certain embodiments, the TRGC1 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 46, which is provided below. In certain embodiments, the TRGC1 polypeptide comprises or consists of the amino acid sequence set forth in SEQ ID NO: 46.

[SEQ ID NO: 46]
DKQLDADVSPKPTIFLPSIAETKLQKAGTYLCLLEKFFPDVIKIHWQEKK
SNTILGSQEGNTMKINDTYMKFSWLTVPEKSLDKEHRCIVRHENNKNGVD
QEIIFPPIKTDVITMDPKDNCSKDANDTLLLQLTNTSAYYMYLLLLLKSV
VYFAIITCCLLRRTAFCCNGEKS

In certain embodiments, the TRGC polypeptide is a native or modified TRGC2 polypeptide. In certain embodiments, the TRGC2 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 47, which is provided below. In certain embodiments, the TRGC2 polypeptide comprises or consists of the amino acid sequence set forth in SEQ ID NO: 47.

[SEQ ID NO: 47]
DKQLDADVSPKPTIFLPSIAETKLQKAGTYLCLLEKFFPDIIKIHWQEKK
SNTILGSQEGNTMKINDTYMKFSWLTVPEESLDKEHRCIVRHENNKNGID
QEIIFPPIKTDVTTVDPKYNYSKDANDVITMDPKDNWSKDANDTLLLQLT
NTSAYYTYLLLLLKSVVYFAIITCCLLRRTAFCCNGEKS

In certain embodiments, the first antigen binding chain or the second antigen binding chain comprises a constant domain comprising a native or modified TRDC polypeptide. In certain embodiments, the TRDC polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 48, which is provided below. In certain embodiments, the TRDC polypeptide comprises or consists of the amino acid sequence set forth in SEQ ID NO: 48.

[SEQ ID NO: 48]
SQPHTKPSVFVMKNGTNVACLVKEFYPKDIRINLVSSKKITEFDPAIVIS
PSGKYNAVKLGKYEDSNSVTCSVQHDNKTVHSTDFEVKTDSTDHVKPKET
ENTKQPSKSCHKPKAIVHTEKVNMMSLTVLGLRMLFAKTVAVNELLTAKL
FFL

In certain embodiments, the TCR-like fusion molecule comprises a hinge/spacer region that links the first antigen binding chain to the constant domain. In certain embodiments, the TCR-like fusion molecule comprises a hinge/spacer region that links the second antigen binding chain to the constant domain. The hinge/spacer region can be flexible enough to allow the antigen binding chain to orient in different directions to facilitate antigen recognition. In certain embodiments, the hinge/spacer region can be the hinge region from IgG1, the CH₂CH₃ region of immunoglobulin and portions of CD3, a portion of a TCRα polypeptide, a portion of a TCRβ polypeptide, a portion of a CD28 polypeptide, a portion of a CD8 polypeptide, or a synthetic spacer sequence. In certain embodiments, the hinge/spacer region comprises a portion of a TCRα polypeptide. In certain embodiments, the hinge/spacer region comprises a portion of the variable region (TRAV), a portion of the diversity region (TRAD), a portion of the joining region (TRAJ), a portion of the constant region (TRAC), or a combination thereof. In certain embodiments, the hinge/spacer region comprises a portion of the TRAJ region and a portion of the TRAC region of the TCRα polypeptide. In certain embodiments, the hinge/spacer region comprises or consists of the amino acid sequence set forth in SEQ ID NO: 49. In certain embodiments, the hinge/spacer region comprises or consists of amino acids 1 to 3 of the sequence set forth in SEQ ID NO: 49. An exemplary nucleic acid sequence encoding the amino acid sequence of SEQ ID NO: 49 is set forth in SEQ ID NO: 50. SEQ ID NO: 49 and 50 are provided below.

[SEQ ID NO: 49]
IPNIQNPDPA
[SEQ ID NO: 50]
ATTCCCAATATCCAGAACCCTGACCCTGCC

In certain embodiments, the hinge/spacer region comprises a portion of a TCRβ polypeptide. In certain embodiments, the hinge/spacer region comprises a portion of the variable region (TRBV), a portion of the diversity region (TRBD), a portion of the joining region (TRBJ), a portion of the constant region (TRBC), or a combination thereof. In certain embodiments, the hinge/spacer region comprises a portion of the TRBJ region and a portion of the TRAC region (C) of the TCRβ polypeptide. In certain embodiments, the hinge/spacer region comprises or consists of the amino acid sequence set forth in SEQ ID NO: 51. In certain embodiments, the hinge/spacer region comprises or consists of amino acid 1 to 2 of the sequence set forth in SEQ ID NO: 51. An exemplary nucleic acid sequence encoding the amino acid sequence of SEQ ID NO: 51 is set forth in SEQ ID NO: 52. SEQ ID NO: 51 and 52 are provided below.

[SEQ ID NO: 51]
LEDLKNVEPPE
[SEQ ID NO: 52]
CTGGAGGATCTGAAAAACGTGTTCCCTCCTGAA

In certain embodiments, the antigen binding chain does not comprise an intracellular domain. In certain embodiments, the antigen binding chain is capable of associating with a CD3ζ polypeptide. In certain embodiments, the antigen binding chain associating with the CD3ζ polypeptide via the constant domain. In certain embodiments, the CD3ζ polypeptide is endogenous. In certain embodiments, the CD3ζ polypeptide is exogenous. In certain embodiments, binding of the antigen binding chain to a target antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR-like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD3ζ polypeptide of the antigen binding chain.

In certain embodiments, the TCR-like fusion molecule comprises an antigen binding chain that comprises an intracellular domain, wherein the intracellular domain comprises a co-stimulatory signaling region. In certain embodiments, the intracellular domain comprises a co-stimulatory signaling region and a CD3ζ polypeptide. In certain embodiments, the intracellular domain comprises a co-stimulatory signaling region and does not comprise a CD3ζ polypeptide. In certain embodiments, the co-stimulatory signaling region comprises at least an intracellular domain of a co-stimulatory molecule disclosed herein.

In certain embodiments, the TCR-like fusion molecule is capable of associating with a CD3 complex (also known as “T-cell co-receptor”). In certain embodiments, the TCR-like fusion molecule and the CD3 complex form an antigen recognizing receptor complex similar to a native TCR/CD3 complex. In certain embodiments, the CD3 complex is endogenous. In certain embodiments, the CD3 complex is exogenous. In certain embodiments, the TCR-like fusion molecule replaces a native and/or an endogenous TCR in the CD3/TCR complex. In certain embodiments, the CD3 complex comprises a CD37 chain, a CD36 chain, and two CD3F chains.

In certain embodiments, the CD37 chain comprises or consists of an amino acid sequence that is at least about 85%, about 90%, about 95%, about 96%, about 97%, about 98%, about 99% or about 100% homologous or identical to the amino acid sequence having a NCBI reference number: NP_000064.1 or a fragment thereof, and/or may optionally comprise up to one or up to two or up to three conservative amino acid substitutions.

In certain embodiments, the CD36 chain comprises or consists of an amino acid sequence that is at least about 85%, about 90%, about 95%, about 96%, about 97%, about 98%, about 99% or about 100% homologous or identical to the amino acid sequence having a NCBI reference numbers: NP_000723.1 or a fragment thereof, or the amino acid sequence having a NCBI reference numbers: NP_001035741.1 or a fragment thereof, and/or may optionally comprise up to one or up to two or up to three conservative amino acid substitutions.

In certain embodiments, the CD3F chain comprises or consists of an amino acid sequence that is at least about 85%, about 90%, about 95%, about 96%, about 97%, about 98%, about 99% or about 100% homologous or identical to the amino acid sequence having a NCBI reference number: NP_000724.1 or a fragment thereof, and/or may optionally comprise up to one or up to two or up to three conservative amino acid substitutions.

In certain embodiments, the TCR-like fusion molecule exhibits a greater antigen sensitivity than a CAR targeting the same antigen. In certain embodiments, the TCR-like fusion molecule is capable of inducing an immune response when binding to an antigen that has a low antigen density on the surface of a tumor cell.

2.2. Single-Chain Variable Fragments (scFvs)

In certain embodiments, the presently disclosed subject matter includes antibodies or antigen-binding fragments thereof that have the scFv sequence fused to one or more constant domains to form an antibody with an Fc region of a human immunoglobulin to yield a bivalent protein, increasing the overall avidity and stability of the antibody. In addition, the Fc portion allows the direct conjugation of other molecules, including but not limited to fluorescent dyes, cytotoxins, radioisotopes, etc. to the antibody for example, for use in antigen quantitation studies, to immobilize the antibody for affinity measurements, for targeted delivery of a therapeutic agent, to test for Fc-mediated cytotoxicity using immune effector cells and many other applications.

The results presented here highlight the specificity, sensitivity, and utility of the presently disclosed antibodies or antigen-binding fragments in targeting an extracellular domain of a chimeric receptor (e.g., a CAR, a HIT, etc.) or an antigen-binding domain (e.g., an antigen-binding domain of a CAR).

In certain embodiments, the presently disclosed scFv is an scFv-Fc fusion protein or a full-length human IgG with V_{H} and V_{L} regions or CDRs selected from Table 1. In certain embodiments, the presently disclosed scFv comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof. SEQ ID NOs: 1-3 are provided in Table 1.

In certain embodiments, the presently disclosed scFv comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. SEQ ID NOs: 4-6 are provided in Table 1.

In certain embodiments, the presently disclosed scFv comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof, and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification, and a CDR3 comprising the amino acid sequence set forth in SEQ ID NO: 6 or a conservative modification thereof.

In certain embodiments, the presently disclosed scFv comprises a V_{H} comprising a CDR1 comprising the amino acid sequence set forth in SEQ ID NO: 1, a CDR2 comprising the amino acid sequence set forth in SEQ ID NO: 2, a V_{H} CDR3 comprising the amino acid sequence set forth in SEQ ID NO: 3; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the presently disclosed scFv comprises a V_{H} comprising the amino acid sequence set forth in SEQ ID NO: 7. An exemplary nucleotide sequence encoding the amino acid sequence of SEQ ID NO: 7 is set forth in SEQ ID NO: 9. In certain embodiments, the presently disclosed scFv comprises a V_{L} comprising the amino acid sequence set forth in SEQ ID NO: 8. An exemplary nucleotide sequence encoding the amino acid sequence of SEQ ID NO: 8 is set forth in SEQ ID NO: 10. SEQ ID NO: 7-10 are provided in Table 1. In certain embodiments, the scFv is designated as “CAR1” or “19E3”.

In certain embodiments, the presently disclosed scFv comprises a V_{H} comprising the amino acid sequence set forth in SEQ ID NO: 7 and a V_{L} comprising the amino acid sequence set forth in SEQ ID NO: 8.

In certain embodiments, the presently disclosed scFv comprises a V_{H} comprising the amino acid sequence set forth in SEQ ID NO: 7, and a V_{L} comprising the amino acid sequence set forth in SEQ ID NO: 8. In certain embodiments, the V_{H} and V_{L} are linked via a linker. In certain embodiments, the linker comprises the amino acid sequence set forth in SEQ ID NO: 21, SEQ ID NO: 22, SEQ ID NO: 23, SEQ ID NO: 24, SEQ ID NO: 25, or SEQ ID NO: 26.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 1

| CDRS | 1 | 2 | 3 |
|---|---|---|---|
| V_{H} | GFTFSDED | INGGSGVI | AREELGRRYYFDY |
| | [SEQ ID NO: 1] | [SEQ ID NO: 2] | [SEQ ID NO: 3] |
| V_{L} | SEHSSYN | LKSDGSH | GAGYTISGQYGYV |
| | [SEQ ID NO: 4] | [SEQ ID NO: 5] | [SEQ ID NO: 6] |

| | |
|---|---|
| Full V_{H} | MNSGLKLVFFVLILKGVQCEVOLVESGGGLVQTGKSLKLSCEASGFTESDEDMN |
| | WVRQAPGKGLEWVAYINGGSGVIFYADAVKGRFTISRDNAKNLLFLQMNNLKSE |
| | DSAMYYCAREELGRRYYFDYWGQGTMVTVSSATTTAPSVYPLAPACDSTTSTTN |
| | TVTLGCLVKGYFPEPVTVSWNSGALTSGVHTFPSVLHSGLYSLSSSVTVPSSTW |
| | [SEQ ID NO: 7] |
| Full V_{L} | MAWIPLLFFLLHCTGSFSQPVLTQSPSASASLSGSVKLTCTLSSEHSSYNIAWY |
| | QQHPDKAPKYVMYLKSDGSHFKGDGIPDRFSGSSSGAHRYLSISNVQSEDDATY |
| | FCGAGYTISGQYGYVFDSGTQLTVLGGPKSSPKVTVFPPSPEELRTNKATLVCL |
| | VNDFYPGSATVTWKANGATINDGVKTTKPSKQGQNYMTSSYLSLTADQWRSH |
| | [SEQ ID NO: 8] |
| DNA for | ATGAACTCAGGACTCAAATTGGTTTTCTTTGTCCTTATTCTGAAAGGTGTCCAG |
| Full V_{H} | TGTGAGGTGCAGTTGGTGGAGTCTGGGGGAGGCTTAGTACAGACTGGAAAGTCC |
| | CTGAAACTCTCATGTGAGGCCTCTGGATTCACCTTCAGTGACTTTGACATGAAC |
| | TGGGTCCGCCAGGCTCCAGGGAAGGGGCTGGAGTGGGTCGCATACATTAATGGT |
| | GGTAGTGGTGTTATCTTTTATGCTGACGCTGTGAAGGGCCGATTCACCATCTCC |
| | AGAGACAACGCCAAGAACTTACTGTTCCTACAGATGAACAATCTCAAGTCTGAG |
| | GACTCAGCCATGTATTACTGTGCAAGAGAGGAACTGGGACGGAGGTACTACTTT |
| | GATTACTGGGGCCAAGGAACCATGGTCACCGTCTCCTCAGCCACAACAACAGCC |
| | CCATCTGTCTATCCCTTGGCCCCTGCCTGTGACAGCACAACCAGCACCACGAAC |
| | ACGGTGACCCTGGGATGCCTGGTCAAGGGCTATTTCCCTGAGCCGGTGACCGTA |
| | AGCTGGAACTCTGGAGCCCTGACCAGCGGCGTGCACACCTTCCCATCTGTCCTG |
| | CATTCTGGGCTCTACTCCCTCAGCAGCTCAGTGACTGTACCTTCCAGCACCTGG |
| | [SEQ ID NO: 9] |
| DNA for | ATGGCCTGGATTCCTCTCCTCTTCTTCCTCCTTCATTGCACAGGGTCTTTCTCT |
| Full V_{L} | CAACCTGTGTTGACTCAGTCACCCTCTGCCTCTGCCTCCCTGAGTGGCTCAGTC |
| | AAACTCACCTGCACCCTGAGTAGTGAGCACAGCTCCTACAACATAGCATGGTAC |
| | CAGCAACATCCAGACAAGGCTCCCAAGTATGTGATGTACCTTAAGAGTGATGGA |
| | AGCCACTTCAAGGGAGATGGGATCCCTGATCGCTTCTCTGGCTCCAGCTCTGGG |
| | GCTCATCGCTACTTAAGCATCTCCAATGTCCAGTCTGAAGATGATGCTACCTAT |
| | TTCTGTGGTGCAGGTTATACCATTTCTGGACAATATGGGTATGTTTTTGACAGC |
| | GGAACCCAGCTCACCGTCCTAGGTGGACCCAAGTCTTCTCCCAAAGTCACAGTG |
| | TTTCCACCTTCACCTGAGGAGCTCCGGACAAACAAAGCCACACTGGTGTGTCTG |
| | GTTAATGACTTCTACCCGGGTTCTGCAACAGTGACCTGGAAGGCAAATGGAGCA |
| | ACTATCAATGATGGGGTGAAGACTACAAAGCCTTCCAAACAGGGCCAAAACTAC |
| | ATGACCAGCAGCTACCTAAGTTTGACAGCAGACCAGTGGAGATCCCAC |
| | [SEQ ID NO: 10] |

In certain embodiments, the presently disclosed scFv is an scFv-Fc fusion protein or a full-length human IgG with V_{H} and V_{L} regions or CDRs selected from Table 2.

In certain embodiments, the presently disclosed scFv comprises a V_{H} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof. SEQ ID NOs: 11-13 are provided in Table 2.

In certain embodiments, the presently disclosed scFv comprises a V_{L} comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof. SEQ ID NOs: 14-16 are provided in Table 2.

In certain embodiments, the presently disclosed scFv comprises a V_{H} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof, and a V_{L} comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification.

In certain embodiments, the presently disclosed scFv comprises a V_{H} comprising a CDR1 comprising the amino acid sequence set forth in SEQ ID NO: 11, a CDR2 comprising the amino acid sequence set forth in SEQ ID NO: 12, a V_{H} CDR3 comprising the amino acid sequence set forth in SEQ ID NO: 13; and a V_{L} comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the presently disclosed scFv comprises a V_{H} comprising the amino acid sequence set forth in SEQ ID NO: 17, as shown in Table 2. An exemplary nucleotide sequence encoding the amino acid sequence of SEQ ID NO: 17 is set forth in SEQ ID NO: 19. In certain embodiments, the presently disclosed scFv comprises a V_{L} comprising the amino acid sequence set forth in SEQ ID NO: 18. An exemplary nucleotide sequence encoding the amino acid sequence of SEQ ID NO: 18 is set forth in SEQ ID NO: 20. In certain embodiments, the presently disclosed scFv comprises a V_{H} comprising the amino acid sequence set forth in SEQ ID NO: 17 and a V_{L} comprising the amino acid sequence set forth in SEQ ID NO: 18. SEQ ID NO: 17-20 are provided in Table 2. In certain embodiments, the scFv is designated as “CAR2” or “12D1 1”.

In certain embodiments, the presently disclosed scFv comprises a V_{H} comprising the amino acid sequence set forth in SEQ ID NO: 17, and a V_{L} comprising the amino acid sequence set forth in SEQ ID NO: 18. In certain embodiments, the V_{H} and V_{L} are linked via a linker. In certain embodiments, the linker comprises the amino acid sequence set forth in SEQ ID NO: 21, SEQ ID NO: 22, SEQ ID NO: 23, SEQ ID NO: 24, SEQ ID NO: 25, or SEQ ID NO: 26.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 2

| CDRS | 1 | 2 | 3 |
|---|---|---|---|
| V_{H} | GFDLSDYF | IYPQSYDYAT | TKDGDVGF |
| | [SEQ ID NO: 11] | [SEQ ID NO: 12] | [SEQ ID NO: 13] |
| | QDISNE | NGK | LOHYSFPLT |
| | [SEQ ID NO: 14] | [SEQ ID NO: 15] | [SEQ ID NO: 16] |

| | |
|---|---|
| Full V_{H} | MGLGLQWVFFVALLKGVHCAVRLLESGGGLVKPEGALKLSCVASGEDLSDYEMG |
| | WVRQAPGKGLEWVAHIYPQSYDYATYYSGSVQGRFTISRDDSRSMVYLQMNNLR |
| | TEDTATYYCTKDGDVGFWGQGTMVTVSSATTTAPSVYPLAPACDSTTSTTNTVT |
| | LGCLVKGYFPEPVTVSWNSGALTSGVHTFPSVLHSGLYSLSSSVTVPSSTW |
| | [SEQ ID NO: 17] |
| Full V_{L} | MANKSPAQALAILLLWLSGVRCDIQVTQSPTLLSASLGDKVTINCLASQDISNE |
| | LNWYQQKSGQSPTLLIYNGKNLQSGVPSRESGQYSGRSFTISINNVEPEDVATY |
| | FCLQHYSFPLTFGDGSKLEMKRADAKPTVSIFPPSSEQLGTGSATLVCFVNNFY |
| | PKDINVKWKVDGSEKRDGVLQSVTDQDSKDSTYS [SEQ ID NO: 18] |
| DNA for | ATGGGATTGGGACTGCAGTGGGTTTTCTTTGTTGCTCTTTTAAAAGGTGTCCAC |
| Full V_{H} | TGTGCGGTGCGGCTTCTGGAGTCGGGTGGAGGATTAGTGAAGCCTGAGGGGGCA |
| | CTGAAACTCTCCTGTGTGGCCTCTGGATTCGACTTAAGTGACTATTTCATGGGC |
| | TGGGTCCGCCAGGCTCCAGGGAAGGGGCTGGAGTGGGTTGCTCACATATACCCT |
| | CAAAGTTATGATTATGCAACCTATTACTCGGGTTCGGTGCAAGGCAGATTCACC |
| | ATCTCCAGAGATGATTCCCGAAGCATGGTCTACCTGCAAATGAACAACCTGAGA |
| | ACTGAGGACACGGCCACTTATTACTGTACAAAAGACGGGGACGTGGGTTTCTGG |
| | GGCCAAGGAACCATGGTCACCGTCTCCTCAGCCACAACAACAGCCCCATCTGTC |
| | TATCCCTTGGCCCCTGCCTGTGACAGCACAACCAGCACCACGAACACGGTGACC |
| | CTGGGATGCCTGGTCAAGGGCTATTTCCCTGAGCCGGTGACCGTAAGCTGGAAC |
| | TCTGGAGCCCTGACCAGCGGCGTGCACACCTTCCCATCTGTCCTGCATTCTGGG |
| | CTCTACTCCCTCAGCAGCTCAGTGACTGTACCTTCCAGCACCTGG |
| | [SEQ ID NO: 19] |
| DNA for | ATGGCCAACAAGTCTCCTGCTCAGGCACTGGCAATTTTGTTACTGTGGCTGTCA |
| Full V_{L} | GGTGTCAGATGTGACATTCAAGTGACACAATCTCCCACCCTCCTGTCAGCATCT |
| | CTAGGAGACAAAGTGACCATCAATTGCCTGGCAAGTCAGGACATTAGCAATGAG |
| | TTAAACTGGTACCAGCAGAAGTCAGGACAATCTCCTACACTGTTGATTTATAAT |
| | GGAAAAAATTTGCAGTCTGGTGTCCCGTCAAGGTTCAGTGGCCAGTATTCAGGG |
| | AGAAGTTTCACTATCAGCATCAACAATGTGGAACCTGAAGATGTTGCAACTTAT |
| | TTTTGTCTTCAGCATTACAGTTTTCCGCTCACGTTCGGTGATGGCTCCAAGCTG |
| | GAGATGAAACGGGCTGATGCTAAGCCAACCGTCTCCATCTTCCCACCATCCAGT |
| | GAGCAGTTGGGCACTGGAAGCGCCACACTTGTGTGCTTCGTGAACAACTTCTAC |
| | CCCAAAGACATCAATGTCAAGTGGAAAGTAGATGGCAGTGAAAAACGAGATGGC |
| | GTCCTGCAGAGTGTCACTGATCAGGACAGCAAAGACAGCACCTACAGC |
| | [SEQ ID NO: 20] |

### 2.3. Monoclonal Antibodies

The presently disclosed subject matter provides antibodies (e.g., human antibodies, e.g., human monoclonal antibodies) that specifically bind to an extracellular domain of a chimeric receptor (e.g., a CAR, a HIT, etc.) or an antigen-binding domain (e.g., an antigen-binding domain of a CAR). The V_{H} amino acid sequences of presently disclosed antibodies are set forth in SEQ ID NOs: 7 and 17. The V_{L} amino acid sequences of the presently disclosed antibodies are set forth in SEQ ID NOs: 8 and 18.

Given that each of the presently disclosed antibodies can bind to CAR, the V_{H} and V_{L} sequences can be “mixed and matched” to create other binding molecules targeting chimeric receptors. Binding of such “mixed and matched” antibodies can be tested using the binding assays known in the art, including for example, ELISAs, Western blots, RIAs, Biacore analysis. Preferably, when V_{H} and V_{L} chains are mixed and matched, a V_{H} sequence from a particular V_{H}/V_{L} pairing is replaced with a structurally similar V_{H} sequence. Likewise, a V_{L} sequence from a particular V_{H}/V_{L} pairing is replaced with a structurally similar V_{L} sequence.

In certain embodiments, the presently disclosed subject matter provides an antibody or an antigen-binding fragment thereof comprising: (a) a heavy chain variable region (V_{H}) comprising an amino acid sequence selected from SEQ ID NOs: 7 and 17; and (b) a light chain variable region (V_{L}) comprising an amino acid sequence selected from SEQ ID NOs: 8 and 18; wherein the antibody or antigen-binding fragment specifically binds to a chimeric receptor, e.g., the extracellular antigen-binding domain of a CAR. In certain embodiments, the V_{H} and V_{L} are selected from the group consisting of:
(a) a heavy chain variable region comprising the amino acid sequence set forth in SEQ ID NO: 7, and a light chain variable region comprising the amino acid sequence set forth in SEQ ID NO: 8; or
(b) a heavy chain variable region comprising the amino acid sequence set forth in SEQ ID NO: 17, and a light chain variable region comprising the amino acid sequence set forth in SEQ ID NO: 18.

In certain embodiments, the presently disclosed subject matter provides antibodies or antigen-binding fragments thereof that comprise the heavy chain and light chain CDR1s, CDR2s and CDR3s of the presently disclosed antibodies.

The amino acid sequences of the V_{H} CDR1s of the presently disclosed antibodies are shown in SEQ ID NOs: 1 and 11. The amino acid sequences of the V_{H} CDR2s of the presently disclosed antibodies are set forth in SEQ ID NOs: 2 and 12. The amino acid sequences of the V_{H} CDR3s of the presently disclosed antibodies are set forth in SEQ ID NOs: 3 and 13.

The amino acid sequences of the V_{L} CDR1s of the presently disclosed antibodies are set forth in SEQ ID NOs: 4 and 14. The amino acid sequences of the V_{L} CDR2s of the presently disclosed antibodies are set forth in SEQ ID NOs: 5 and 15. The amino acid sequences of the V_{L} CDR3s of the presently disclosed antibodies are set forth in SEQ ID NOs: 6 and 16. The CDR regions are delineated using the IMGT system. In certain embodiments, the CDR regions are delineated using the IMGT numbering system accessible at http://www.imgt.org/IMGT_vquest/input.

Given that each of these antibodies or antigen-binding fragments thereof can bind to an antigen-binding domain (e.g., an antigen-binding domain of a CAR) and that antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and match, although each antibody must contain a V_{H} CDR1, CDR2, and CDR3 and a V_{L} CDR1, CDR2, and CDR3) to create other binding molecules. Binding of such “mixed and matched” antibodies can be tested using the binding assays described above. When V_{H} CDR sequences are mixed and matched, the CDR1, CDR2, and/or CDR3 sequence from a particular V_{H} sequence is replaced with a structurally similar CDR sequence(s). Likewise, when V_{L} CDR sequences are mixed and matched, the CDR1, CDR2, and/or CDR3 sequence from a particular V_{L} sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences of the antibodies or antigen-binding fragments thereof disclosed herein.

In certain embodiments, the presently disclosed subject matter provides an antibody or an antigen-binding fragment thereof comprising:
(a) a heavy chain variable region CDR1 comprising an amino acid sequence selected from SEQ ID NOs: 1 and 11;
(b) a heavy chain variable region CDR2 comprising an amino acid sequence selected from SEQ ID NOs: 2 and 12;
(c) a heavy chain variable region CDR3 comprising an amino acid sequence selected from SEQ ID NOs: 3 and 13;
(d) a light chain variable region CDR1 comprising an amino acid sequence selected from SEQ ID NOs: 4 and 14;
(e) a light chain variable region CDR2 comprising an amino acid sequence selected from SEQ ID Nos: 5 and 15; and
(f) a light chain variable region CDR3 comprising an amino acid sequence selected from SEQ ID NOs: 6 and 16.

In certain embodiments, the antibody or antigen-binding fragment thereof comprises:
(a) a heavy chain variable region CDR1 comprising the amino acid sequence set forth in SEQ ID NO: 1;
(b) a heavy chain variable region CDR2 comprising the amino acid sequence set forth in SEQ ID NO: 2;
(c) a heavy chain variable region CDR3 comprising the amino acid sequence set forth in SEQ ID NO: 3;
(d) a light chain variable region CDR1 comprising the amino acid sequence set forth in SEQ ID NO: 4;
(e) a light chain variable region CDR2 comprising the amino acid sequence set forth in SEQ ID NO: 5; and
(f) a light chain variable region CDR3 comprising the amino acid sequence set forth in SEQ ID NO: 6.

In certain embodiments, the antibody or antigen-binding fragment thereof comprises:
(a) a heavy chain variable region CDR1 comprising the amino acid sequence set forth in SEQ ID NO: 11;
(b) a heavy chain variable region CDR2 comprising the amino acid sequence set forth in SEQ ID NO: 12;
(c) a heavy chain variable region CDR3 comprising the amino acid sequence set forth in SEQ ID NO: 13;
(d) a light chain variable region CDR1 comprising the amino acid sequence set forth in SEQ ID NO: 14;
(e) a light chain variable region CDR2 comprising the amino acid sequence set forth in SEQ ID NO: 15; and
(f) a light chain variable region CDR3 comprising the amino acid sequence set forth in SEQ ID NO: 16.

The constant region/framework region of the antibodies or antigen-fragments thereof disclosed herein can be altered, for example, by amino acid substitution, to modify the properties of the antibody (e.g., to increase or decrease one or more of: antigen binding affinity, Fc receptor binding, antibody carbohydrate, for example, glycosylation, fucosylation, etc., the number of cysteine residues, effector cell function, effector cell function, complement function or introduction of a conjugation site).

The use of phage display libraries has made it possible to select large numbers of antibody repertoires for unique and rare Abs against very defined epitopes (for more details on phage display see McCafferty et al., Phage antibodies: filamentous phage displaying antibody variable domains. Nature, 348: 552-554.) The rapid identification of human Fab or single chain Fv (scFv) fragments highly specific for tumor antigen-derived peptide-MHC complex molecules has thus become possible. In addition, by engineering full-length monoclonal antibody (mAb) using the Fab fragments, it is possible to directly generate a therapeutic human mAb, bypassing months of time-consuming work, normally needed for developing therapeutic mAbs.

### 2.4. Homologous Antibodies

In certain embodiments, a presently disclosed antibody or antigen-binding fragment thereof comprises heavy and light chain variable regions comprising amino acid sequences that are homologous or identical to the amino acid sequences of the antibodies described herein (e.g., 19E3 and 12D11 antibodies), and wherein the antibodies or antigen-binding fragments thereof retain the desired functional properties of the antibodies or antigen-binding fragments thereof of the presently disclosed subject matter.

For example, the presently disclosed subject matter provides an antibody or an antigen-binding fragment thereof, comprising a heavy chain variable region and a light chain variable region, wherein:
(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7 and SEQ ID NO: 17;
(b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8 and SEQ ID NO: 18.

In certain embodiments, the V_{H} and/or V_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having V_{H} and V_{L} regions having high (i.e., 80% or greater) homology or identity to the V_{H} and V_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

As used herein, the percent homology between two amino acid sequences is equivalent to the percent identity between the two sequences. The percent identity or homology between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology or identity between two amino acid sequences can be determined using the algorithm of E. Meyers and W. Miller (Comput Appl Biosci (1988); 14:11-17) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J Mol Biol (1970); 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the XBLAST program (version 2.0) of Altschul et al., JMol Biol (1990); 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., Nucleic Acids Res (1997); 25(17):3389-3402. When utilizing BLAST and Gapped BLAST programs, the default parameters of the respective programs (e.g., XBLAST and NBLAST) can be used.

2.5. Antibodies with Conservative Modifications

In certain embodiments, a presently disclosed antibody or an antigen-binding fragment thereof comprises a heavy chain variable region comprising CDR1, CDR2, and CDR3 sequences and a light chain variable region comprising CDR1, CDR2, and CDR3 sequences, wherein one or more of these CDR sequences comprise specified amino acid sequences based on the preferred antibodies described herein (e.g., 19E3 and 12D11 antibodies), or a conservative modification thereof, and wherein the antibodies retain the desired functional properties of the presently disclosed subject matter. The presently disclosed subject matter provides an antibody or an antigen-binding fragment thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises an amino acid sequence selected from the amino acid sequences of SEQ ID NOs: 3 and 13, and conservative modifications thereof;
(b) the light chain variable region CDR3 sequence comprises an amino acid sequence selected from the amino acid sequence of SEQ ID NOs: 6 and 16, and conservative modifications thereof.

In certain embodiments, the heavy chain variable region CDR3 sequence comprises an amino acid sequence selected from the amino acid sequences of SEQ ID NOs: 3 and 13, and conservative modifications thereof, and the light chain variable region CDR3 sequence comprises an amino acid sequence selected from the amino acid sequences of SEQ ID NOs: 6 and 16, and conservative modifications thereof.

In certain embodiments, the heavy chain variable region CDR2 sequence comprises an amino acid sequence selected from the amino acid sequences of SEQ ID NOs: 2 and 12, and conservative modifications thereof; and the light chain variable region CDR2 sequence comprises an amino acid sequence selected from the amino acid sequences of SEQ ID NOs: 5 and 15, and conservative modifications thereof.

In certain embodiments, the heavy chain variable region CDR1 sequence comprises an amino acid sequence selected from the amino acid sequences of SEQ ID NOs: 1 and 11, and conservative modifications thereof, and the light chain variable region CDR1 sequence comprises an amino acid sequence selected from the amino acid sequences of SEQ ID NOs: 4 and 14, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions, and deletions. Modifications can be introduced into an antibody of the presently disclosed subject matter by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. Exemplary conservative amino acid substitutions are shown in Table 3. Amino acid substitutions may be introduced into an antibody of interest and the products screened for a desired activity, e.g., retained/improved antigen binding, decreased immunogenicity, or improved ADCC or CDC. In certain embodiments, a sequence disclosed herein, e.g., a CDR sequence, a V_{H} sequence or a V_{L} sequence, can have up to about one, up to about two, up to about three, up to about four, up to about five, up to about six, up to about seven, up to about eight, up to about nine or up to about ten amino acid residues that are modified and/or substituted.

TABLE 3

| | Exemplary conservative amino acid |
|---|---|
| Original Residue | Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

2.6. Antibodies that Cross-Compete for Binding to Chimer Receptor with the Presently Disclosed Antibodies

The presently disclosed subject matter provides antibodies or antigen-binding fragments thereof that cross-compete with any of the disclosed antibodies for binding to an extracellular domain of a chimeric receptor (e.g., a CAR, a HIT, etc.) or an antigen-binding domain (e.g., an antigen-binding domain of a CAR). For example, and not by way of limitation, the cross-competing antibodies can bind to the same epitope region, e.g., same epitope, adjacent epitope, or overlapping as any of the antibodies or antigen-binding fragments thereof of the presently disclosed subject matter. In certain embodiments, the reference antibody or reference antigen-binding fragments thereof for cross-competition studies can be any one of the antibodies or antigen-binding fragments thereof disclosed herein, e.g., antibodies including an scFv disclosed in Section 2.2.

Such cross-competing antibodies can be identified based on their ability to cross-compete with any one of the presently disclosed antibodies or antigen-binding fragments thereof in standard binding assays. For example, Biacore analysis, ELISA assays, or flow cytometry can be used to demonstrate cross-competition with the antibodies of the presently disclosed subject matter. The ability of a test antibody to inhibit the binding of, for example, any one of the presently disclosed antibodies (e.g., antibodies including an scFv disclosed in Section 2.2) to an antigen-binding domain (e.g., an antigen-binding domain of a CAR) demonstrates that the test antibody can compete with any one of the presently disclosed antibodies or antigen-binding fragments thereof for binding to an antigen-binding domain (e.g., an antigen-binding domain of a CAR) and thus binds to the same epitope region on an antigen-binding domain (e.g., an antigen-binding domain of a CAR) as any one of the presently disclosed antibodies or antigen-binding fragments thereof.

In certain embodiments, the cross-competing antibody or antigen-binding fragment thereof binds to the same epitope on an antigen-binding domain (e.g., an antigen-binding domain of a CAR) as any one of the presently disclosed antibodies or antigen-binding fragments thereof (e.g., antibodies including an scFv disclosed in Section 2.2).

### 2.7. Characterization of Antibody Binding to Antigen

Antibodies or antigen-binding fragments thereof of the presently disclosed subject can be tested for binding to a chimeric receptor by, for example, standard ELISA. To determine if the selected antibodies bind to unique epitopes, each antibody can be biotinylated using commercially available reagents (Pierce, Rockford, IL). Competition studies using unlabeled monoclonal antibodies and biotinylated monoclonal antibodies can be performed using chimeric receptor coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. IgGs can be further tested for reactivity with a chimeric receptor (e.g., antigen) by Western blotting.

In certain embodiments, the K_{D} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol Biol (1999); 293:865-881).

In certain embodiments, the K_{D} is measured using a BIACORE® surface plasmon resonance assay. For example, an assay using a BIACORE®-2000 or a BIACORE®-3000 (BIAcore, Inc., Piscataway, NJ)

### 2.8. Multi-Specific Molecules

The presently disclosed subject matter provides multi-specific molecules comprising a presently disclosed antibody, or a fragment thereof, disclosed herein. A presently disclosed or an antigen-binding fragment thereof can be derivatized or linked to one more functional molecules, e.g., one or more peptides or proteins (e.g., one or more antibodies or ligands for a receptor) to generate a multi-specific molecule that binds to two or more different binding sites or target molecules. The presently disclosed antibody or antigen-binding fragment thereof can be derivatized or linked to more than one other functional molecules to generate multi-specific molecules that bind to more than two different binding sites and/or target molecules. To create a multi-specific molecule, a presently disclosed antibody or an antigen-binding fragment thereof can be functionally linked (e.g., by chemical coupling, genetic fusion, noncovalent association or otherwise) to one or more other binding molecules, such as another antibody, antibody fragment, peptide or binding mimetic, such that a bispecific molecule.

In certain embodiments, the multi-specific molecule is a bispecific molecule. In certain embodiments, the bispecific molecules comprise at least a first binding specificity for a chimeric receptor and a second binding specificity for a second target epitope region. The second target epitope region can be an epitope of a chimeric receptor, or a different epitope, e.g., a different antigen. In certain embodiments, the multi-specific molecule comprises a first binding specificity for an antigen-binding domain (e.g., an antigen-binding domain of a CAR), a second binding specificity for a second target, and a third binding specificity for a third target. In certain embodiments, the second target is an antigen expressed on the surface of an immune cell (e.g., a T cell, or a human immune effector cell).

In certain embodiments, the multi-specific molecule comprises a first binding specificity for a chimeric receptor (e.g., a CAR, a HIT, etc.) and a second binding specificity for a 4-1BB polypeptide. In certain embodiments, the second binding specificity is a binding specificity of urelumab. Additional information regarding urelumab can be found, for example, in Ho et al., Molecular cancer therapeutics 19.4 (2020): 1040-1051.

In certain embodiments, the first binding specificity is a binding specificity of the 19E3 antibody and the second binding specificity is a binding specificity of urelumab. In certain embodiments, the first binding specificity is a binding specificity of the 12D11 antibody and the second binding specificity is a binding specificity of urelumab.

The multi-specific molecules of the presently disclosed subject matter can be prepared by conjugating the constituent binding specificities using methods known in the art. For example, each binding specificity of the multi-specific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the multi-specific molecule is a mAb×mAb, mAb×Fab, Fab×F(ab′)₂ or ligand x Fab fusion protein.

Binding of the multi-specific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### 2.9. Antibody Fragments

The presently disclosed subject matter provides antibody fragments of an antibody disclosed herein. In certain embodiments, the antibody fragment comprises an scFv as disclosed herein in Section 2.2. In certain embodiments, the antibody fragment is an scFv as disclosed herein in Section 2.2. In certain embodiments, the antibody fragment is a Fab fragment, a Fab′ fragment, a Fab′-SH fragment, or a F(ab′)2 fragment.

In certain embodiments, the antibody fragment is a “Fab” fragments. “Fab” fragments can be produced by papain digestion of full length antibodies. Traditionally, Fab fragments contain the heavy-chain variable domain (VH) and light-chain variable domain (VH) and also the constant domain of the light chain (CL) and the first constant domain of the heavy chain (CH1).

In certain embodiments, the antibody fragment is a “Fab′” fragments. Fab′ fragments can be distinguished from Fab fragments because including additional residues at the carboxy terminus of the CH1 domain. In certain embodiments, the Fab′ fragments include one or more cysteines from the antibody hinge region.

In certain embodiments, the antibody fragment is a “Fab′-SH” fragments. Fab′-SH are Fab′ fragments where at least one cysteine residue of the constant domains includes a free thiol group.

In certain embodiments, the antibody fragment is a “F(ab′)2” fragment. F(ab′)2 fragments can be obtained by pepsin digestion of full length antibodies. F(ab′)2 fragments have two antigen-binding sites (e.g., two Fab fragments) and a portion of the Fc region.

In certain embodiments, the antibody fragment is a single-domain antibody. Single-domain antibodies are antibody fragments including the heavy chain variable domain or a portion thereof of an antibody or the light chain variable domain or a portion thereof of an antibody.

### 2.10. Humanized or Chimeric Antibodies

The presently disclosed subject matter further provides chimeric and/or humanized versions of an antibody, or a fragment thereof, disclosed herein.

In certain embodiments, the antibody is a chimeric antibody. In certain embodiments, the chimeric antibody comprises a variable region derived from a non-human species (e.g., a variable region derived from a mouse, a rat, a hamster, a rabbit, or a non-human primate) and a human constant region. Alternatively or additionally, a chimeric antibody can be a “class-switched” antibody. In certain embodiments, the class-switched antibody is an antibody wherein the class or subclass has been modified from that of the parent antibody.

In certain embodiments, the antibody provided herein is a humanized antibody. In certain embodiments, the humanized antibody comprises at least one variable domain. In certain embodiments, the variable domain comprises CDRs derived from a non-human antibody, e.g., a mouse antibody.

In certain embodiments, the variable domain comprises framework regions (FR) derived from human antibody sequences. In certain embodiments, the FR includes substitutions and/or modifications. In certain embodiments, residues in a humanized antibody can be substituted with corresponding residues from a non-human antibody (e.g., the antibody from which the CDR residues are derived), to restore or improve antibody specificity or affinity.

In certain embodiments, the humanized antibody can also include a human constant region. In certain embodiments, humanization of a non-human antibody, e.g. a mouse antibody, reduces immunogenicity of the antibody in humans. In certain embodiments, humanization of a non-human antibody, e.g. a mouse antibody, does not impair the specificity and affinity of the parental non-human antibody.

### 2.11. Activities of Antibodies

The presently disclosed antibodies or antigen-binding fragments thereof are capable of engaging a chimeric antigen receptor (CAR) in order to induce phosphorylation of the CD3ζ intracellular domain. By “engages a CAR” is meant induction of phosphorylation of CD3 Chains cluster in response to ligand binding and immunoreceptor tyrosine-based inhibition motifs (ITAMs) upon which a signal transduction cascade is produced. In certain embodiments, the engages a CAR refers to induction of phosphorylation of the ITAM1 domain of a CD3ζ polypeptide. In certain embodiments, the presently disclosed antibodies or antigen-binding fragments thereof are capable of engaging any CAR including an scFv targeting any antigen (e.g., tumor antigen).

In certain embodiments, the presently disclosed antibodies or antigen-binding fragments thereof are capable of activating an immunoresponsive cell expressing a CAR. By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane-bound signaling molecules allows for ITAM motifs contained within the CD3 chains to become phosphorylated. This phosphorylation in turn initiates a T cell activation pathway ultimately activating transcription factors, such as NF-kB and AP-1. These transcription factors induce global gene expression of the T cell to increase IL-2 production for proliferation and expression of master regulator T cell proteins in order to initiate a T cell mediated immune response. In certain embodiments, the presently disclosed antibodies or antigen-binding fragments thereof are capable of activating an immunoresponsive cell expressing a CAR including an scFv targeting any antigen (e.g., tumor antigen).

In certain embodiments, the presently disclosed antibodies or antigen-binding fragments thereof are capable of stimulating an immunoresponsive cell expressing a CAR. By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40, ICOS, DAP-10, CD27, NKG2D, CD2, CD150, CD226. Receiving multiple stimulatory signals can be important to mount a robust and long-term T cell mediated immune response. T cells can quickly become inhibited and unresponsive to antigens. While the effects of these co-stimulatory signals may vary, they generally result in increased gene expression in order to generate long lived, proliferative, and anti-apoptotic T cells that robustly respond to antigens for complete and sustained eradication. In certain embodiments, the presently disclosed antibodies or antigen-binding fragments thereof are capable of stimulating an immunoresponsive cell expressing a CAR including an scFv targeting any antigen (e.g., tumor antigen).

The presently disclosed antibodies or antigen-binding fragments thereof are capable of engaging a TCR like fusion molecule (HIT) in order to induce phosphorylation of the endogenous CD3ζ. By “engages a HIT” is meant induction of phosphorylation of endogenous CD3 Chains cluster in response to ligand binding and immunoreceptor tyrosine-based inhibition motifs (ITAMs) upon which a signal transduction cascade is produced. In certain embodiments, the presently disclosed antibodies or antigen-binding fragments thereof are capable of engaging any HIT including a first antigen-binding chain and a second antigen-binding chain targeting any antigen (e.g., tumor antigen).

Additionally, in certain embodiments, the presently disclosed antibodies or antigen-binding fragments thereof are capable of activating an immunoresponsive cell expressing a HIT. In certain embodiments, the presently disclosed antibodies or antigen-binding fragments thereof are capable of activating an immunoresponsive cell expressing a HIT including a first antigen-binding chain and a second antigen-binding chain targeting any antigen (e.g., tumor antigen).

In certain embodiments, the presently disclosed antibodies or antigen-binding fragments thereof are capable of stimulating an immunoresponsive cell expressing a HIT. In certain embodiments, the presently disclosed antibodies or antigen-binding fragments thereof are capable of stimulating an immunoresponsive cell expressing a HIT including a first antigen-binding chain and a second antigen-binding chain targeting any antigen (e.g., tumor antigen).

### 3. Nucleic Acids encoding the Antibodies or Antigen-binding Fragments

The presently disclosed subject matter provides nucleic acids encoding the antibodies or antigen-binding fragments thereof disclosed herein (e.g., antibodies including an scFv disclosed in Section 2.2).

Further provided are vectors comprising the presently disclosed nucleic acids. In certain embodiments, the vector is an expression vector. The presently disclosed subject matter further provides host cells comprising the vectors disclosed herein. In certain embodiments, the host cells are T cells.

### 4. Pharmaceutical Compositions

The presently disclosed subject matter provides compositions comprising a presently disclosed antibody or an antigen-binding fragment thereof, a presently disclosed immunoconjugate, or a presently disclosed multi-specific molecule. In certain embodiments, the composition is a pharmaceutical composition further comprising a pharmaceutically acceptable carrier.

The antibodies or antigen-binding fragments thereof of the presently disclosed subject matter can be present in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof.

Pharmaceutically acceptable carriers may further comprise minor amounts of auxiliary substances such as wetting or emulsifying agents, preservatives or buffers, which enhance the shelf life or effectiveness of the binding proteins. The compositions can, as is well known in the art, be formulated so as to provide quick, sustained or delayed release of the active ingredient.

The terms “pharmaceutically acceptable,” “physiologically tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. The term “pharmaceutically acceptable salts and esters” means salts and esters that are pharmaceutically acceptable and have the desired pharmacological properties.

Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include sters formed from carboxy, sulfonyloxy, and phosphonoxy groups present in the presently disclosed antibody, e.g., C1-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. An anti-idiotype antibody named in this technology can be present in unsalified or unesterified form, or in salified and/or esterified form, and the naming of such presently disclosed antibody is intended to include both the original (unsalified and unesterified) compound and its pharmaceutically-acceptable salts and esters.

The antibodies of the present technology can be present in the form of a depot injection or implant preparation which can be formulated in such a manner as to permit a sustained or pulsatile release of the active ingredient.

In certain embodiments, the presently disclosed antibodies are prepared with carriers that protect against degradation. Biodegradable, biocompatible polymers can be used, such as ethylene vinyl acetate, polyanhydrides, polyglycolic acid, collagen, polyorthoesters, and polylactic acid.

Methods for preparation of such formulations will be apparent to those skilled in the art.

### 5. Diagnostic and Prognostic Methods

The presently disclosed antibodies and antigen-binding fragments thereof can be used for diagnostic and prognostic applications as well as used as quality control tools for the manufacture of cell lines (e.g., T cells comprising a CAR, TCR, HIT, etc.). The presently disclosed subject matter provides methods for detecting an idiotope (e.g., an antigen-binding domain of a CAR, TCR, HIT, etc.) in a biological sample (e.g., a cell, a tissue, a blood sample, a cell culture, an iPS culture). Additionally or alternatively, the presently disclosed subject matter provides methods for detecting chimeric receptor (e.g., an extracellular domain of a CAR, HIT, etc.) in a biological sample (e.g., a cell, a tissue, a blood sample, a cell culture, an iPS culture).

In certain embodiments, the method comprises: contacting a cell, a tissue, or a blood sample with an antibody or antigen-binding fragment thereof disclosed herein, wherein the antibody or antigen-binding fragment thereof comprises a detectable label; and determining the amount of the antibody or antigen-binding fragment thereof bound to the cell, tissue, or blood sample by measuring the amount of detectable label associated with the cell or tissue, wherein the amount of bound antibody or antigen-binding fragment thereof indicates the amount of idiotope/idiotype (e.g., CAR, TCR, HIT, etc.) in the cell, tissue, or blood sample. In certain embodiments, the method comprises: contacting a cell, a tissue, or a blood sample with an antibody or antigen-binding fragment thereof disclosed herein, wherein the antibody or antigen-binding fragment thereof comprises a detectable label; and determining the amount of the antibody or antigen-binding fragment thereof bound to the cell, tissue, or blood sample by measuring the amount of detectable label associated with the cell or tissue, wherein the amount of bound antibody or antigen-binding fragment thereof indicates the amount of chimeric receptor (e.g., CAR, HIT, etc.) in the cell, tissue, or blood sample.

The cell or tissue can be any cell or tissue, including any normal, healthy, or cancerous cells and tissues, or a cell in culture. In certain embodiments, the blood sample is a peripheral blood sample. In certain embodiments, the cell culture is used for the manufacture of CAR T cells. In certain embodiments, the cell is an immunoresponsive cell (e.g., T cell, NK cell, B cell, stem cell from which lymphoid cells may be differentiated) expressing a CAR or TCR.

The presently disclosed anti-idiotype antibodies and antigen-binding fragments thereof can be used in methods known in the art relating to the localization and/or quantitation of idiotopes (e.g., for use in measuring expression levels of a CAR within genetically engineered T cells, for use in measuring the persistence of CAR T cells in patient samples, for use in diagnostic methods, and the like). The presently disclosed anti-idiotype antibodies and antigen-binding fragments thereof can be used to isolate an idiotype/idiotope complex by standard techniques, such as affinity chromatography or immunoprecipitation. The presently disclosed anti-idiotype antibodies and antigen-binding fragments thereof can facilitate the purification of cells comprising an idiotope (e.g., T cells expressing a CAR) from biological samples including in vitro cell cultures. The presently disclosed anti-idiotype antibodies and antigen-binding fragments thereof can be used diagnostically to monitor idiotope levels (e.g., CAR, TCR, HIT, etc.) in tissue as part of a clinical testing procedure, e.g., to determine the efficacy of a given treatment regimen. As noted above, the detection can be facilitated by coupling (i.e., physically linking) the presently disclosed antibodies and antigen-binding fragments to a detectable substance.

Additionally or alternatively, the presently disclosed antibodies and antigen-binding fragments thereof can be used in methods known in the art relating to the localization and/or quantitation of chimeric receptors (e.g., for use in measuring expression levels of a CAR or HIT within genetically engineered T cells, for use in measuring the persistence of CAR T cells in patient samples, for use in diagnostic methods, and the like). The presently disclosed antibodies and antigen-binding fragments thereof can be used to isolate a chimeric receptor (e.g., CAR, HIT, etc.) by standard techniques, such as affinity chromatography or immunoprecipitation. The presently disclosed antibodies and antigen-binding fragments thereof can facilitate the purification of cells comprising a chimeric receptor (e.g., T cells expressing a CAR or T cells expressing a HIT) from biological samples including in vitro cell cultures. The presently disclosed antibodies and antigen-binding fragments thereof can be used diagnostically to monitor levels of cells expressing a chimeric receptor (e.g., CAR, HIT, etc.) in tissue as part of a clinical testing procedure, e.g., to determine the efficacy of a given treatment regimen. As noted above, the detection can be facilitated by coupling (i.e., physically linking) the presently disclosed antibodies and antigen-binding fragments to a detectable substance.

An exemplary method for detecting the presence or absence of an idiotope or a chimeric receptor in a biological sample comprises contacting a biological sample from a subject with a presently disclosed antibody or antigen-binding fragment thereof, wherein the presence of an idiotope or a chimeric receptor (e.g., CAR, TCR, HIT, etc.) is detected in the biological sample.

Detection may be accomplished by means of a detectable label attached to the antibody.

The term “labeled,” with regard to the anti-idiotype antibody or antigen-binding fragment thereof and anti-chimeric receptor antibody or antigen-binding fragment thereof, is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In certain embodiments, the presently disclosed anti-idiotype antibodies and antigen-binding fragments thereof are conjugated to one or more detectable labels. In certain embodiments, the presently disclosed anti-chimeric receptor antibodies and antigen-binding fragments thereof are conjugated to one or more detectable labels. For such uses, the presently disclosed antibodies and antigen-binding fragments thereof may be detectably labeled by covalent or non-covalent attachment of a chromogenic, enzymatic, radioisotopic, isotopic, fluorescent, toxic, chemiluminescent, nuclear magnetic resonance contrast agent or other label.

The presently disclosed detection methods can be used to detect an idiotope (e.g., antigen-binding domain of a CAR, HIT, etc.) in a biological sample in vitro as well as in vivo. Additionally or alternatively, the presently disclosed detection methods can be used to detect a chimeric receptor (e.g., CAR, HIT, etc.) in a biological sample in vitro as well as in vivo. Non-limiting examples of in vitro techniques for detection of an idiotope (e.g., antigen-binding domain of a CAR, TCR, HIT, etc.) or a chimeric receptor (e.g., CAR, HIT) include enzyme linked immunosorbent assays (ELISAs), Western blots, immunoprecipitations, radioimmunoassay, and immunofluorescence.

Furthermore, in vivo techniques for detection of an idiotope or a chimeric receptor include introducing into a subject a labeled anti-idiotype antibody or antigen-binding fragment thereof or an anti-chimeric receptor antibody or antigen-binding fragment thereof. For example, the anti-idiotype antibody or antigen-binding fragment thereof can be labeled with a radioactive marker whose presence and location in a subject can be detected by standard imaging techniques. In certain embodiments, the biological sample comprises cells expressing the idiotope or the chimeric receptor (e.g., CAR T cells) from the test subject.

In certain embodiments, the presently disclosed anti-idiotype antibodies and antigen-binding fragments thereof may be used for in vivo imaging of an idiotope (e.g., CAR T cells infused into a patient). In certain embodiments, the presently disclosed antibodies and antigen-binding fragments thereof may be used for in vivo imaging of a chimeric receptor (e.g., CAR T cells infused into a patient). Antibodies useful for this method include those detectable by X-radiography, NMR or ESR. For X-radiography, suitable labels include radioisotopes such as barium or cesium, which emit detectable radiation but are not overtly harmful to the subject.

Suitable markers for NMR and ESR include those with a detectable characteristic spin, such as deuterium, which can be incorporated into the anti-idiotype antibodies by labeling of nutrients for the relevant scFv clone.

The presently disclosed anti-idiotype or anti-chimeric receptor antibodies or antigen-binding fragments thereof, which are labeled with an appropriate detectable imaging moiety (such as a radioisotope (e.g., ¹³¹I, ¹¹¹I, ⁹⁹mTc, ¹⁸F, ⁸⁹Zr), a radio-opaque substance, or a material detectable by nuclear magnetic resonance) are introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled anti-idiotype or anti-chimeric receptor antibody or antigen-binding fragment thereof then accumulates at the location of cells which contain the specific target polypeptide (e.g., idiotope).

For example, the labeled anti-idiotype or anti-chimeric receptor antibody or antigen-binding fragment thereof accumulates within the subject in cells and tissues in which the idiotope has localized (e.g., infused CAR T cells comprising the idiotope and to cancer cells or tissues expressing the target antigen of the CAR).

Thus, the presently disclosed subject matter provides diagnostic and prognostic methods related to immunotherapy (e.g., T cell immunotherapy). In certain embodiments, the method comprises: (a) assaying the expression of an idiotope protein, e.g., antigen-binding domain of a CAR (e.g., by measuring binding of a presently disclosed labeled anti-idiotype antibody or an antigen-binding fragment thereof in cells or body fluid of an individual at a first and second time point; and (b) comparing the amount of idiotope protein present at the first and second time point, wherein an increase or decrease in the idiotope protein level between the first and second time point is indicative of the efficacy of a given treatment regimen (e.g., persistence CAR T cells).

In certain embodiments, the method comprises: (a) assaying the expression of a chimeric receptor (e.g., CAR, HIT, etc.) (e.g., by measuring binding of a presently disclosed labeled antibody or an antigen-binding fragment thereof in cells or body fluid of an individual at a first and second time point); and (b) comparing the amount of chimeric receptor protein present at the first and second time point, wherein an increase or decrease in the chimeric receptor protein level between the first and second time point is indicative of the efficacy of a given treatment regimen (e.g., persistence CAR T cells).

In certain embodiments, the presently disclosed anti-idiotype or anti-chimeric receptor antibody or antigen-binding fragment thereof is conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells comprising the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). In certain embodiments, the diagnostic agents are selected from the group consisting of radioisotopes, enhancing agents for use in magnetic resonance imaging, and fluorescent compounds. Chelates may be coupled to the presently disclosed antibodies or antigen-binding fragments thereof using standard chemistries. The chelate is normally linked to the antibody by a group which enables formation of a bond to the molecule with minimal loss of immunoreactivity and minimal aggregation and/or internal cross-linking.

### 6. Kits

The presently disclosed subject matter provides kits comprising the antibodies or antigen-binding fragments thereof the multi-specific molecule, or the composition disclosed herein. In certain embodiments, the kit comprises a sterile container that contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for the use of the antibodies or antigen-binding fragments thereof the multi-specific molecule, or the composition disclosed herein. The instructions can generally include information about the use of the antibodies or antigen-binding fragments thereof the multi-specific molecule, and the composition disclosed herein for differentiation of pluripotent stem cells into T cells. In certain embodiments, the instructions include at least one of the following: description of the antibodies, protocols, precautions, warnings, animal pharmacology, sequences, and/or references. The instructions may be printed directly on the container (when present), or as a label applied to the container, or as a separate sheet, pamphlet, card, or folder supplied in or with the container.

### 7. Exemplary Embodiments

A1. In certain non-limiting embodiments, the presently disclosed subject matter provides an antibody or an antigen-binding fragment thereof, comprising a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7 or SEQ ID NO: 17.

A2. In certain non-limiting embodiments, the presently disclosed subject matter provides an antibody or an antigen-binding fragment thereof, comprising a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8 or SEQ ID NO: 18.

A3. In certain non-limiting embodiments, the presently disclosed subject matter provides an antibody or an antigen-binding fragment thereof, comprising:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7 or SEQ ID NO: 17; and
b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 8 or SEQ ID NO: 18.

A4. In certain non-limiting embodiments, the presently disclosed subject matter provides an antibody or an antigen-binding fragment thereof, comprising a heavy chain variable region and a light chain variable region, wherein the heavy chain variable region and the light chain variable region are selected from the group consisting of:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO:7, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8; and
b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 17, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 18.

A5. In certain non-limiting embodiments, the presently disclosed subject matter provides an antibody or an antigen-binding fragment thereof, comprising a heavy chain variable region comprising the amino acid sequence set forth in SEQ ID NO: 7 or SEQ ID NO: 17.

A6. In certain non-limiting embodiments, the presently disclosed subject matter provides an antibody or an antigen-binding fragment thereof, comprising a light chain variable region comprising the amino acid sequence set forth in SEQ ID NO: 8 or SEQ ID NO: 18.

A7. In certain non-limiting embodiments, the presently disclosed subject matter provides an antibody or an antigen-binding fragment thereof, comprising
a) a heavy chain variable region comprising the amino acid sequence set forth in SEQ ID NO: 7 or SEQ ID NO: 17; and
b) a light chain variable region comprising the amino acid sequence set forth in SEQ ID NO: 8 or SEQ ID NO: 18.

A8. The foregoing antibody or antigen-binding fragment thereof of any one of A1-A7, wherein
a) the heavy chain variable region comprises the amino acid sequence set forth in SEQ ID NO: 7, and the light chain variable region comprises the amino acid sequence set forth in SEQ ID NO: 8; or
b) the heavy chain variable region comprises the amino acid sequence set forth in SEQ ID NO: 17, and the light chain variable region comprises the amino acid sequence set forth in SEQ ID NO: 18.

A9. In certain non-limiting embodiments, the presently disclosed subject matter provides an antibody or an antigen-binding fragment thereof, comprising a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
a) a heavy chain variable region CDR3 comprising the amino acid sequence set forth in SEQ ID NO: 3 and a conservative modification thereof, and a light chain variable region CDR3 comprising the amino acid sequence set forth in SEQ ID NO: 6 and a conservative modification thereof; and
b) a heavy chain variable region CDR3 comprising the amino acid sequence set forth in SEQ ID NO: 13 and a conservative modification thereof, and a light chain variable region CDR3 comprising the amino acid sequence set forth in SEQ ID NO: 16 and a conservative modification thereof.

A10. The foregoing antibody or antigen-binding fragment thereof of A9, wherein the heavy chain variable region and light chain variable region CDR2 domains are selected from the group consisting of:
a) a heavy chain variable region CDR2 comprising the amino acid sequence set forth in SEQ ID NO: 2 and a conservative modification thereof, and a light chain variable region CDR2 comprising the amino acid sequence set forth in SEQ ID NO: 5 and a conservative modification thereof; and
b) a heavy chain variable region CDR2 comprising the amino acid sequence set forth in SEQ ID NO: 12 and a conservative modification thereof, and a light chain variable region CDR2 comprising the amino acid sequence set forth in SEQ ID NO: 15 and a conservative modification thereof.

All. The foregoing antibody or antigen-binding fragment thereof of A9 or A10, wherein the heavy chain variable region and light chain variable region CDR1 domains are selected from the group consisting of:
a) a heavy chain variable region CDR1 comprising the amino acid sequence set forth in SEQ ID NO: 1 and a conservative modification thereof, and a light chain variable region CDR1 comprising the amino acid sequence set forth in SEQ ID NO: 4 and a conservative modification thereof; and
b) a heavy chain variable region CDR1 comprising the amino acid sequence set forth in SEQ ID NO: 11 and a conservative modification thereof, and a light chain variable region CDR1 comprising the amino acid sequence set forth in SEQ ID NO: 14 and a conservative modification thereof.

A12. The foregoing antibody or antigen-binding fragment thereof of any one of A9-A11, wherein one or more of the CDR sequences have up to about 5 amino acid substitutions.

A13. The foregoing antibody or antigen-binding fragment thereof of any one of A9-A11, wherein one or more of the CDR sequences have up to about 3 amino acid substitutions.

A14. In certain non-limiting embodiments, the presently disclosed subject matter provides an antibody or an antigen-binding fragment thereof, comprising:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; or
b) a heavy chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13.

A15. In certain non-limiting embodiments, the presently disclosed subject matter provides an antibody or an antigen-binding fragment thereof, comprising:
a) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
b) a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

A16. In certain non-limiting embodiments, the presently disclosed subject matter provides an antibody or an antigen-binding fragment thereof, comprising:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

A17. In certain non-limiting embodiments, the presently disclosed subject matter provides an antibody or an antigen-binding fragment thereof comprising a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

A18. In certain non-limiting embodiments, the presently disclosed subject matter provides an antibody or an antigen-binding fragment thereof comprising a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

A19. The foregoing antibody or antigen-binding fragment thereof of any one of A1-A18, wherein the antibody or antigen-binding fragment thereof is an anti-idiotype antibody.

A20. The foregoing antibody or antigen-binding fragment thereof of any one of A1-A19, wherein the antibody comprises a human variable region framework region.

A21. The foregoing antibody or antigen-binding fragment thereof of any one of A1-A20, which is a fully human or an antigen-binding fragment thereof.

A22. The foregoing antibody or antigen-binding fragment thereof of any one of A1-A20, which is a chimeric antibody or an antigen-binding fragment thereof.

A23. The foregoing antibody or antigen-binding fragment thereof of any one of A1-A20, which is a humanized antibody or an antigen-binding fragment thereof.

A24. The foregoing antibody or antigen-binding fragment thereof of any one of A1-A23, wherein the antigen-binding fragment is a Fab, Fab′, F(ab′)2, variable fragment (Fv), or single chain variable region (scFv).

A25. The foregoing antibody or antigen-binding fragment thereof of A24, wherein the antigen antigen-binding fragment is an scFv.

B1. In certain non-limiting embodiments, the presently disclosed subject matter provides an antibody or an antigen-binding fragment thereof, which cross-competes for binding to the antibody or an antigen-binding fragment thereof of any one of A1-A25.

B2. In certain non-limiting embodiments, the presently disclosed subject matter provides an antibody or an antigen-binding fragment thereof, which binds to the same epitope region of an antibody or an antigen-binding fragment thereof of any one of A1-A25.

C1. In certain non-limiting embodiments, the presently disclosed subject matter provides a composition comprising the antibody or antigen-binding fragment thereof of any one of A1-A25 or B1-B2.

C2. The foregoing composition of C1, which is a pharmaceutical composition that further comprises a pharmaceutically acceptable carrier.

D1. In certain non-limiting embodiments, the presently disclosed subject matter provides a multi-specific molecule comprising the antibody or antigen-binding fragment thereof of any one of claims 1-27, linked to one or more functional moieties.

D2. The foregoing multi-specific molecule of D1, wherein the one or more functional moieties have a different binding specificity than the antibody or antigen binding fragment thereof.

E1. In certain non-limiting embodiments, the presently disclosed subject matter provides a composition comprising the multi-specific molecule of D1 or D2.

F1. In certain non-limiting embodiments, the presently disclosed subject matter provides a kit comprising the antibody or antigen-binding fragment thereof of any one of A1-A25 or B1-B2, the multi-specific molecule of D1 or D2, or the composition of any one of claims C1, C2, or E1.

F2. The foregoing kit of F1, wherein the kit further comprises written instructions for using the antibody or antigen-binding fragment thereof, the multi-specific molecule, or the composition.

### EXAMPLES

The following examples are put forth so as to provide those of ordinary skill in the art with a complete disclosure and description of how to make and use the antibodies, multi-specific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### Example 1

T cells that are engineered to express a chimeric antigen receptor (CAR) can direct potent therapeutic responses in patients with chemorefractory hematologic malignancies (June & Sadelain, N Engl J Med 379, 64-73 (2018)). CARs are synthetic receptors that redirect T cell specificity and augment T cell functions to overcome tumor resistance. CAR T cells are now under investigation in a range of diseases, including solid tumors, infectious disease, autoimmunity and senescence-associated pathologies. CAR T cells are generally produced in autologous fashion, which is effective but presents some challenges. Manufacturing time is critical for patients with rapidly progressing disease; cell product variability is high because patient T cells may be reduced in number or functionality due to progressive disease or prior therapies; manufacturing processes and release testing are required on an individual basis and costly. Alternate T cell sources are under investigation to enable ‘off’-the-shelf cellular therapy, including donor-derived lymphoid progenitors, T cells lacking allo-reactive potential (e.g., virus-specific T cells, γδTCR-T cells, invariant NKT cells and T cell receptor (TCR)-edited T cells) and pluripotent stem cell-derived T cells (Themeli et al., Cell Stem Cell 16, 357-366 (2015); Depil et al., Nat Rev Drug Discov 19, 185-199 (2020)). The use of allogeneic T cells harvested from healthy donors is the most explored alternative source, with some promising clinical results already obtained (Benjamin, et al., Lancet 396, 1885-1894 (2020)). However, maintaining product consistency after genetic engineering and manufacturing large cell batches of sufficient cell purity to avert graft versus host disease (GvHD) remain a challenge (Qasim et al., Sci Transl Med 9 (2017)). Pluripotent stem cells provide an attractive solution to overcome challenges associated with the use of autologous or allogeneic blood cells. Their self-renewing capacity should facilitate the selection of clones of a desired genotype, eventually combining multiple edits to enhance anti-tumor functions and pre-empt allo-reactivity and allo-rejection, and support large-scale production (Themeli et al., Nat Biotechnol 31, 928-933 443 (2013); Wang et al., Nat Biomed Eng 5, 429-440 (2021)).

It was previously demonstrated that CAR T cells can be generated from reprogrammed TiPS (Themeli et al., Nat Biotechnol 31, 928-933 (2013)). These original TiPS were retrovirally transduced to express a CD19-specific CAR (CAR-TiPS), and re-differentiated into T cells. The CAR T cells induced therefrom (CAR-TiPS-iT) homogeneously expressed both the transduced CAR (1928z) and the endogenous αβTCR. The CAR-TiPS-iT cells were specific for CD19, highly lytic and controlled tumor growth in an intra-peritoneal lymphoma model in NSG mice (Themeli et al., Nat Biotechnol 31, 928-933 (2013)). Despite expressing their endogenous αβTCR, these CAR-TiPS-iT cells did not acquire a conventional CD4 or CD8αβ T cell phenotype, but rather an innate-like CD8αβ-double-negative (DN) or CD8αα single-positive (SP) phenotype.

Transcriptional analyses confirmed that these CAR-TiPS-iT cells more closely resembled γδ-T cells than αβ-T cells (Themeli et al., Nat Biotechnol 31, 928-933 (2013)). These findings were recently corroborated by Harada et al., who found that constitutive expression of a LMP2-specific CAR in TiPS likewise yielded DN and CD8α T cell phenotypes (Harada et al., Mol Ther (2021)). In another report, the lymphoid differentiation of induced pluripotent stem cells expressing a GPC3-specific CAR failed to induce characteristic T cell markers such as CD5 and CD3 and rather produced natural killer (NK) or innate lymphoid cells (ILC) (Ueda et al., Cancer Sci 111, 1478-1490 (2020)). These reports raise the questions of why T lineage commitment is deferred towards innate phenotypes and what is required to induce CD8αβ CAR T cells. Here, the impact of TCR and CAR expression on the commitment of TiPS-derived lymphoid progenitors to adaptive T cell lineages is analyzed.

Physiological ap TCR-T cell development is regulated by TCR gene recombination, Notch and pre-TCR/TCR signaling (Yui & Rothenberg, Nat Rev Immunol 14, 529-545 (2014)). The TCR and Notch are central to the adoption of either γδTCR or αβTCR-T cell fates. The earlier rearrangement of the γ- and δ-chains results in maturation of γδTCR-T cells with a DN or CD8αα SP phenotype (Pardoll et al., J Immunol 140, 4091-4096 (1988)), whereas successful pre-TCR and αβTCR assembly drive progression from the DN to the CD4⁺CD8⁺ double positive (DP) stage before yielding SP T cells (Fehling et al., Nature 375, 795-798 (1995); Hogquist et al., J Exp Med 177, 1469-1473 (1993)). The presently disclosed subject matter shows that premature αβTCR or constitutive CAR expression interfere with DP formation, depending on the strength of Notch stimulation. Delaying CAR expression through TRAC promoter-controlled expression and calibration of CAR signaling through CD3ζ immunoreceptor tyrosine activation motif (ITAM) mutations enable DP CART iT cell development. In absence of an αβTCR, the CAR drives T cell maturation and yields CD8αβ⁺ CAR⁺ iT cells that mediate durable remissions in a systemic leukemia model.

### 1. DLL4 Stimulation Facilitates CD4⁺CD8αβ⁺ DP T Cell Development from WT-TiPS but not CAR-TiPS

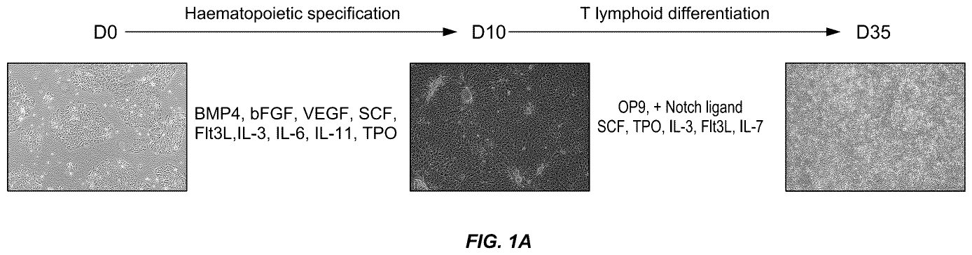

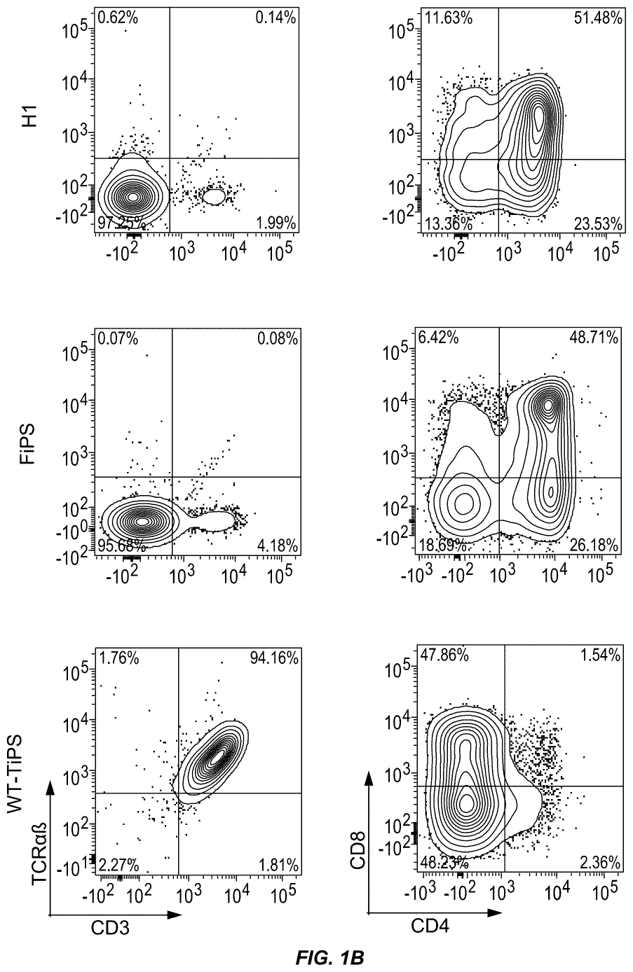

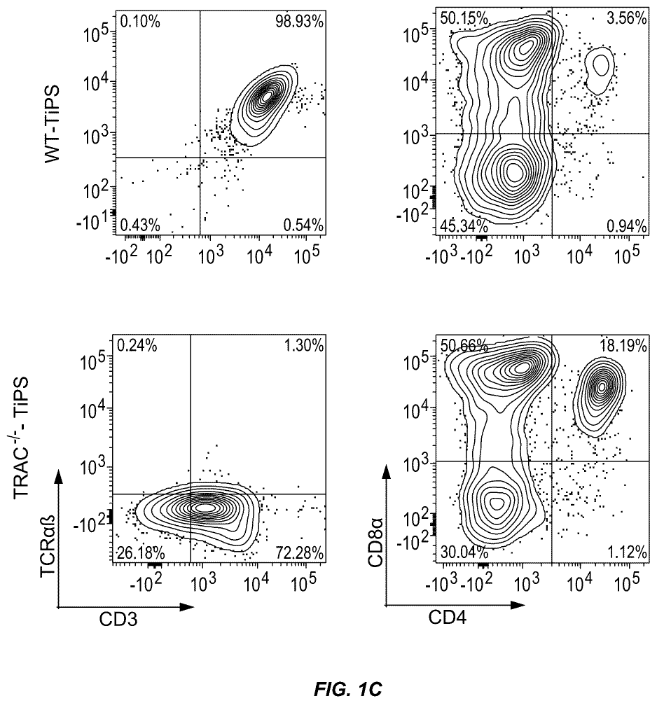

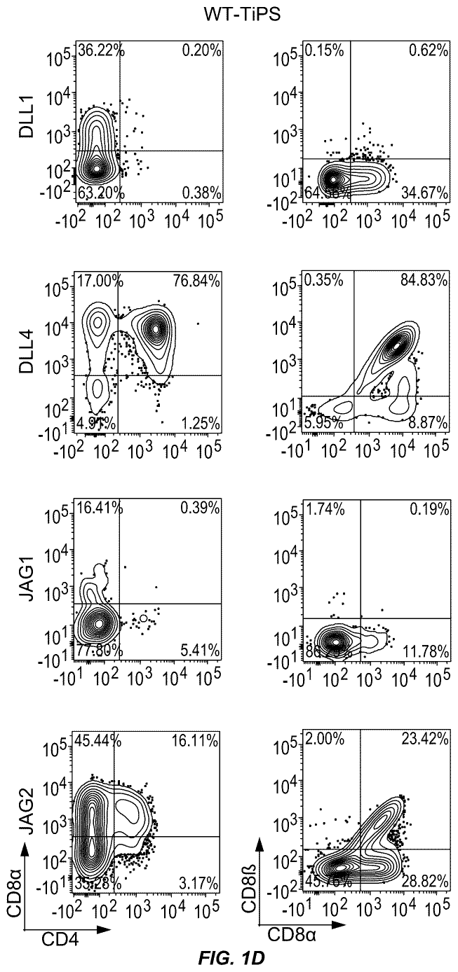

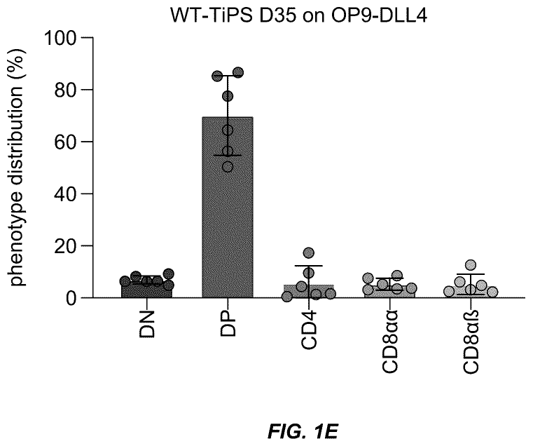

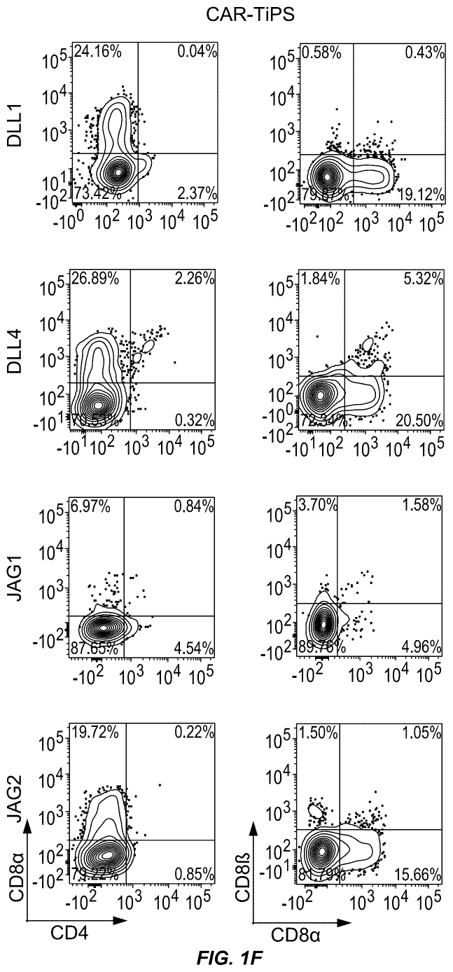

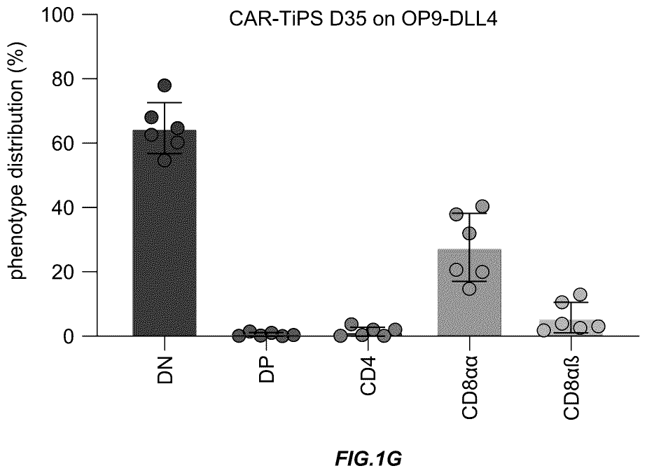

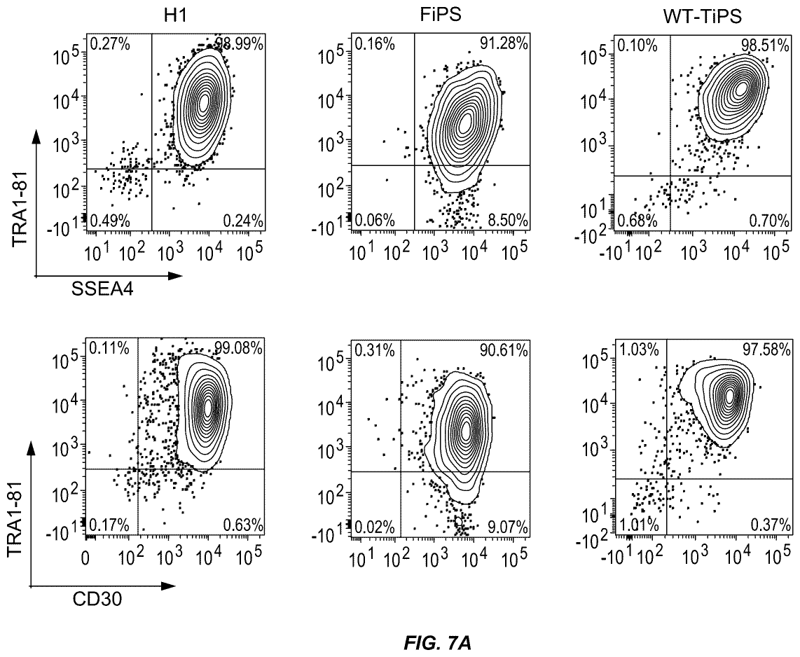

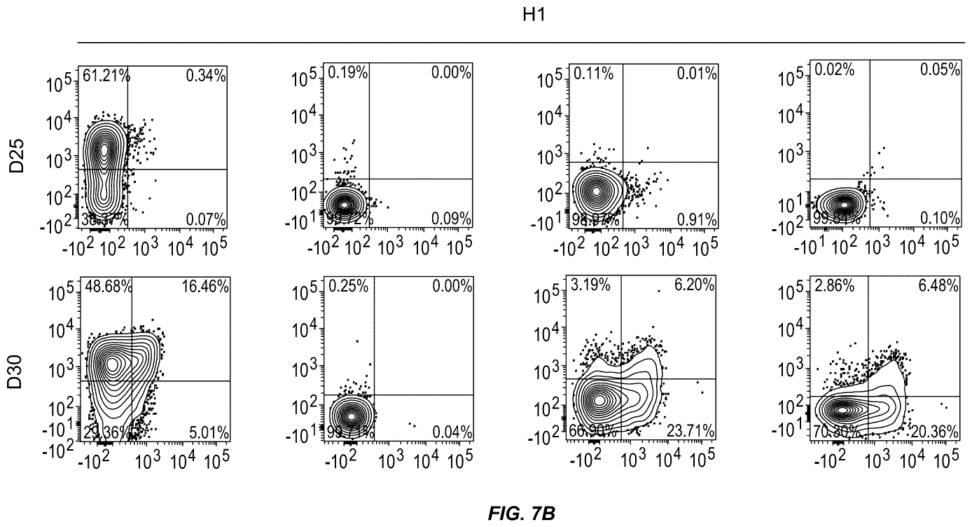

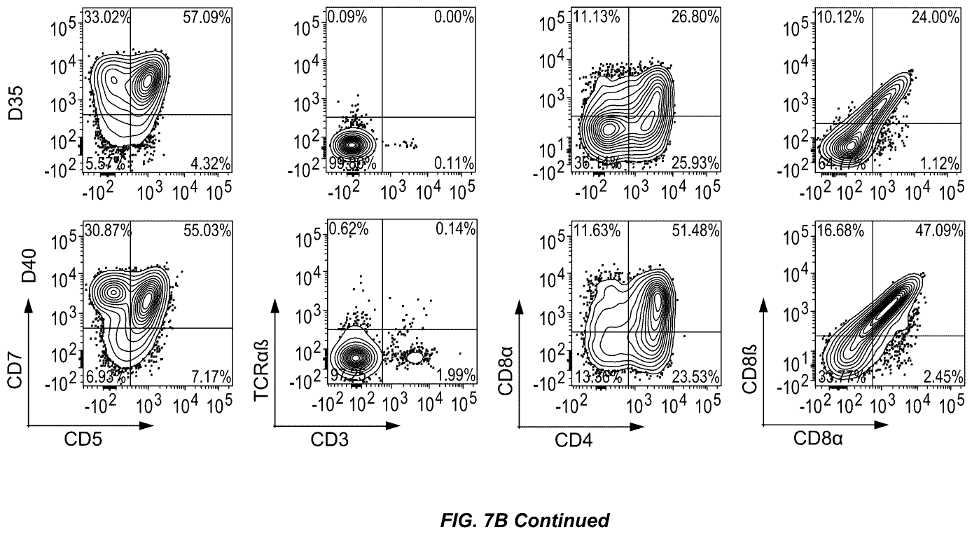

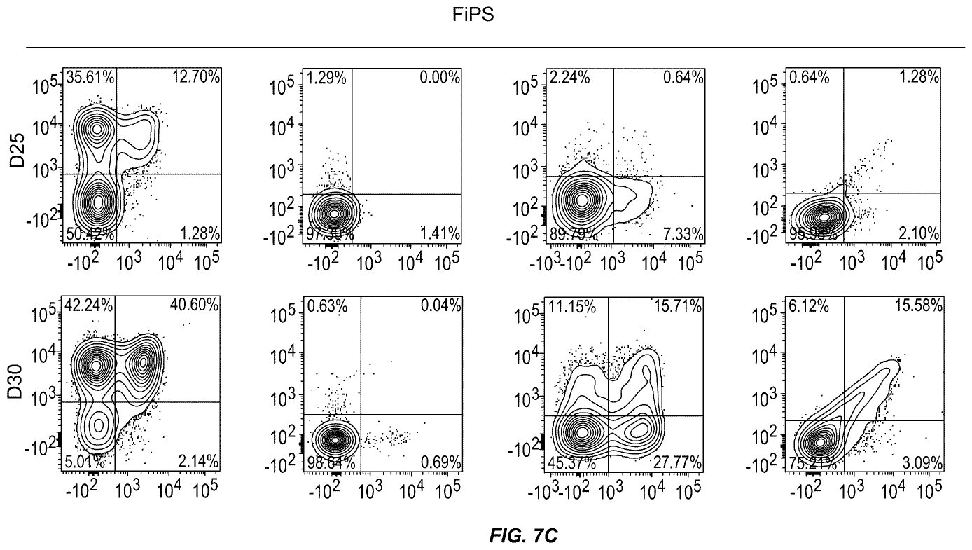

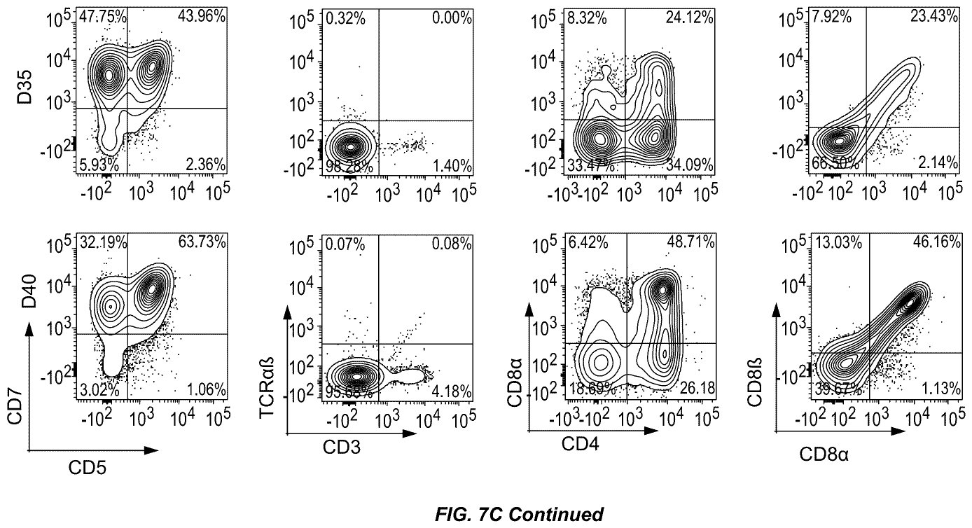

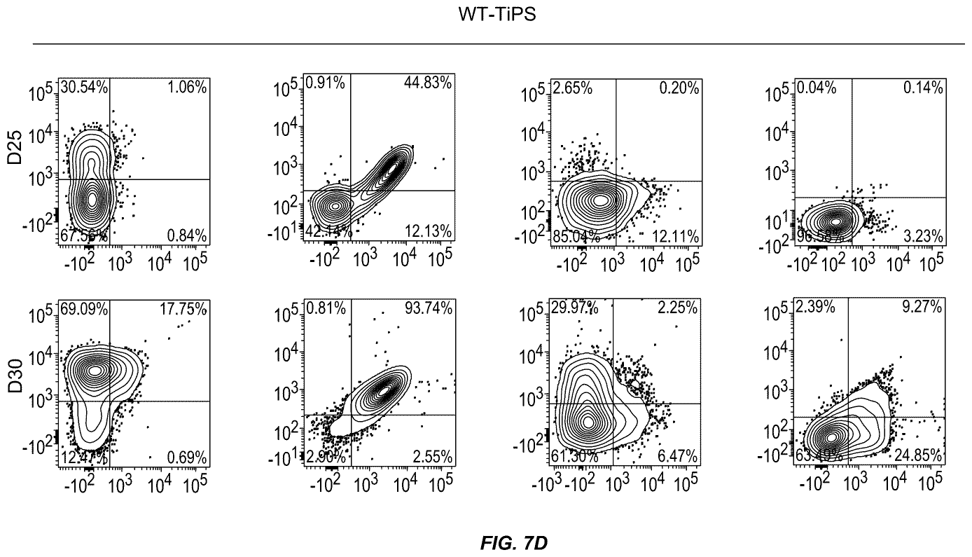

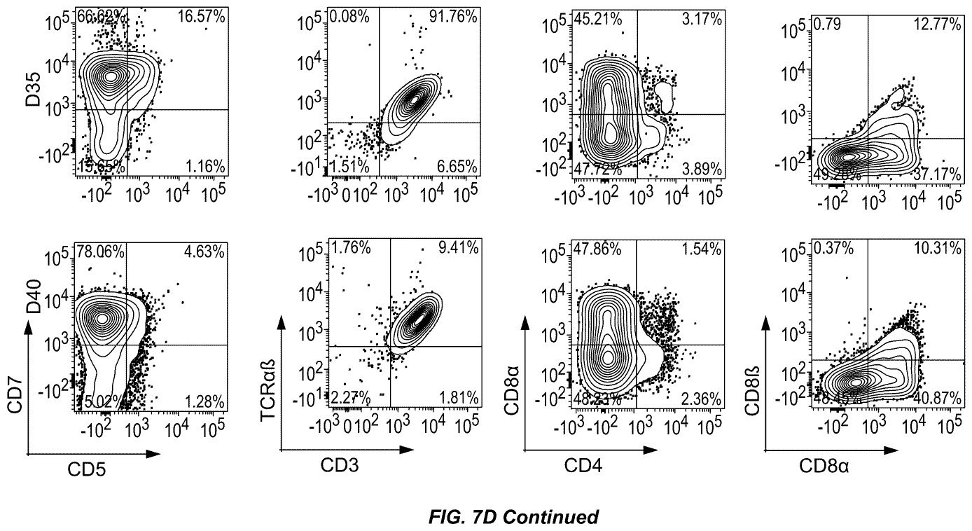

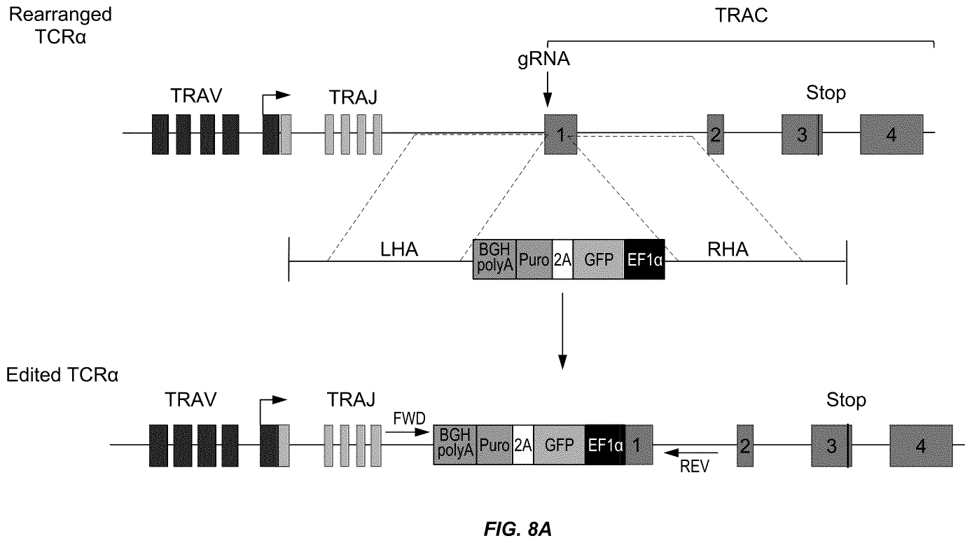

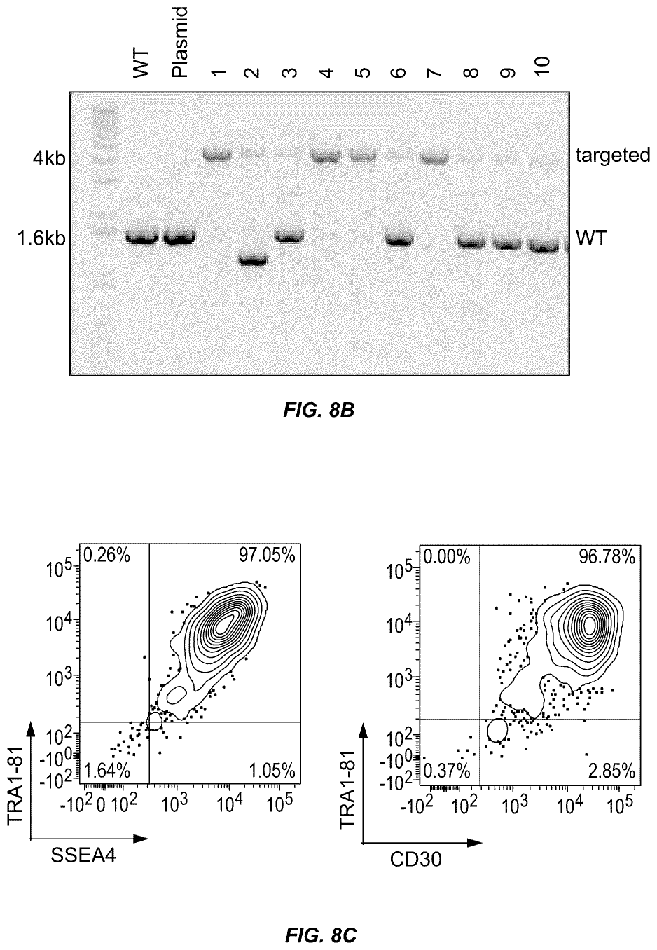

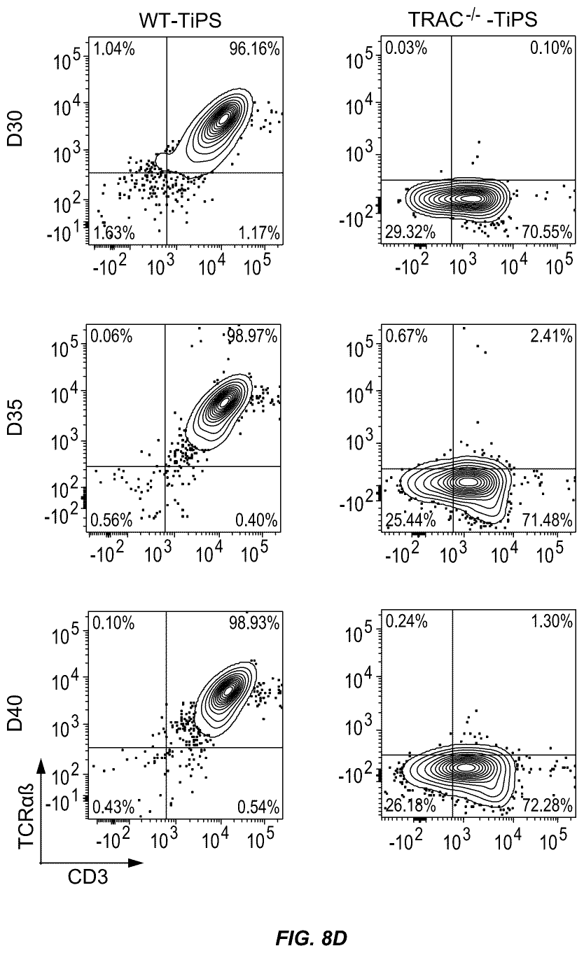

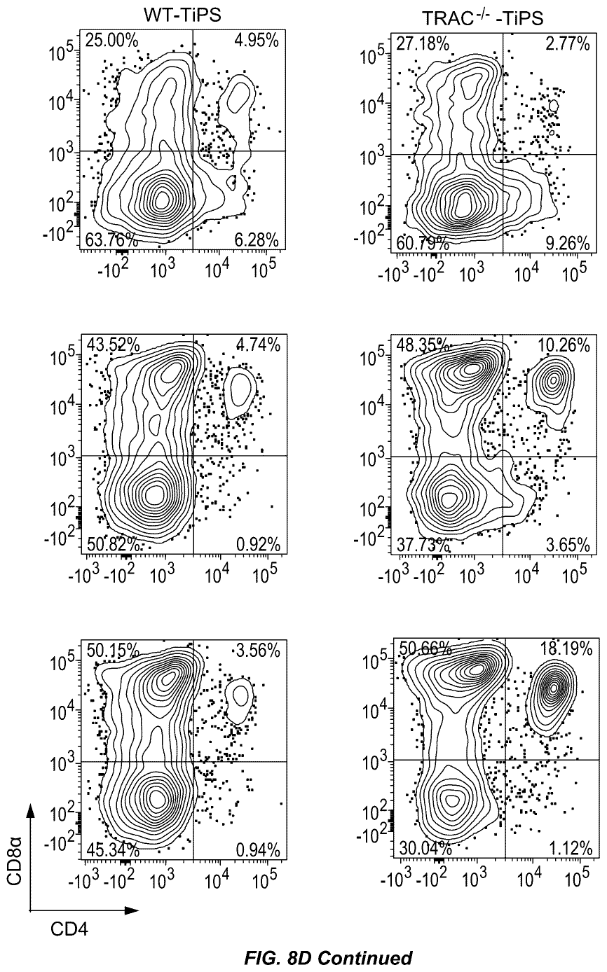

The presently disclosed subject matter determined the yield of CD4⁺CD8αβ⁺ DP αβTCR-T cell precursors from pluripotent stem cells using the OP9-mDLL1 stromal cell line in the differentiation protocol shown in FIG. 1A. The human embryonic stem (ES) cell line H1 and fibroblast-derived iPS (FiPS) cells consistently yielded a DP population, typically arising by day 35 and followed by the appearance of CD3⁺ cells by day 40 (FIGS. 1B and 7A-7C). When differentiating unmodified, wild-type TiPS (WT-TiPS, FIG. 7A) under the same conditions, very few DP cells were induced (FIG. 1). Nonetheless, CD3⁺αβTCR⁺ cells were generated, appearing much earlier, typically by day 25 (FIGS. 1B and 7D) and eschewing the DP intermediate state. As the variable (V), diversity (D), and junctional (J) αβTCR genes are pre-rearranged in WT-TiPS, in contrast to their germline configuration in ES and FiPS cells, it was reasoned that V gene transcription, which normally precedes VDJ recombination, would result in the premature expression of rearranged αβTCR genes in TiPS. It was further hypothesized that the early formation of an αβTCR would mimic the earlier timing of a productive γδTCR rearrangement, and thus bypass DP cell formation and impart an innate phenotype (Pardoll et al., J Immunol 140, 4091-4096 (1988)). To preclude early TCR assembly, TCRα chain expression was abolished by disrupting the TRAC locus (FIGS. 8A-8C). Disrupting the TRAC locus (TRAC^{-/-}-TiPS) indeed allowed for increased DP cell formation from TRAC^{-/-}-TiPS compared to WT-TiPS (FIGS. 1C, 2D, and 8D), supporting the notion that early TCR signaling prevents αβTCR-T cell lineage commitment (Baldwin et al., J Exp Med 202, 111-121 (2005). The altered lineage commitment also corroborated that the presently disclosed differentiation protocol (FIG. 1A) supports DP cell development and pointed to the critical importance of the timing of TCR assembly in determining the fate of TiPS-derived T cells.

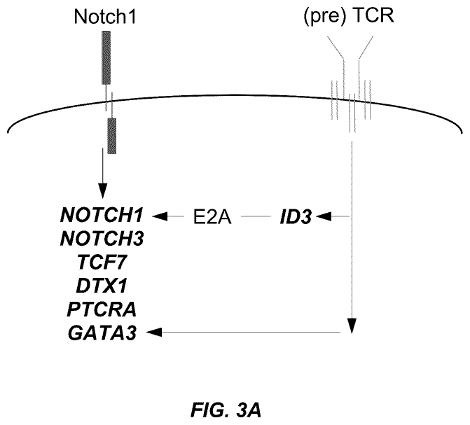

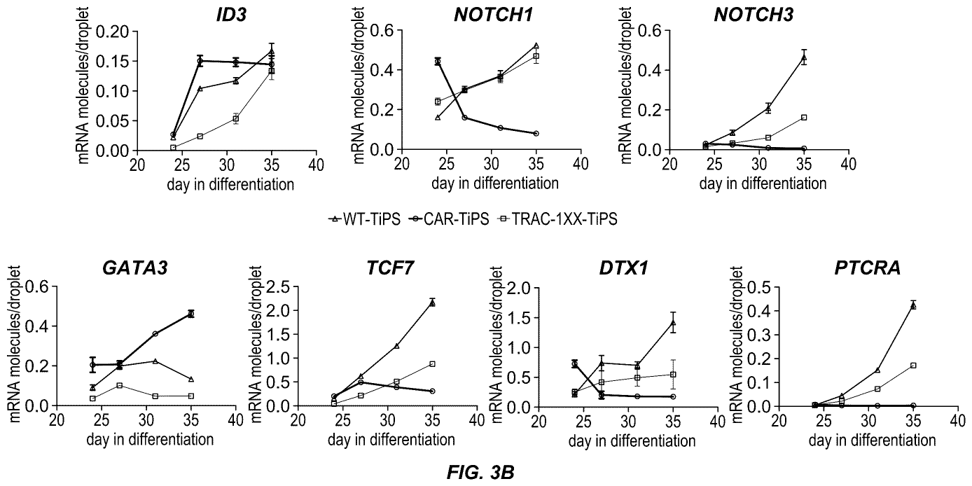

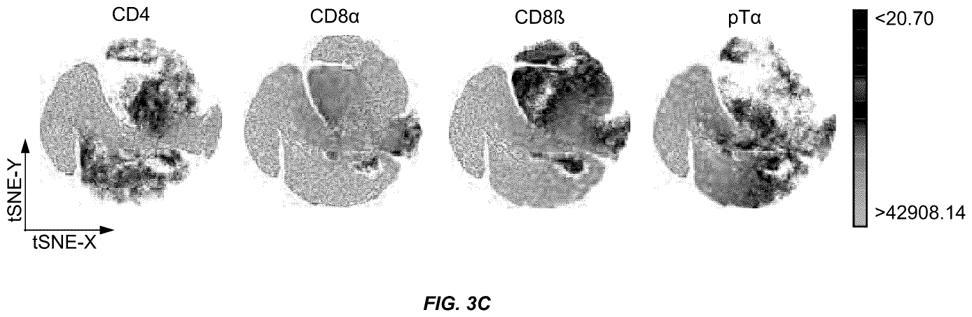

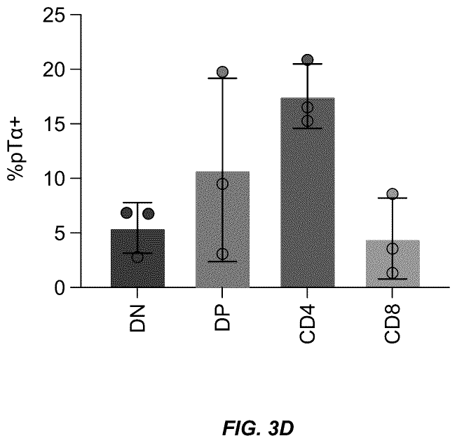

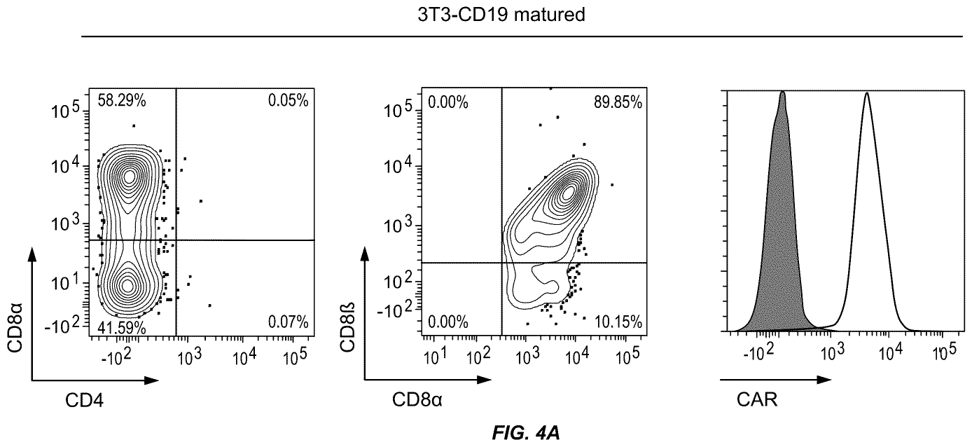

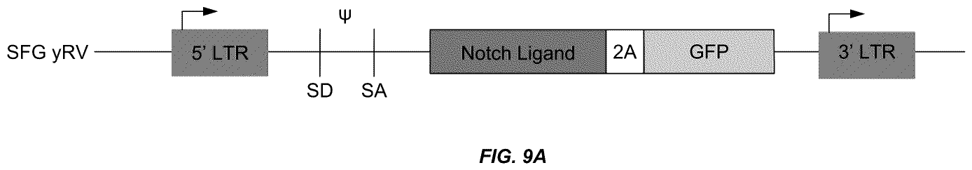

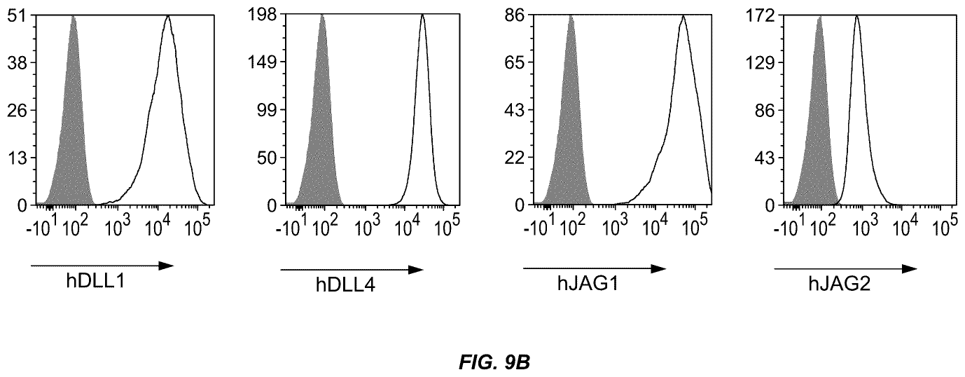

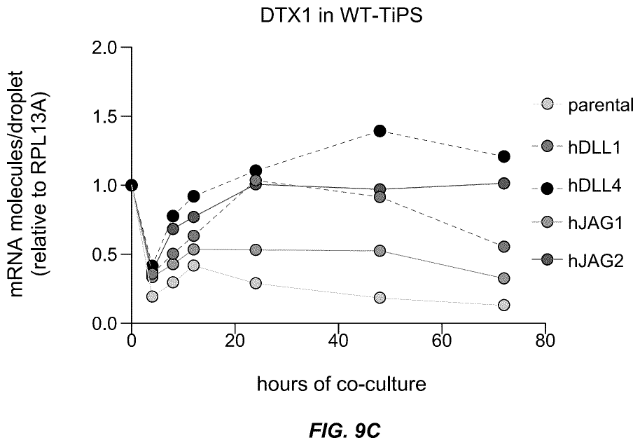

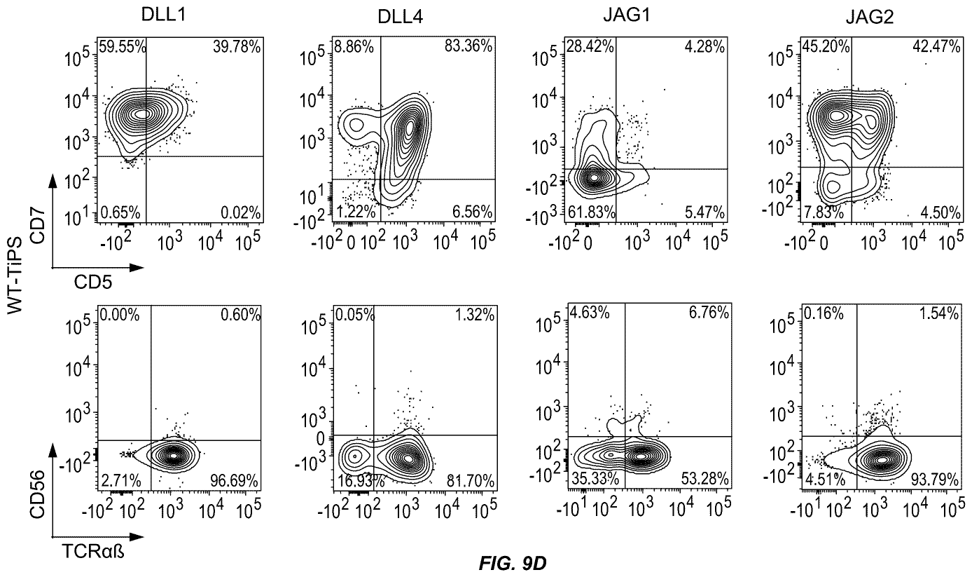

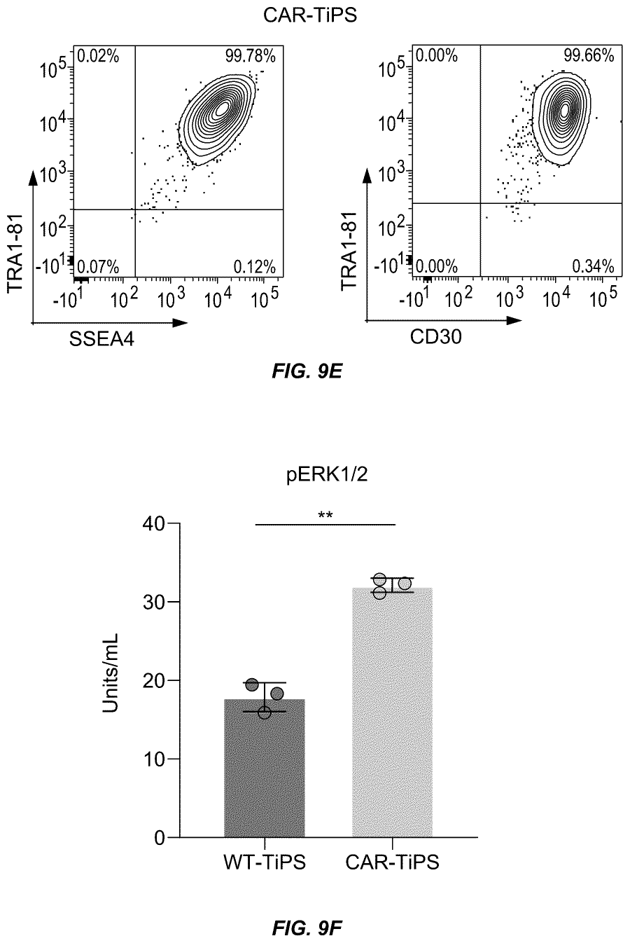

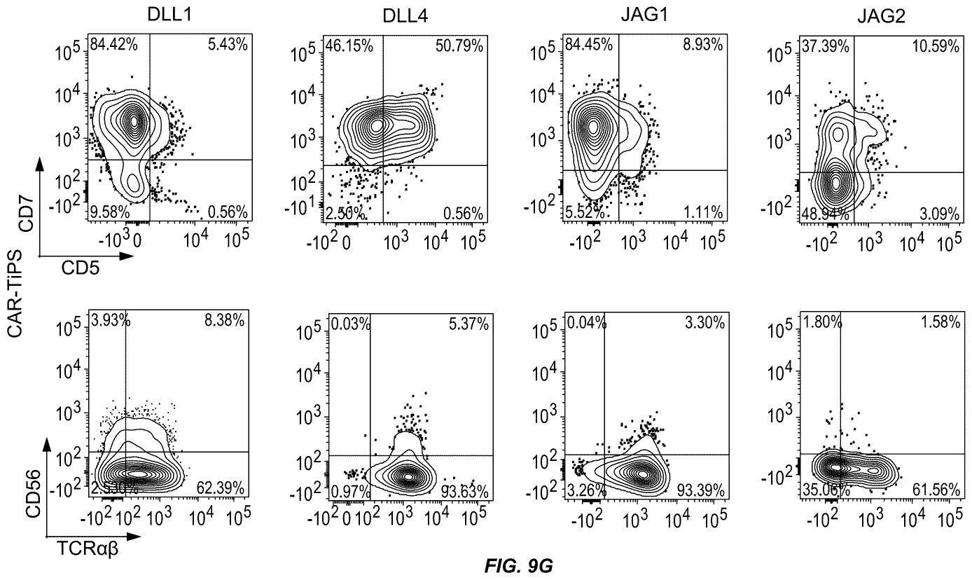

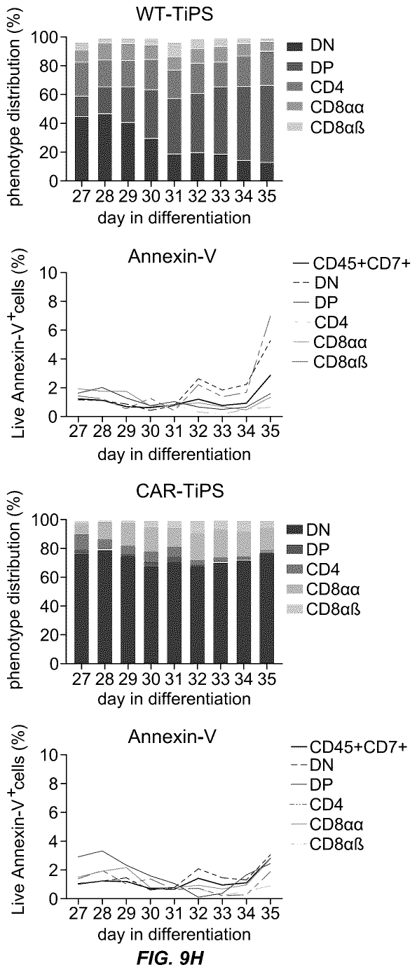

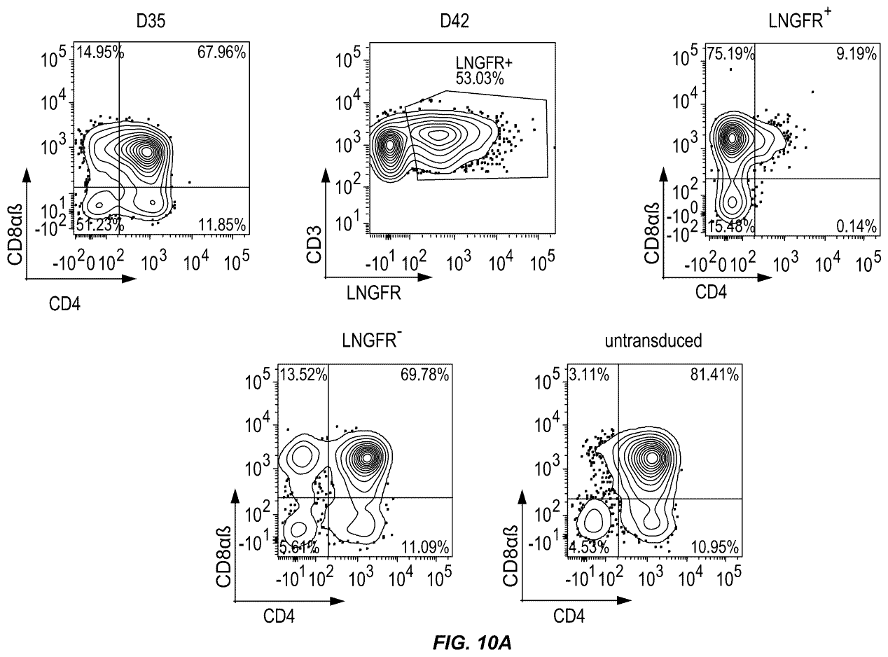

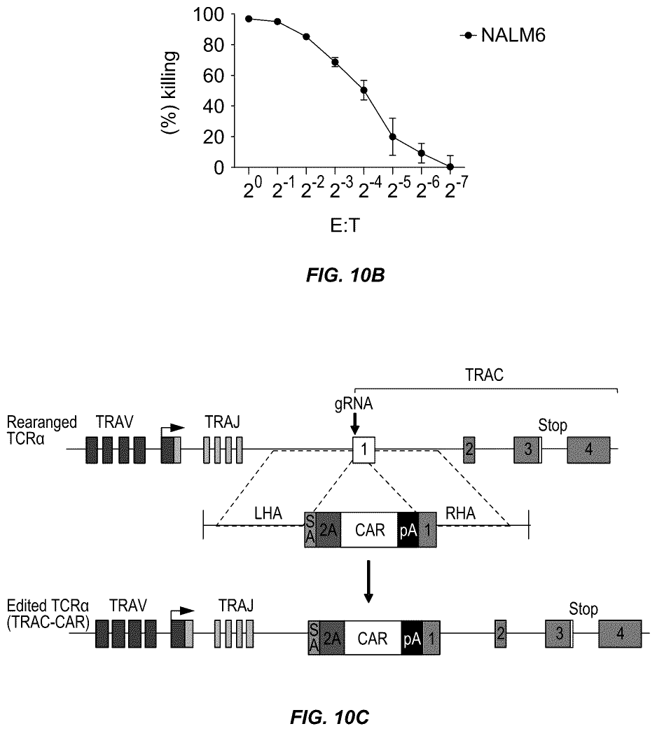

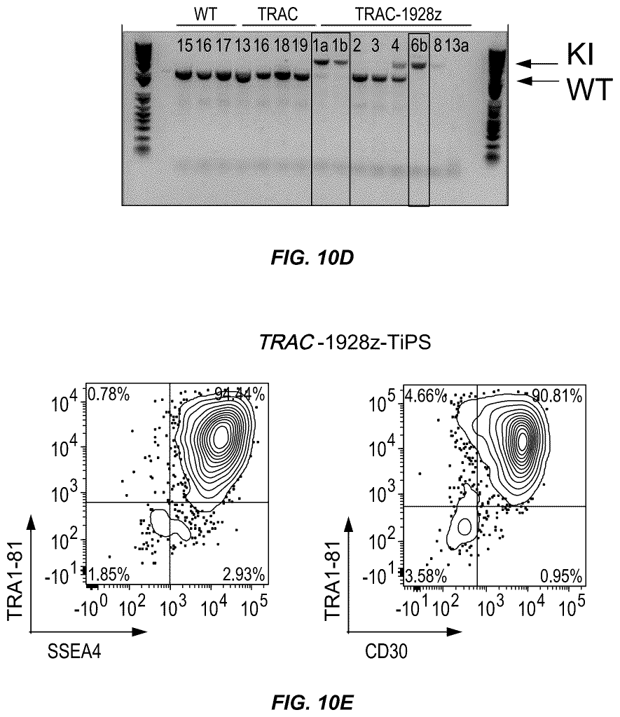

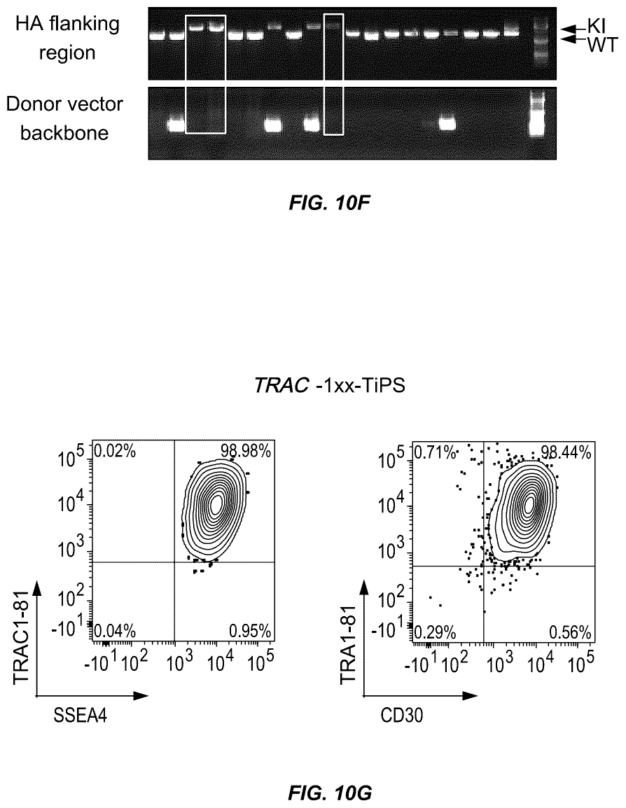

As Notch signaling also plays a critical role at the c43- versus 76-lineage commitment junction (Washburn et al., Cell 88, 833-843 (1997)), its role was investigated by first assessing different Notch ligands for their ability to support T cell differentiation from TiPS. OP9 stromal cells were engineered to express either one of the four human Notch ligands, Delta-like ligand 1 (DLL1), Delta-like ligand 4 (DLL4), Jagged-1 (JAG1) or Jagged-2 (JAG2) (FIGS. 9A and 9B). These ligands displayed a gradation in their level of Notch signaling induction (Extended Data FIG. 3c) and, correspondingly, their ability to support T lineage commitment and DP formation from WT-TiPS (FIGS. 1D and 9D). DLL1 and JAG1 were unable to support DP T cell development, in contrast to JAG2 and DLL4, the latter showing the greatest efficiency in both T lineage commitment (CD7⁺CD5⁺ positive cells, FIG. 9D) and DP formation (FIGS. 1D and 1E). Interestingly, DLL4 has been previously shown to efficiently support in vitro T cell differentiation after TCR gene rearrangement (Mohtashami et al., J Immunol 185, 867-876 (2010)) and induces the strongest signaling from Notchi (Van de Walle et al., J Exp Med 210, 683-697 (2013)) (FIG. 9C). These findings support a model wherein DP cell formation depends on an intricate interaction between TCR and Notch stimulation, in which a potent DLL4-mediated signal is required in WT-TiPS because of the earlier expression of a functional TCR, whereas DLL1 suffices for H1 and TRAC^{-/-}-TiPS. The requirement for stronger Notch engagement in the context of earlier TCR assembly suggests that the TCR interferes with Notch signaling, which can be over-ridden by more potent Notch ligands, and that a strong activation signal from the CAR (Ramello et al., Sci Signal 12 (2019); Maluski et al., J Clin Invest 129, 5108-5122 (2019)) and the TCR would offset stronger Notch signaling.

Consistent with this model, it was found that TiPS that constitutively expressed the 1928z CAR (CAR-TiPS, FIG. 9E), had increased levels of ERK1/2 phosphorylation (FIG. 9F), induced fewer CD7⁺CD5⁺ cells, and did not produce DP cells, even in the presence of DLL4, instead generating DN and CD8αα T cells (FIGS. 1F, 1G, and 9G). To assess whether premature CAR signaling induces apoptosis in emerging DP cells, apoptotic cells were measured at the different developmental stages (DN, CD4 induced single positive (ISP), DP, CD8αβ SP) in WT-TiPS and CAR-TiPS from D27-D35, when the induction of the DP population occurs in WT-TiPS (FIG. 9H). Levels of apoptosis were uniformly low (<5%) in both WT-TiPS and CAR-TiPS, and similar in all different developmental stages, indicating that the lack of DP establishment from CAR-TiPS is not due to global apoptosis of the DP population. To verify whether the development of CD8αβ T cells is feasible in the presence of a CAR, we transduced the CAR into DP cells arising from WT-TiPS on D35. Delaying the onset of CAR expression in this manner resulted in the development of functional SP cells, including CD8αβ⁺ CAR T cells (FIGS. 10A and 10B). This finding not only established that CD8αβ CAR T cells can be generated from TiPS, but also confirmed that the lack of DP formation from CAR-TiPS was likely due to interference with DP commitment arising from the early CAR expression afforded by the constitutive Ubiquitin C promoter in CAR-TiPS.

### 2. Regulated CAR Expression Facilitates CD4⁺CD8α⁺ DP T Cell Development

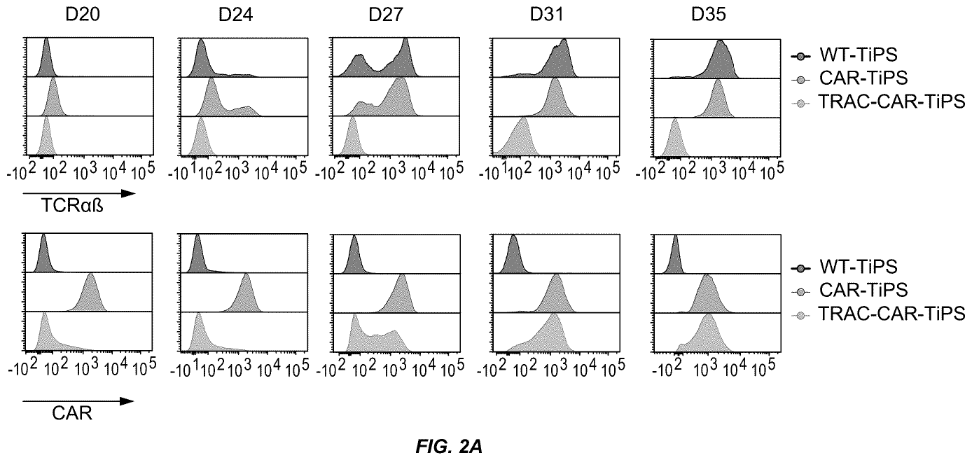

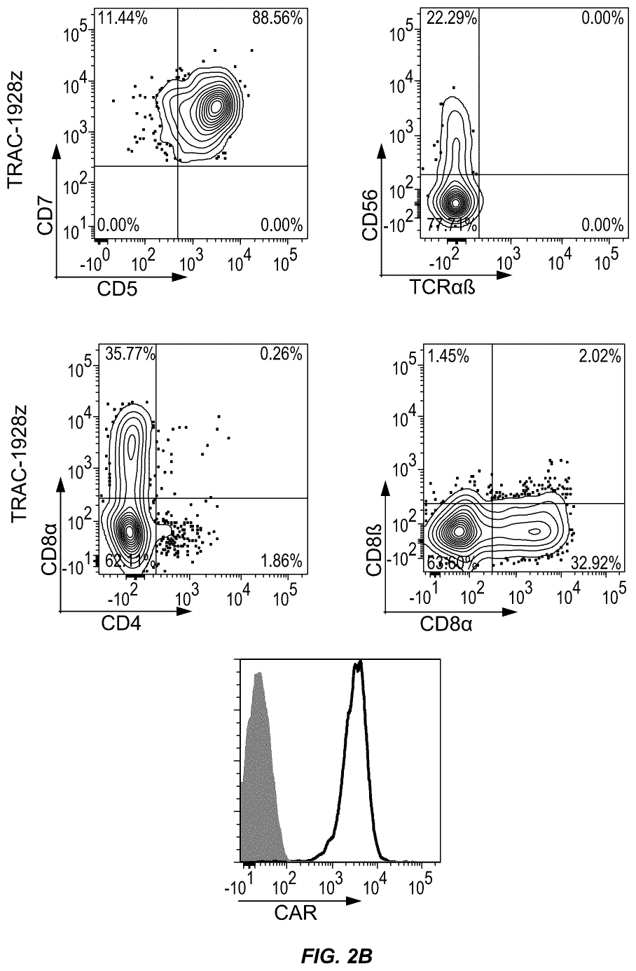

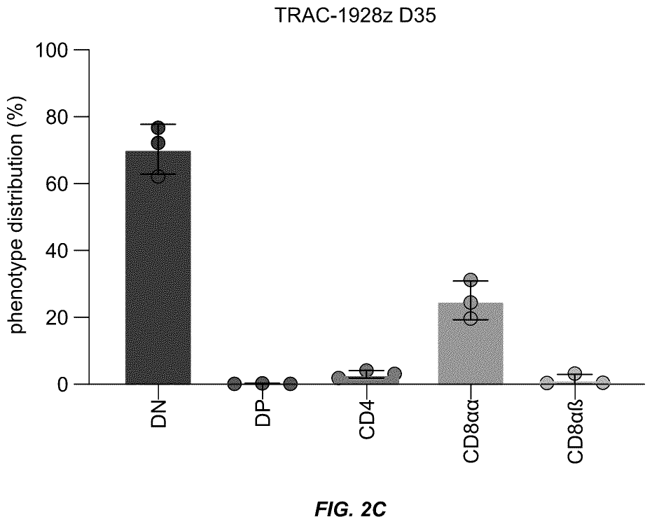

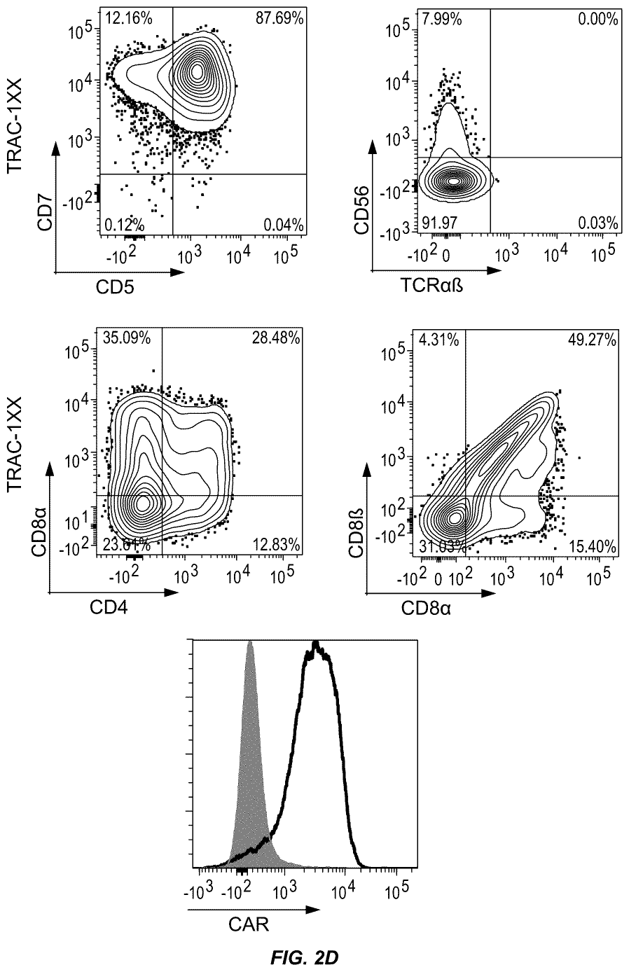

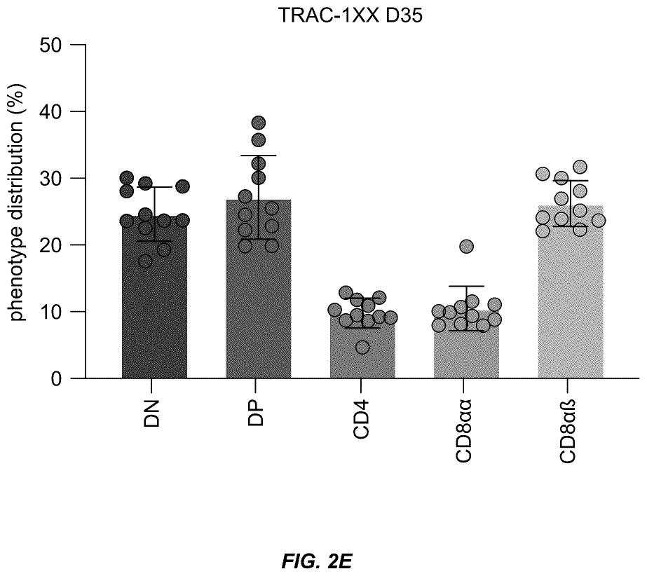

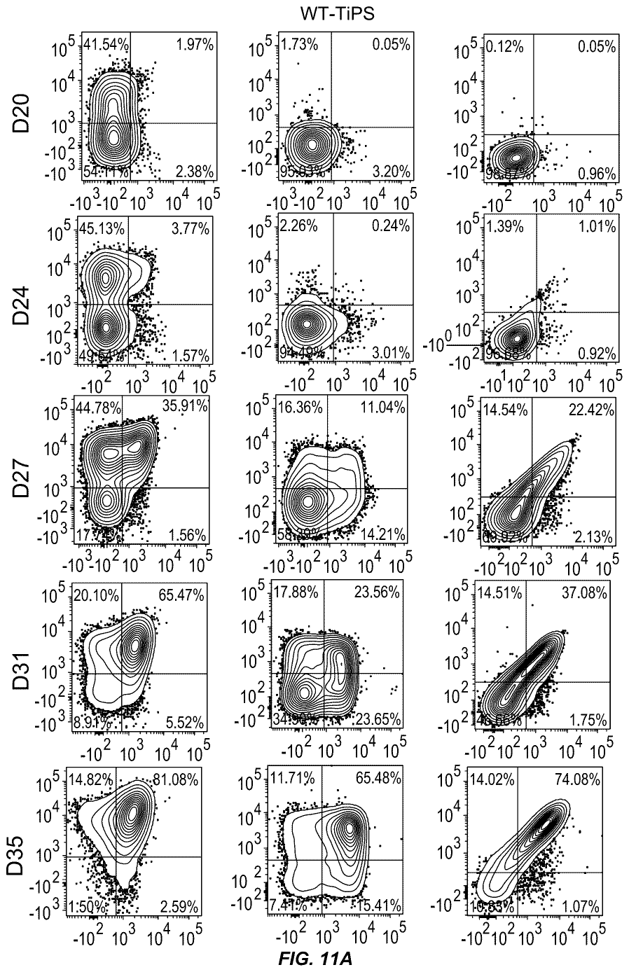

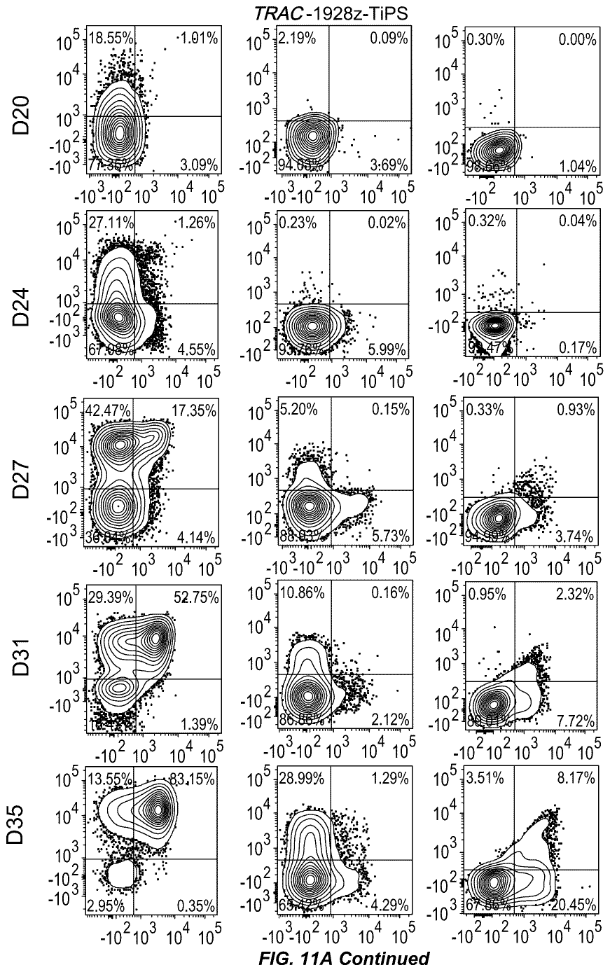

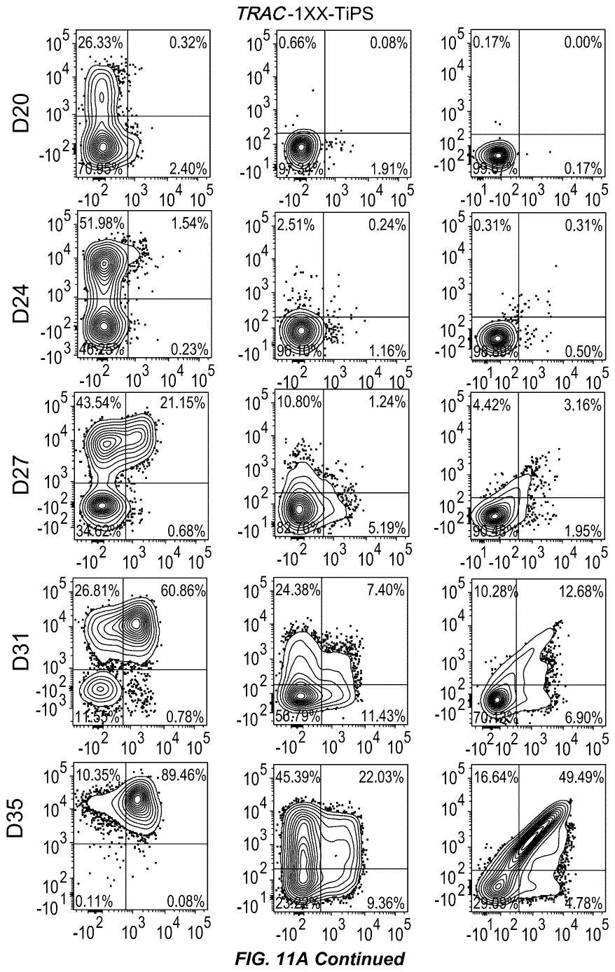

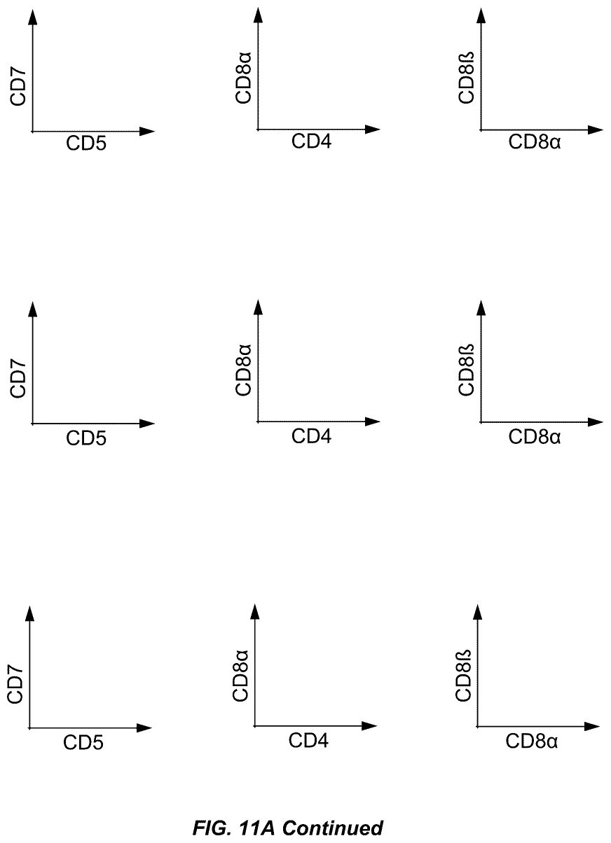

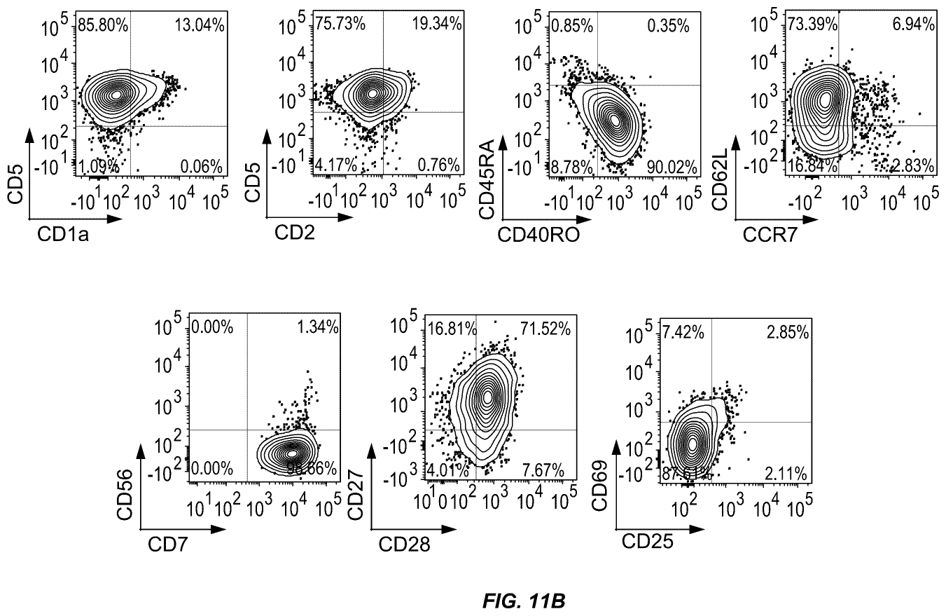

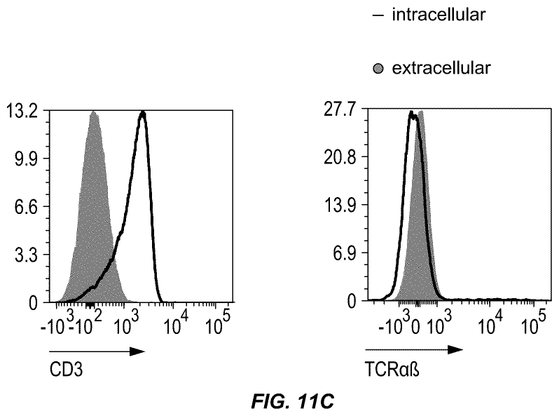

To restrict CAR expression, the sequence encoding CAR was placed under the transcriptional control of the TRAC promoter (TRAC-1928z-TiPS, FIGS. 10C-10E). CAR expression from the TRAC locus not only resulted in the expected absence of TCR expression throughout differentiation (FIG. 2A, top panel), but also showed the remarkable similarity in temporal cell surface expression between TRAC-CAR and the αβTCR in both WT-TiPS and CAR-TiPS (FIG. 2A). The differentiation of TRAC-1928z-TiPS towards early T cell lineage commitment improved, as reflected in a greater CD7⁺CD5⁺ population (FIG. 2B) compared to CAR-TiPS (FIG. 9G). DP induction, however, was still not enhanced (FIGS. 2B, 2C, and 11A).

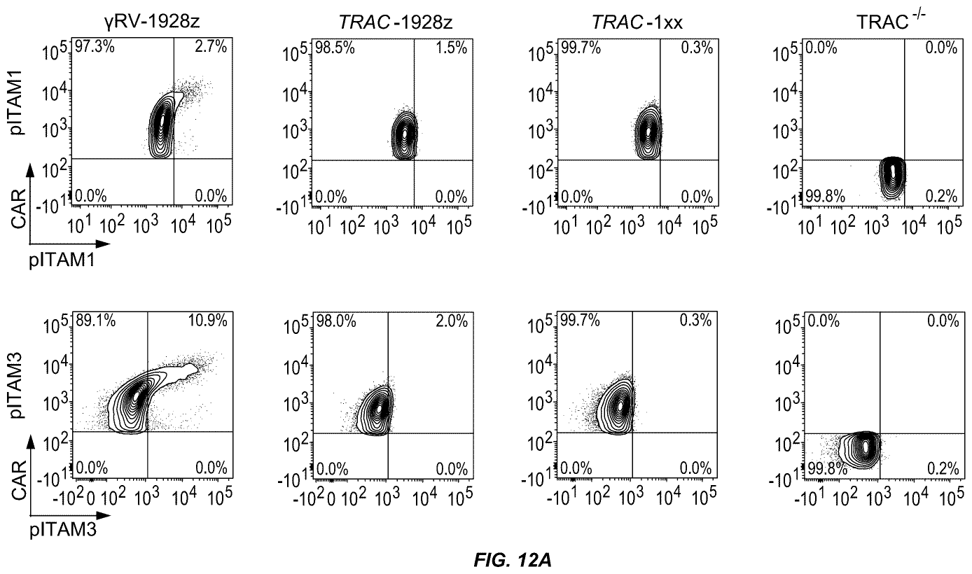

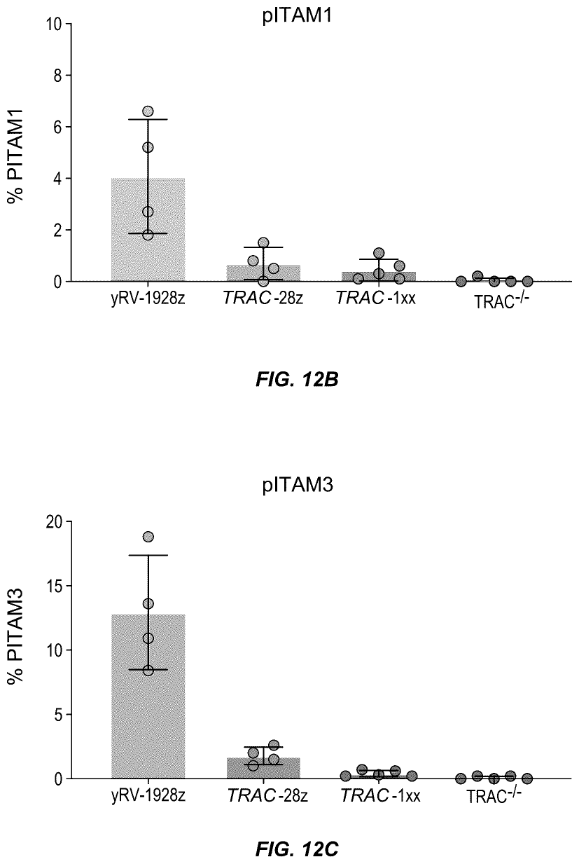

It was hypothesized that despite its delayed onset of expression, the TRAC-encoded 1928z still interfered enough to prevent DP commitment. It was therefore sought to attenuate CAR signaling strength by substituting the 1928z with 1928z-1XX, a CAR in which the second and third ITAM have been inactivated. In PBMC-derived T cells, CAR expression through the endogenous TRAC promoter reduces signaling in absence of antigen exposure and the mutation of the second and third ITAMs pre-empts their phosphorylation. Phosphorylation of ITAM1 and ITAM3 is readily detected in retrovirally expressed CARs (γRV-1928z) in the absence of antigen, increasingly in T cells with the highest CAR expression (FIGS. 12A-12C). TRAC-encoded 1928z showed reduced phosphorylation of ITAM1 and ITAM3, while the latter was abolished in TRAC-1XX T cells (FIGS. 12A-12C).

Upon their differentiation, TRAC-1XX-TiPS ((FIGS. 10F and 10G) not only maintained the same heightened propensity to induce CD7 and CD5 expression as TRAC-1928z-TiPS, but additionally increased their progression to the DP stage (FIGS. 2D, 2E, and 11A). By day 35, these DP cells express CD1a, CD2 and CD45RO, consistent with the phenotype of human DP thymocytes (Res et al., J Exp Med 185, 141-151 (1997); Haynes et al., J Immunol 141, 3776-3784 (1988); Fujii et al., Eur J Immunol 22, 1843-1850 (1992)) (FIG. 11B). Intracellular CD3 confirmed their T lineage commitment despite the absence of CD3/αβTCR expression at their cell surface (FIG. 11C).

### 3. CAR Expression Affects Notch and TCR Downstream Target Gene Induction

To further support the hypothesis that the induction to the DP stage is controlled by CAR and Notch interactions, the impact of CAR regulation on Notch target transcript levels was assessed during the T lineage commitment phase of the presently disclosed in vitro differentiation protocol. Expression level of several genes which have been reported to be associated with T lymphoid development and αβ/γδ lineage commitment was assessed, including NOTCHI, NOTCH3, ID3, TCF7, DTX1, GATA3 and PTCRA (FIG. 3A). Constitutive CAR expression did indeed grossly perturb the expression pattern observed in WT-TiPS. CAR-TiPS show an early increase in the expression of the (pre)TCR target ID3 between D24 and D27 (FIG. 3B), with correspondingly reduced levels of NOTCHI, NOTCH3 and Notch targets TCF7, DTX1 and PTCRA. In the TRAC-1XX-TiPS, ID3 expression was decreased while NOTCHI, NOTCH3 and their downstream targets increased to levels nearing those found in WT-TiPS (FIG. 3B). GATA3 expression, which is a direct target gene of not just Notch but also the pTα, was noticeably upregulated in CAR-TiPS relative to WT-TiPS and reduced in TRAC-1XX-TiPS (FIG. 3B), suggesting that constitutive CAR expression results in stronger GATA3 induction compared to the TCR or pTα.

Notably, one of the key Notch targets, PTCRA, which encodes the pTα, is repressed in CAR-TiPS, but induced in differentiating WT-TiPS as well as in TRAC-1XX-TiPS (FIG. 3B). In thymic development, pTα pairs with the rearranged β-chain to allow for a chain rearrangement and progression to the DP stage (Fehling et al., Nature 375, 795-798 (1995)). To assess whether PTCRA induction in TRAC-1XX-TiPS was associated with pTα protein expression, cell surface-protein levels were measured by flow cytometry and found cell surface pTα expression in the induced-CD4 SP and DP populations (FIGS. 3C and 3D), consistent with its physiological pattern (Dolens et al., EMBO Rep 21, e49006 (2020)).

### 4. CAR Engagement Facilitates Maturation to CD8a13 Single Positive T Cells

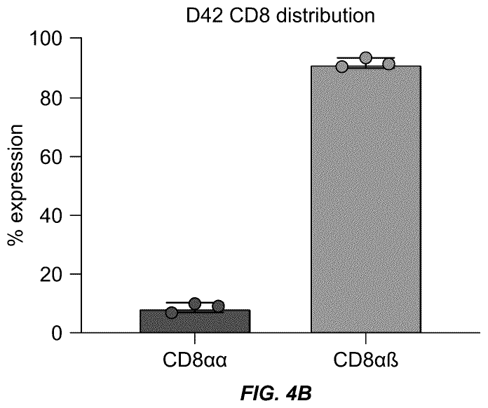

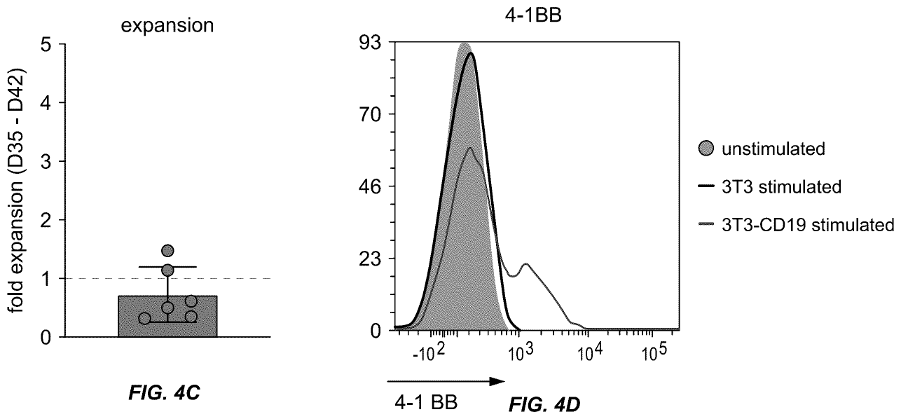

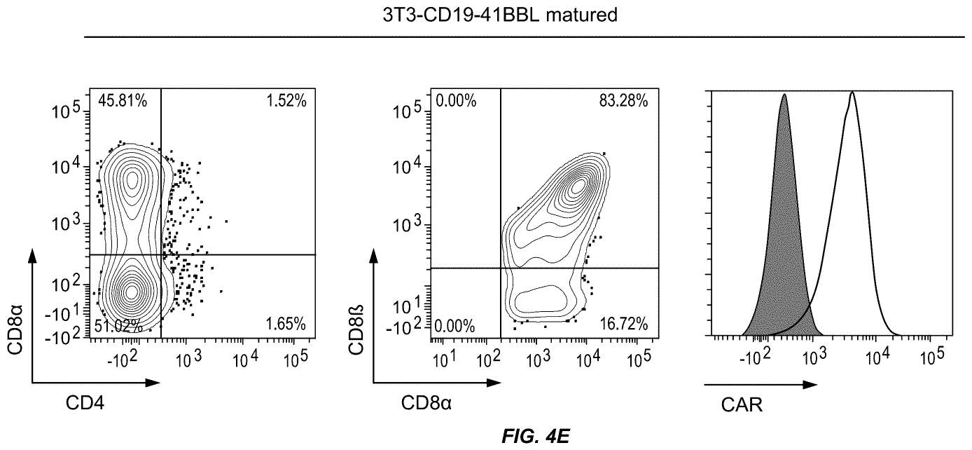

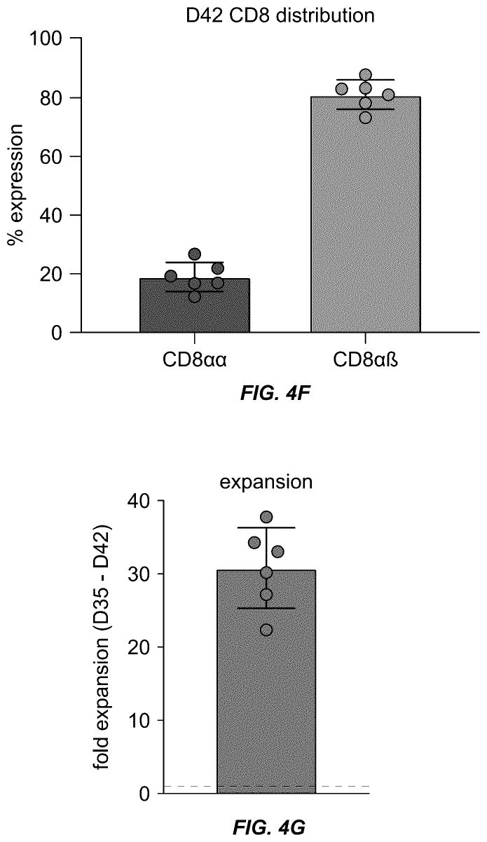

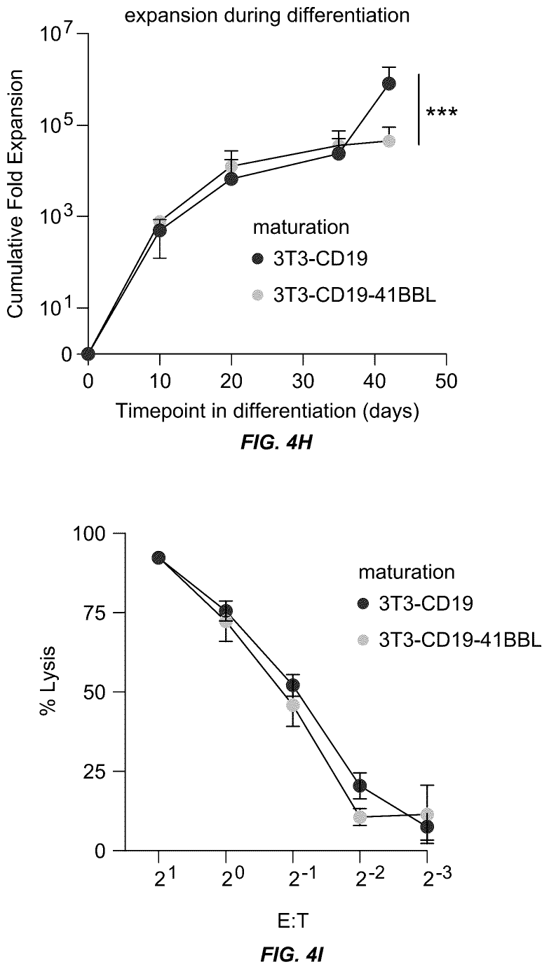

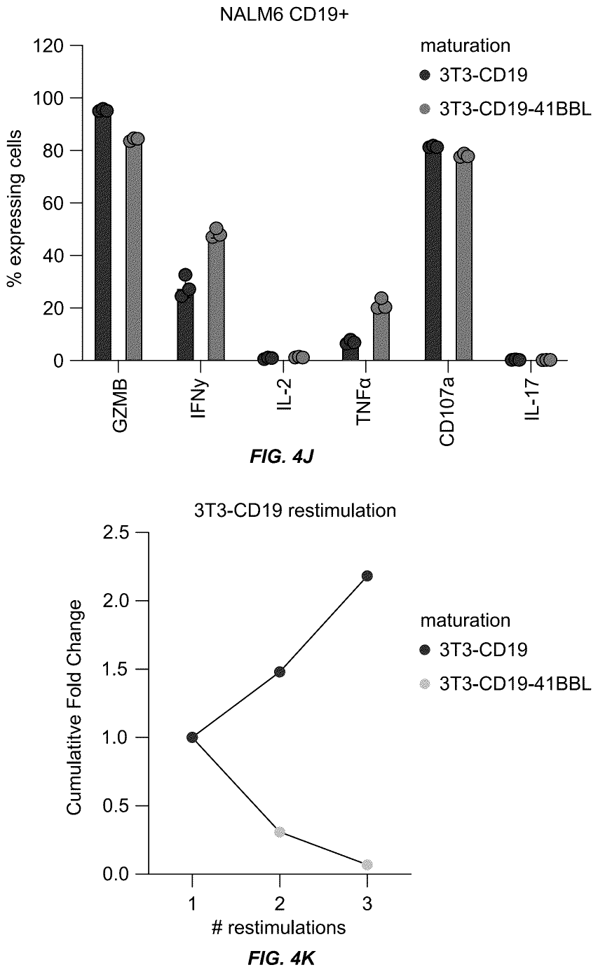

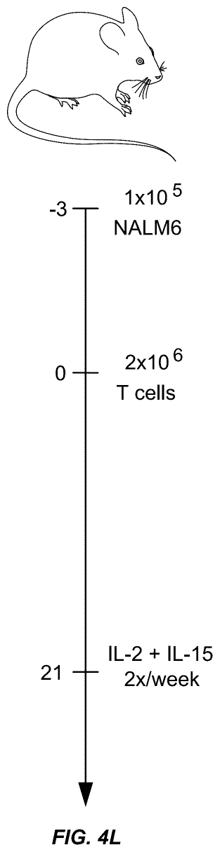

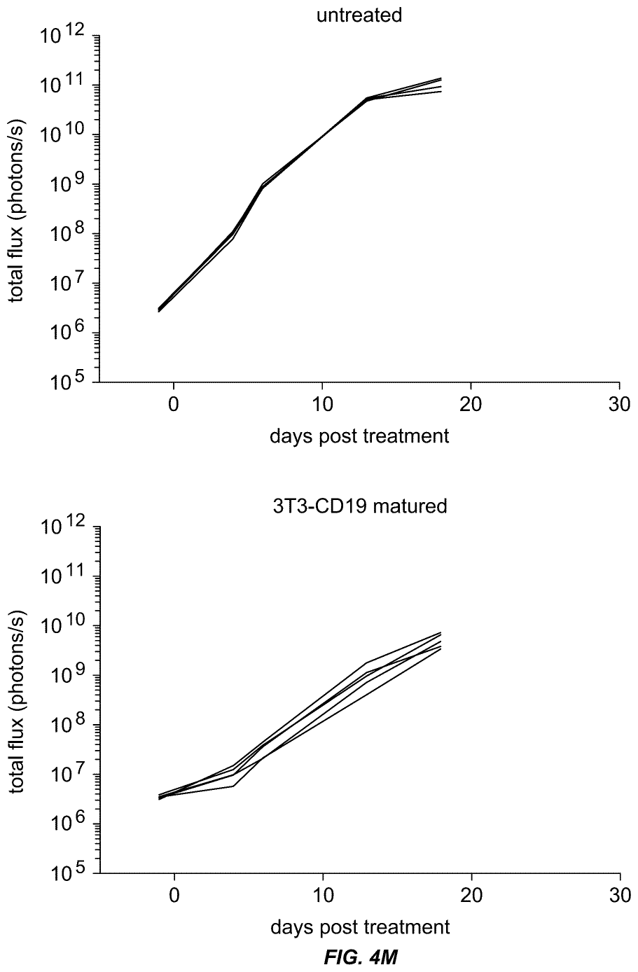

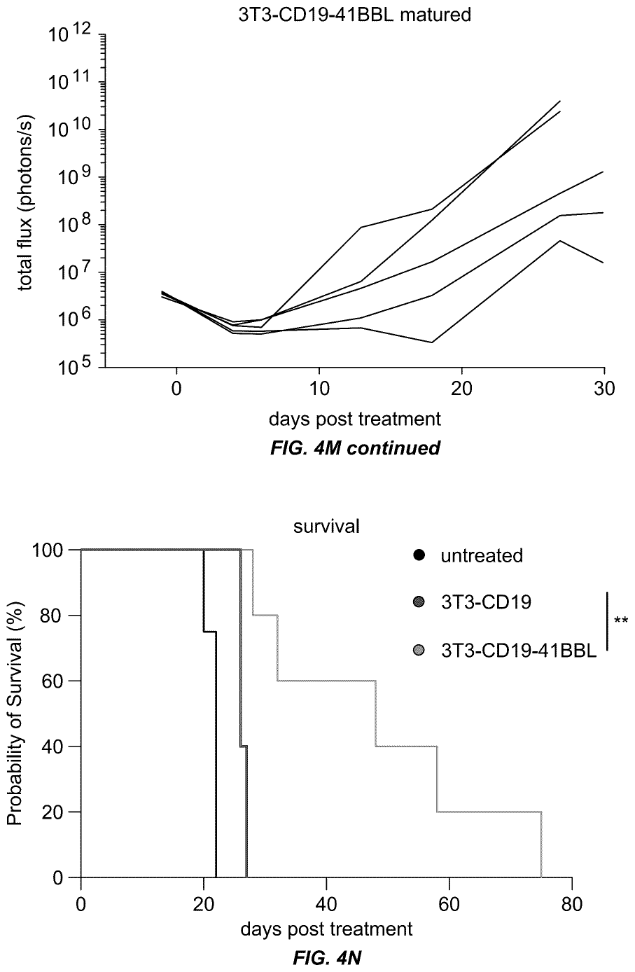

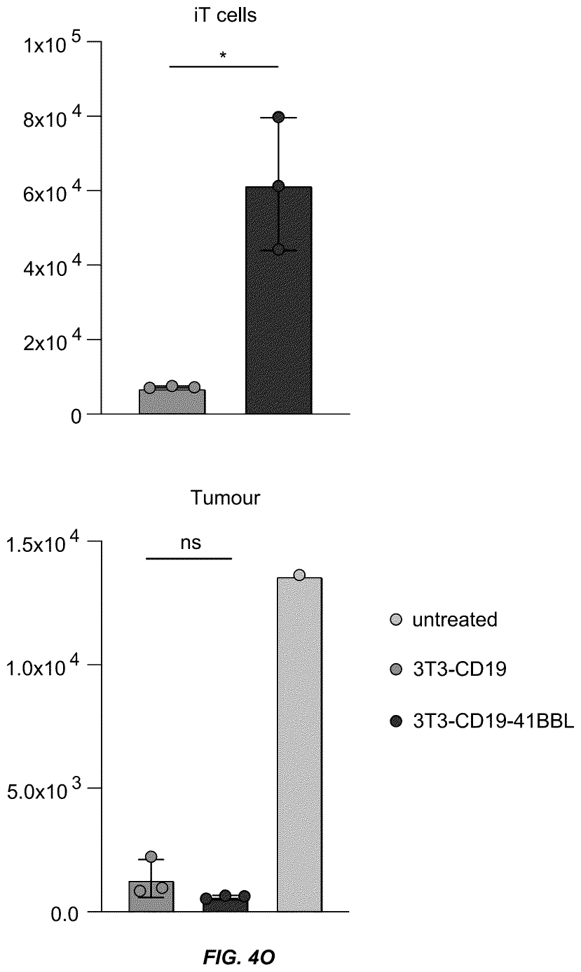

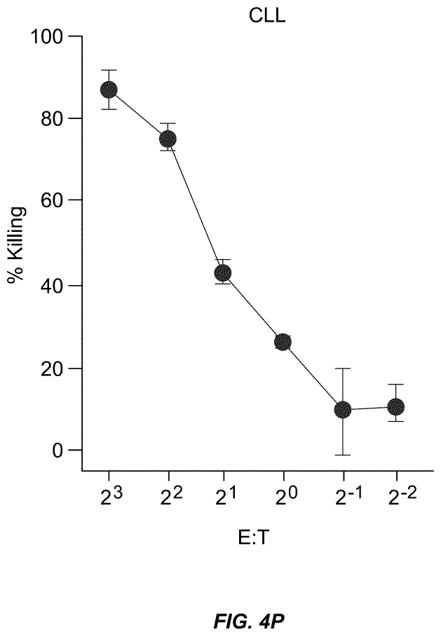

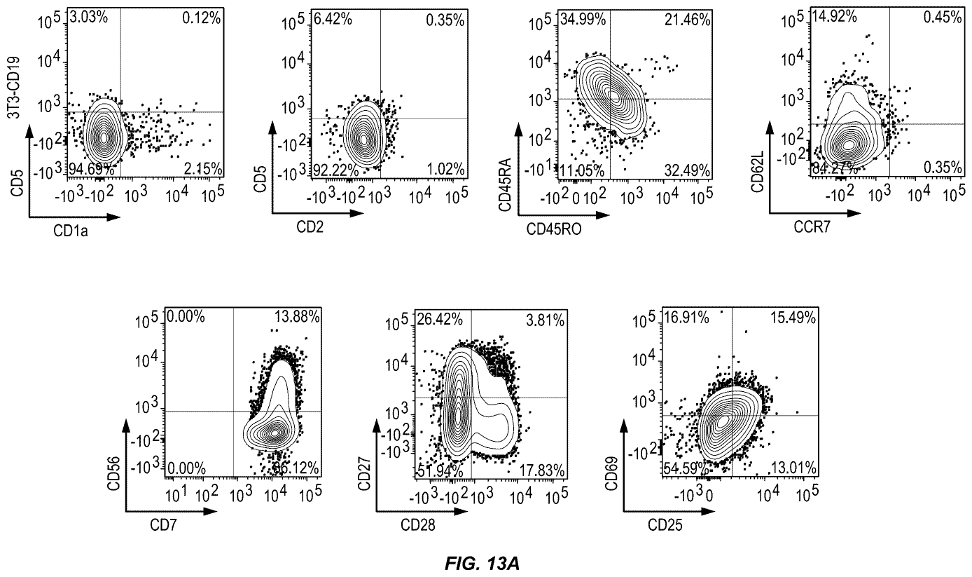

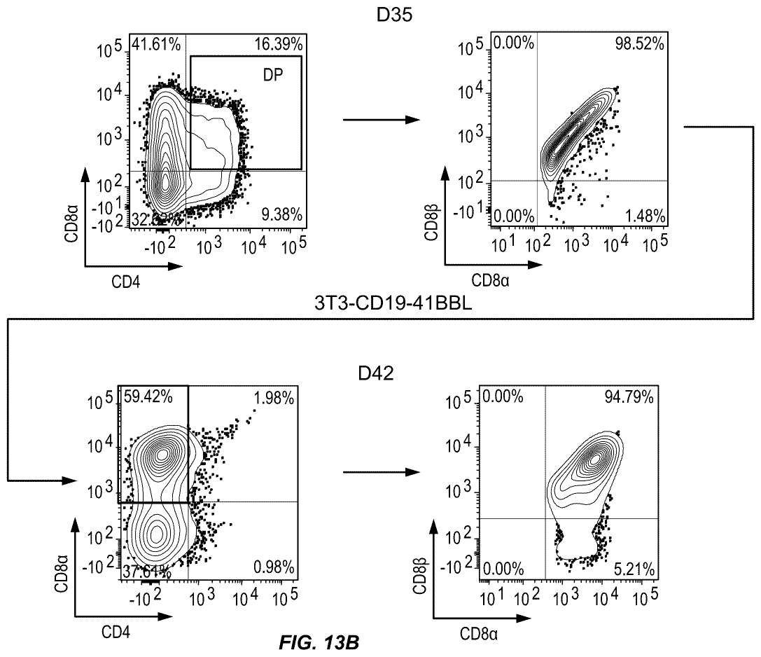

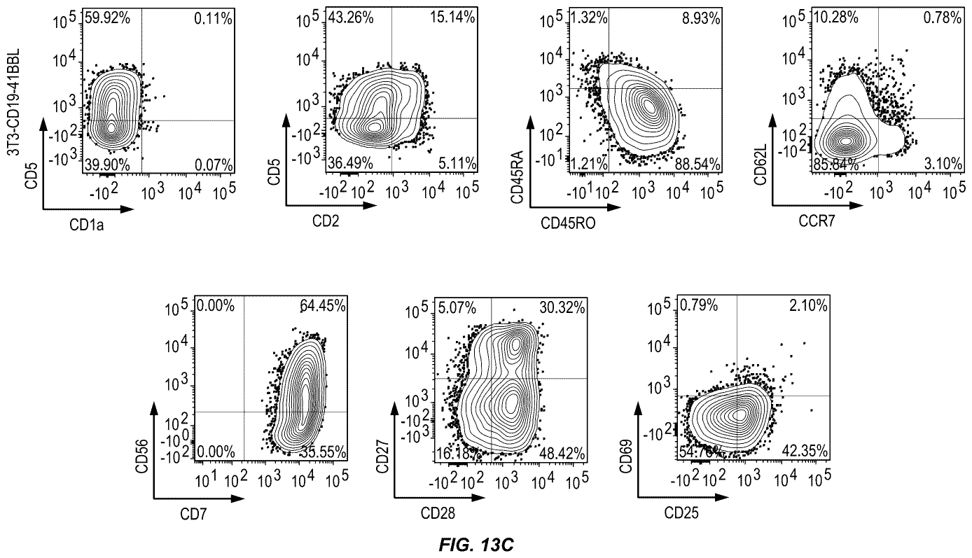

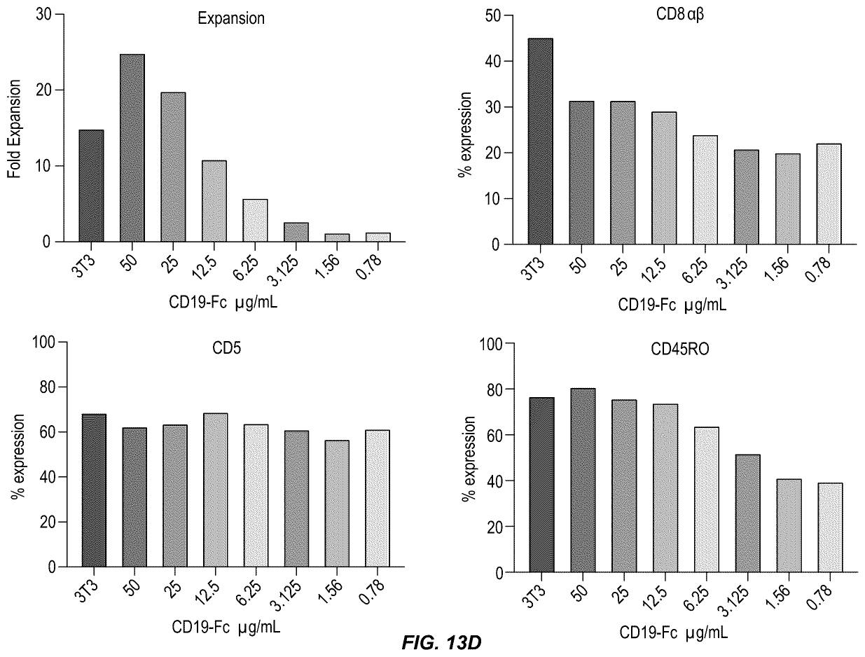

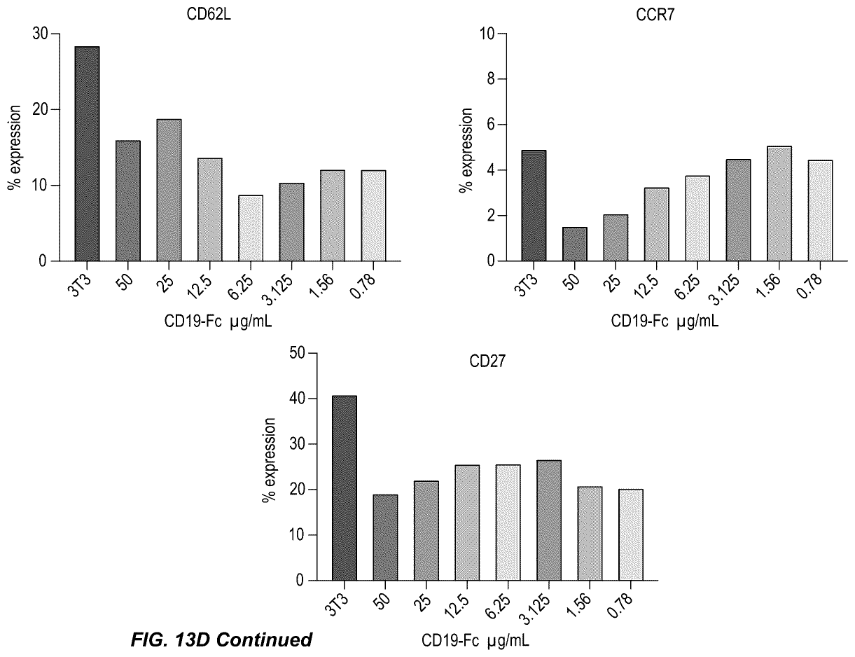

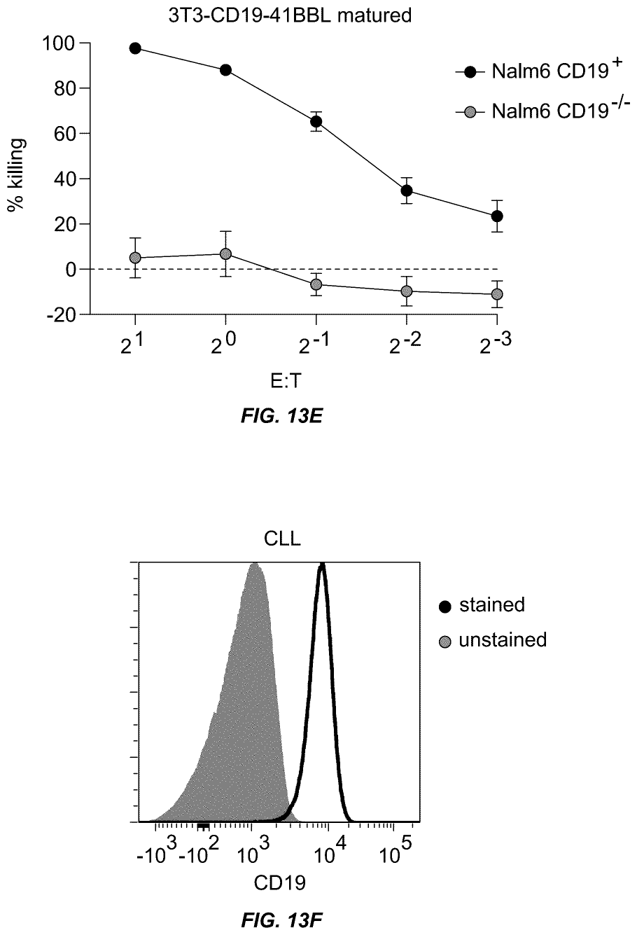

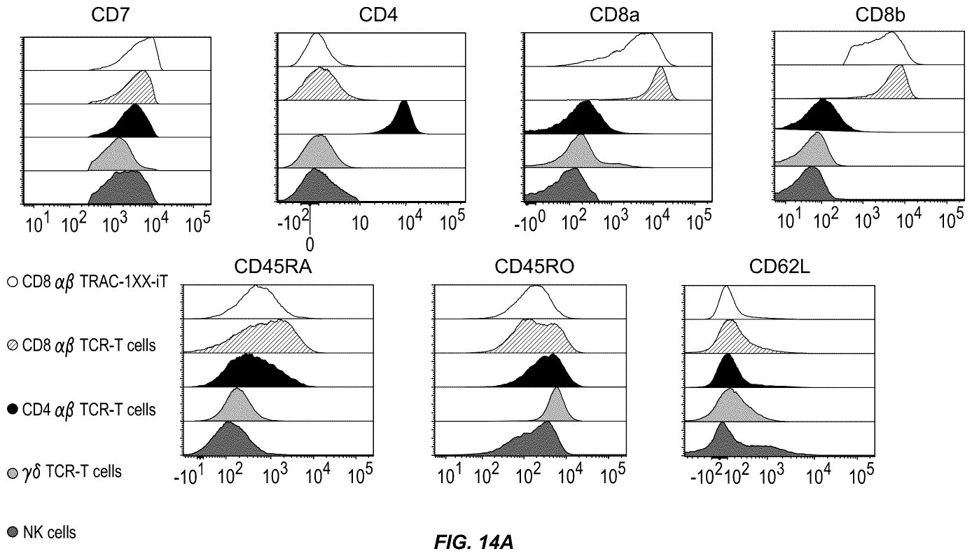

Having established that CAR regulation allows for DP cell development in the absence of a TCR, it was set out to determine whether the CAR could substitute for the TCR in further driving maturation of DP cells into CD8αβ SP T cells. In the absence of a TCR-dependent positive selection process, D35 DP T cells were stimulated on cells expressing the CAR target antigen (NIH/3T3 fibroblasts expressing CD19) (Brentjens et al., Nat Med 9, 279-286 (2003)). CAR engagement was required for T cell survival as co-culture with parental fibroblasts lacking CD19 did not yield viable iT cells. Phenotypic analysis on CD19 stimulated TRAC-1XX-iT cells after a week (day 42) showed that CD4 and CD1a expression had waned and that a population of CD8αβ SP T cells was induced, consistent with maturation from the DP to the SP stage (FIGS. 4A and 13A). Some DN cells and a small population of CD8αα T cells still coexisted (FIG. 4B). The matured SP D42 TRAC-1XX-iT cells displayed a phenotype resembling activated T cells, including the upregulation of CD25, CD69, CD56 and transition from CD45RO to CD45RA (FIG. 13A). However, the cells also downregulated CD5 and expanded poorly (FIGS. 4C and 13A).

Analyzing the phenotype and induction of activation and costimulatory markers upon antigen exposure, it was found that 4-1BB was induced transiently 8 h after antigen exposure (FIG. 4D) and hypothesized that engaging the 4-1BB pathway at this stage may promote T cell expansion. Thus, the 3T3-CD19 cells were engineered to co-express 4-1BB ligand (3T3-CD19-41BBL). When D35 TRAC-1XX-iT cells were stimulated on 3T3-CD19-41BBL, they maintained the ability to form SP cells (FIGS. 4E and 4F) and expanded 30-fold (FIG. 4G). To confirm that the resulting SP cells at D42 were indeed derived from D35 DP precursors, the DP cells were sorted at D35 and then exposed to 3T3-CD19-41BBL. After 7 days, the DP cells had lost CD4 expression and matured to CD8αβ SP cells (FIG. 13B). Phenotypically, 3T3-CD19-41BBL matured cells retained CD5 and CD2 expression, more so than 3T3-CD19 matured cells, and showed higher expression of CD45RO, CD28 and CD56 (FIG. 13C).

To determine whether CD19 levels may influence acquisition of an effector-like phenotype of the TRAC-1XX-iT cells, iT cells were matured on titrated levels of recombinant CD19 (FIG. 13D). Increasing CD19 positively affected the expansion and CD8ab SP iT cell content, but did not reduce the effector-like phenotype. To determine whether exposure to 4-1BB costimulation qualitatively affected TRAC-1XX-iT maturation, the functions of the 3T3-CD19 and 3T3-CD19-41BBL matured TRAC-1XX-iT cells were compared. In vitro cytolytic function (FIG. 4I) and cytokine production (FIG. 4J) were similar between the two groups. However, whereas 3T3-CD19 matured cells failed to expand upon repeated exposure to antigen, 3T3-CD19-41BBL maturation improved their expansion and survival (FIG. 4K). The presently disclosed subject matter proceeded to compare these two populations in the NALM6 leukemia model (FIG. 4L), wherein iT cells matured on 3T3-CD19-41BBL showed improved tumor control and survival (FIGS. 4M-4O), which was associated with an increased persistence of TRAC-1XX-iT cells (FIG. 4O). The cytolytic function of 3T3-CD19-41BBL matured TRAC-1XX-iT cells was also demonstrated in vitro to be antigen specific (FIG. 13E), and responsive not only to NALM6, but also to primary patient-derived CD19⁺ CLL cells (FIGS. 4P and 13F).

### 5. TRAC-1XX-iT Cells Overall Resemble Peripheral-Blood Derived CD8αβ T Cells

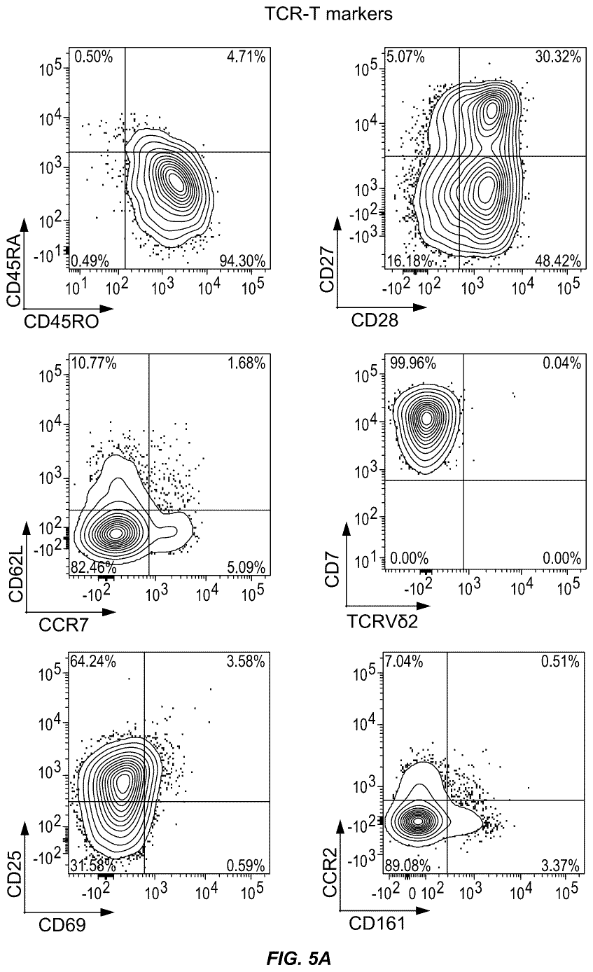

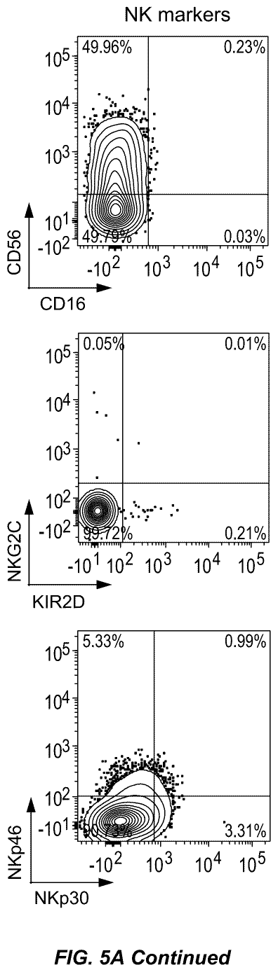

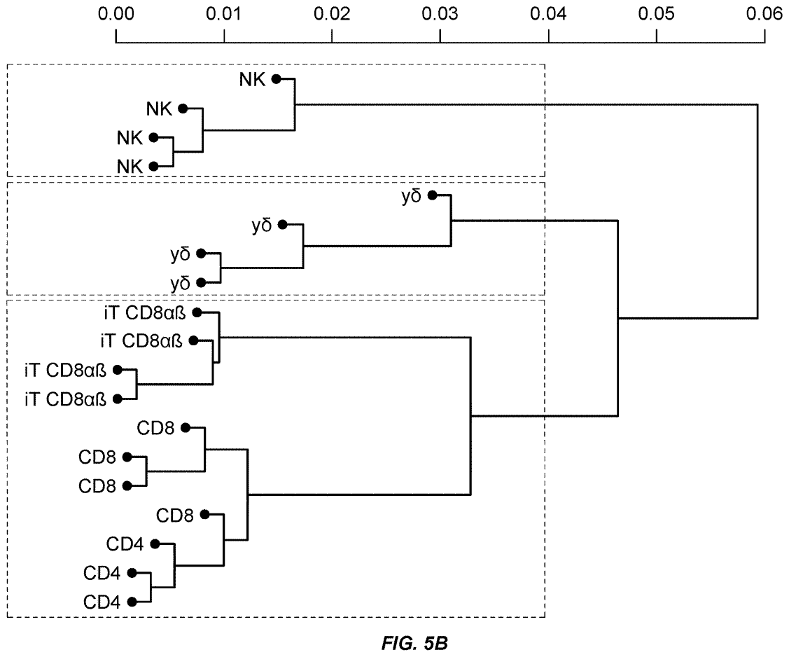

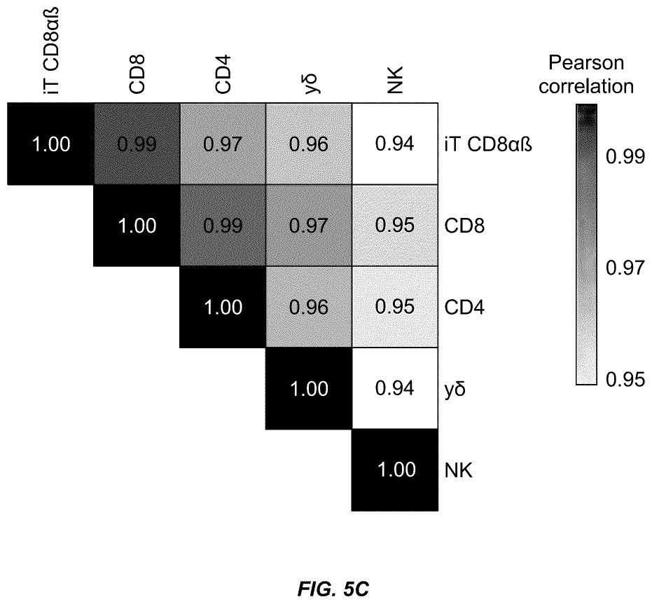

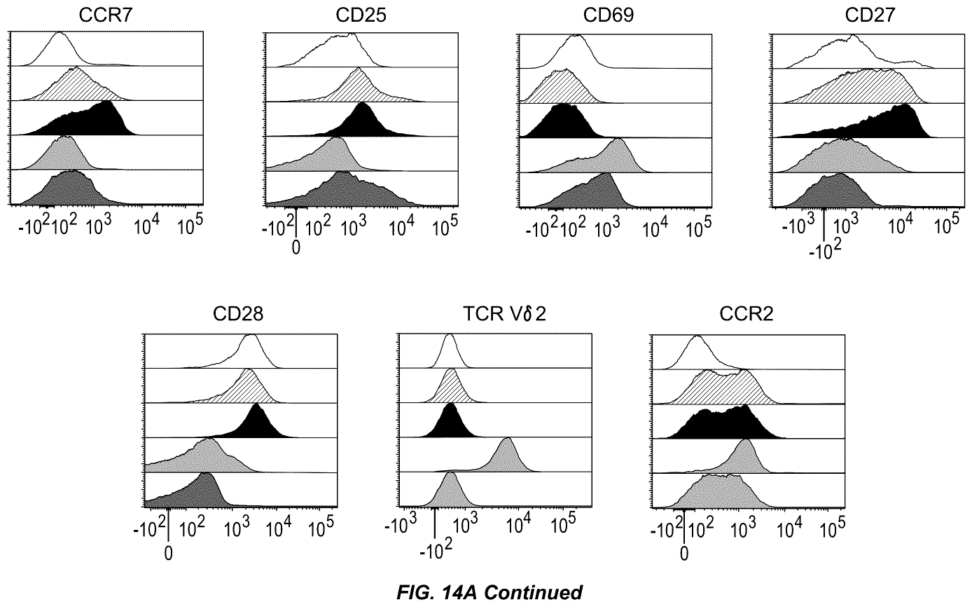

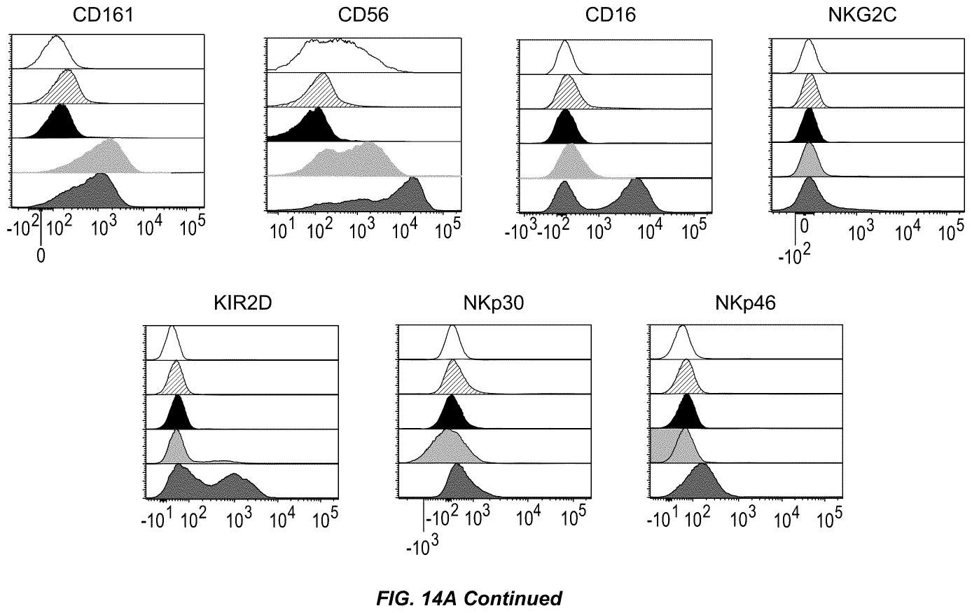

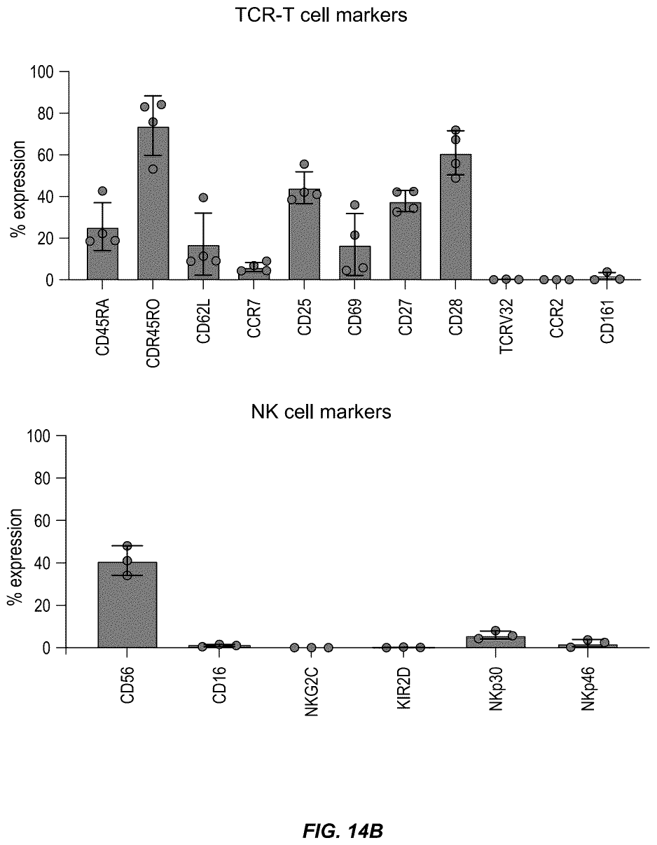

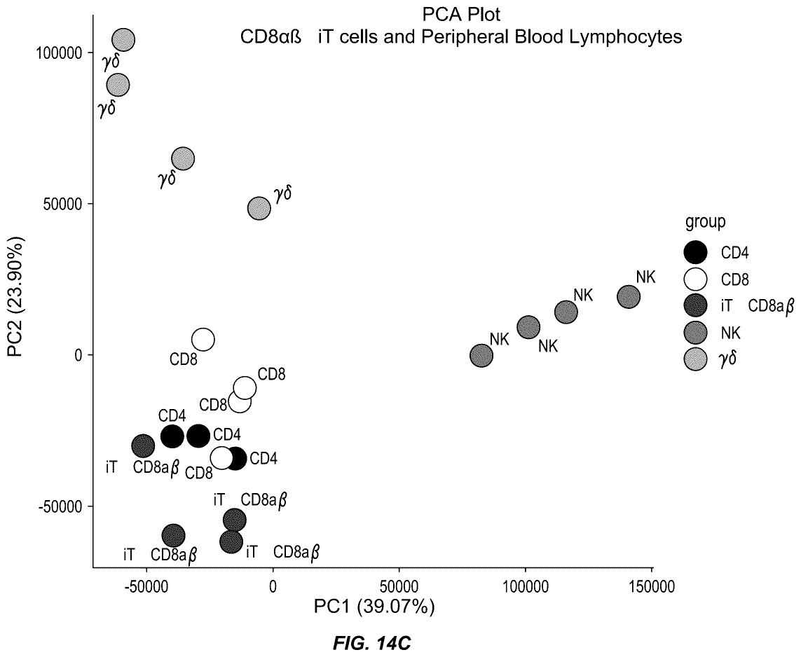

Having established that T cell commitment, differentiation, maturation, and expansion of CD8αβ CAR T cells can be driven by CAR expression in the absence of a TCR, it was sought out to compare the resulting CD8ab TRAC-1XX-iT cells (CD8ab iT) to naturally occurring peripheral blood lymphocytes. Analysis of cell-surface markers associated with αβTCR-T cells, NK cells or γδTCR-T cells, showed that CD8αβ iT cells express classical T cell markers including CD45RO, CD25, CD27, CD28 and low levels of CD62L and CCR7 (FIGS. 5A, 14A, and 14B). The stimulated cells expressed the T cell activation/NK cell marker CD56 but lack canonical NK markers such as CD16 and KIR2D (FIGS. 5A, 14A, and 14B). They also did not express γδTCR-T cell associated markers including the γδTCR and CD161 (FIGS. 5A, 14A, and 14B). To assess the nature of the cells more closely, the transcriptomic profile of the CD8αβ iT cells was compared to that of healthy donor PBMC-derived lymphocytes. To enhance comparability, CD4 αβTCR-T (CD4), CD8 αβTCR-T (CD8), γδTCR-T (γδT) and NK cells were engineered to express the 1928z-1XX CAR (either through TRAC targeted integration in CD4 and CD8 or retroviral expression in γδT and NK cells) and purified for the CAR⁺ populations. Unsupervised hierarchical clustering analysis based on a dissimilarity matrix showed that, based on overall gene expression, the CD8αβ iT cells were most related to the CD4 and CD8 T cells (FIG. 5B). Principal component (PC) analysis confirmed that within the first two PCs, CD8αβ iT cell cluster more closely with peripheral blood γδTCR-T cells than γδTCR-T cells (FIG. 14C). Pearson's correlation distinguished that CD8αβ iT cells are more closely related to the peripheral blood CD8 T cells (r=0.99, FIG. 5C).

### 6. TRAC-1XX-iT Cells Achieve Tumor Control in a Systemic In Vivo Leukemia Model

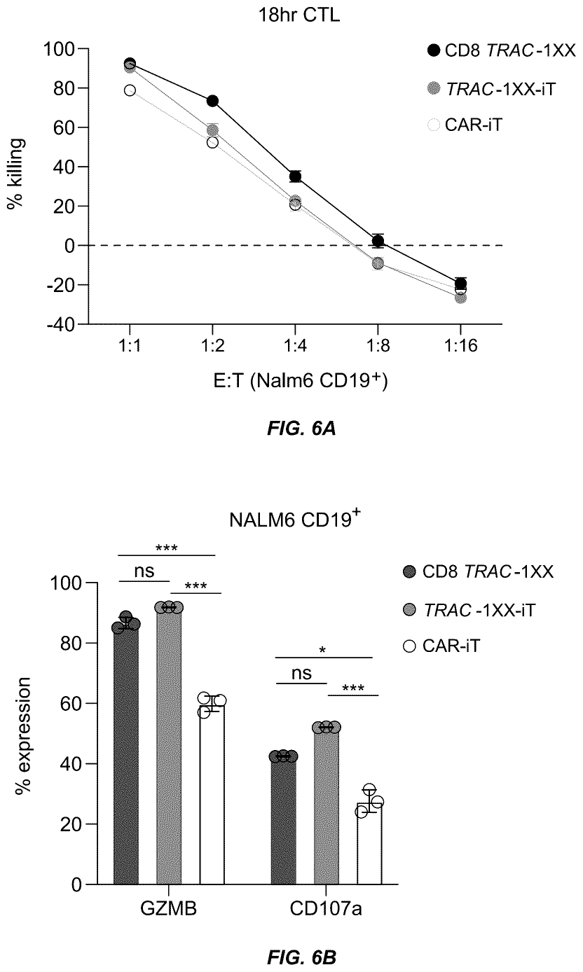

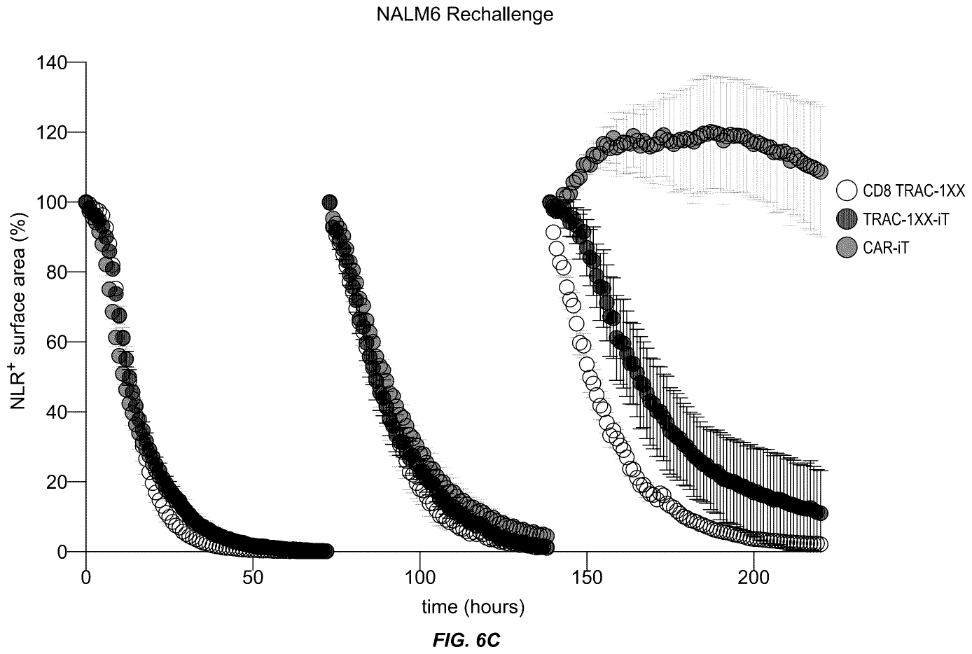

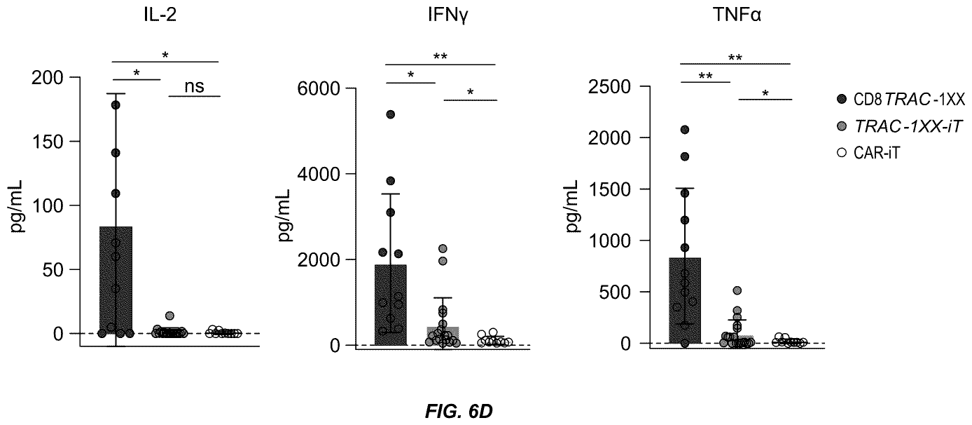

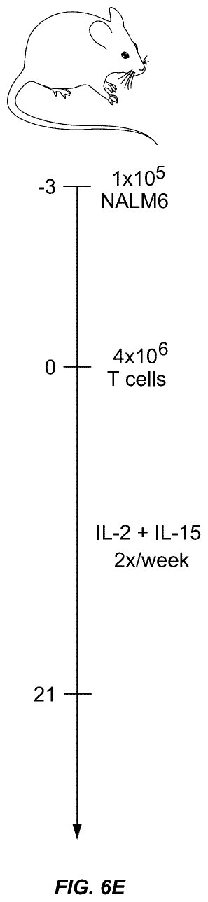

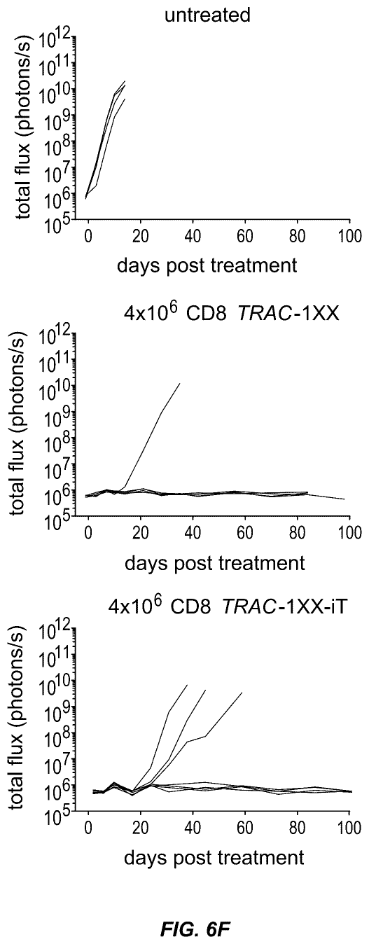

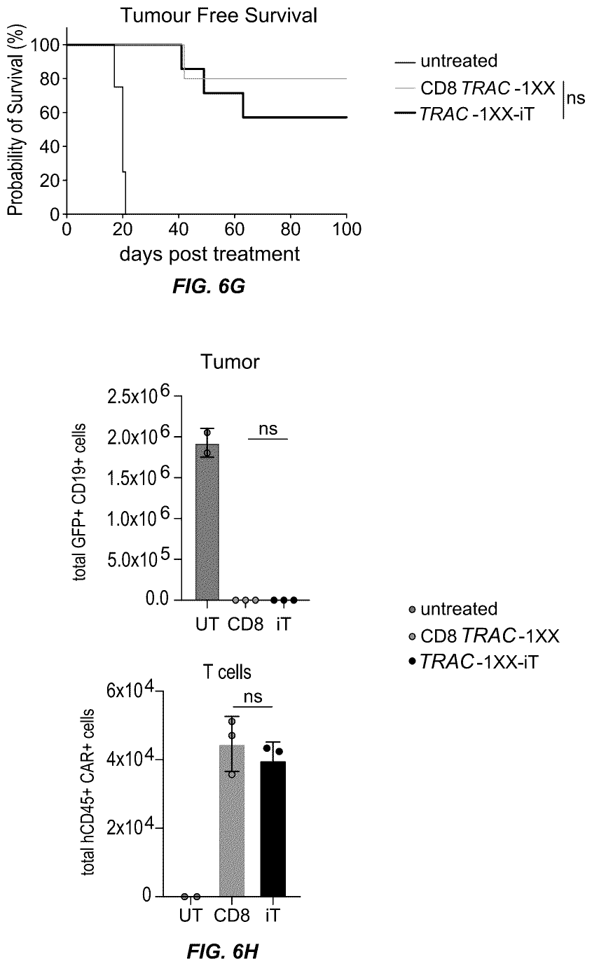

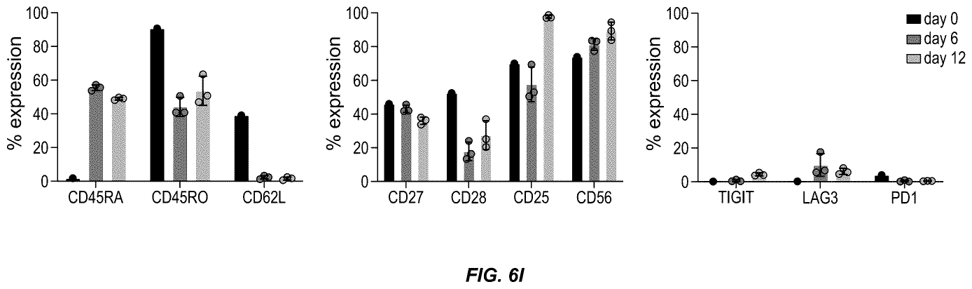

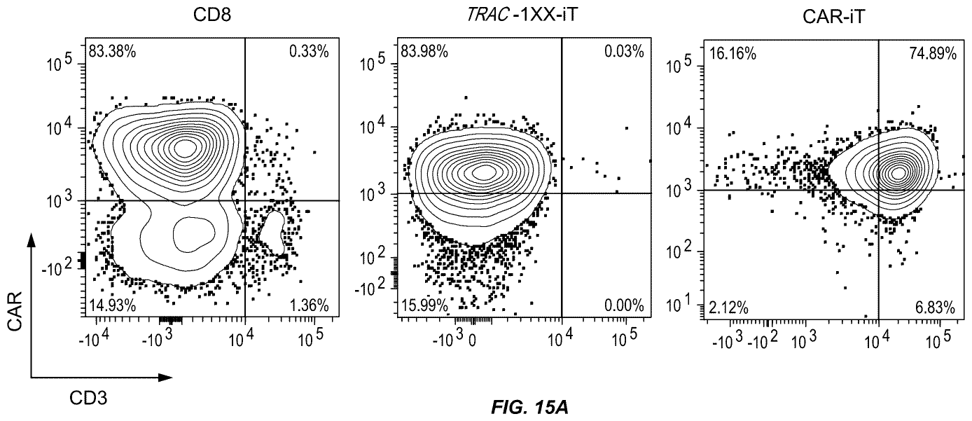

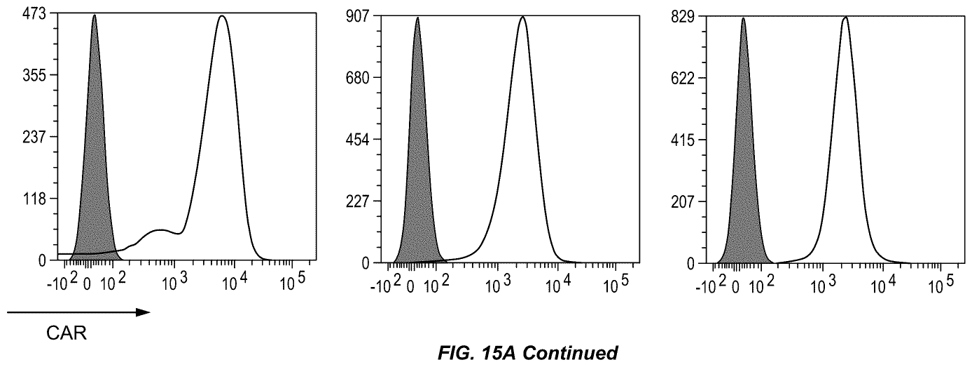

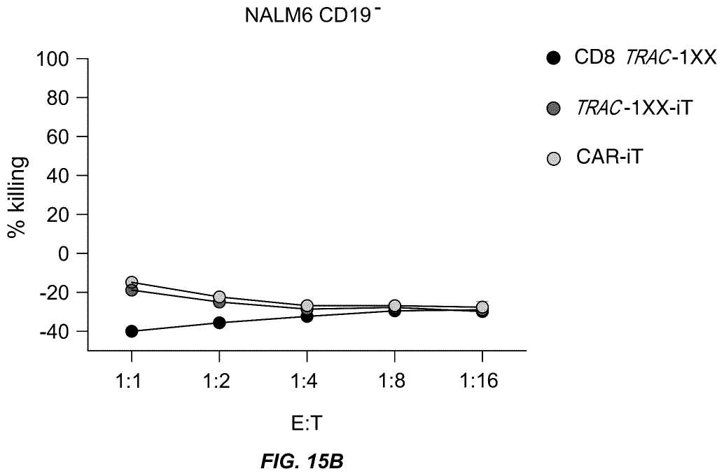

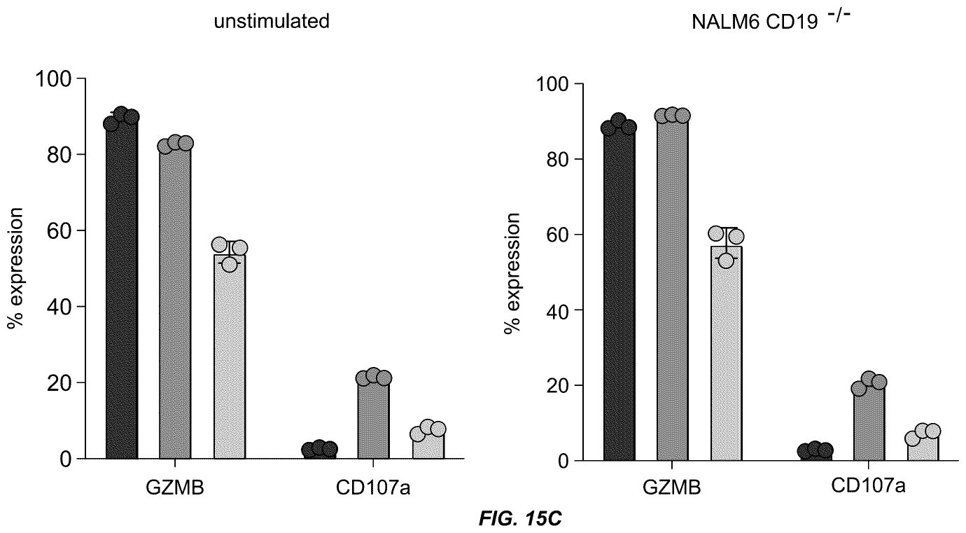

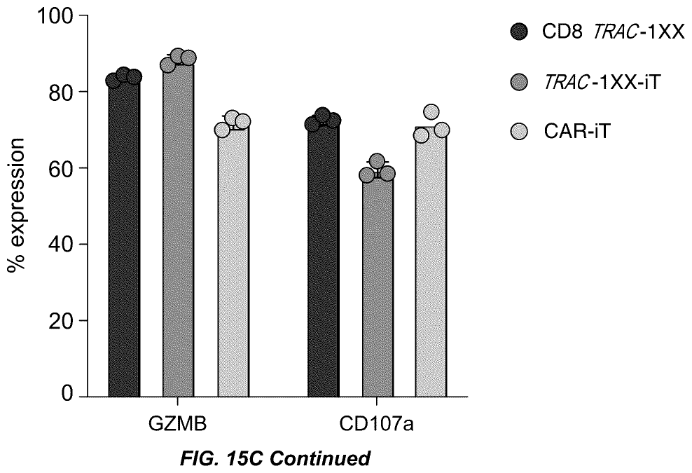

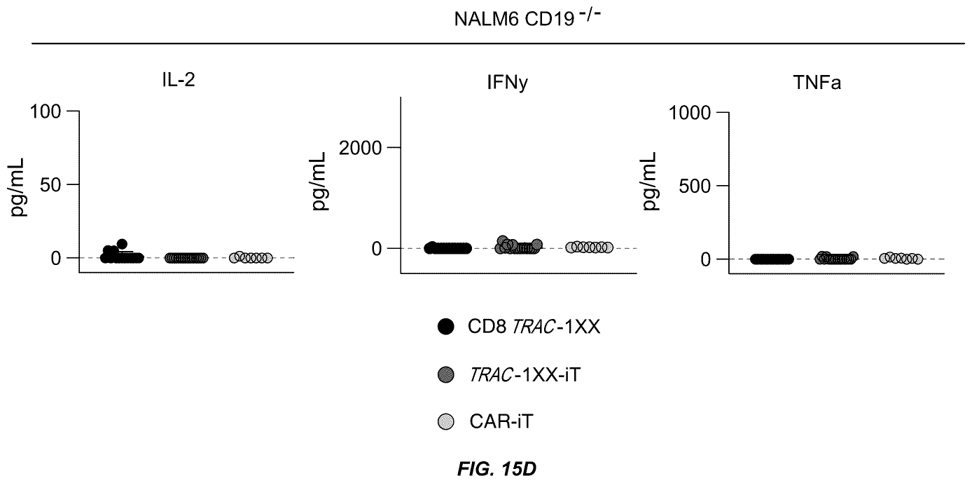

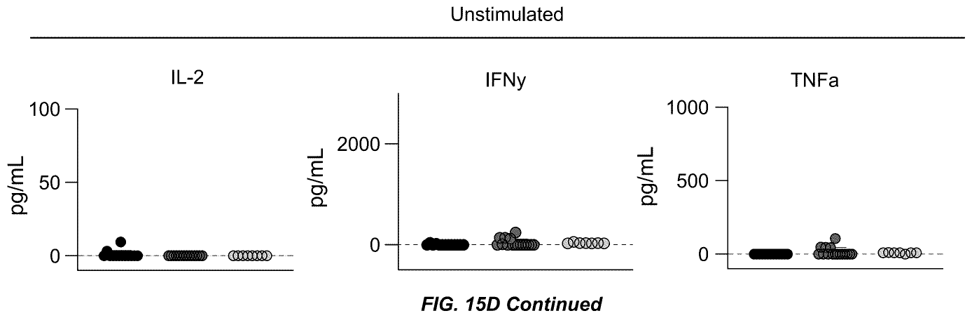

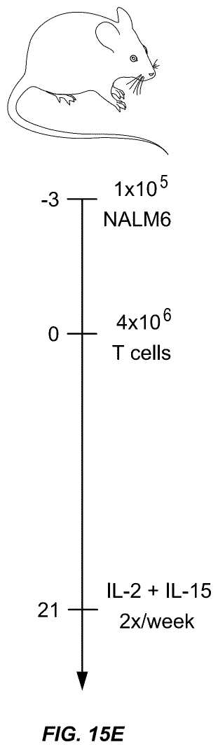

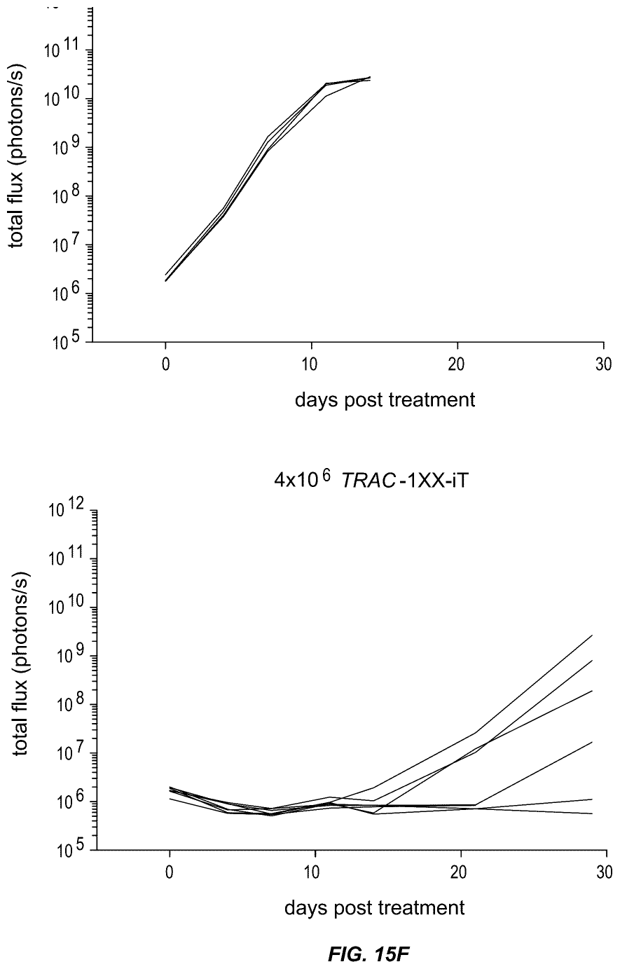

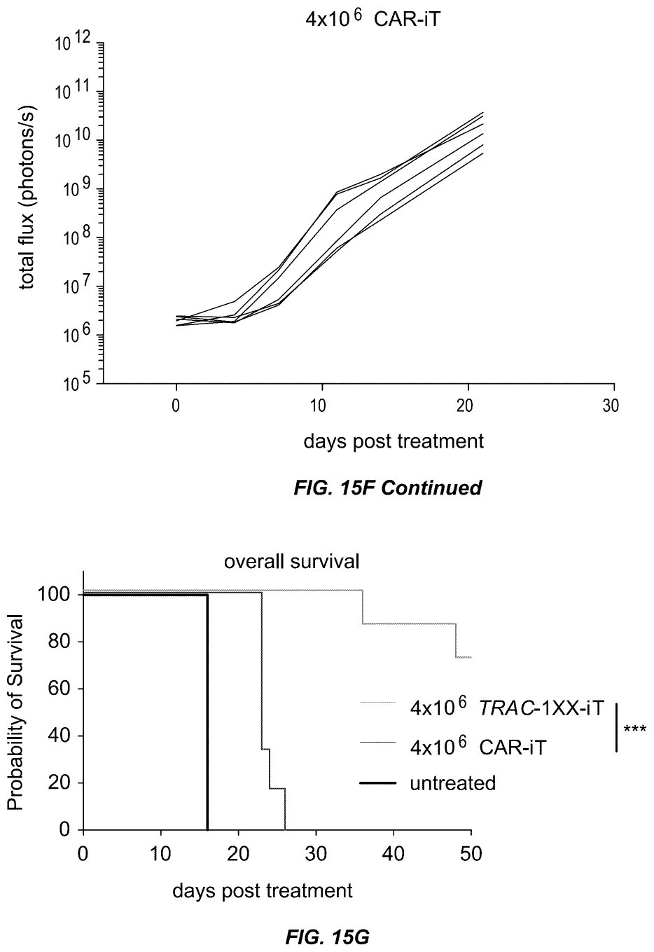

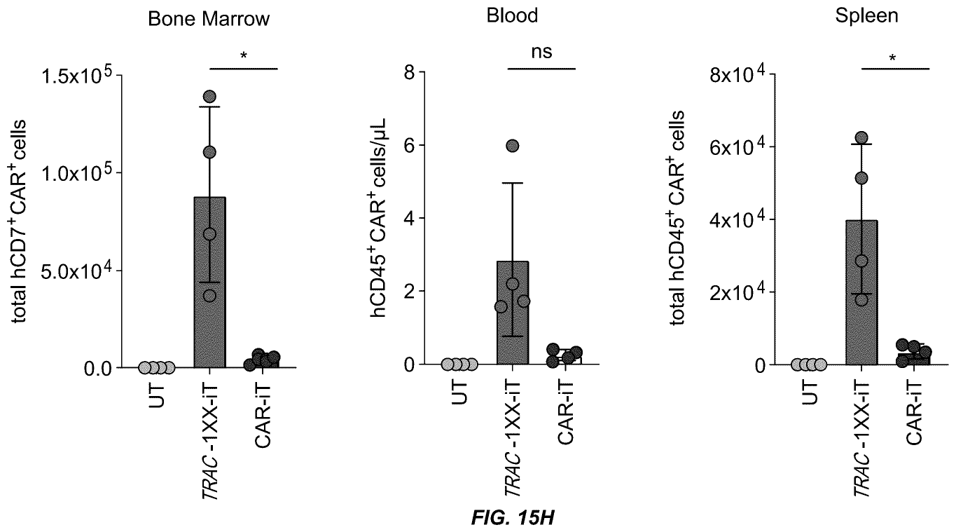

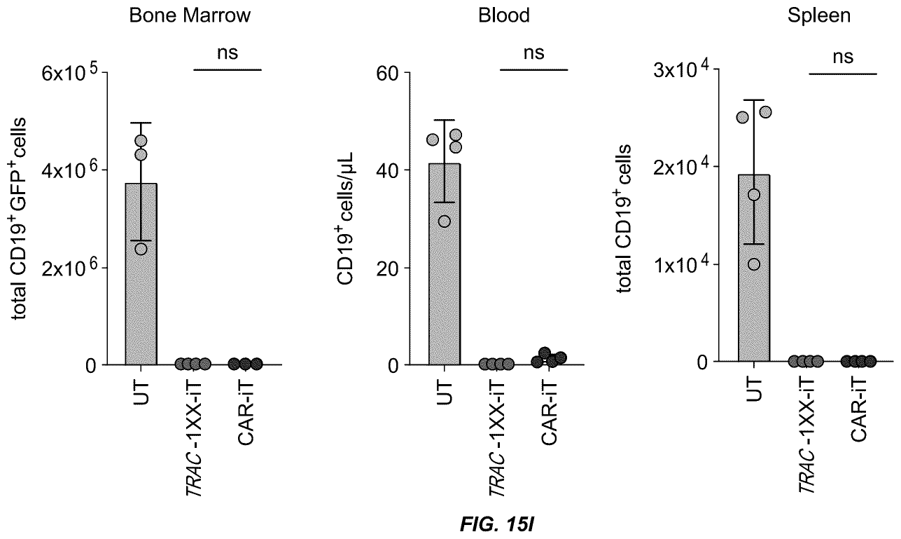

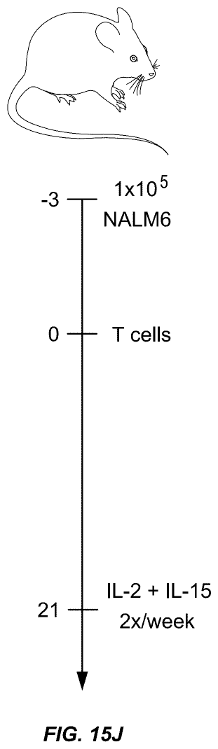

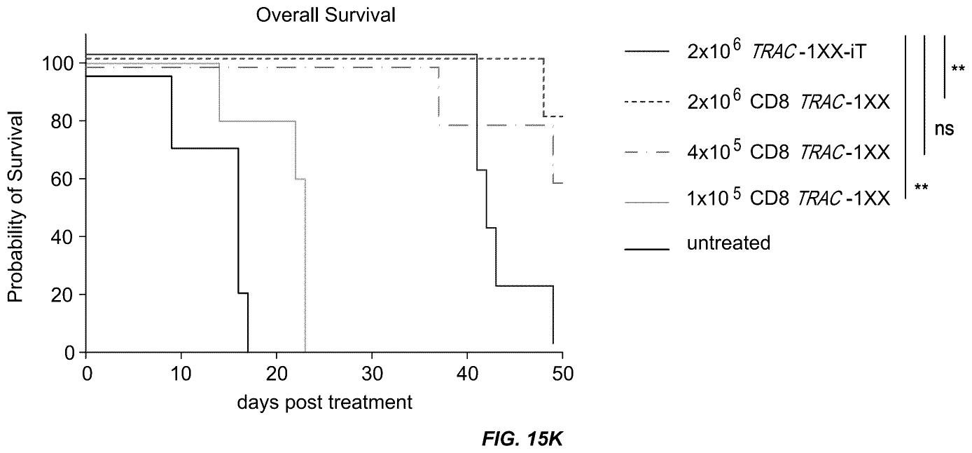

To determine whether the αβTCR-T lineage commitment of TRAC-1XX-iT cells enhanced their functional capabilities, their in vitro and in vivo functions were compared to CAR-iT cells and healthy-donor PBMC-derived CD8⁺ TRAC-1XX αβTCR-T cells (CD8 TRAC-1XX) (FIG. 15A). In vitro cytolytic activity was antigen-specific and similar between the three cell populations in a 18 h cytotoxicity assay (FIGS. 15A and 15B). However, Granzyme B and CD107a production was reduced in CAR-iT cells (FIGS. 6B and 15C). Importantly, TRAC-1XX-iT and CD8 TRAC-1XX cells were able to control repeated in vitro exposure to tumor cells, whereas CAR-iT cells failed after a third challenge (FIG. 6C). In vitro cytokine secretion was reduced in both iT populations compared to CD8 TRAC-1XX T cells (FIGS. 6D and 15D). TRAC-1XX-iT cells were able to produce IFNγ and TNFα, whereas minimal secretion was detected in CAR-iT cells. Notably, both TRAC-1XX-iT and CAR-iT cells lacked the ability to produce IL-2 in response to antigen (FIGS. 6D and 15D). To determine whether the differences in in vitro function between the TRAC-1XX-iT and CAR-iT cells translated into differences in in vivo tumor control, these were compared in the systemic NALM6 leukemia model (FIG. 15E), wherein TRAC-1XX-iT cells showed improved tumor control (FIGS. 15F and 15G), associated with increased iT cell persistence in the bone marrow, spleen and blood (FIGS. 15H and 15I).

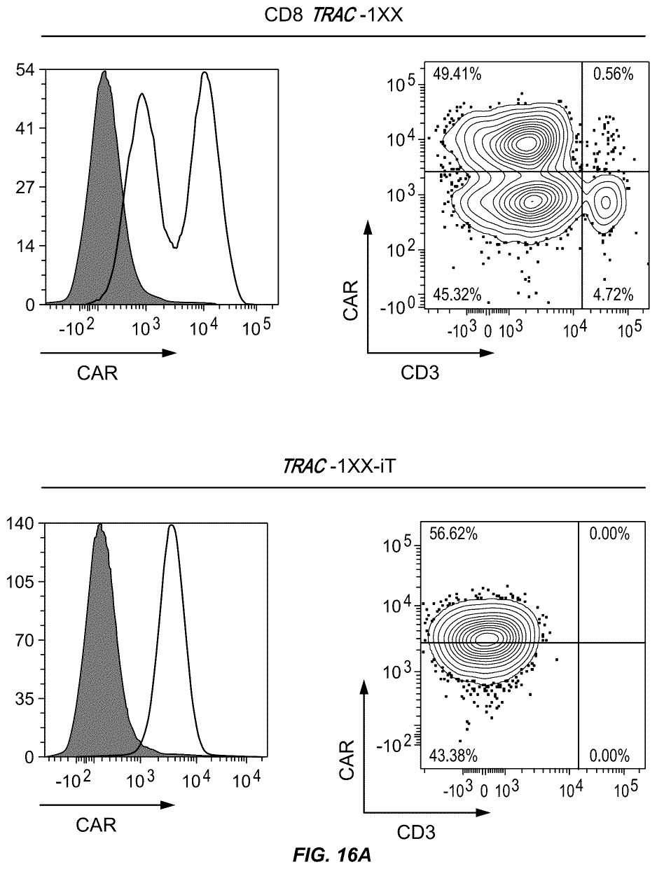

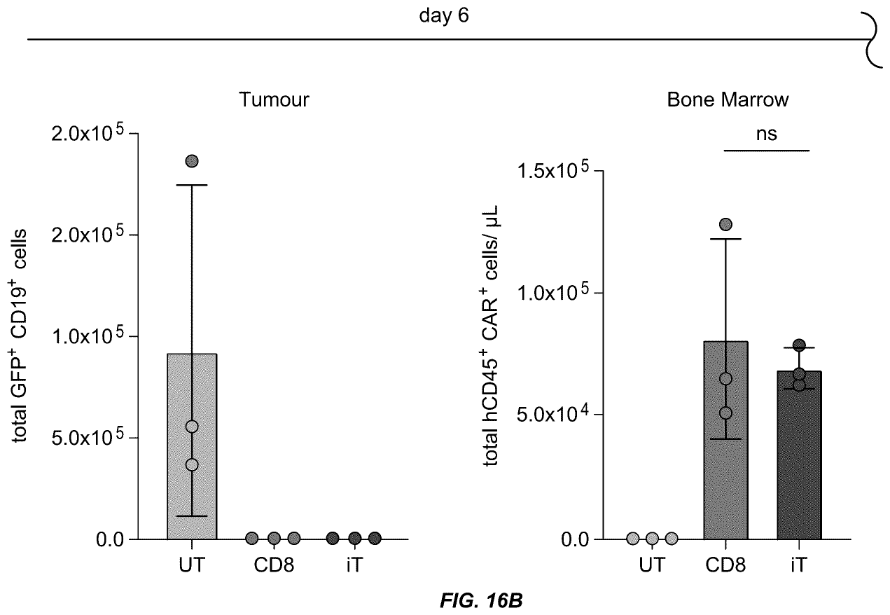

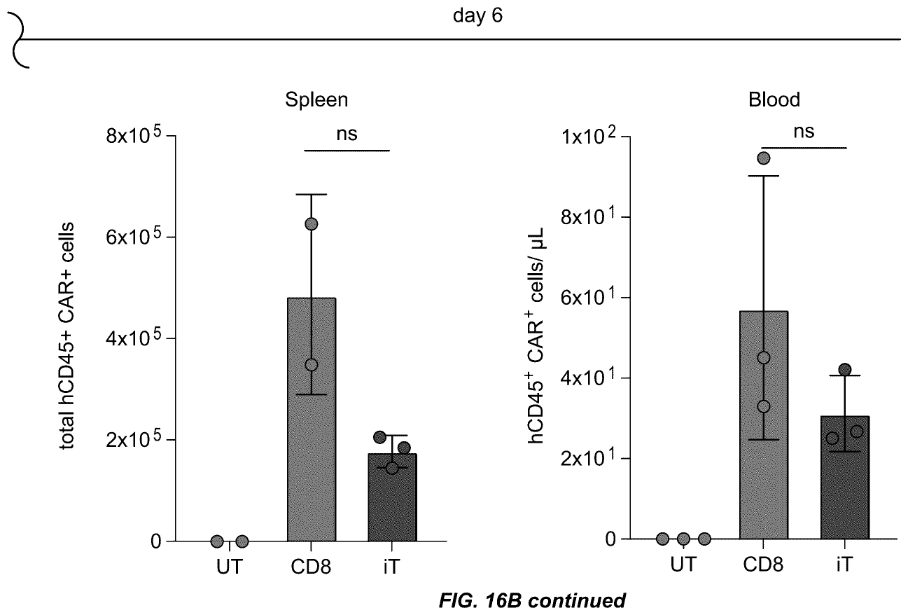

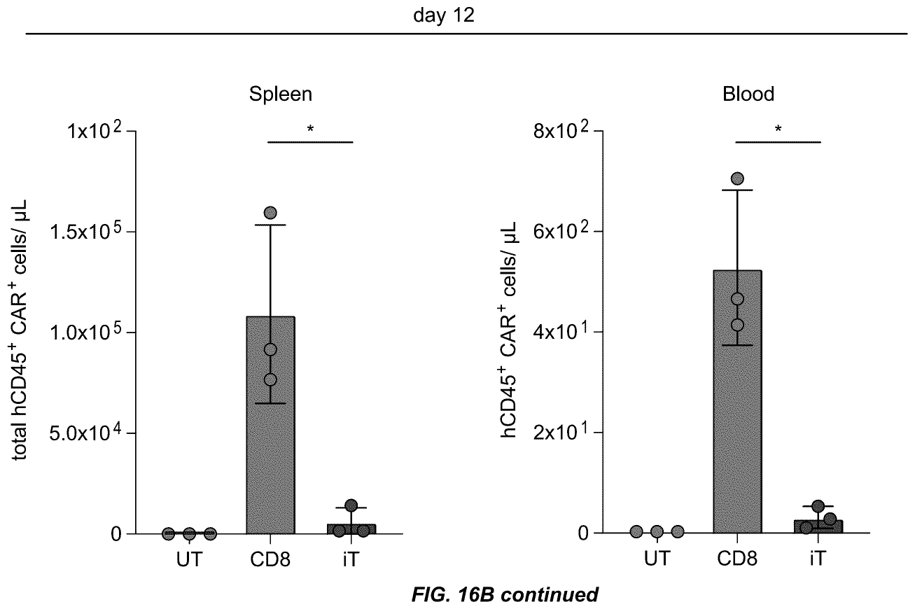

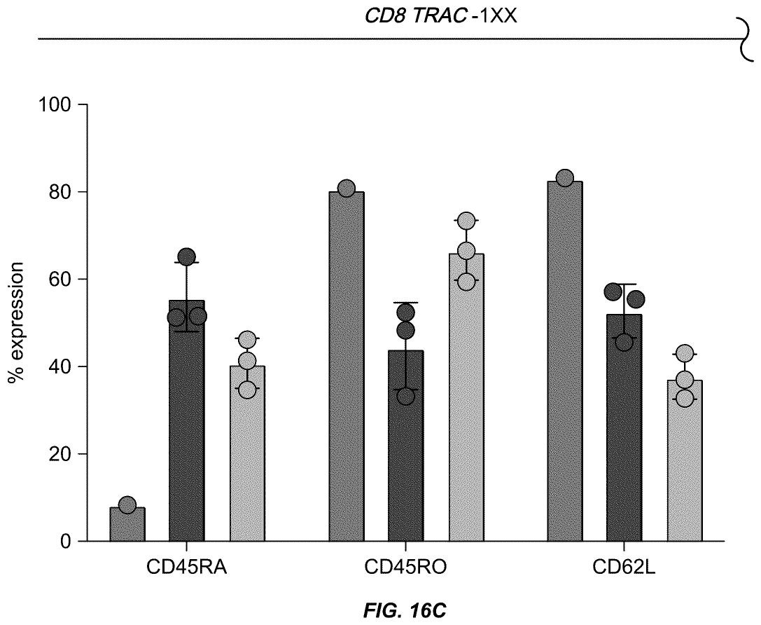

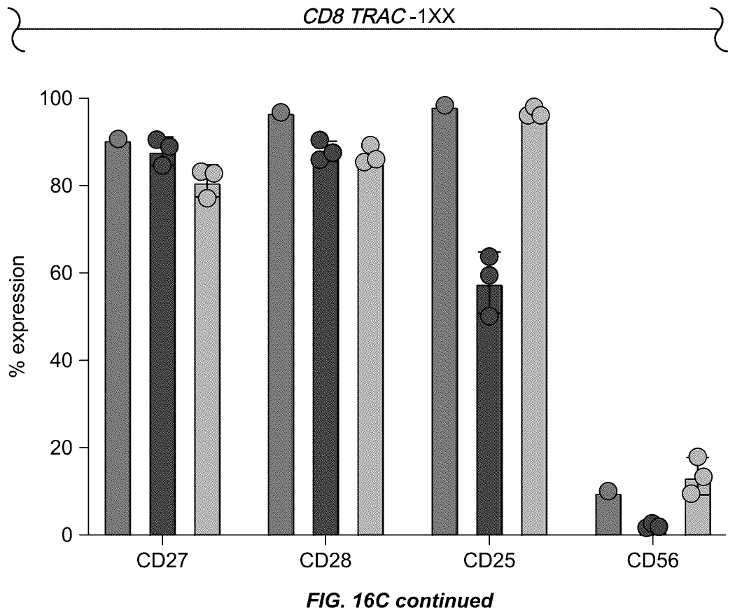

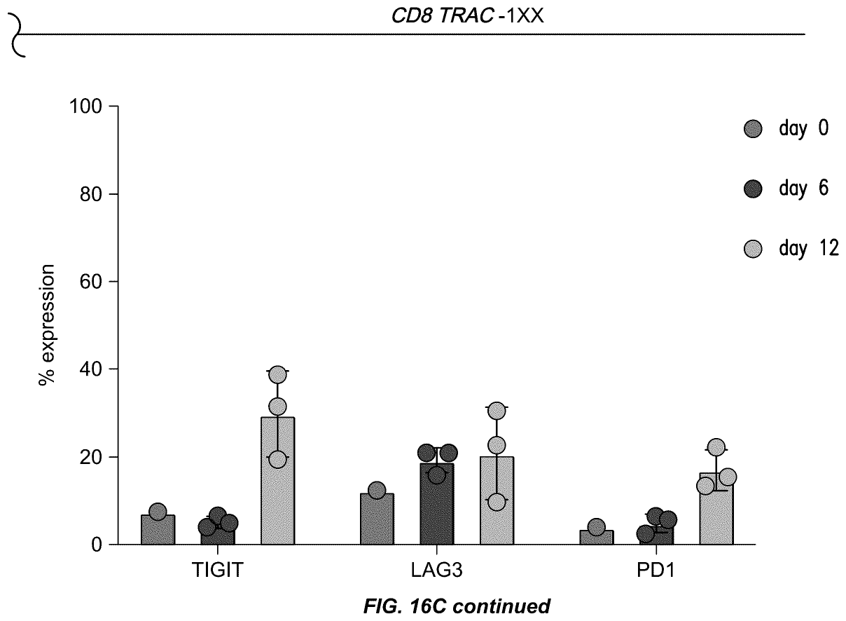

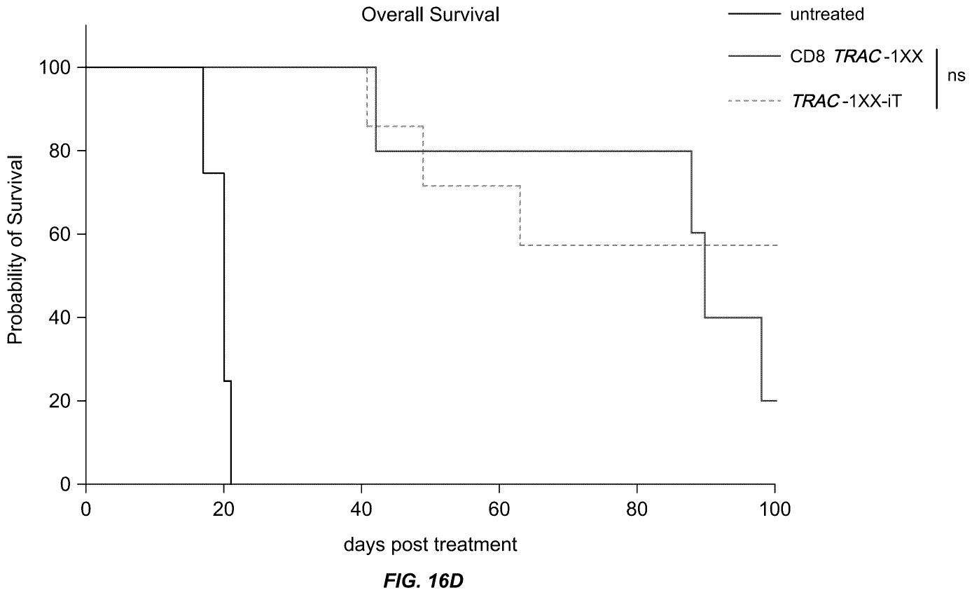

To provide a potency benchmark under these conditions, diminishing doses of CD8 TRAC-1XX T cells were administered and it was found that 2×10⁶ TRAC-1XX-iT cells provided a similar response to 4×10⁵ CD8 TRAC-1XX T cells (FIGS. 15J and 15H). 4×10⁶ cells TRAC-1XX-iT cells induced complete and durable responses (FIGS. 6E-6G). Enumeration of tumor cells in bone marrow, spleen and blood 6 and 12 days after infusion, showed complete absence of detectable tumor in the bone marrow for both treatment groups at either time point (FIGS. 6H and 16B). TRAC-1XX-iT cells showed similar persistence to CD8 TRAC-1XX T cells in bone marrow, spleen and blood by day 6 and in bone marrow on day 12, but were less abundant in spleen and blood by day 12 (FIGS. 6H and 16B). Phenotypically, both populations increased CD45RA expression in vivo, and diminished CD62L expression (FIGS. 6I and 16C). TRAC-1XX-iT cells down-regulated CD27 and CD28 but did not display increased exhaustion markers compared to peripheral blood CD8 TRAC-1XX T cells. Notably, the latter succumbed while presenting GvHD-like symptoms including weight loss, diarrhea and loss of fur, likely caused by the remaining small population of TCR⁺ cells (Qasim et al., Sci Transl Med 9 (2017)), which is not present in TRAC-1XX-iT cells (FIGS. 16A and 16D).

### 7. Discussion

The present example reports here on the generation of therapeutic CD8αβ CAR iT cells from TiPS. It was investigated how premature TCR or CAR expression interferes with adaptive T cell maturation and demonstrated that delayed expression and calibrated CAR signaling enable DP T cell development and terminal CD8αβ iT cell expansion in the absence of a TCR.

In vitro T cell development from TiPS that constitutively express a CAR yields T cells with an innate-like CD8αα T cell phenotype (Themeli et al., Nat Biotechnol31, 928-933 (2013); Harada et al., Mol Ther (2021)) or NK-like features (Ueda et al., Cancer Sci 111, 1478-1490 (2020); Maluski et al., J Clin Invest 129, 5108-5122 (2019)). The present example shows that premature CAR expression at the DN stage interferes with Notch signaling, skewing differentiation away from DP differentiation and towards the acquisition of an innate-like phenotype. The Notch ligand DLL1 was sufficient to induce DP cell formation during T lineage development from precursor cells that bear TCR VDJ genes in germline configuration, but not from WT-TiPS, which encode a rearranged αβTCR and required DLL4 to progress to the DP stage (FIG. 1D). In the presence of constitutive CAR expression however, DLL4 was no longer sufficient to evoke DP differentiation (FIG. 1F). Constitutive CAR expression diminished NOTCH1 expression and deregulated downstream gene expression, including DTXJ, TCF7 and PTCRA (FIG. 3B). Consistent with in vitro models of lymphopoiesis, stronger TCR signals or interference with Notch signaling impairs the DN to DP transition in αβTCR T cells (Ciofani et al., Immunity 25, 105-116 (2006); Hayes et al., Immunity 22, 583-593 (2005)) and defaults the cells towards an innate/γδTCR-like fate.

The combination of regulating CAR expression under the control of the TRAC locus (Eyquem et al., Nature 543, 113-117 (2017)) and inactivating the CAR's second and third ITAM (Feucht et al., Nat Med 25, 82-88 (2019)) brought down the potential for constitutive signaling (FIGS. 12A-12C) and rescued the induction of genes downstream of Notch and DP development (FIGS. 2D and 3B). The partially restored induction of PTCRA in differentiating TRAC-1XX-TiPS is noteworthy (FIGS. 3B-3D), given the crucial role the preTCR-complex plays in the development of αβTCR-T cells (but not γδTCR-T cells (Fehling et al., Nature 375, 795-798 (1995))). PreTCR expression is required for R chain selection and its absence diverts T cell differentiation towards a γδTCR-T cell phenotype (Terrence et al., J Exp Med 192, 537-548 (2000)). PTCRA expression is absent in the presence of a constitutively expressed CAR (FIG. 3B), consistent with Notch downregulation (Reizis & Leder, Genes Dev 16, 295-300 (2002)). Facilitating DP development through attenuation of CAR signaling is also consistent with the finding that attenuated γδTCR signaling can allow for DP maturation in absence of an αβTCR (Haks et al., Immunity 22, 595-606 (2005)).

TRAC-1XX-TiPS cannot assemble a functional αβTCR and therefore depend on the CAR to direct T cell maturation past the DP stage. Exposure to the CAR antigen resulted in the maturation of CD8αβ SP by day D42, but failed to support their expansion (FIGS. 4A and 4C). Provision of 4-1BB costimulation together with antigen enabled the emerging SP CAR T cells to expand. Up-regulation of 4-1BB has been observed in murine DP cells undergoing positive selection in vivo (Kim et al., Exp Mol Med 41, 896-911 (2009)). The benefit of providing costimulation is consistent with TCR/MHC interaction alone not sufficing to induce complete CD8 T cell maturation (Groves et al., J Immunol 158, 65-75 (1997); Anderson et al., Immunol Today 20, 463-468 (1999)). The emerging SP TRAC-1XX-iT cells exposed to 4-1BBL not only expanded but also acquired robust effector functions (FIGS. 4I-4M) and the ability to expand upon repeated antigen stimulation (FIG. 4K). However, these iT cells do not have a classical naïve phenotype, as they maintain CD5 and CD7 expression but do not homogeneously express CD45RA, CD62L and CCR7, as would be expected in naïve T cells and recent thymic emigrants (McFarland et al., Proc Nat Acad Sci USA 97, 4215-4220 (2000)). They rather express CD45RO, CD28, CD25 and CD56, hallmarks of recently activated T cells (FIG. 13C). This effector-like phenotype is commonly observed following extrathymic differentiation of T cells, irrespective of CAR expression or maturation protocol (Iriguchi et al., Nat Commun 12, 430 (2021); Ito et al., Commun Biol 4, 694 (2021)). Whist the TRAC-1XX-iT described here do not display a canonical naïve phenotype, it is noteworthy that they also do not express common exhaustion markers. Transcriptional studies confirmed that CAR-induced maturation produces CD8αβ TRAC-1XX-iT cells that are more similar to peripheral blood-derived TRAC-1XX CD8αβ T cells (CD8 TRAC-1XX) than CD4 αβTCR-T cells and are more distinct yet from γδTCR-T cells and NK cells (FIGS. 5B and 5C).

When comparing TRAC-1XX-iT function to CAR-iT and peripheral blood-derived CD8 TRAC-1XX, it was found that TRAC-1XX-iT had improved cytolytic capacity and cytokine secretion compared to CAR-iT (FIGS. 6D and 6D), as well as improved anti-tumor activity in vivo (FIGS. 15E-15H). TRAC-1XX-iT cells still produced significantly lower levels of cytokines than CD8 TRAC-1XX cells, notably lacking IL-2 production (FIG. 6D). TRAC-1XX-iT cells nonetheless provide substantial anti-tumor activity in a systemic NALM6 model, which CAR-iT cannot achieve (FIGS. 6G and 15G), while requiring a higher dosage than CD8 TRAC-1XX T cells (FIG. 15K). Phenotypically, both TRAC-1XX-iT and CD8 TRAC-1XX cells differentiated towards an effector phenotype upon encounter with the tumor in the bone marrow. TRAC-1XX-iT cells downregulated CD62L, CD27 and CD28, but did not show accelerated acquisition of exhaustion markers (FIGS. 6I and 16C). TRAC-1XX-iT cells showed reduced persistence in spleen and blood over time (FIG. 16B). Despite those differences, TRAC-1XX-iT cells were able to induce long-term remission and survival following intravenous infusion of a single dose of 4×10⁶ iT cells (FIGS. 6F and 6G). Tumor control by CAR-iT cells has only been hitherto achieved in intraperitoneal models (Themeli et al., Nat Biotechnol 31, 928-933 (2013); Harada et al., Mol Ther (2021); Ueda et al., Cancer Sci 111, 1478-1490 (2020)).

TiPS are a highly attractive resource for allogeneic, “off-the-shelf” immunotherapy (Themeli et al., Cell Stem Cell 16, 357-366 (2015)). The self-renewing capacity of TiPS allows for the establishment of gene edited, clonally selected master cell banks (Valamehr et al., Stem Cell Reports 2, 366,381 (2014); Nagano et al., Mol Ther Methods Clin Dev 16, 126-135 (2020)) which can be utilized to mass produce genetically homogeneous T cell populations, eliminating donor-dependent T cell variability and thereby standardizing treatment. Combined with genome editing, the TiPS platform allows for careful selection of a desired genotype, screening for insertional mutagenesis (Fraietta et al., Nature 558, 307-312 (2018)), off-target editing and translocations (Poirot et al., Cancer Res 75, 3853-3864 (2015); Stadtmauer et al., Science 367 (2020)), and facilitates the complete elimination of TCR expression to prevent GvHD (Qasim et al., Sci Transl Med 9 (2017)) or the accidental transduction of malignant cells in apheresis products (Ruella et al., Blood 135, 505-509 (2020)). Genotype selection, including detection of off-target editing events, renders the TiPS platform particularly suitable for multiplexed gene editing strategies, such as combining TRAC locus editing with the ablation of CD52 (Poirot et al., Cancer Res 75, 3853-3864 (2015)), CD70 (Mansilla-Soto et al., Nat Med 28, 345-352 (2022)), or PD1 (Stadtmauer et al., Science 367 (2020)). The use of TiPS-derived T cells is not limited to targeting CD19 as described here and is applicable to other target tumor associated antigens, barring that potential interaction between the CAR with an antigen expressed during T cell development does not interfere with T lineage commitment, as well as applications beyond cancer immunotherapy (Amor et al., Nature 583, 127-132 (2020)). In the presently disclosed protocol, 1 million TRAC-1XX-iT cells are generated from a single TiPS in 42 days (FIG. 4H). This engineering flexibility and expansion potential provide a platform that can be feasibly scaled to generate clinically relevant CAR iT cell numbers, which in principle may allow for off-the-shelf application from batches of uniform and consistent CAR iT cells produced from the same engineered master cell bank.

In summary, the present example demonstrate that synthetic receptors like CARs can substitute for the TCR in driving directed T cell differentiation and show that induction of TCR^{-/-}, αβTCR-T cell-like CD8αβ CAR T cells is feasible and holds great potential for large scale production of potent T cell-based immunotherapies.

### 8. Methods

Cell Lines: OP9-mDLL1. OP9-mDLL1 cells were cultured as previously described (Themeli et al., Nat Biotechnol 31, 928-933 (2013)).

Cell Lines: OP9-DLL1, -DLL4, -JAG1, -JAG2. Parental OP9 cells were obtained from ATCC. Plasmids encoding the Moloney murine leukemia virus-based SFGγ retroviral vector (Riviere et al., Proc Natl Acad Sci USA 92, 6733-6737 (1995)) were used to clone bi-cistronic constructs to express one of the human Notch ligands (DLL1, DLL4, JAG1 or JAG2) and GFP. Plasmids containing sequences of hDLL1, hDLL4, hJAG1 and hJAG2 were obtained from GenScript (NM_005618, NM_019074, NM_000214 and NM_002226 respectively) and were cloned using standard molecular biology techniques by replacing the FFLuc element in the SFG-FFLuc-P2A-GFP retroviral vector with the desired Notch ligand. Vesicular stomatitis virus glycoprotein G (VSV-G) pseudotyped retroviral supernatants derived from transduced gpg29 fibroblasts (H29) was used to transduce OP9. Transduced cells were purified by flow cytometry based on Notch ligand and GFP expression. Antibodies used to detect Notch ligand expression were hDLL1—PE (MHD1-314; BioLegend), hDLL4—PE (MH1D4-46; BioLegend), hJagged-1—PE (MHJ1-152; BD) and hJagged-2—PE (MHJ2-523; BioLegend) respectively. Purified OP9 cells were cultured in MEMa (Gibco) media with 20% Fetal Bovine Serum (FBS, Hyclone), 1×MEM Non-Essential Amino Acids (NEAA, Corning), 2 mM GlutaMAX (Gibco), 100U/mL Penicillin (Pen) and 100 μg/mL Streptomycin (Strep, Corning), 55 tM 2-Mercaptoethanol (2-ME, Gibco) and 50 mg/mL ascorbic acid (Sigma) as previously described (Themeli et al., Nat Biotechnol 31, 928-933 (2013)).

Cell Lines: NALM6. NALM6 cells were obtained from ATCC. NALM6 CD19^{-/-} were generated as previously described (Hamieh et al., Nature 568, 112-116 (2019)). NALM6 were transduced to express GFP and firefly Luciferase (FFLuc) for in vitro and in vivo detection (Zhao et al., Cancer Cell 28, 415-428 (2015)). Cells were cultured in RPMI 1640 (Corning) with 10% FBS (Hyclone) 1×NEAA, 2 mM GlutaMAX, 100U/mL Pen, 100 tg/mL Strep, 2 mM HEPES (Corning) and 55 tM 2-ME). For Incucyte-based analysis, NALM6 CD19⁺ and NALM6 CD19^{-/-} were transduced with Incucyte NucLight Red lentiviral reagent (NLR, Essen BioScience) and selected with puromycin according to manufacturer's instructions.

Cell Lines: 3T3-CD19. NUH 3T3 cells expressing CD19 were used as artificial antigen presenting cells as previously described (Zhao et al., Cancer Cell 28, 415-428 (2015)). 3T3-CD19-4-1BBL were generated utilizing a previously described SFGg retroviral vector encoding the 4-1BBL transgene (Riviere et al., Proc Natl Acad Sci USA 92, 6733-6737 (1995)). VSV-G pseudotyped retroviral supernatant derived from transduced H29 was used to transduce 3T3-CD19. Transduced cells were purified by flow cytometry based on 4-1BBL (4-1BBL—PE, 5F4; BioLegend) expression. Cells were cultured in DMEM media (Corning) with 10% Cosmic Calf Serum (Hyclone).

Cell Lines: K562-mbIL-21-4-1BBL. K562 cells were transduced to express membrane-bound IL-21 and 4-1BBL as previously described (Cichocki et al., Sci Transl Med 12 (2020)). Cells were cultured in RPMI 1640 (Corning) with 10% FBS (Hyclone), 2 mM GlutaMAX, 100U/mL Pen 100 tg/mL Strep, 55 tM 2-ME.

Cell Lines: H1. The human embryonic stem cell line was cultured on MEF in hES media (DMEM-F12 (Corning) with 20% knock-out serum replacement (KSR), 1×NEAA, 2 mM GlutaMAX, 100U/mL Pen 100 μg/mL Strep, 55 μM 2-ME) supplemented with 8 ng/mL hbFGF (R&D systems).

Cell Lines: Primary CLL cells. Apheresis product before CAR T cell infusion were obtained from patients thatwere consented and enrolled in phase I 1928z CAR T cell clinical trials.

Generation of iPS: FiPS. Fibroblast-derived iPS (FiPS) were generated as previously described (Valamehr et al., Stem Cell Reports 2, 366¬381 (2014)), transfected with a transient plasmid-based reprogramming system to initiate cellular reprogramming. In brief, cells were transfected with reprogramming vector backbone (pCEP4, Life Technologies) containing OCT4, NANOG, and SOX2 under regulation of the EF1a promoter. Transfected cells were plated on Matrigel and selected with hygromycin until FiPS colonies were established.

Generation of iPS: WT-TiPS. WT-TiPS were generated as previously described (Clone T-iPSC-1.10) (Themeli et al., Nat Biotechnol 31, 928-933 (2013)). In brief, healthy-donor peripheral blood mononuclear cells (PBMCs) were activated with phytohemagglutinin (PHA, 2 μg/mL) and transduced with two tri-cistronic SFGγ retroviral vectors, each vector encoding reprogramming factors and a different fluorescent marker (f-Citrine-P2A-cMYC-E2A-SOX2 and f-vexGFP-P2A-OCT4-T2A-KLF4). Transduced cells were seeded on MEF feeder cells and TiPS colonies were established.

Generation of iPS: CAR-TiPS. CAR-TIPS were generated as previously described (1928z-T-iPSC) (Themeli et al., Nat Biotechnol 31, 928-933 (2013)). In brief, clone T-iPSC-1.10 was stably transduced with a bi-cistronic lentiviral vector (mCherry-P2A-1928z) and purified for mCherry expression by flow cytometry.

Generation of iPS: TRAC^{-/-}-TiPS. TRAC^{-/-}-TiPS were generated through CRISPR/Cas9-targeted integration of a EF1a-GFP-P2A-Puromycin-bGHpA (G2AP) expression unit into the TRAC locus leading to knockout of the TCRα gene into the WT-TiPS (Clone T-iPSC-1.10). WT-TiPS were electroporated using Lonza Cell Line Nucleofector Kit V solution and Lonza Nucleofector-II. Five million cells were resuspended in 100 uL nucleofection solution, with 2.6 μg pBS-TRAC gRNA1, 2.6 μg pBS-TRAC-HR-G2AP and 2.6 tg hCas9 plasmid, and electroporated using program B-025. Transfected cells were plated on Matrigel-coated plates using TiPS complete medium containing 10 μM ROCK inhibitor. After 48 h cells were selected with 0.8 μg/ml Puromycin and sorted by flow cytometry for GFP expression. pBS-TRAC gRNA1 was generated by cloning the TRAC gRNA target sequence (5′-CAGGGTTCTGGATATCTGT) into pBS-gRNA MCS plasmid, which contains the human U6 promoter and the gRNA fold described in Mali et al., Science 2013. pBS-TRAC-HR-G2AP was generated by cloning the left (~0.9 kb) and right (1 kb) TRAC homology arms (HAs) into pBluescript II SK (+), followed by the insertion of the EF1a-GFP-P2A-Puromycin-bGHpA expression unit in between of the HAs. hCas9 plasmid (Mali et al., Science 339, 823-826 (2013)) was obtained from Addgene (41815).

Generation of iPS: TRAC-1928z-TiPS. TRAC-1928z T cells were generated as previously described (Eyquem et al., Nature 543, 113-117 (2017); Mansilla-Soto et al., Nat Med 28, 345-352 (2022)). In brief, αβTCR-T cells were purified from PBMCs with the Pan T cell Isolation kit (Miltenyi Biotec) on the AutoMACS Pro according to manufacturer instructions. Purified cells were activated with CD3/CD28 Dynabeads (1:1 beads:cell) in X-Vivo 15 media (Lonza) supplemented with 5% human serum (HS) (Gemini Bioproducts) with 5 ng/mL rhIL-7 (R&D Systems) and 5 ng/mL rhIL-15 (R&D Systems). 48 h after αβTCR-T cell activation, CD3/CD28 beads were magnetically removed, and T cells were transfected by electrotransfer of TRAC ribonucleoprotein (RNP) using the Nucleofector II device (Lonza). Then 2×10⁶ T cells were resuspended in P3 buffer (Lonza) and mixed with 60 pmol TRAC RNP in a total volume of 20 μL. Following electroporation and considering 66.7% viability, cells were diluted into culture medium and 1×10⁶/mL and incubated at 37° C., 5% CO2. Recombinant AAV6 donor vector pAAV-TRAC-1928z (Eyquem et al., Nature 543, 113-117 (2017)) was added to the culture 30 min after electroporation at a multiplicity of infection of 3×10⁵ genome copies (GC). Twenty-four h after targeting, T cells were reprogrammed as described above (WT-TiPS) and TRAC-1928z-TiPS colonies were established and cloned on MEF feeder cells. PCRs were performed to determine biallelic, specific target transgene integration into the TRAC locus.

Generation of iPS: TRAC-LXX-TiPS. αβTCR-T cells were isolated, activated and transfected as described above (TRAC-1928z-TiPS). Following electroporation cells were transduced with the previously described recombinant AAV6 donor vector pAAV-TRAC-1XX containing the 1928z-1XX CAR construct. The 1928z-1XX CAR contains tyrosine-to-phenylalanine point mutations within ITAM2 and ITAM3 of the CD3z domain rendering only ITAM1 functional (Feucht et al., Nat Med 25, 82-88 (2019)) TRAC-1XX-T cells were reprogrammed as described above for the FiPS. Emerging iPSCs colonies were expanded and cloned by limiting dilution. PCRs were performed to determine biallelic, specific target transgene integration into the TRAC locus.

Generation of iPS: iPS culture. iPS lines were maintained on MEF prior to EB-based differentiation, in serum-free hES medium supplemented with 8 ng/mL hbFGF. Prior to monolayer-based iCD34 differentiation, iPS lines were cultured on Matrigel in hES media containing 0.4 μM PD032590, 1 μM CHIR99021, 5 μM Thiazovivin, 2 μM SB431542 (all Biovision), 10 μM ROCK-inhibitor (Ascent) and 10 ng/mL hbFGF (R&D Systems) as previously described (Valamehr et al., Sci Rep 2, 213 (2012)). Fresh media was provided every day and cells were passaged every 3-4 days as previously described (Themeli et al., Nat Biotechnol 31, 928-933 (2013); Valamehr et al., Stem Cell Reports 2, 366-381 (2014)). iPS lines were tested for Mycoplasma contamination every 2 months. iPSC surface marker expression. iPS lines were assessed for cell surface pluripotency marker expression including SSEA4—FITC (MC813-70; BD), TRA-1-81—af647 (TRA-1-81; BD) and CD30—PE (BerH8; BD).

Verification of transgene integration into the TRAC locus. Genomic DNA was isolated using QuickExtract DNA Extraction Solution (Lucigen) following manufacturer's protocols.

PCRs were performed using KAPA 2×HiFi Hot Start Ready Mix following manufacturer's recommended conditions. PCR products were analyzed using ethidium bromide-stained agarose gel electrophoresis and imaged using the Bio-Rad ChemiDoc.

Verification of transgene integration into the TRAC locus: TRAC^{-/-}-TiPS. Successful disruption of the TRAC locus through insertion of the G2AP expression unit was verified through PCR of the region spanning between the left- and right homology arms. Primers: 5′-GATGATACGCGTCTCTTCTCCTTTCTCATTGAGC and 5′-TCGAGTAAACGGTAGTGCTG. Non-targeted allelles produce a 1603 bp PCR product, targeted alleles a 4434 bp product.

Verification of transgene integration into the TRAC locus: TRAC-1928z-TiPS and TRAC—1XX-TiPS. Successful integration of the 1928z and 1928z-1XX CAR construct respectively were assessed as previously described (Feucht et al., Nat Med 25, 82-88 (2019); Eyquem et al., Nature 543, 113-117 (2017)).

T cell differentiation from iPS and expansion of TRAC-1XX-iT cells. For the differentiation of iPS to hematopoietic precursors, optimized serum- and feeder-free in vitro differentiation protocols were used based on Embryoid Body (EB) formation (Themeli et al., Nat Biotechnol 31, 928-933 (2013)) or monolayer-based (iCD34) (Valamehr et al., Stem Cell Reports 2, 366,381 (2014)).

Hematopoietic precursor differentiation. EB-based precursor differentiation was performed as previously described (Themeli et al., Nat Biotechnol 31, 928-933 (2013)). Undifferentiated TRAC^{-/-}-TiPS or WT-TiPS colonies were transferred to ultra-low attachment plates to allow for EB formation in serum-free differentiation medium (StemPro-34 (Invitrogen), with 2 mM GlutaMAX, 1×NEAA, 100U/mL Pen, 100 μg/mL Strep, 55 μM 2-ME, and 50 mg/mL ascorbic acid). Mesoderm induction was facilitated through EB culture with 30 ng/mL hBMP-4 and 5 ng/mL hbFGF until day 4. Next, hematopoietic specification and expansion was achieved in the presence of 20 ng/mL hVEGF and a cocktail of hematopoietic cytokines (100 ng/mL rhSCF, 20 ng/mL rhFlt3L, 20 ng/mL rhIL-3 and 5 ng/mL hbFGF). Cells were transferred to fresh media with cytokines every 48 h. All cytokines were obtained from R&D systems. Day 10 EBs containing hematopoietic progenitor cells were dissociated with Accutase (StemCell Technologies) prior to culture on OP9 for T lymphoid commitment and expansion.

Monolayer-based iCD34 differentiation was performed as previously described (Cichocki et al., Sci Transl Med 12 (2020)). H1, FiPS or TiPS were differentiated to mesoderm and subsequently to CD34+ hematopoietic progenitors on matrigel in StemPro differentiation media supplemented with a combination of 5 ng/mL hBMP-4, 10 ng/mL hbFGF, 10 ng/mL rhVEGF, 50 ng/mL hSCF, 10 ng/mL hIL-6, 10 ng/mL hIL-11 for 10 days. Media with fresh cytokines was supplemented every 48 h. All cytokines were obtained from R&D systems. On day 10, CD34+ cells were enriched through positive selection with CD34 microbeads (Miltenyi Biotec) on the AutoMACS Pro according to manufacturer instructions, prior to T lymphoid differentiation on OP9.

T lymphoid commitment and expansion. Day 10 single cells were seeded on OP9 monolayers in OP9 medium (MEMa with 20% FBS), supplemented with 10 ng/mL rhTPO, 5 ng/mL rhIL-3, 30 ng/mL rhSCF, 10 ng/mL rhIL-7 and 10 ng/mL rhFlt3L to initiate lymphoid lineage commitment until day 20, and with 30 ng/mL rhSCF, 10 ng/mL rhIL-7 and 10 ng/mL rhFlt3L to complete T lineage commitment until day 35. Differentiating T lymphoid cells were passaged onto fresh OP9 monolayers every four days, fresh media with cytokines was supplemented 48 h after each passage. For the stimulation and expansion of TRAC-1XX-iT cells, differentiated cells were harvested from OP9 monolayers on day 35 and seeded on a monolayer of irradiated 3T3-CD19±4-1BBL at a 3:1 E:T ratio in T cell expansion media (CTS Optimizer Media (Gibco) with 1×CTS T cell maturation/expansion supplement and 1×CTS Immune cell serum replacement, 5 ng/mL rhIL-7 and 25 ng/mL rhIL-21). Cells were fed with fresh expansion media every 48 h. For maturation of iT cells on CD19 recombinant protein, flat-bottom tissue culture plates were coated with recombinant human CD19-Fc chimeric protein (R&D Systems), in 100 mM sodium-bicarbonate coating buffer, overnight at 4° C. Plate were blocked with PBS+5% FBS for 30 min at room temperature and washed twice with PBS. iT cells were resuspended at 0.25×10⁶ cells/mL in T cell expansion media with 5 ng/mL rhIL-7, 25 ng/mL rhIL-21 and 3 μg/mL Urelumab (Creative Biolabs). Cells were passaged after 48 h and fresh media supplemented with cytokines was added every 2 days.

PBMC derived cell isolation, activation, culture, and transduction. Buffy coats from healthy volunteer donors were obtained from the New York Blood Center. PBMCs were isolated by density gradient centrifugation.

αβTCR-T cells. αβTCR-T cells were purified and engineered as described above (TRAC-1928z-TiPS). After AAV6 transduction with pAAV-TRAC-1XX, cells were cultured in media supplemented with 5 ng/mL IL-7 and 5 ng/mL IL-15 for 3-5 days. After expansion, CD4 and CD8 cells were purified using EasySep™ CD4+ or CD8⁺ negative selection T cell isolation kit (Stem Cell Technologies) and purified for CAR expression by flow cytometry.

γδTCR-T cells. PBMCs were resuspended in lymphocyte media (RPMI 1640 media with 10% FBS) supplemented with 1 μg/mL Zoledronic Acid (Stem Cell Technologies) 10 ng/mL rhIL-15 (R&D systems) and 100U/mL IL-2 (Proleukin). After 72 h cells were fed with additional media and cytokines, after 6 days γδTCR-T cells were purified with the Miltenyi Biotec TCRγ/δ⁺ T cell isolation kit. Purified γδTCR-T cells were transduced with SFGg-1928z-1XX-P2A-LNGFR to induce CAR expression. Cells were expanded for 5-7 days in media supplemented with cytokines and purified for LNGFR expression using magnetic isolation with LNGFR—PE (C401457; BD) and anti-PE microbeads (Miltenyi Biotec).

NK cells. NK cells were purified from PBMCs with the NK Cell Isolation Kit (Miltenyi Biotec). NK cells were resuspended in lymphocyte medium and activated with K562-mbIL21-41BBL at a 1:1 E:T, supplemented with 1000U/mL IL-2. 48 h after activation NK cells were transduced with SFGg-1928z-1XX-P2A-LNGFR to induce CAR expression. Cells were expanded for five days in media supplemented with cytokines and purified for LNGFR expression using magnetic isolation with LNGFR—PE (C401457; BD) and anti-PE microbeads (Miltenyi Biotec) on the AutoMACS Pro.

Retroviral vector constructs, retroviral production, and transduction. Plasmids encoding the SFGγ-retroviral vector (Riviere et al., Proc Natl Acad Sci USA 92, 6733-6737 (1995)) were prepared as previously described (Brentjens et al., Nat Med 9, 279-286 (2003); Maher et al., Nat Biotechnol 20, 70-75 (2002)). VSV-G pseudotyped retroviral supernatants derived from transduced H29 were used to construct stable retroviral-producing cells lines as previously described (Gong et al., Neoplasia 1, 123-127 (1999)). T and NK cells were transduced by centrifugation on Retronectin (Takara)-coated plates.

iT transduction. Day 35 WT-TiPS iT cells were harvested and transduced by centrifugation on Retronectin-coated plates in the presence of 100U/mL IL-2. Cells were fed every 48 h with fresh lymphocyte media and cytokines.

Flow cytometry: Cell surface proteins. The following conjugated antibodies were used to monitor T lymphocyte lineage development during differentiation. CD45—BV605 (2D1; BioLegend), CD3—BUV737 (UCHT1; BD), TCRab—PE-Cy7 (IP16; Invitrogen), CD4—BV785 (SK3; BioLegend), CD8a—BUV395 (HIT8a; BD), CD8b—PE (SIDI8BEE; Invitrogen), CD8ab—APC (2ST8.5H7; BD), CD7—APC-H7 (M-T701; BD), CD5—PerCP-Cy5.5 (UCHT2; BioLegend), CD56—BV421 (HCD56; BioLegend), CD1a—PE-Cy7 (HI149; BioLegend), CD2—BV711 (RPA-2.10; BD). The αβTCR-T, γδTCR-T and NK cell phenotypes were determined with CD45RA—BV605 (HI100; BioLegend), CD45RO—BV421 (UCHL1; BioLegend), CD62L—BV711 (DREG-56; BioLegend), CCR7—PE-Cy7 (G043H7; BioLegend), CD25—BB515 (2A3; BD), CD69—PerCP-Cy5.5 (FN50; BioLegend), CD27—BUV737 (M-T271; BD), CD28—PE-Cy7 (CD28.2; BioLegend), CD56—BV605 (HCD56; BioLegend), CD16—BUV737 (3G8; BD), NKG2C—PE (S19005E; BioLegend), KIR2D—FITC (NKVF Si; Miltenyi Biotec), NKp46—FITC (9E2; BioLegend), NKp44—PE-Cy7 (P44-8; BioLegend), NKp80—PE (5D12; BioLegend), NKp30—PerCP-Cy5.5 (P30-15; BioLegend), TCRgd—FITC (B1; BioLegend), TCRVd2—PerCP-Cy5.5 (B6; Biolegend) and CD161—BV421 (HP-3G10; BioLegend), CCR2—PE (K036C2; BioLegend) in addition to the aforementioned T lymphocyte lineage commitment markers. 4-1BB induction was measured with 4-1BB—BV605 (4B4-1; BioLegend). CAR expression was measured with biotin-conjugated goat anti-mouse F(ab′)2 antibody (GaM-biotin; Jackson ImmunoResearch), followed by a blocking incubation with 2% mouse serum (MP Biomedicals) and streptavidin-PE (BioLegend) or streptavidin-APC (BioLegend).

Flow cytometry: pTα stain. Cells were incubated with anti-pTα antibody (2F1; BD), biotin-labelled anti-mouse IgG1 (RMG1-1; BioLegend) and Streptavidin-PE (BD). Staining for additional cell surface proteins was performed after completion of pTα staining.

Flow cytometry: Intracellular phospho-protein analyses. SFGg-1928z, TRAC-1928z and TRAC-1XX T cells were fixed with Phosflow Fix Buffer I (BD) and stained for the CAR with Gam-af647 (Jackson ImmunoResearch), followed by 2% mouse serum, and subsequently permeabilized with Phosflow Perm Buffer III (BD) following the manufacturer's procedure. Permeabilized samples were stained with antibodies detecting phosphorylated CD3z ITAM1 (EP776(2)Y; Abcam) or phosphorylated CD3z ITAM3 (K25-407.69; BD).

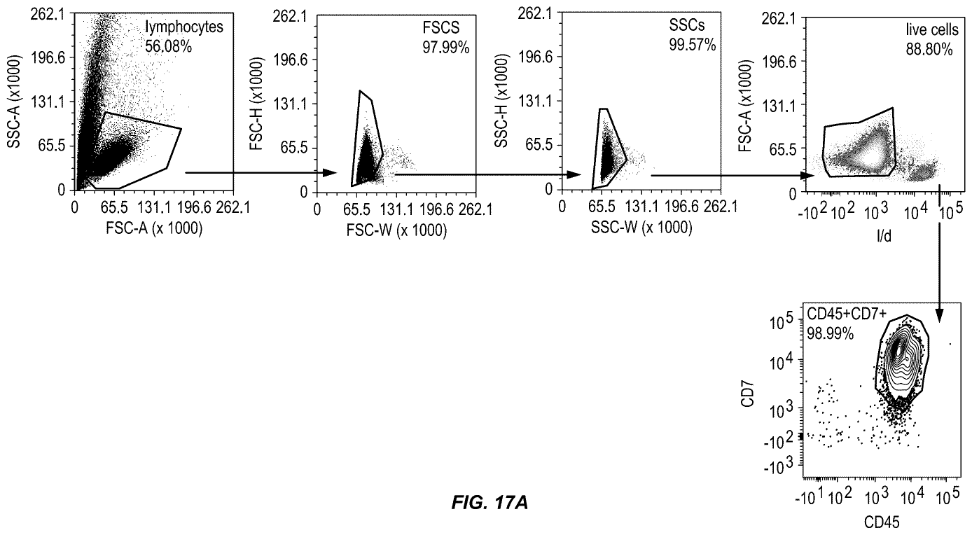

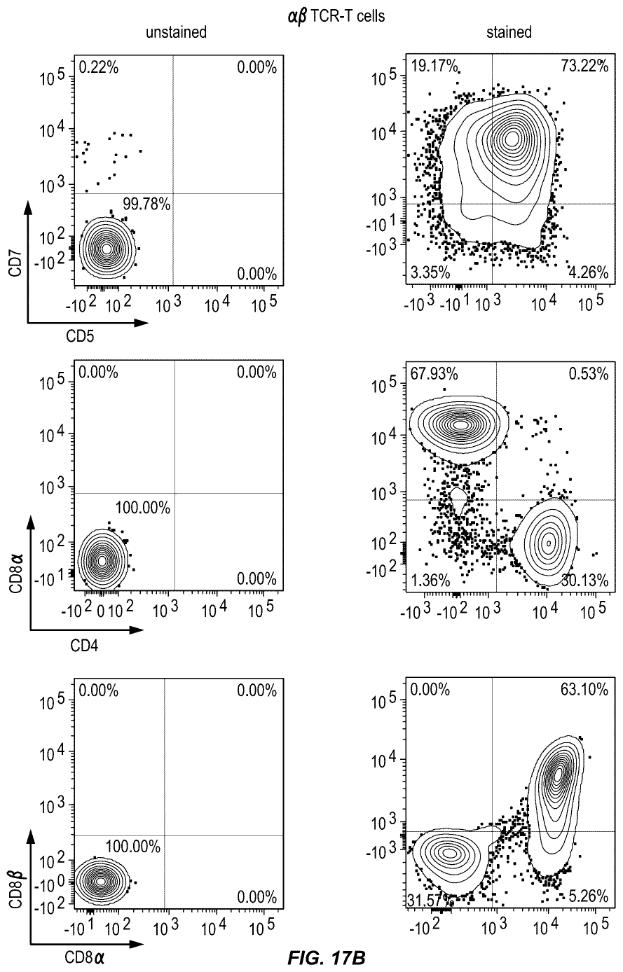

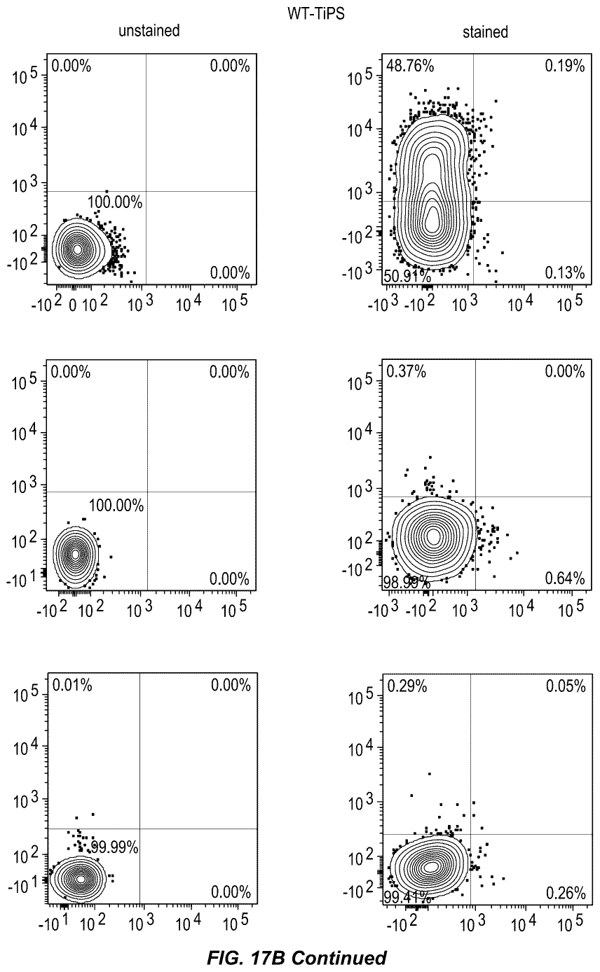

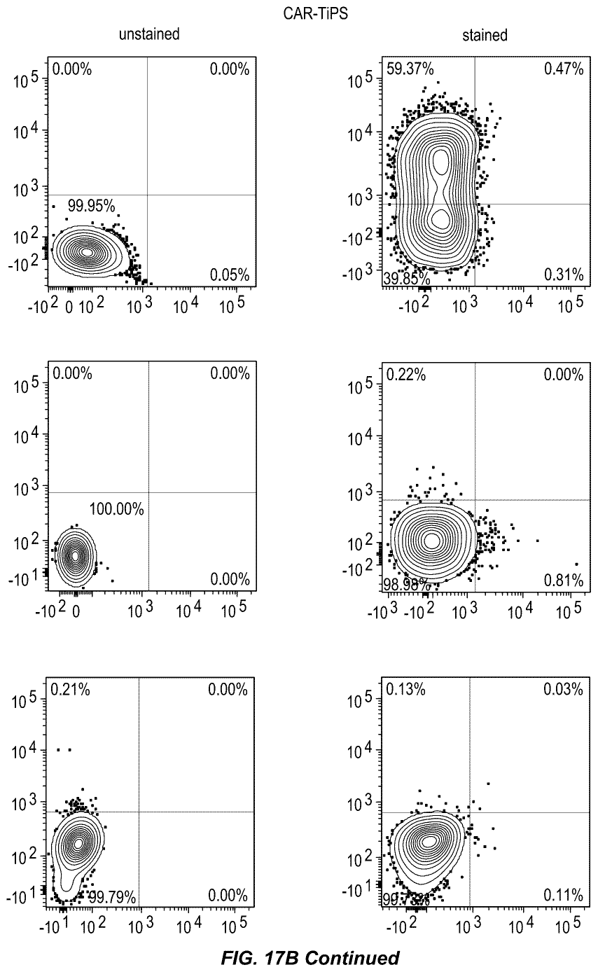

Flow cytometry. All antibodies were titrated prior to use. Flow cytometric data were acquired on Fortessa X-20 (BD) or 5-laser Aurora (Cytek Biosciences) Flow cytometer voltages were calibrated with Ultra Rainbow Calibration Kit (SpheroTech, URCP-38-2K) prior to every acquisition. Analysis was performed using FCS Express 7 (De Novo Software). Negative and positive gates were set based on (un)stained PBMC and TiPS controls (FIGS. 17A and 17B).

Apoptosis Analysis. WT-TiPS and CAR-TiPS cells were harvested daily between D27 and D35 of the differentiation and stained for viability and Annexin-V—PE-Cy7 (Invitrogen) according to manufacturer's instructions, followed by cell surface staining for CD45, CD7, CD4, CD8a and CD8β as described above. Percentage of apoptotic cells within populations (CD45⁺CD7⁺, DN, DP, CD4, CD8αα or CD8αβ) was calculated based on live Annexin-V+ stain.

Notch induction in differentiating TiPS cells. WT-TiPS D20 lymphoid progenitor cells were co-cultured with parental OP9, OP9-hDLL1, OP9-hDLL4, OP9-hJAG1 or OP9-hJAG2. At 0, 4, 8, 12, 24, 48 and 72 h of co-culture, cells were harvested, cell pellets were snap-frozen and stored at −80° C. for until RNA extraction. Gene induction was measured by ddPCR as described below. Relative level of DTX1 induction was normalized to 0 h.

Notch TCR target gene induction during iT differentiation. TiPS (WT-TiPS, CAR-TiPS and TRAC-1XX-TiPS) were differentiated as described. During the T lymphoid commitment phase of the differentiation (D24, D27, D31 and D35) suspension cells were harvested, cell pellets were snap-frozen and stored at −80° C. until RNA extraction. Gene induction was measured by ddPCR as described below.

Digital droplet PCR. Digital droplet PCR (ddPCR) gene expression assays for Notch1 (dHsaCPE5050282), Notch 3 (dHsaCPE5046836), TCF7 (dHsaCPE5031804), DTX1 (dHsaCPE5192773), GATA3 (dHsaCPE5034292), ID3 (dHsaCPE5027720), PTCRA (dHsaCPE5031466) and RPL13A (dHsaCPE5037592) were obtained from Bio-Rad. ddPCR reactions were set up according to One-Step RT-ddPCR Advanced Kit for Probes protocol on a QX200 ddPCR system (Bio-Rad). Each sample was evaluated in technical triplicates. Reactions were partitioned into a median of ~15,000 droplets per well using the QX200 droplet generator. Emulsified reactions were amplified on a 96-well thermal cycler. Plates were read and analyzed with the QuantaSoft software to assess the number of droplets positive for the target gene. The number of mRNA molecules per droplet relative to RPL13A was calculated assuming a Poisson distribution.

RNA extraction, library generation and sequencing. Total RNA was isolated from 0.3-0.5×10⁶ cells using the RNeasy 96 Kit (Qiagen, 74181) according to the manufacturer's protocol. RNA quality was measured by High Sensitivity RNA ScreenTape (Agilent) on the Agilent 42000 TapeStation System. RNA quantity was measured using the Thermo Scientific® Qubit® Flex Fluorometer (Invitrogen). 200 ng of total RNA was used per sample to generate mRNA library using NEBNext® Ultra® II Directional RNA Library Kit for Illumina® (New England BioLabs) per sample. Final libraries were quantified using the Qubit® 1×dsDNA HS Assay kit on the QubitÔ Flex Fluorometer. Library quality and size were measured using Agilent High Sensitivity D1000 ScreenTape. Libraries were calculated to nM, diluted to 4 nM, and pooled evenly for high throughput sequencing. Sequencing was performed on Illumina NextSeq 500 Instrument (Illumina) with 2×76 pair-end reads targeting a minimum of 16 million pair-end reads per sample.

RNA sequencing analysis. Sequencing data were trimmed using Trim Galore!0.6.0 to remove Illumina adapters. Resulting reads were mapped to the human reference genome (assembly GRCh38.86) using Salmon v0.13.1 in quasi-mapping-based mode, with GC bias correction, selective alignment, and range factorization. The data was analyzed using the statistical software R. The aggregated read counts were normalized for sequencing depth and RNA composition with DESeq2. Pseudogenes identified by the GENCODE project and lowly expressed genes were filtered out prior to downstream analysis. Principal Component Analysis (PCA) was performed with normalized read counts in R. Hierarchical Clustering Analysis was carried out with UPGMA method on Euclidean distance matrix. Correlation matrix was generated using Pearson's statistics.

Cytotoxicity Assays. The in vitro toxicity of TRAC-1XX T cells was determined by a standard firefly luciferase (FFLuc)-based assay (Hamieh et al., Nature 568, 112-116 (2019)) or by NLR⁺ imaging on the Incucyte Live Cell Analysis System (Sartorius). For FFLuc based cytotoxicity, FFLuc-expressing NALM6 served as target cells. The effector (E) and tumor target (T) cells were co-cultured in triplicates at the indicated E:T ratio using black-walled 96-well plates with 1×10⁵ target cells in a total volume of 100.iL per well in T cell expansion medium. Four hours later, 50.iL luciferase substrate (Bright-Glo, Promega) was directly added to each well. Emitted light (RLU) was detected in a luminescence plate reader (Agilent BioTek SlL), and lysis was calculated using the formula 100×(1−(RLUsample)/(RLUtarget alone)). For the Incucyte cytotoxicity assay, flat-bottom 96-well plates were pre-coated with 5.ig/mL Fibronectin (Sigma) at 4° C. overnight. The E:T ratios were plated in triplicates with 3×10⁴ target cells in a total volume of 200.iL per well in T cell expansion medium. Hourly brightfield and fluorescence imaging was performed for a 72 h period. Cell survival was quantified based on NLR⁺ surface area by Incucyte S3 software (Essen BioScience) and normalized to the NLR⁺ surface area at 0 h. For the flow-cytometry based CTL, CD19⁺ cells were purified from apheresis product (EasySep CD19 Positive selection kit, StemCell Technologies) and cultured overnight in RPMI media with 10% human serum (HS), 1×NEAA, 2 mM GlutaMAX, 100U/mL Pen, 100.ig/mL Strep. TRAC-1XX-iT cells were counted and plated in triplicate at the indicated E:T ratios with 1×10⁵ CD19⁺ CLL target cells in a total volume of 100 μL per well in T cell expansion media. Six hours later, cells were stained with CD19—PE-Cy7 (SJ25C1, BioLegend), CD45—BV605 (HI30, BioLegend), CD7—APC-H7 and Sytox Blue Dead Cell Stain (Invitrogen) and the number of remaining, target cells (live, CD7-CD19⁺ cells) were enumerated by flow using AccuCount beads (Spherotech). Percentage lysis was calculated using the formula (sample count×100)/(target alone count).

Antigen restimulation assay. Restimulation assays were performed as previously described (Zhao et al., Cancer Cell 28, 415-428 (2015)). In brief, 1×10⁶ T cells were co-cultured with 3×10⁵ 3T3-CD19 in 1 mL T cell expansion media. Fresh media was supplied every 48 h. Cells were counted after seven days and restimulated on fresh 3T3-CD19 monolayers.

In vitro NALM6 rechallenge assay. 3×10⁴ iT cells were co-cultured with 3×10⁴ NLR⁺ NALM6 CD19⁺ tumor cells in 200 μL T cell expansion media. Hourly brightfield and fluorescence imaging was performed for a 10-day period. At day 3 and day 6, plates were removed from the Incucyte, and 50 μL media was replaced with 50 μL media supplemented with 3×10⁴ fresh NLR⁺ NALM6 cells and 4× cytokines. Cell survival was quantified based on NLR⁺ surface area by Incucyte S3 software (Essen BioScience) and normalized to the NLR⁺ surface area at 0, 72 and 144 h respectively.

Cytokine analyses. To measure intracellular levels, iT cells were cultured for 4 h at 1×10⁶ cells/mL together with NALM6 at a 1:1 ratio in the presence of Brefeldin A (BD) monensin (BioLegend) and CD107a—BV421 (H4A3; BD). Cells were stained with ef506 Fixable Viability dye (ThermoFisher) prior to fixation and permeabilization using BD Cytofix/Cytoperm Plus kit as per manufacturer's instructions, followed by staining with anti-cytokine and cell-surface antibodies GranzymeB—APC (GB12; Invitrogen), IFN1—PE-Cy7 (4S.B3; Invitrogen), IL-2—BUV737 (MQ1-17H12; BD), TNFa—PE (Mabl1; Invitrogen), IL-17—af488 (BL168, BioLegend), CD45—BV605 (2D1; BioLegend). Percentage of cytokine producing cells was determined by flow cytometry. To measure secreted cytokine levels, 0.5×10⁶ T cells were cultured together with NALM6 at a 1:1 ratio or without target cells for 24 h. Supernatants were collected and stored at −80° C. Secreted cytokines were quantified using BD Cytometric Bead Array kits (IL-2—558270, IFN1—560111, TNFa—560112) and flow cytometry.

ERK1/2 phosphorylation analysis. Phosphorylated-ERK1/2 was quantified in day 35 WT-TiPS and CAR-TiPS. Cells were lysed in 1× denaturation buffer supplemented with 10 μg/mL aprotonin, leupeptin and pepstatin at lmg/mL total protein content. Phosphorylated ERK1/2 was quantified using the BD Cell Signaling Master Buffer Kit (560005) and Phospho ERK1/2 (560012) according to manufacturer's instructions.

Mouse systemic tumor model. 8-12 week-old NOD/SCID/IL-2R1-null (NSG) mice were obtained from Jackson Laboratory. A dose of 0.1×10⁶ FFLuc-NALM6 was administered by tail vein injection and three days later a dose of 2×10⁶ or 4×10⁶ T cells were administered by tail vein injection per mouse. Mice received IL-2 (Proleukin 100 KU/mouse) and rhIL-15 (150 ng/mouse) in 200 μL PBS intraperitoneally twice per week for three weeks post T cell injection. Tumor burden was measured by bioluminescence imaging using the Xenogen IVIS Imaging System (Xenogen). Living Image software (Xenogen) was used to analyze the acquired bioluminescence data. No blinding method was used. All animal experiments were conducted in accordance with protocols approved by MSKCC Institutional Animal Care and Use Committee (IACUC) and following National Institutes of Health (NIH) guidelines for animal welfare.

Cell enumeration. Three mice per group were euthanized at day 6 or day 12 post T cell injection and cells were isolated from the blood, bone marrow and spleen as described³⁷. Cells were stained for viability (ef506), mCD45—BV421 (30-F11; BioLegend), hCD45—BV605 (HI30; BioLegend), CAR—GaM-af647 (Jackson ImmunoResearch), CD4—BV785 (SK3; BioLegend), CD8a—BUV395 (HIT8a; BD), CD8b—PE (SIDI8BEE; Invitrogen), CD45RA—BV605 (HI100; BioLegend), CD45RO—BV421 (UCHL1; BioLegend), CD27—BUV737 (M-T271; BD), CD28 —PE-Cy7 (CD28.2; BioLegend), CD25—BB515 (2A3; BD), CD62L—BV711 (DREG-56; BioLegend), TIGIT—BV605 (A15153G; BioLegend), LAG3—PE-Cy7 (11C3c65; BioLegend), PD1—BV711 (EH12.2H7; BioLegend), CD56—BV421 (NCAM16.2; BD), CD19—PE-Cy7 (SJ25C1; BioLegend) and GFP (tumor cells) and analyzed by flow cytometry in the presence of counting beads (Countbright, Invitrogen).

Statistics. All experimental data are presented as mean±S.D. No statistical methods were used to predetermine the sample size. Appropriate statistical tests were used to analyze data, as described in the figure legends. Statistical analysis was performed on GraphPad Prism 7 software and R. Significance was set at p<0.05.

### Example 2

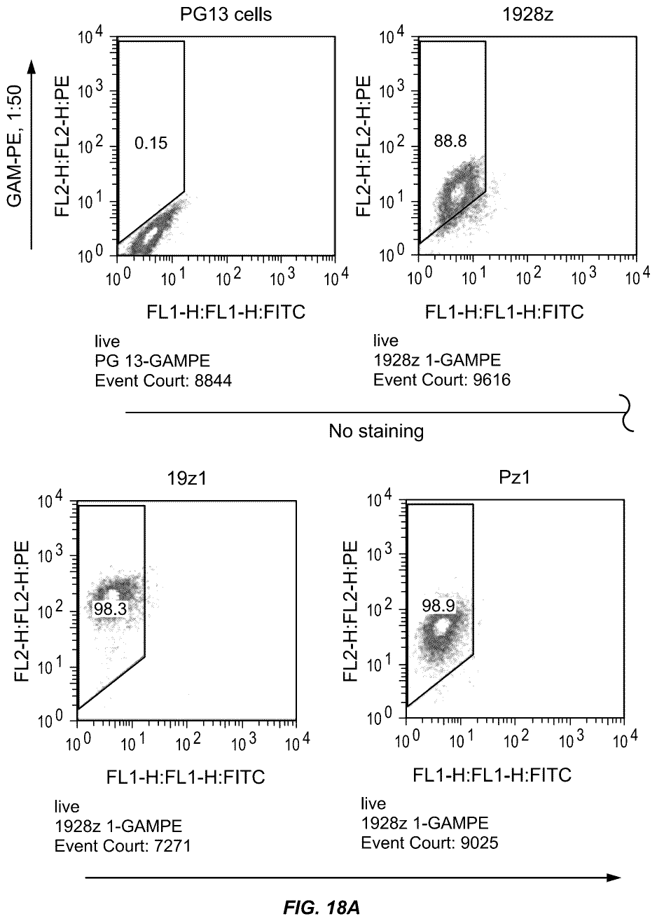

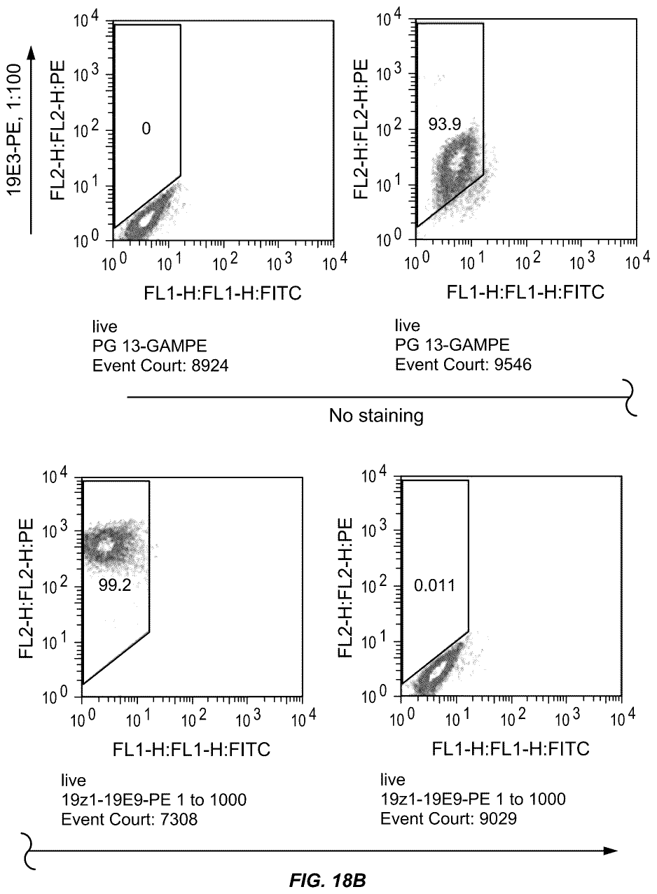

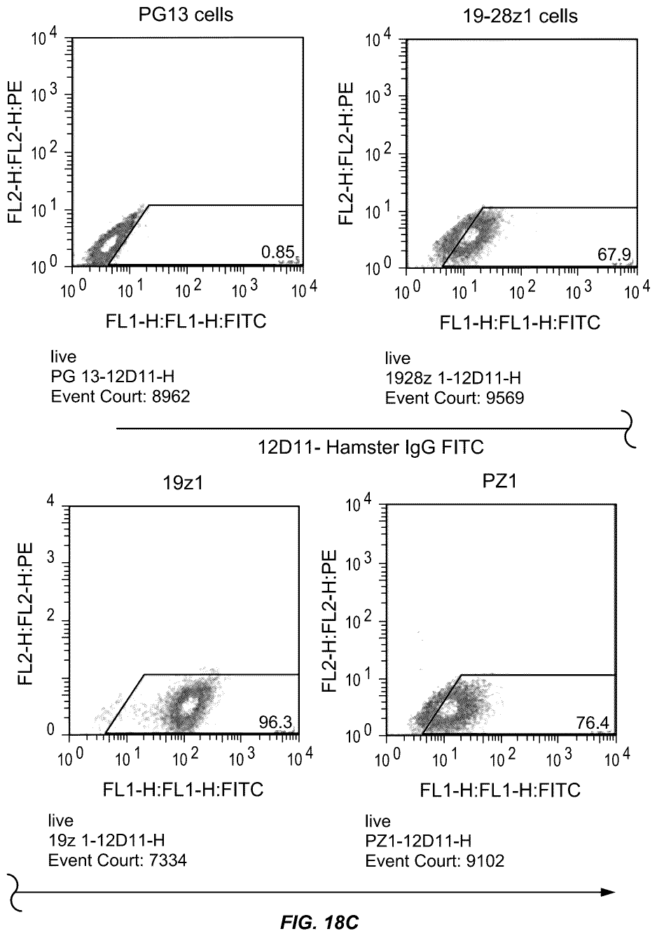

The presented example relates to the field of immunotherapy, specifically the development of allogeneic, point-of-care immunotherapy, by facilitating the maturation of induced pluripotent stem cell (iPSC)-derived T cells. Stimulation, through anti-idiotype antibodies against the antigen-binding domain of a chimeric antigen receptor (CAR), can initiate CAR-signaling, allowing for the maturation of iPSC-derived T cells, or differentiation of peripheral blood-derived T cells. The development of mature CAR T cells from T-iPSC was facilitated through cytokine- and feeder-cell (OP9-DL4) based induction of hematopoietic and T lymphoid lineage differentiation. At the final stage of T cell development, positive selection relies on carefully titrated TCR-signaling, which can be mimicked with the presently disclosed anti-idiotype antibodies 19E3 (“CAR1”) or 12D11 (“CAR2”). The 19E3 monoclonal antibody binds to the SJ25C1 scFv in the CD19-targeting CAR, whereas the 12D11 monoclonal antibody binds to both CD19 and PSMA-targeting CARs (FIGS. 18A-18C)

### 1. Development of anti-idiotvpe antibodies

Antibodies were developed at the Memorial Sloan Kettering Cancer Center's Antibody Core Facility. Armenian Hamsters were inoculated intra-peritoneally with 50 μg of the monoclonal (mouse anti-human) CD19 antibody SJ25C1. Animals were immunized four times and hybridomas were generated through the fusion with mouse myeloma P3x63 cells. The antibody-producing hybridomas were screened by ELISA and flow cytometry (FIG. 18).

Flow cytometric assessment of antibody specificity was determined utilizing the CAR⁺ PG13 fibroblast. Expression of the CD19-targeting second-generation 1928z CAR and first generation 19z1 CAR, as well as the first-generation PSMA-targeting Pz1 CAR was confirmed with the polyclonal Goat-anti-Mouse F(ab)′ fragment (FIG. 18A). Staining with 19E3 (FIG. 18B) showed specific SJ25C1 recognition in the 1928z and 19z1 CARs. Notably, staining with 12D11 (FIG. 18C) showed scFv-independent CAR recognition, detecting both CD19- and PSMA-targeting CARs.

### 2. Anti-Idiotype Antibody Stimulation Facilitates CAR T Cell Maturation

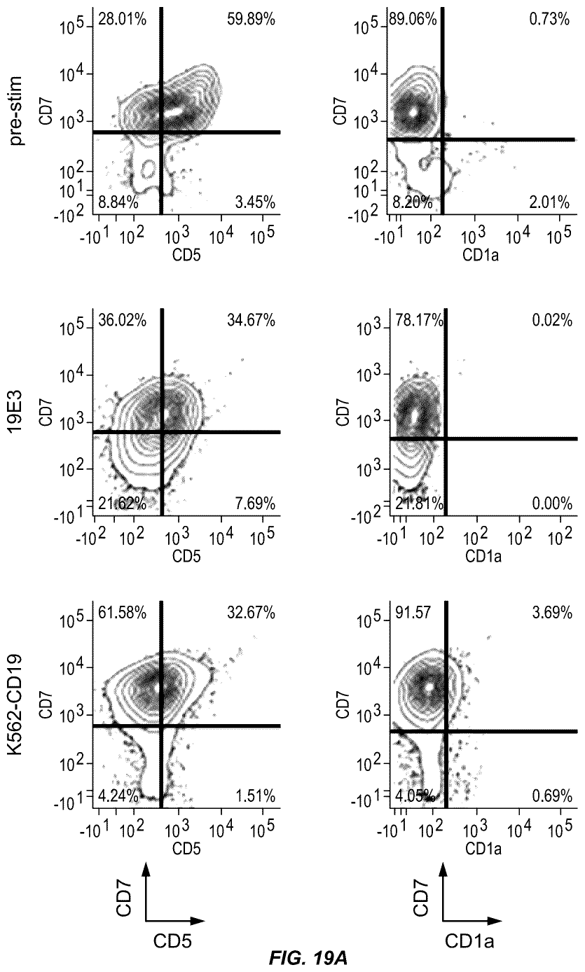

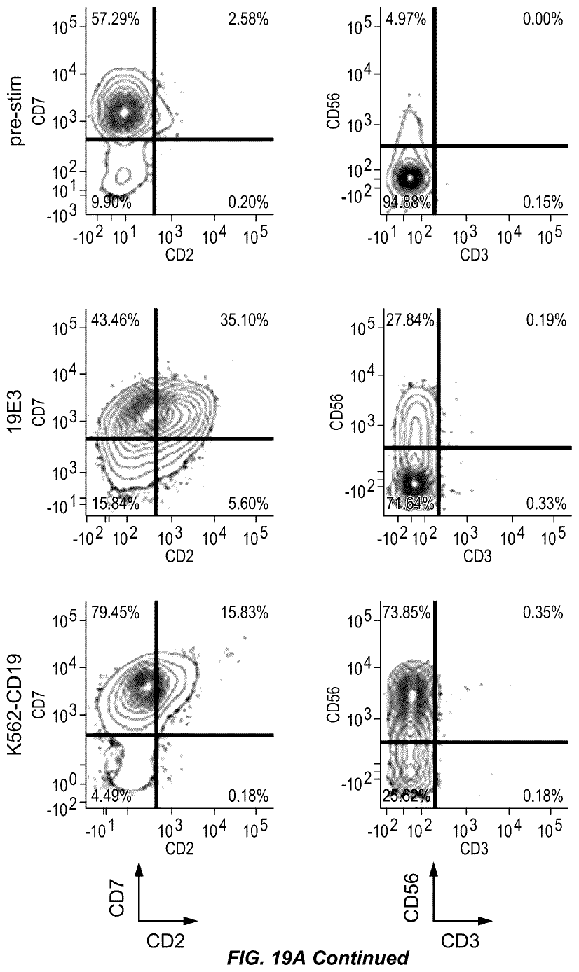

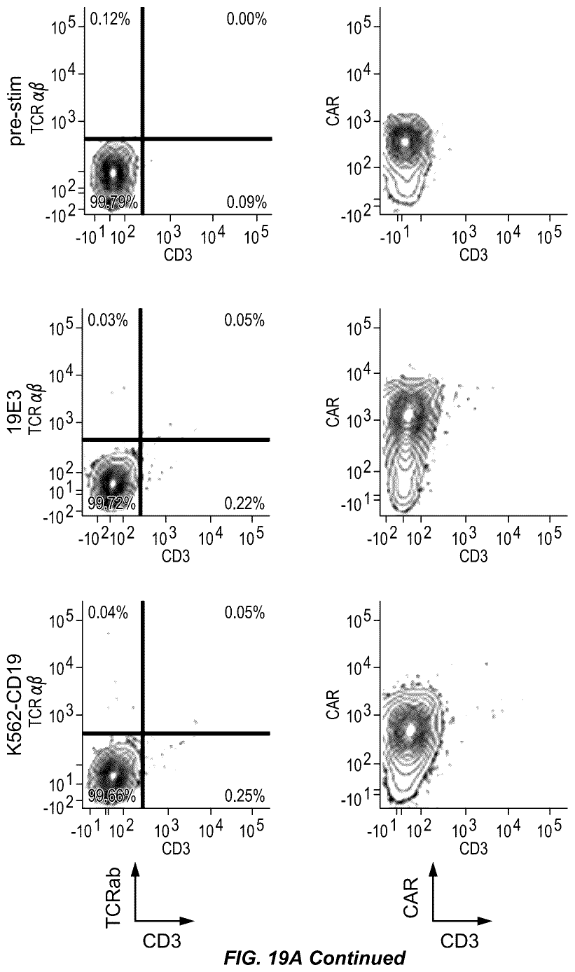

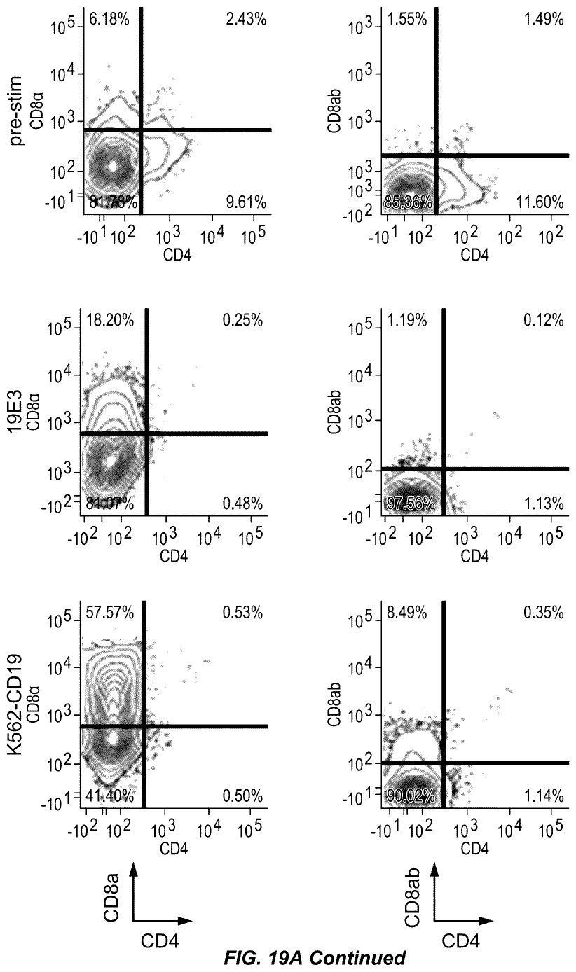

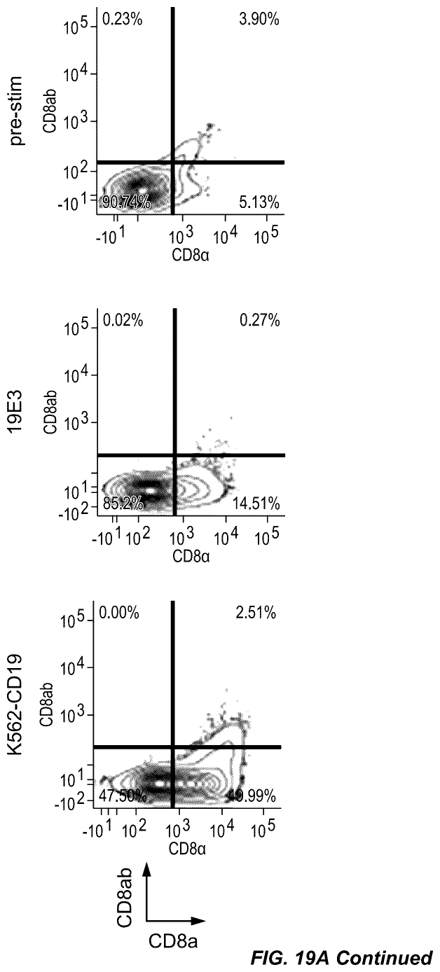

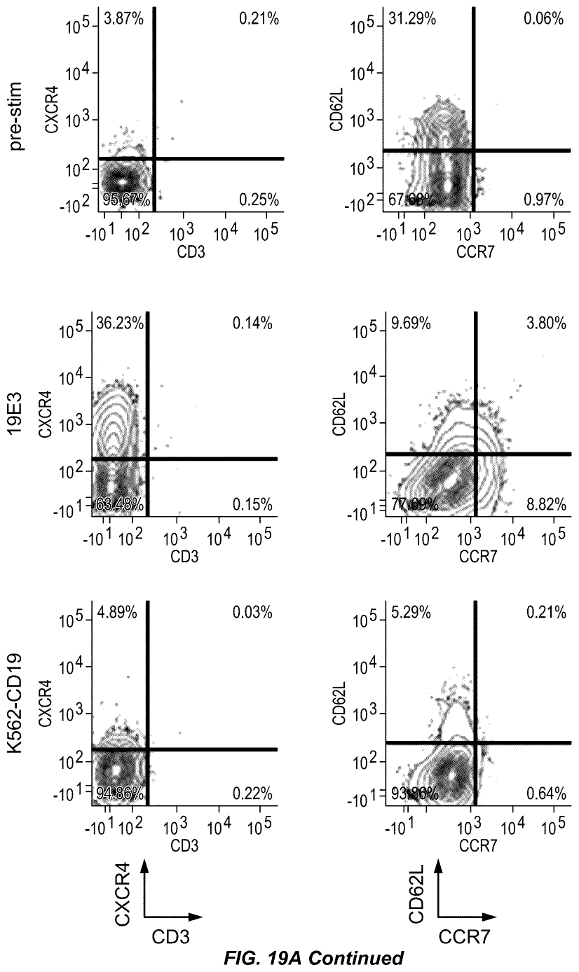

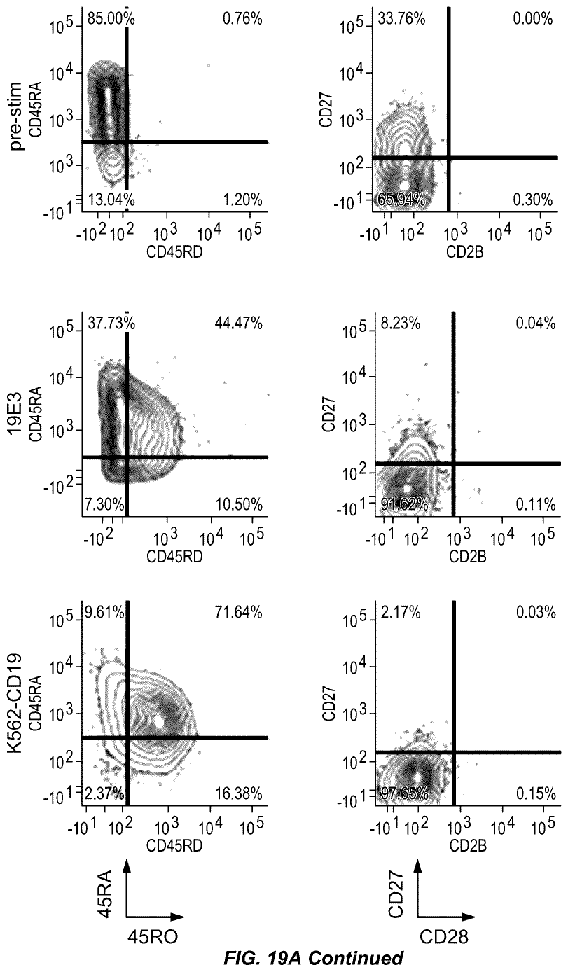

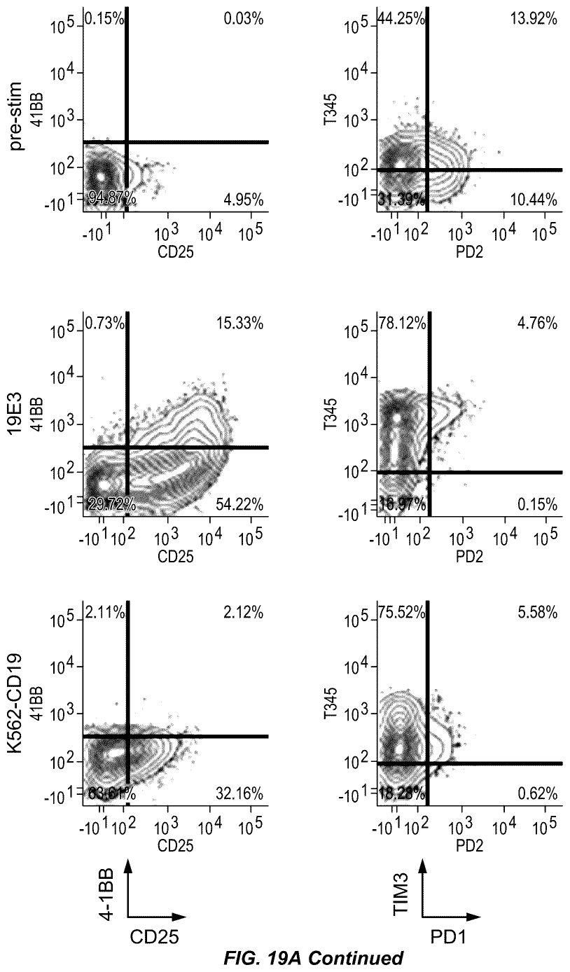

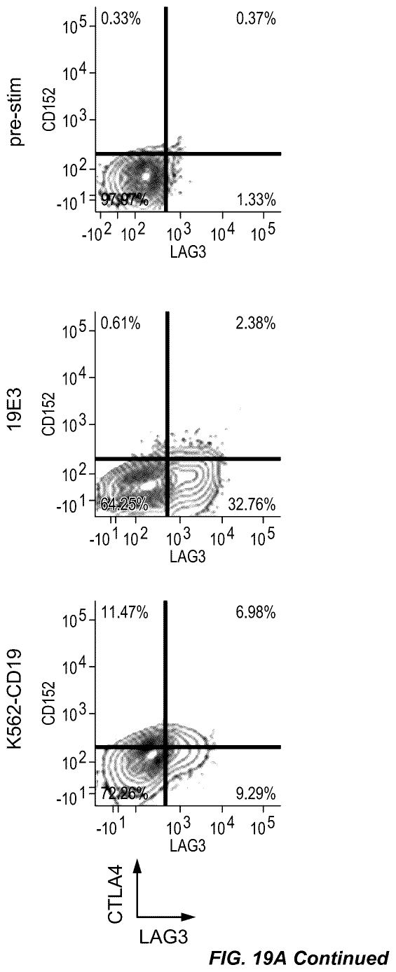

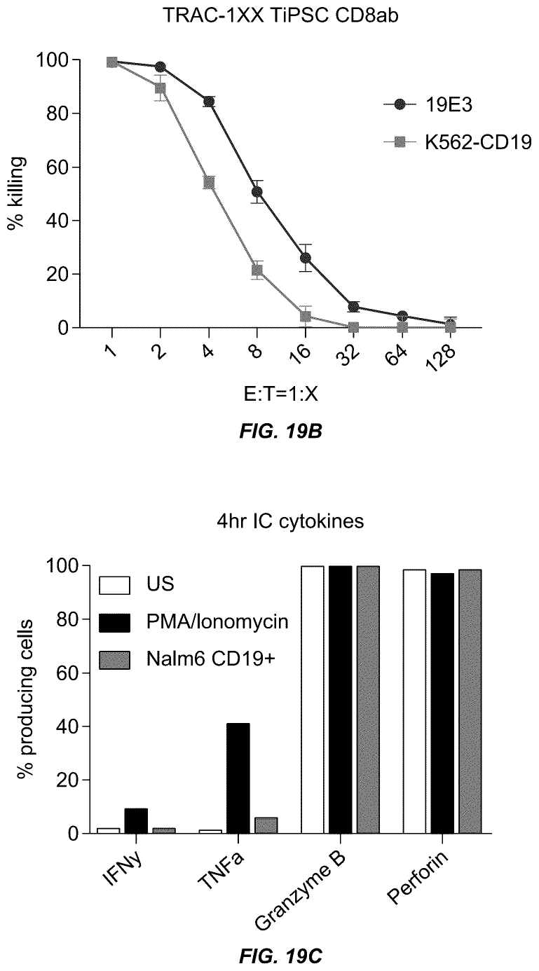

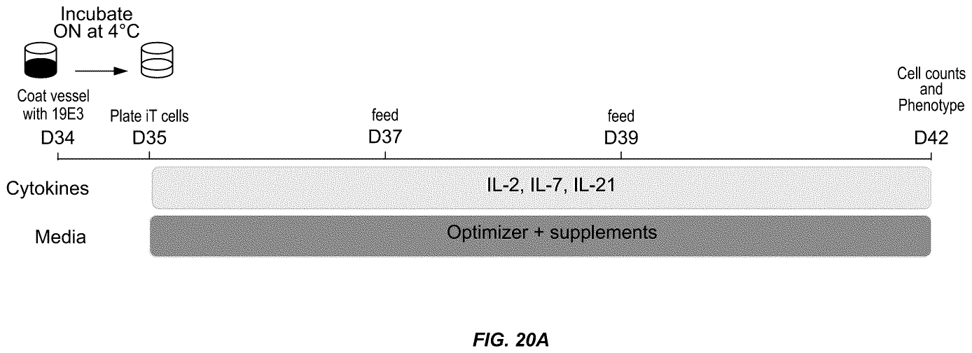

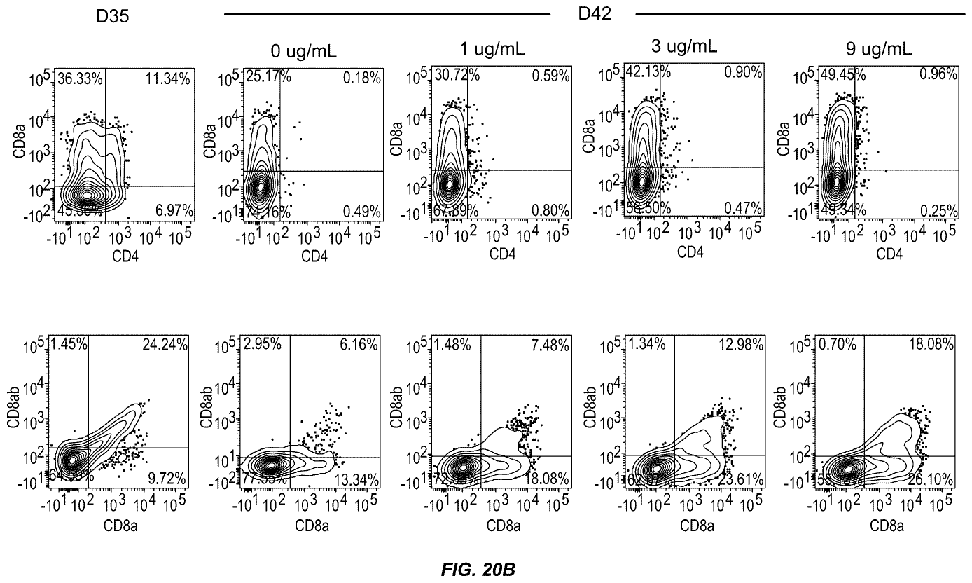

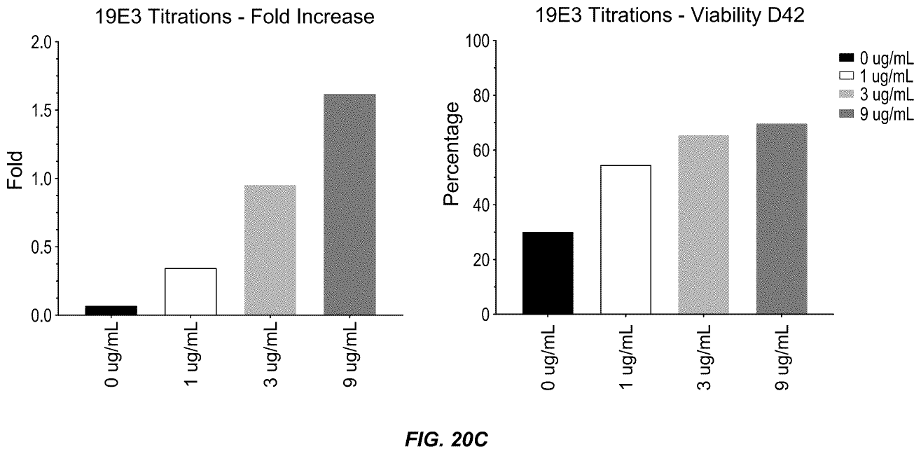

The presently disclosed anti-idiotype antibodies facilitated the maturation of iPSC-derived CAR⁺ T cells. Stimulation of TiPSC-derived T cells with 19E3 or 12D11 antibodies mimicked positive selection through the engagement of CAR signaling (FIGS. 19A-19C) and induced proliferation in control peripheral-blood-derived CD8⁺ CAR T cells. The use of 19E3 or 12D11 anti-idiotype antibodies resulted in the final maturation and the development of a favorable effector phenotype (CD2+, CD56^{lo}, CD45RA⁺, CD62L⁺, CCR7^{lo}, CXCR4⁺, CD25⁺), compared to stronger stimulation with CD19-protein expressing feeder cells (K562-CD19) (FIG. 19A). In vitro-assessed cytolytic capacity (FIG. 19B) over 18 hours showed that 19E3-maturated T cells had superior functional capacity and the ability to produce cytokines in a stimulation-dependent manner (FIG. 19C). Moreover, 19E3 promoted T-cell maturation and expansion in a dose-dependent manner (FIG. 20A-20C).

### 3. CAR T Cell Maturation Obtained by the Anti-Idiotype Antibodies was Enhanced by Co-Stimulation with 4-1BB Agonists

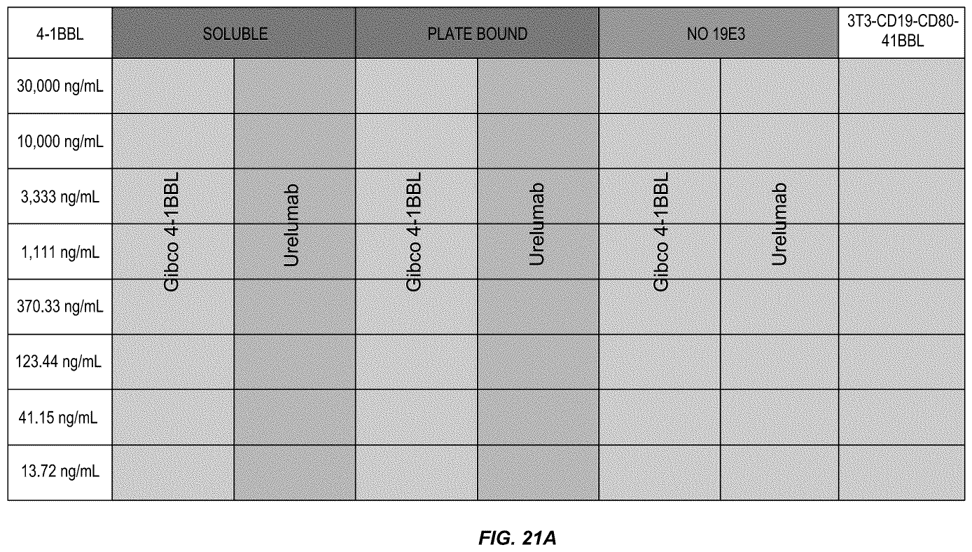

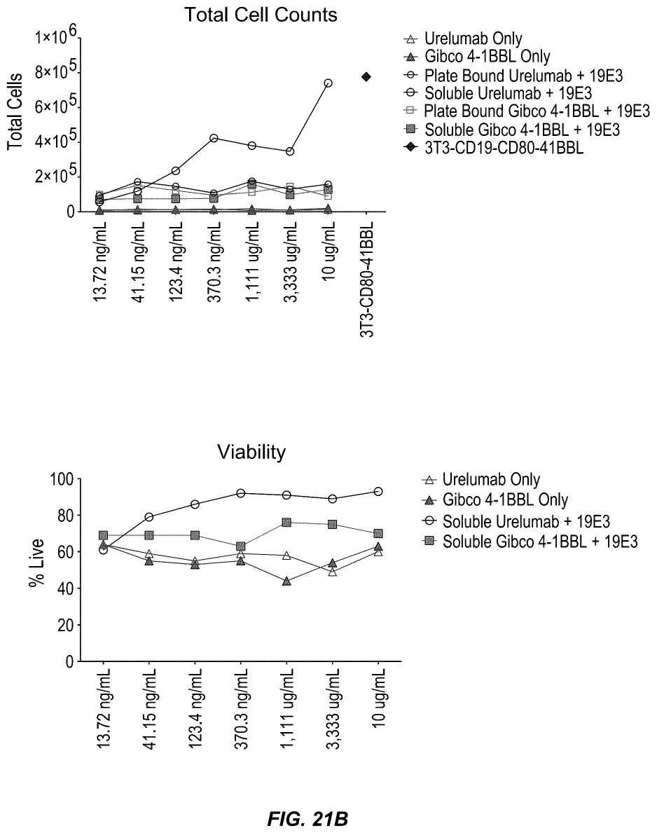

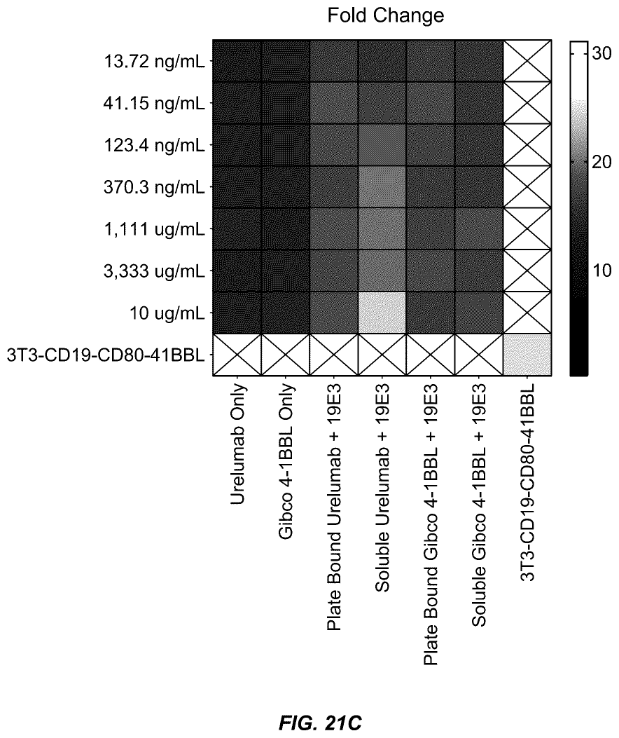

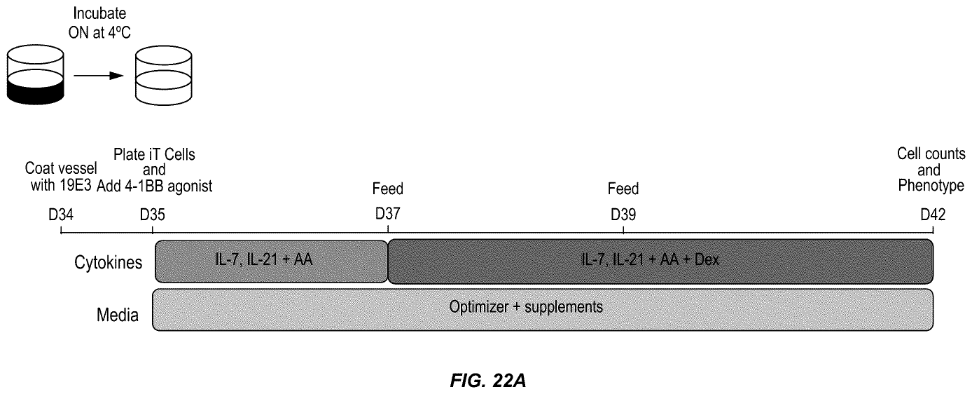

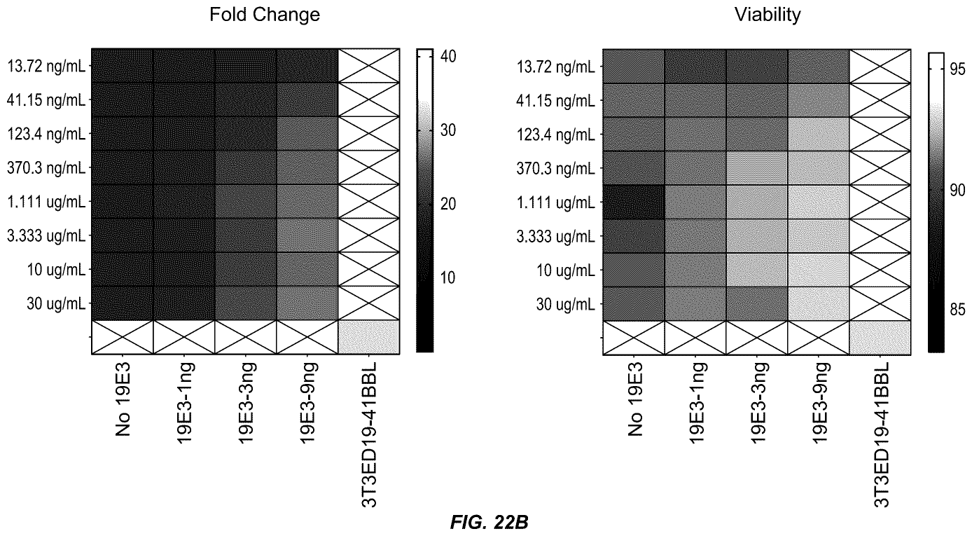

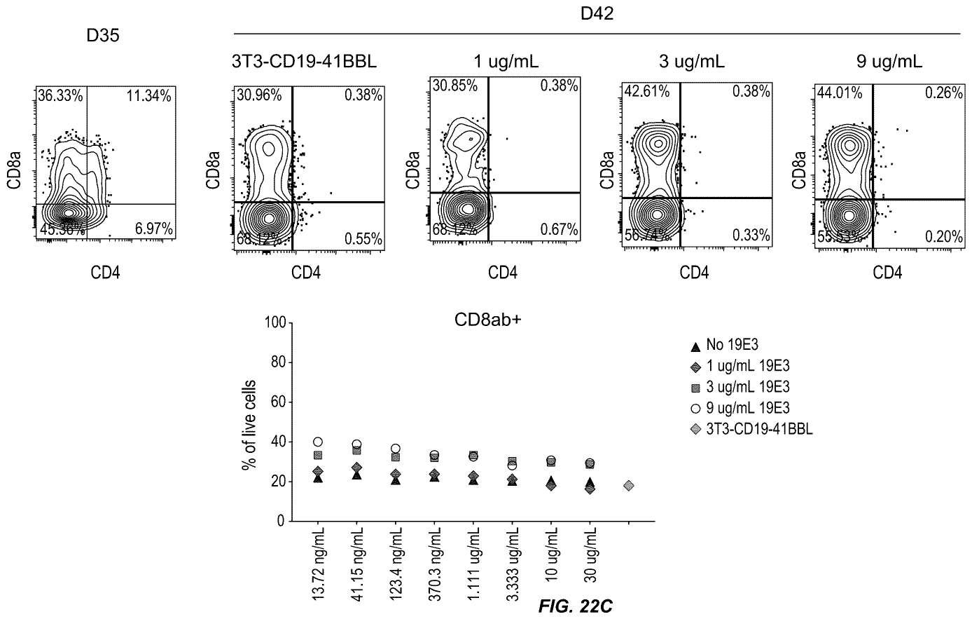

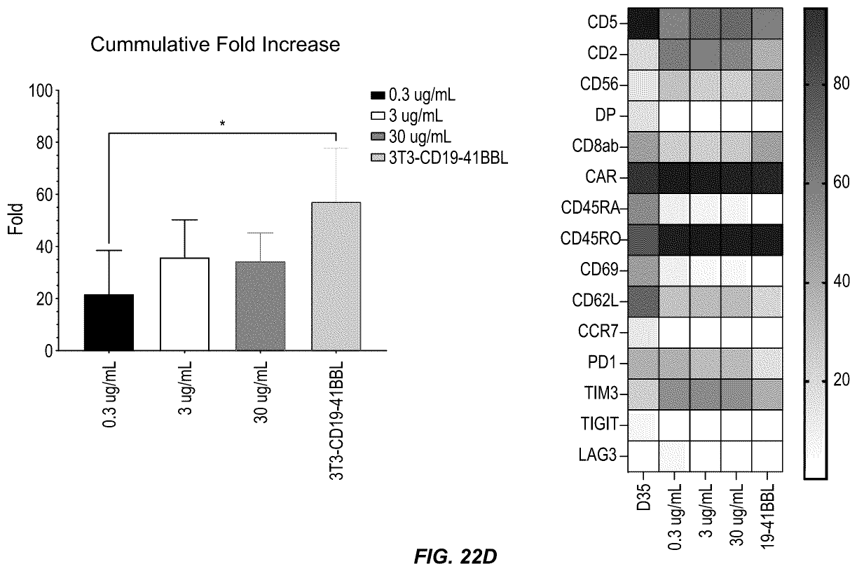

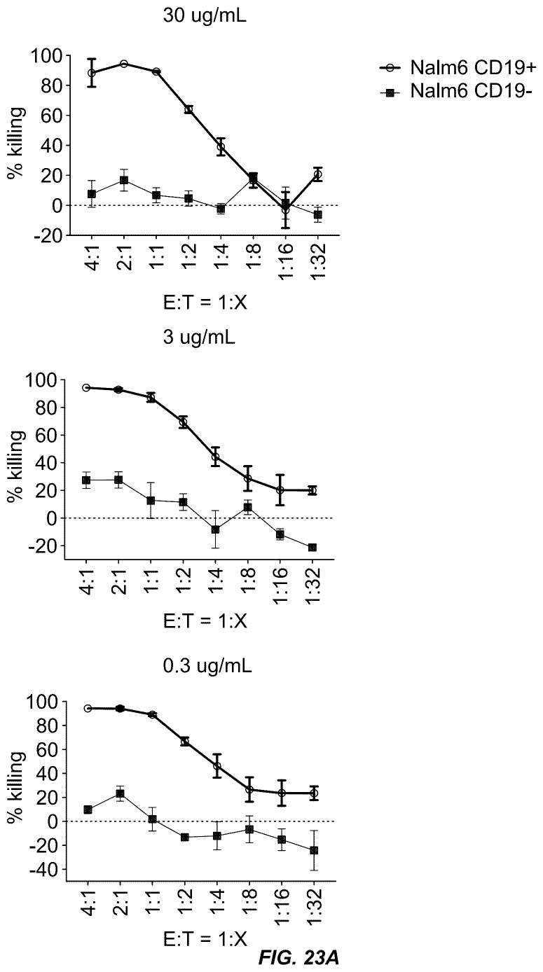

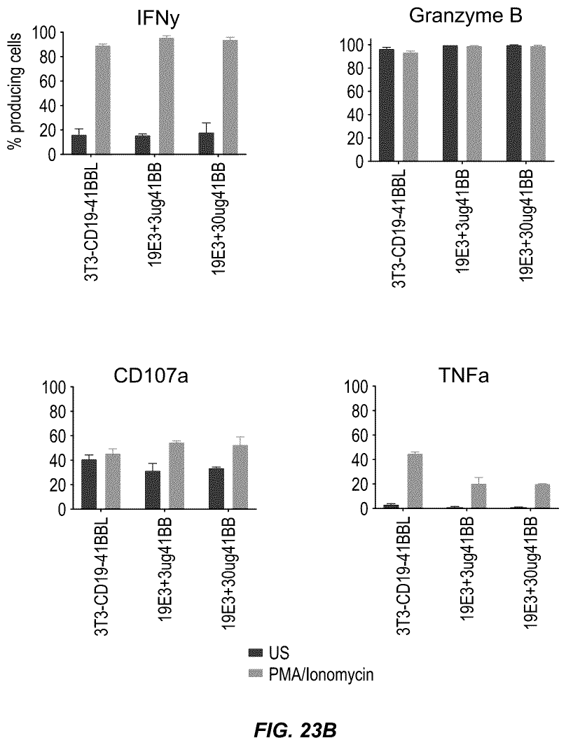

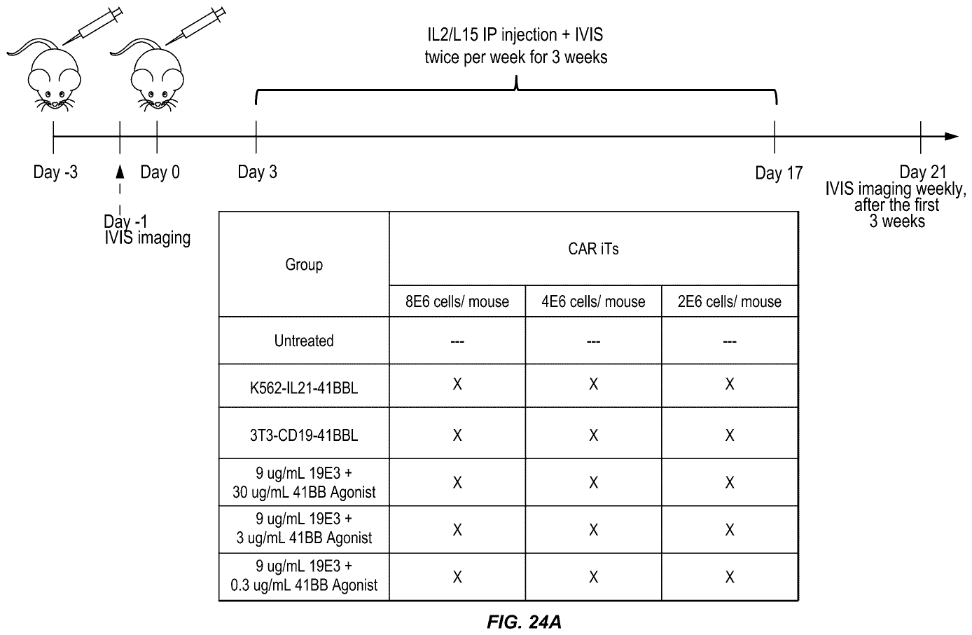

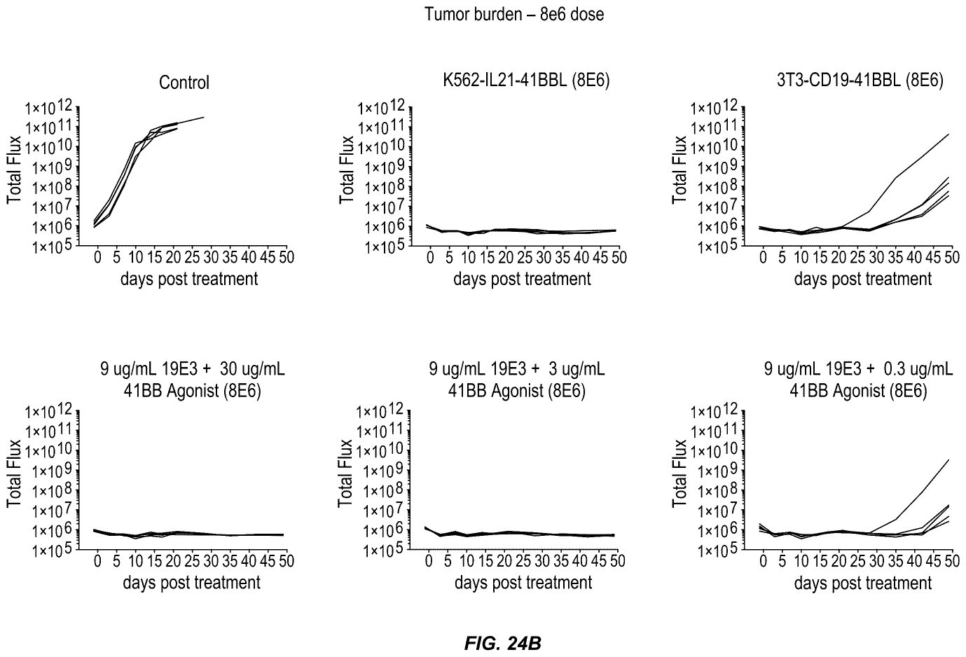

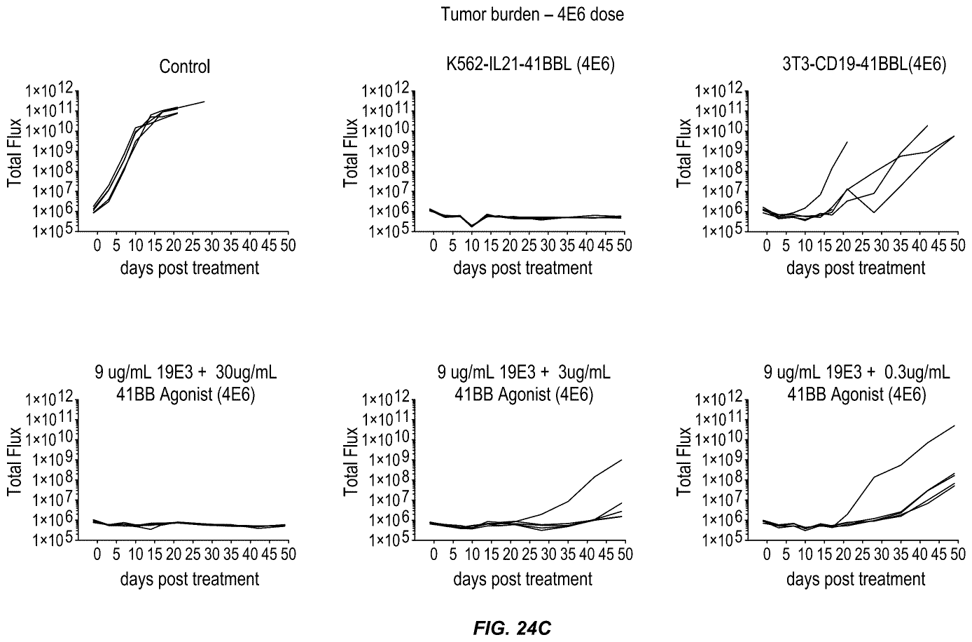

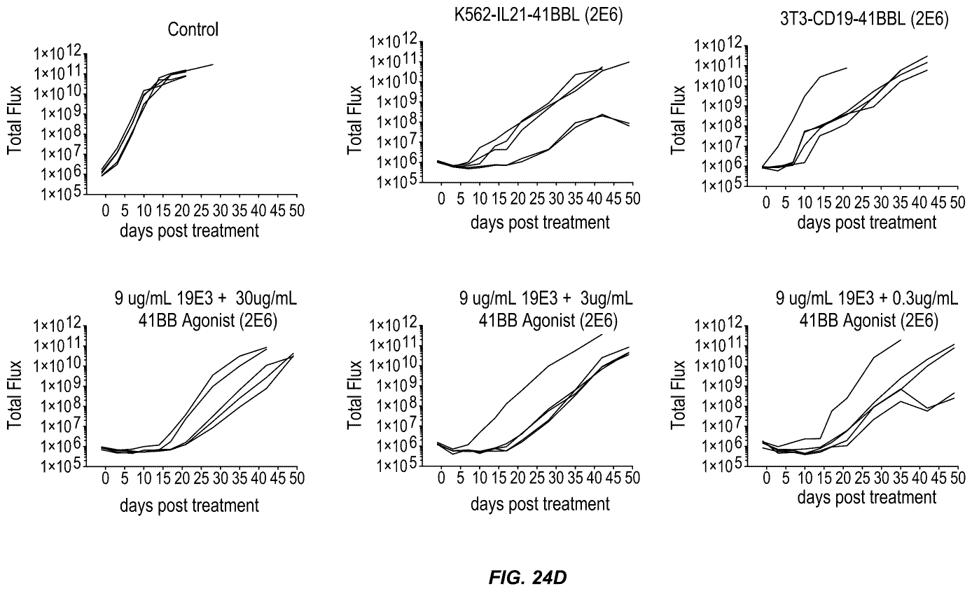

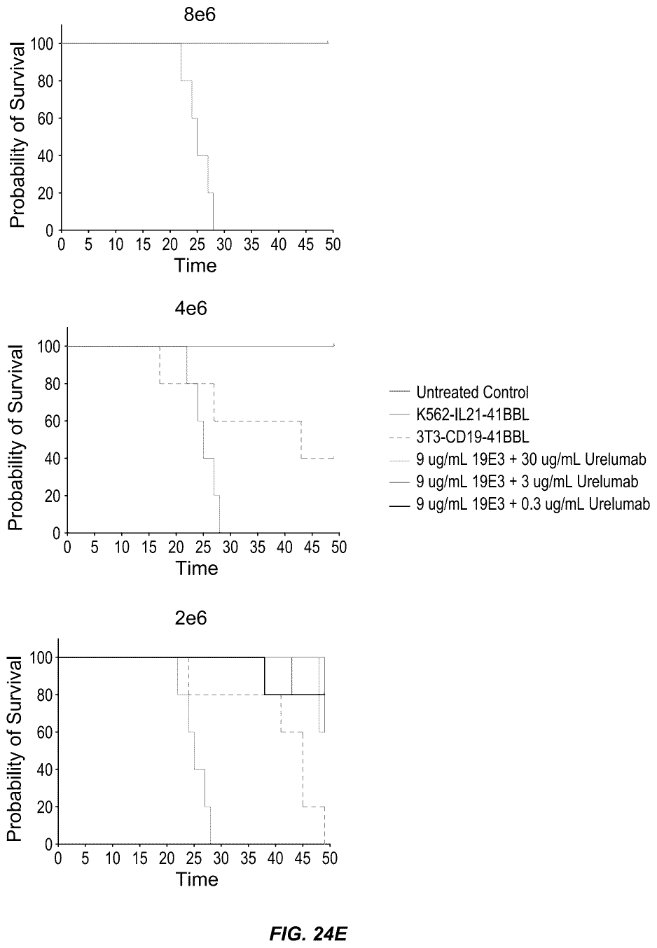

The effects of the presently disclosed anti-idiotype antibodies were enhanced by 4-1BB agonists. As outlined in FIG. 21A, premature T cells were treated with different doses of 4-1BB, either in soluble or plate-bound form. Alternately, cells were treated with urelumab, either in soluble or plate-bound form. It was observed that T cells developed in presence of 19E3 and a 4-1BB agonist (e.g., 4-1BB or urelumab) had improved proliferation and increased cell count and cell viability (FIGS. 21B and 21C). Importantly, it was observed a dose-response effect of 19E3 and 4-1BB agonist (e.g., urelumab) in the cell maturation process, cell viability, and cell proliferation (FIGS. 22A-22D). Moreover, use of both 19E3 and urelumab provided improved the polyfunctionality of iT cells (FIGS. 23A and 23B). Next, it was determined T cells obtained by using the 19E3 antibody and a 4-1BB agonist were capable of reducing tumor burden in vivo. As demonstrated in FIGS. 24A-24E, iT cells obtained using 19E3 and urelumab improved tumor control even at the lowest tested dose (e.g., 4×10⁶ cells).

### 4. Discussion

The present example reports here on the generation of mature and functional CAR T cells facilitated by the use of the presently disclosed antibodies. Specifically, it was investigated how the presently disclosed antibodies can engage chimeric receptors (e.g., CAR) and induce maturation of iPSC-derived T cells.

Exposure to the presently disclosed antibodies resulted in the maturation of CAR-iT which had cytolytic capacity, cytokine secretion, and anti-tumor activity in vivo. Importantly, the use of the presently disclosed antibodies allowed the maturation of CAR-iT cells able to induce long-term remission and survival following intravenous infusion of a single dose of 4×10⁶ iT cells.

The presently disclosed antibodies provide a tool that can be used to generate clinically relevant CAR iT cell numbers, which in principle may allow for off-the-shelf application from batches of uniform and consistent CAR iT cells produced from the same engineered master cell bank.

In summary, the present example demonstrates that the presently disclosed antibodies can engage synthetic receptors like CARs which substitute for the TCR in driving directed T cell differentiation allowing for large-scale production of potent T cell-based immunotherapies.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An antibody or an antigen-binding fragment thereof, comprising:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

2. An antibody or an antigen-binding fragment thereof comprising a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

3. An antibody or an antigen-binding fragment thereof comprising a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

4. The antibody or an antigen-binding fragment thereof of claim 1, wherein:
a) the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO:7, and the light chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8; and
b) the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 17, and the light chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 18.

5. The antibody or antigen-binding fragment thereof of claim 1, wherein
a) the heavy chain variable region comprises the amino acid sequence set forth in SEQ ID NO: 7, and the light chain variable region comprises the amino acid sequence set forth in SEQ ID NO: 8; or
b) the heavy chain variable region comprises the amino acid sequence set forth in SEQ ID NO: 17, and the light chain variable region comprises the amino acid sequence set forth in SEQ ID NO: 18.

6. The antibody or antigen-binding fragment thereof of claim 1, wherein the antibody or antigen-binding fragment thereof is an anti-idiotype antibody.

7. The antibody or antigen-binding fragment thereof of claim 1, wherein the antibody comprises a human variable region framework region.

8. The antibody or antigen-binding fragment thereof of claim 1, which is a fully human or an antigen-binding fragment thereof, a chimeric antibody or an antigen-binding fragment thereof, or a humanized antibody or an antigen-binding fragment thereof.

9. The antibody or antigen-binding fragment thereof of claim 1, wherein the antigen-binding fragment is a Fab, Fab′, F(ab′)2, variable fragment (Fv), or single chain variable region (scFv).

10. The antibody or antigen-binding fragment thereof of claim 11, wherein the antigen-binding fragment is an scFv.

11. A nucleic acid encoding the antibody or antigen-binding fragment thereof of claim 1.

12. A vector comprising the nucleic acid of claim 11.

13. A composition comprising the antibody or antigen-binding fragment thereof of claim 1.

14. The composition of claim 13, which is a pharmaceutical composition that further comprises a pharmaceutically acceptable carrier.

15. A multi-specific molecule comprising the antibody or antigen-binding fragment thereof of claim 1, linked to one or more functional moieties.

16. The multi-specific molecule of claim 15, wherein the one or more functional moieties have a different binding specificity than the antibody or antigen binding fragment thereof.

17. A composition comprising the multi-specific molecule of claim 15.

18. The composition of claim 17, which is a pharmaceutical composition that further comprises a pharmaceutically acceptable carrier.

19. A kit comprising the antibody or antigen-binding fragment thereof of claim 1.

20. The kit of claim 19, wherein the kit further comprises written instructions for using the antibody or antigen-binding fragment thereof, the multi-specific molecule, or the composition.


SEQUENCE LISTING (52 sequences)

SEQ ID NO: 1 - protein, 8 aa, synthetic construct
  REGION 1..8: note = Description of sequence: Anti-CAR1
GFTFSDFD

SEQ ID NO: 2 - protein, 8 aa, synthetic construct
  REGION 1..8: note = Description of sequence: Anti-CAR1
INGGSGVI

SEQ ID NO: 3 - protein, 13 aa, synthetic construct
  REGION 1..13: note = Description of sequence: Anti-CAR1
AREELGRRYYFDY

SEQ ID NO: 4 - protein, 7 aa, synthetic construct
  REGION 1..7: note = Description of sequence: Anti-CAR1
SEHSSYN

SEQ ID NO: 5 - protein, 7 aa, synthetic construct
  REGION 1..7: note = Description of sequence: Anti-CAR1
LKSDGSH

SEQ ID NO: 6 - protein, 13 aa, synthetic construct
  REGION 1..13: note = Description of sequence: Anti-CAR1
GAGYTISGQYGYV

SEQ ID NO: 7 - protein, 216 aa, synthetic construct
  REGION 1..216: note = Description of sequence: Anti-CAR1
MNSGLKLVFFVLILKGVQCEVQLVESGGGLVQTGKSLKLSCEASGFTFSDFDMNWVRQAPGKGLEWVAYINGGSGVIFYADAVKGRFTISRDNAKNLLFLQMNNLKSEDSAMYYCAREELGRRYYFDYWGQGTMVTVSSATTTAPSVYPLAPACDSTTSTTNTVTLGCLVKGYFPEPVTVSWNSGALTSGVHTFPSVLHSGLYSLSSSVTVPSSTW

SEQ ID NO: 8 - protein, 214 aa, synthetic construct
  REGION 1..214: note = Description of sequence: Anti-CAR1
MAWIPLLFFLLHCTGSFSQPVLTQSPSASASLSGSVKLTCTLSSEHSSYNIAWYQQHPDKAPKYVMYLKSDGSHFKGDGIPDRFSGSSSGAHRYLSISNVQSEDDATYFCGAGYTISGQYGYVFDSGTQLTVLGGPKSSPKVTVFPPSPEELRTNKATLVCLVNDFYPGSATVTWKANGATINDGVKTTKPSKQGQNYMTSSYLSLTADQWRSH

SEQ ID NO: 9 - DNA, 648 bp, synthetic construct
  misc_feature 1..648: note = Description of sequence: Anti-CAR1
  source 1..648: mol_type = other DNA
atgaactcaggactcaaattggttttctttgtccttattctgaaaggtgtccagtgtgaggtgcagttggtggagtctgggggaggcttagtacagactggaaagtccctgaaactctcatgtgaggcctctggattcaccttcagtgactttgacatgaactgggtccgccaggctccagggaaggggctggagtgggtcgcatacattaatggtggtagtggtgttatcttttatgctgacgctgtgaagggccgattcaccatctccagagacaacgccaagaacttactgttcctacagatgaacaatctcaagtctgaggactcagccatgtattactgtgcaagagaggaactgggacggaggtactactttgattactggggccaaggaaccatggtcaccgtctcctcagccacaacaacagccccatctgtctatcccttggcccctgcctgtgacagcacaaccagcaccacgaacacggtgaccctgggatgcctggtcaagggctatttccctgagccggtgaccgtaagctggaactctggagccctgaccagcggcgtgcacaccttcccatctgtcctgcattctgggctctactccctcagcagctcagtgactgtaccttccagcacctgg

SEQ ID NO: 10 - DNA, 642 bp, synthetic construct
  misc_feature 1..642: note = Description of sequence: Anti-CAR1
  source 1..642: mol_type = other DNA
atggcctggattcctctcctcttcttcctccttcattgcacagggtctttctctcaacctgtgttgactcagtcaccctctgcctctgcctccctgagtggctcagtcaaactcacctgcaccctgagtagtgagcacagctcctacaacatagcatggtaccagcaacatccagacaaggctcccaagtatgtgatgtaccttaagagtgatggaagccacttcaagggagatgggatccctgatcgcttctctggctccagctctggggctcatcgctacttaagcatctccaatgtccagtctgaagatgatgctacctatttctgtggtgcaggttataccatttctggacaatatgggtatgtttttgacagcggaacccagctcaccgtcctaggtggacccaagtcttctcccaaagtcacagtgtttccaccttcacctgaggagctccggacaaacaaagccacactggtgtgtctggttaatgacttctacccgggttctgcaacagtgacctggaaggcaaatggagcaactatcaatgatggggtgaagactacaaagccttccaaacagggccaaaactacatgaccagcagctacctaagtttgacagcagaccagtggagatcccac

SEQ ID NO: 11 - protein, 8 aa, synthetic construct
  REGION 1..8: note = Description of sequence: Anti-CAR2
GFDLSDYF

SEQ ID NO: 12 - protein, 10 aa, synthetic construct
  REGION 1..10: note = Description of sequence: Anti-CAR2
IYPQSYDYAT

SEQ ID NO: 13 - protein, 8 aa, synthetic construct
  REGION 1..8: note = Description of sequence: Anti-CAR2
TKDGDVGF

SEQ ID NO: 14 - protein, 6 aa, synthetic construct
  REGION 1..6: note = Description of sequence: Anti-CAR2
QDISNE

SEQ ID NO: 15 - skipped

SEQ ID NO: 16 - protein, 9 aa, synthetic construct
  REGION 1..9: note = Description of sequence: Anti-CAR2
LQHYSFPLT

SEQ ID NO: 17 - protein, 213 aa, synthetic construct
  REGION 1..213: note = Description of sequence: Anti-CAR2
MGLGLQWVFFVALLKGVHCAVRLLESGGGLVKPEGALKLSCVASGFDLSDYFMGWVRQAPGKGLEWVAHIYPQSYDYATYYSGSVQGRFTISRDDSRSMVYLQMNNLRTEDTATYYCTKDGDVGFWGQGTMVTVSSATTTAPSVYPLAPACDSTTSTTNTVTLGCLVKGYFPEPVTVSWNSGALTSGVHTFPSVLHSGLYSLSSSVTVPSSTW

SEQ ID NO: 18 - protein, 196 aa, synthetic construct
  REGION 1..196: note = Description of sequence: Anti-CAR2
MANKSPAQALAILLLWLSGVRCDIQVTQSPTLLSASLGDKVTINCLASQDISNELNWYQQKSGQSPTLLIYNGKNLQSGVPSRFSGQYSGRSFTISINNVEPEDVATYFCLQHYSFPLTFGDGSKLEMKRADAKPTVSIFPPSSEQLGTGSATLVCFVNNFYPKDINVKWKVDGSEKRDGVLQSVTDQDSKDSTYS

SEQ ID NO: 19 - DNA, 639 bp, synthetic construct
  misc_feature 1..639: note = Description of sequence: Anti-CAR2
  source 1..639: mol_type = other DNA
atgggattgggactgcagtgggttttctttgttgctcttttaaaaggtgtccactgtgcggtgcggcttctggagtcgggtggaggattagtgaagcctgagggggcactgaaactctcctgtgtggcctctggattcgacttaagtgactatttcatgggctgggtccgccaggctccagggaaggggctggagtgggttgctcacatataccctcaaagttatgattatgcaacctattactcgggttcggtgcaaggcagattcaccatctccagagatgattcccgaagcatggtctacctgcaaatgaacaacctgagaactgaggacacggccacttattactgtacaaaagacggggacgtgggtttctggggccaaggaaccatggtcaccgtctcctcagccacaacaacagccccatctgtctatcccttggcccctgcctgtgacagcacaaccagcaccacgaacacggtgaccctgggatgcctggtcaagggctatttccctgagccggtgaccgtaagctggaactctggagccctgaccagcggcgtgcacaccttcccatctgtcctgcattctgggctctactccctcagcagctcagtgactgtaccttccagcacctgg

SEQ ID NO: 20 - DNA, 588 bp, synthetic construct
  misc_feature 1..588: note = Description of sequence: Anti-CAR2
  source 1..588: mol_type = other DNA
atggccaacaagtctcctgctcaggcactggcaattttgttactgtggctgtcaggtgtcagatgtgacattcaagtgacacaatctcccaccctcctgtcagcatctctaggagacaaagtgaccatcaattgcctggcaagtcaggacattagcaatgagttaaactggtaccagcagaagtcaggacaatctcctacactgttgatttataatggaaaaaatttgcagtctggtgtcccgtcaaggttcagtggccagtattcagggagaagtttcactatcagcatcaacaatgtggaacctgaagatgttgcaacttatttttgtcttcagcattacagttttccgctcacgttcggtgatggctccaagctggagatgaaacgggctgatgctaagccaaccgtctccatcttcccaccatccagtgagcagttgggcactggaagcgccacacttgtgtgcttcgtgaacaacttctaccccaaagacatcaatgtcaagtggaaagtagatggcagtgaaaaacgagatggcgtcctgcagagtgtcactgatcaggacagcaaagacagcacctacagc

SEQ ID NO: 21 - protein, 19 aa, synthetic construct
  REGION 1..19: note = Description of sequence: Linker No. 1
GGGGSGGGGSGGGSGGGGS

SEQ ID NO: 22 - protein, 15 aa, synthetic construct
  REGION 1..15: note = Description of sequence: Linker No. 2
GGGGSGGGGSGGGGS

SEQ ID NO: 23 - protein, 24 aa, synthetic construct
  REGION 1..24: note = Description of sequence: Linker No. 3
GGGGSGGGGSGGGGSGGGSGGGGS

SEQ ID NO: 24 - protein, 29 aa, synthetic construct
  REGION 1..29: note = Description of sequence: Linker No. 4
GGGGSGGGGSGGGGSGGGGSGGGSGGGGS

SEQ ID NO: 25 - protein, 5 aa, synthetic construct
  REGION 1..5: note = Description of sequence: Linker No. 5
GGGGS

SEQ ID NO: 26 - protein, 10 aa, synthetic construct
  REGION 1..10: note = Description of sequence: Linker No. 6
GGGGSGGGGS

SEQ ID NO: 27 - protein, 20 aa, Homo sapiens
  REGION 1..20: note = Description of sequence: a human IL-2 signal sequence
MYRMQLLSCIALSLALVTNS

SEQ ID NO: 28 - protein, 20 aa, Mus musculus
  REGION 1..20: note = Description of sequence: a mouse IL-2 signal sequence
MYSMQLASCVTLTLVLLVNS

SEQ ID NO: 29 - protein, 20 aa, Homo sapiens
  REGION 1..20: note = Description of sequence: a human kappa leader sequence
METPAQLLFLLLLWLPDTTG

SEQ ID NO: 30 - protein, 20 aa, Mus musculus
  REGION 1..20: note = Description of sequence: a mouse kappa leader sequence
METDTLLLWVLLLWVPGSTG

SEQ ID NO: 31 - protein, 21 aa, Homo sapiens
  REGION 1..21: note = Description of sequence: a human CD8 leader sequence
MALPVTALLLPLALLLHAARP

SEQ ID NO: 32 - protein, 18 aa, Homo sapiens
  REGION 1..18: note = Description of sequence: a truncated human CD8 signal peptide
MALPVTALLLPLALLLHA

SEQ ID NO: 33 - protein, 16 aa, Homo sapiens
  REGION 1..16: note = Description of sequence: a human albumin signal sequence
MKWVTFISLLFSSAYS

SEQ ID NO: 34 - protein, 30 aa, Homo sapiens
  REGION 1..30: note = Description of sequence: a human prolactin signal sequence
MDSKGSSQKGSRLLLLLVVSNLLLCQGVVS

SEQ ID NO: 35 - protein, 140 aa, synthetic construct
  REGION 1..140: note = Description of sequence: TRAC polypeptide
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - DNA, 420 bp, synthetic construct
  misc_feature 1..420: note = Description of sequence: Nucleotide coding of TRAC polypeptide
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 37 - protein, 143 aa, synthetic construct
  REGION 1..143: note = Description of sequence: TRAC polypeptide
IPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - DNA, 429 bp, synthetic construct
  misc_feature 1..429: note = Description of sequence: Nucleotide coding of TRAC polypeptide
  source 1..429: mol_type = other DNA
attcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 39 - protein, 178 aa, synthetic construct
  REGION 1..178: note = Description of sequence: TRBC2 polypeptide
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 40 - DNA, 534 bp, synthetic construct
  misc_feature 1..534: note = Description of sequence: Nucleotide coding of TRBC2 polypeptide
  source 1..534: mol_type = other DNA
gatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 41 - protein, 180 aa, synthetic construct
  REGION 1..180: note = Description of sequence: TRBC2 polypeptide
LEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 42 - DNA, 540 bp, synthetic construct
  misc_feature 1..540: note = Description of sequence: Nucleotide coding of TRBC2 polypeptide
  source 1..540: mol_type = other DNA
ctggaggatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 43 - protein, 175 aa, synthetic construct
  REGION 1..175: note = Description of sequence: TRBC1 polypeptide
LNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 44 - protein, 176 aa, synthetic construct
  REGION 1..176: note = Description of sequence: TRBC1 polypeptide
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 45 - DNA, 528 bp, synthetic construct
  misc_feature 1..528: note = Description of sequence: Nucleotide coding of TRBC1 polypeptide
  source 1..528: mol_type = other DNA
gacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 46 - protein, 173 aa, synthetic construct
  REGION 1..173: note = Description of sequence: TRGC1 polypeptide
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 47 - protein, 189 aa, synthetic construct
  REGION 1..189: note = Description of sequence: TRGC2 polypeptide
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 48 - protein, 153 aa, synthetic construct
  REGION 1..153: note = Description of sequence: TRDC polypeptide
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 49 - protein, 10 aa, synthetic construct
  REGION 1..10: note = Description of sequence: TCR-alpha joining region hinge/spacer
IPNIQNPDPA

SEQ ID NO: 50 - DNA, 30 bp, synthetic construct
  misc_feature 1..30: note = Description of sequence: Nucleotide coding of TCR-alpha joining region hinge/spacer
  source 1..30: mol_type = other DNA
attcccaatatccagaaccctgaccctgcc

SEQ ID NO: 51 - protein, 11 aa, synthetic construct
  REGION 1..11: note = Description of sequence: TCR-beta joining region hinge/spacer
LEDLKNVFPPE

SEQ ID NO: 52 - DNA, 33 bp, synthetic construct
  misc_feature 1..33: note = Description of sequence: Nucleotide coding of TCR-beta joining region hinge/spacer
  source 1..33: mol_type = other DNA
ctggaggatctgaaaaacgtgttccctcctgaa